SEQUENCE LISTING (911 sequences)

SEQ ID NO: 1 - RNA, 80 nt, Homo sapiens
uauaaaucuaguggaaacauuucugcacaaacuagauucuggacaccagugugcggaaaugcuucugcuacauuuuuagg

SEQ ID NO: 2 - RNA, 22 nt, Homo sapiens
gugugcggaaaugcuucugcua

SEQ ID NO: 3 - RNA, 6 nt, Artificial Sequence
  Synthetic Construct
ccuaaa

SEQ ID NO: 4 - RNA, 6 nt, Artificial Sequence
  Synthetic Construct
cuaaaa

SEQ ID NO: 5 - RNA, 6 nt, Artificial Sequence
  Synthetic Construct
uaaaaa

SEQ ID NO: 6 - RNA, 6 nt, Artificial Sequence
  Synthetic Construct
aaaaau

SEQ ID NO: 7 - RNA, 6 nt, Artificial Sequence
  Synthetic Construct
aaaaug

SEQ ID NO: 8 - RNA, 6 nt, Artificial Sequence
  Synthetic Construct
aaaugu

SEQ ID NO: 9 - RNA, 6 nt, Artificial Sequence
  Synthetic Construct
aaugua

SEQ ID NO: 10 - RNA, 6 nt, Artificial Sequence
  Synthetic Construct
auguag

SEQ ID NO: 11 - RNA, 6 nt, Artificial Sequence
  Synthetic Construct
uguagc

SEQ ID NO: 12 - RNA, 6 nt, Artificial Sequence
  Synthetic Construct
guagca

SEQ ID NO: 13 - RNA, 6 nt, Artificial Sequence
  Synthetic Construct
uagcag

SEQ ID NO: 14 - RNA, 6 nt, Artificial Sequence
  Synthetic Construct
agcaga

SEQ ID NO: 15 - RNA, 6 nt, Artificial Sequence
  Synthetic Construct
gcagaa

SEQ ID NO: 16 - RNA, 6 nt, Artificial Sequence
  Synthetic Construct
cagaag

SEQ ID NO: 17 - RNA, 6 nt, Artificial Sequence
  Synthetic Construct
agaagc

SEQ ID NO: 18 - RNA, 6 nt, Artificial Sequence
  Synthetic Construct
gaagca

SEQ ID NO: 19 - RNA, 6 nt, Artificial Sequence
  Synthetic Construct
aagcau

SEQ ID NO: 20 - RNA, 6 nt, Artificial Sequence
  Synthetic Construct
agcauu

SEQ ID NO: 21 - RNA, 6 nt, Artificial Sequence
  Synthetic Construct
gcauuu

SEQ ID NO: 22 - RNA, 6 nt, Artificial Sequence
  Synthetic Construct
cauuuc

SEQ ID NO: 23 - RNA, 6 nt, Artificial Sequence
  Synthetic Construct
auuucc

SEQ ID NO: 24 - RNA, 6 nt, Artificial Sequence
  Synthetic Construct
uuuccg

SEQ ID NO: 25 - RNA, 6 nt, Artificial Sequence
  Synthetic Construct
uuccgc

SEQ ID NO: 26 - RNA, 6 nt, Artificial Sequence
  Synthetic Construct
uccgca

SEQ ID NO: 27 - RNA, 6 nt, Artificial Sequence
  Synthetic Construct
ccgcac

SEQ ID NO: 28 - RNA, 6 nt, Artificial Sequence
  Synthetic Construct
cgcaca

SEQ ID NO: 29 - RNA, 6 nt, Artificial Sequence
  Synthetic Construct
gcacac

SEQ ID NO: 30 - RNA, 6 nt, Artificial Sequence
  Synthetic Construct
cacacu

SEQ ID NO: 31 - RNA, 6 nt, Artificial Sequence
  Synthetic Construct
acacug

SEQ ID NO: 32 - RNA, 6 nt, Artificial Sequence
  Synthetic Construct
cacugg

SEQ ID NO: 33 - RNA, 6 nt, Artificial Sequence
  Synthetic Construct
acuggu

SEQ ID NO: 34 - RNA, 6 nt, Artificial Sequence
  Synthetic Construct
cuggug

SEQ ID NO: 35 - RNA, 6 nt, Artificial Sequence
  Synthetic Construct
uggugu

SEQ ID NO: 36 - RNA, 6 nt, Artificial Sequence
  Synthetic Construct
gguguc

SEQ ID NO: 37 - RNA, 6 nt, Artificial Sequence
  Synthetic Construct
gugucc

SEQ ID NO: 38 - RNA, 6 nt, Artificial Sequence
  Synthetic Construct
ugucca

SEQ ID NO: 39 - RNA, 6 nt, Artificial Sequence
  Synthetic Construct
guccag

SEQ ID NO: 40 - RNA, 6 nt, Artificial Sequence
  Synthetic Construct
uccaga

SEQ ID NO: 41 - RNA, 6 nt, Artificial Sequence
  Synthetic Construct
ccagaa

SEQ ID NO: 42 - RNA, 6 nt, Artificial Sequence
  Synthetic Construct
cagaau

SEQ ID NO: 43 - RNA, 6 nt, Artificial Sequence
  Synthetic Construct
agaauc

SEQ ID NO: 44 - RNA, 6 nt, Artificial Sequence
  Synthetic Construct
gaaucu

SEQ ID NO: 45 - RNA, 6 nt, Artificial Sequence
  Synthetic Construct
aaucua

SEQ ID NO: 46 - RNA, 6 nt, Artificial Sequence
  Synthetic Construct
aucuag

SEQ ID NO: 47 - RNA, 6 nt, Artificial Sequence
  Synthetic Construct
ucuagu

SEQ ID NO: 48 - RNA, 6 nt, Artificial Sequence
  Synthetic Construct
cuaguu

SEQ ID NO: 49 - RNA, 6 nt, Artificial Sequence
  Synthetic Construct
uaguuu

SEQ ID NO: 50 - RNA, 6 nt, Artificial Sequence
  Synthetic Construct
aguuug

SEQ ID NO: 51 - RNA, 6 nt, Artificial Sequence
  Synthetic Construct
guuugu

SEQ ID NO: 52 - RNA, 6 nt, Artificial Sequence
  Synthetic Construct
uuugug

SEQ ID NO: 53 - RNA, 6 nt, Artificial Sequence
  Synthetic Construct
uugugc

SEQ ID NO: 54 - RNA, 6 nt, Artificial Sequence
  Synthetic Construct
ugugca

SEQ ID NO: 55 - RNA, 6 nt, Artificial Sequence
  Synthetic Construct
gugcag

SEQ ID NO: 56 - RNA, 6 nt, Artificial Sequence
  Synthetic Construct
ugcaga

SEQ ID NO: 57 - RNA, 6 nt, Artificial Sequence
  Synthetic Construct
gcagaa

SEQ ID NO: 58 - RNA, 6 nt, Artificial Sequence
  Synthetic Construct
cagaaa

SEQ ID NO: 59 - RNA, 6 nt, Artificial Sequence
  Synthetic Construct
agaaau

SEQ ID NO: 60 - RNA, 6 nt, Artificial Sequence
  Synthetic Construct
gaaaug

SEQ ID NO: 61 - RNA, 6 nt, Artificial Sequence
  Synthetic Construct
aaaugu

SEQ ID NO: 62 - RNA, 6 nt, Artificial Sequence
  Synthetic Construct
aauguu

SEQ ID NO: 63 - RNA, 6 nt, Artificial Sequence
  Synthetic Construct
auguuu

SEQ ID NO: 64 - RNA, 6 nt, Artificial Sequence
  Synthetic Construct
uguuuc

SEQ ID NO: 65 - RNA, 6 nt, Artificial Sequence
  Synthetic Construct
guuucc

SEQ ID NO: 66 - RNA, 6 nt, Artificial Sequence
  Synthetic Construct
uuucca

SEQ ID NO: 67 - RNA, 6 nt, Artificial Sequence
  Synthetic Construct
uuccac

SEQ ID NO: 68 - RNA, 6 nt, Artificial Sequence
  Synthetic Construct
uccacu

SEQ ID NO: 69 - RNA, 6 nt, Artificial Sequence
  Synthetic Construct
ccacua

SEQ ID NO: 70 - RNA, 6 nt, Artificial Sequence
  Synthetic Construct
cacuag

SEQ ID NO: 71 - RNA, 6 nt, Artificial Sequence
  Synthetic Construct
acuaga

SEQ ID NO: 72 - RNA, 6 nt, Artificial Sequence
  Synthetic Construct
cuagau

SEQ ID NO: 73 - RNA, 6 nt, Artificial Sequence
  Synthetic Construct
uagauu

SEQ ID NO: 74 - RNA, 6 nt, Artificial Sequence
  Synthetic Construct
agauuu

SEQ ID NO: 75 - RNA, 6 nt, Artificial Sequence
  Synthetic Construct
gauuua

SEQ ID NO: 76 - RNA, 6 nt, Artificial Sequence
  Synthetic Construct
auuuau

SEQ ID NO: 77 - RNA, 6 nt, Artificial Sequence
  Synthetic Construct
uuuaua

SEQ ID NO: 78 - RNA, 7 nt, Artificial Sequence
  Synthetic Construct
ccuaaaa

SEQ ID NO: 79 - RNA, 7 nt, Artificial Sequence
  Synthetic Construct
cuaaaaa

SEQ ID NO: 80 - RNA, 7 nt, Artificial Sequence
  Synthetic Construct
uaaaaau

SEQ ID NO: 81 - RNA, 7 nt, Artificial Sequence
  Synthetic Construct
aaaaaug

SEQ ID NO: 82 - RNA, 7 nt, Artificial Sequence
  Synthetic Construct
aaaaugu

SEQ ID NO: 83 - RNA, 7 nt, Artificial Sequence
  Synthetic Construct
aaaugua

SEQ ID NO: 84 - RNA, 7 nt, Artificial Sequence
  Synthetic Construct
aauguag

SEQ ID NO: 85 - RNA, 7 nt, Artificial Sequence
  Synthetic Construct
auguagc

SEQ ID NO: 86 - RNA, 7 nt, Artificial Sequence
  Synthetic Construct
uguagca

SEQ ID NO: 87 - RNA, 7 nt, Artificial Sequence
  Synthetic Construct
guagcag

SEQ ID NO: 88 - RNA, 7 nt, Artificial Sequence
  Synthetic Construct
uagcaga

SEQ ID NO: 89 - RNA, 7 nt, Artificial Sequence
  Synthetic Construct
agcagaa

SEQ ID NO: 90 - RNA, 7 nt, Artificial Sequence
  Synthetic Construct
gcagaag

SEQ ID NO: 91 - RNA, 7 nt, Artificial Sequence
  Synthetic Construct
cagaagc

SEQ ID NO: 92 - RNA, 7 nt, Artificial Sequence
  Synthetic Construct
agaagca

SEQ ID NO: 93 - RNA, 7 nt, Artificial Sequence
  Synthetic Construct
gaagcau

SEQ ID NO: 94 - RNA, 7 nt, Artificial Sequence
  Synthetic Construct
aagcauu

SEQ ID NO: 95 - RNA, 7 nt, Artificial Sequence
  Synthetic Construct
agcauuu

SEQ ID NO: 96 - RNA, 7 nt, Artificial Sequence
  Synthetic Construct
gcauuuc

SEQ ID NO: 97 - RNA, 7 nt, Artificial Sequence
  Synthetic Construct
cauuucc

SEQ ID NO: 98 - RNA, 7 nt, Artificial Sequence
  Synthetic Construct
auuuccg

SEQ ID NO: 99 - RNA, 7 nt, Artificial Sequence
  Synthetic Construct
uuuccgc

SEQ ID NO: 100 - RNA, 7 nt, Artificial Sequence
  Synthetic Construct
uuccgca

SEQ ID NO: 101 - RNA, 7 nt, Artificial Sequence
  Synthetic Construct
uccgcac

SEQ ID NO: 102 - RNA, 7 nt, Artificial Sequence
  Synthetic Construct
ccgcaca

SEQ ID NO: 103 - RNA, 7 nt, Artificial Sequence
  Synthetic Construct
cgcacac

SEQ ID NO: 104 - RNA, 7 nt, Artificial Sequence
  Synthetic Construct
gcacacu

SEQ ID NO: 105 - RNA, 7 nt, Artificial Sequence
  Synthetic Construct
cacacug

SEQ ID NO: 106 - RNA, 7 nt, Artificial Sequence
  Synthetic Construct
acacugg

SEQ ID NO: 107 - RNA, 7 nt, Artificial Sequence
  Synthetic Construct
cacuggu

SEQ ID NO: 108 - RNA, 7 nt, Artificial Sequence
  Synthetic Construct
acuggug

SEQ ID NO: 109 - RNA, 7 nt, Artificial Sequence
  Synthetic Construct
cuggugu

SEQ ID NO: 110 - RNA, 7 nt, Artificial Sequence
  Synthetic Construct
ugguguc

SEQ ID NO: 111 - RNA, 7 nt, Artificial Sequence
  Synthetic Construct
ggugucc

SEQ ID NO: 112 - RNA, 7 nt, Artificial Sequence
  Synthetic Construct
gugucca

SEQ ID NO: 113 - RNA, 7 nt, Artificial Sequence
  Synthetic Construct
uguccag

SEQ ID NO: 114 - RNA, 7 nt, Artificial Sequence
  Synthetic Construct
guccaga

SEQ ID NO: 115 - RNA, 7 nt, Artificial Sequence
  Synthetic Construct
uccagaa

SEQ ID NO: 116 - RNA, 7 nt, Artificial Sequence
  Synthetic Construct
ccagaau

SEQ ID NO: 117 - RNA, 7 nt, Artificial Sequence
  Synthetic Construct
cagaauc

SEQ ID NO: 118 - RNA, 7 nt, Artificial Sequence
  Synthetic Construct
agaaucu

SEQ ID NO: 119 - RNA, 7 nt, Artificial Sequence
  Synthetic Construct
gaaucua

SEQ ID NO: 120 - RNA, 7 nt, Artificial Sequence
  Synthetic Construct
aaucuag

SEQ ID NO: 121 - RNA, 7 nt, Artificial Sequence
  Synthetic Construct
aucuagu

SEQ ID NO: 122 - RNA, 7 nt, Artificial Sequence
  Synthetic Construct
ucuaguu

SEQ ID NO: 123 - RNA, 7 nt, Artificial Sequence
  Synthetic Construct
cuaguuu

SEQ ID NO: 124 - RNA, 7 nt, Artificial Sequence
  Synthetic Construct
uaguuug

SEQ ID NO: 125 - RNA, 7 nt, Artificial Sequence
  Synthetic Construct
aguuugu

SEQ ID NO: 126 - RNA, 7 nt, Artificial Sequence
  Synthetic Construct
guuugug

SEQ ID NO: 127 - RNA, 7 nt, Artificial Sequence
  Synthetic Construct
uuugugc

SEQ ID NO: 128 - RNA, 7 nt, Artificial Sequence
  Synthetic Construct
uugugca

SEQ ID NO: 129 - RNA, 7 nt, Artificial Sequence
  Synthetic Construct
ugugcag

SEQ ID NO: 130 - RNA, 7 nt, Artificial Sequence
  Synthetic Construct
gugcaga

SEQ ID NO: 131 - RNA, 7 nt, Artificial Sequence
  Synthetic Construct
ugcagaa

SEQ ID NO: 132 - RNA, 7 nt, Artificial Sequence
  Synthetic Construct
gcagaaa

SEQ ID NO: 133 - RNA, 7 nt, Artificial Sequence
  Synthetic Construct
cagaaau

SEQ ID NO: 134 - RNA, 7 nt, Artificial Sequence
  Synthetic Construct
agaaaug

SEQ ID NO: 135 - RNA, 7 nt, Artificial Sequence
  Synthetic Construct
gaaaugu

SEQ ID NO: 136 - RNA, 7 nt, Artificial Sequence
  Synthetic Construct
aaauguu

SEQ ID NO: 137 - RNA, 7 nt, Artificial Sequence
  Synthetic Construct
aauguuu

SEQ ID NO: 138 - RNA, 7 nt, Artificial Sequence
  Synthetic Construct
auguuuc

SEQ ID NO: 139 - RNA, 7 nt, Artificial Sequence
  Synthetic Construct
uguuucc

SEQ ID NO: 140 - RNA, 7 nt, Artificial Sequence
  Synthetic Construct
guuucca

SEQ ID NO: 141 - RNA, 7 nt, Artificial Sequence
  Synthetic Construct
uuuccac

SEQ ID NO: 142 - RNA, 7 nt, Artificial Sequence
  Synthetic Construct
uuccacu

SEQ ID NO: 143 - RNA, 7 nt, Artificial Sequence
  Synthetic Construct
uccacua

SEQ ID NO: 144 - RNA, 7 nt, Artificial Sequence
  Synthetic Construct
ccacuag

SEQ ID NO: 145 - RNA, 7 nt, Artificial Sequence
  Synthetic Construct
cacuaga

SEQ ID NO: 146 - RNA, 7 nt, Artificial Sequence
  Synthetic Construct
acuagau

SEQ ID NO: 147 - RNA, 7 nt, Artificial Sequence
  Synthetic Construct
cuagauu

SEQ ID NO: 148 - RNA, 7 nt, Artificial Sequence
  Synthetic Construct
uagauuu

SEQ ID NO: 149 - RNA, 7 nt, Artificial Sequence
  Synthetic Construct
agauuua

SEQ ID NO: 150 - RNA, 7 nt, Artificial Sequence
  Synthetic Construct
auuuaua

SEQ ID NO: 151 - RNA, 8 nt, Artificial Sequence
  Synthetic Construct
ccuaaaaa

SEQ ID NO: 152 - RNA, 8 nt, Artificial Sequence
  Synthetic Construct
cuaaaaau

SEQ ID NO: 153 - RNA, 8 nt, Artificial Sequence
  Synthetic Construct
uaaaaaug

SEQ ID NO: 154 - RNA, 8 nt, Artificial Sequence
  Synthetic Construct
aaaaaugu

SEQ ID NO: 155 - RNA, 8 nt, Artificial Sequence
  Synthetic Construct
aaaaugua

SEQ ID NO: 156 - RNA, 8 nt, Artificial Sequence
  Synthetic Construct
aaauguag

SEQ ID NO: 157 - RNA, 8 nt, Artificial Sequence
  Synthetic Construct
aauguagc

SEQ ID NO: 158 - RNA, 8 nt, Artificial Sequence
  Synthetic Construct
auguagca

SEQ ID NO: 159 - RNA, 8 nt, Artificial Sequence
  Synthetic Construct
uguagcag

SEQ ID NO: 160 - RNA, 8 nt, Artificial Sequence
  Synthetic Construct
guagcaga

SEQ ID NO: 161 - RNA, 8 nt, Artificial Sequence
  Synthetic Construct
uagcagaa

SEQ ID NO: 162 - RNA, 8 nt, Artificial Sequence
  Synthetic Construct
agcagaag

SEQ ID NO: 163 - RNA, 8 nt, Artificial Sequence
  Synthetic Construct
gcagaagc

SEQ ID NO: 164 - RNA, 8 nt, Artificial Sequence
  Synthetic Construct
cagaagca

SEQ ID NO: 165 - RNA, 8 nt, Artificial Sequence
  Synthetic Construct
agaagcau

SEQ ID NO: 166 - RNA, 8 nt, Artificial Sequence
  Synthetic Construct
gaagcauu

SEQ ID NO: 167 - RNA, 8 nt, Artificial Sequence
  Synthetic Construct
aagcauuu

SEQ ID NO: 168 - RNA, 8 nt, Artificial Sequence
  Synthetic Construct
agcauuuc

SEQ ID NO: 169 - RNA, 8 nt, Artificial Sequence
  Synthetic Construct
gcauuucc

SEQ ID NO: 170 - RNA, 8 nt, Artificial Sequence
  Synthetic Construct
cauuuccg

SEQ ID NO: 171 - RNA, 8 nt, Artificial Sequence
  Synthetic Construct
auuuccgc

SEQ ID NO: 172 - RNA, 8 nt, Artificial Sequence
  Synthetic Construct
uuuccgca

SEQ ID NO: 173 - RNA, 8 nt, Artificial Sequence
  Synthetic Construct
uuccgcac

SEQ ID NO: 174 - RNA, 8 nt, Artificial Sequence
  Synthetic Construct
uccgcaca

SEQ ID NO: 175 - RNA, 8 nt, Artificial Sequence
  Synthetic Construct
ccgcacac

SEQ ID NO: 176 - RNA, 8 nt, Artificial Sequence
  Synthetic Construct
cgcacacu

SEQ ID NO: 177 - RNA, 8 nt, Artificial Sequence
  Synthetic Construct
gcacacug

SEQ ID NO: 178 - RNA, 8 nt, Artificial Sequence
  Synthetic Construct
cacacugg

SEQ ID NO: 179 - RNA, 8 nt, Artificial Sequence
  Synthetic Construct
acacuggu

SEQ ID NO: 180 - RNA, 8 nt, Artificial Sequence
  Synthetic Construct
cacuggug

SEQ ID NO: 181 - RNA, 8 nt, Artificial Sequence
  Synthetic Construct
acuggugu

SEQ ID NO: 182 - RNA, 8 nt, Artificial Sequence
  Synthetic Construct
cugguguc

SEQ ID NO: 183 - RNA, 8 nt, Artificial Sequence
  Synthetic Construct
uggugucc

SEQ ID NO: 184 - RNA, 8 nt, Artificial Sequence
  Synthetic Construct
ggugucca

SEQ ID NO: 185 - RNA, 8 nt, Artificial Sequence
  Synthetic Construct
guguccag

SEQ ID NO: 186 - RNA, 8 nt, Artificial Sequence
  Synthetic Construct
uguccaga

SEQ ID NO: 187 - RNA, 8 nt, Artificial Sequence
  Synthetic Construct
guccagaa

SEQ ID NO: 188 - RNA, 8 nt, Artificial Sequence
  Synthetic Construct
uccagaau

SEQ ID NO: 189 - RNA, 8 nt, Artificial Sequence
  Synthetic Construct
ccagaauc

SEQ ID NO: 190 - RNA, 8 nt, Artificial Sequence
  Synthetic Construct
cagaaucu

SEQ ID NO: 191 - RNA, 8 nt, Artificial Sequence
  Synthetic Construct
agaaucua

SEQ ID NO: 192 - RNA, 8 nt, Artificial Sequence
  Synthetic Construct
gaaucuag

SEQ ID NO: 193 - RNA, 8 nt, Artificial Sequence
  Synthetic Construct
aaucuagu

SEQ ID NO: 194 - RNA, 8 nt, Artificial Sequence
  Synthetic Construct
aucuaguu

SEQ ID NO: 195 - RNA, 8 nt, Artificial Sequence
  Synthetic Construct
ucuaguuu

SEQ ID NO: 196 - RNA, 8 nt, Artificial Sequence
  Synthetic Construct
cuaguuug

SEQ ID NO: 197 - RNA, 8 nt, Artificial Sequence
  Synthetic Construct
uaguuugu

SEQ ID NO: 198 - RNA, 8 nt, Artificial Sequence
  Synthetic Construct
aguuugug

SEQ ID NO: 199 - RNA, 8 nt, Artificial Sequence
  Synthetic Construct
guuugugc

SEQ ID NO: 200 - RNA, 8 nt, Artificial Sequence
  Synthetic Construct
uuugugca

SEQ ID NO: 201 - RNA, 8 nt, Artificial Sequence
  Synthetic Construct
uugugcag

SEQ ID NO: 202 - RNA, 8 nt, Artificial Sequence
  Synthetic Construct
ugugcaga

SEQ ID NO: 203 - RNA, 8 nt, Artificial Sequence
  Synthetic Construct
gugcagaa

SEQ ID NO: 204 - RNA, 8 nt, Artificial Sequence
  Synthetic Construct
ugcagaaa

SEQ ID NO: 205 - RNA, 8 nt, Artificial Sequence
  Synthetic Construct
gcagaaau

SEQ ID NO: 206 - RNA, 8 nt, Artificial Sequence
  Synthetic Construct
cagaaaug

SEQ ID NO: 207 - RNA, 8 nt, Artificial Sequence
  Synthetic Construct
agaaaugu

SEQ ID NO: 208 - RNA, 8 nt, Artificial Sequence
  Synthetic Construct
gaaauguu

SEQ ID NO: 209 - RNA, 8 nt, Artificial Sequence
  Synthetic Construct
aaauguuu

SEQ ID NO: 210 - RNA, 8 nt, Artificial Sequence
  Synthetic Construct
aauguuuc

SEQ ID NO: 211 - RNA, 8 nt, Artificial Sequence
  Synthetic Construct
auguuucc

SEQ ID NO: 212 - RNA, 8 nt, Artificial Sequence
  Synthetic Construct
uguuucca

SEQ ID NO: 213 - RNA, 8 nt, Artificial Sequence
  Synthetic Construct
guuuccac

SEQ ID NO: 214 - RNA, 8 nt, Artificial Sequence
  Synthetic Construct
uuuccacu

SEQ ID NO: 215 - RNA, 8 nt, Artificial Sequence
  Synthetic Construct
uuccacua

SEQ ID NO: 216 - RNA, 8 nt, Artificial Sequence
  Synthetic Construct
uccacuag

SEQ ID NO: 217 - RNA, 8 nt, Artificial Sequence
  Synthetic Construct
ccacuaga

SEQ ID NO: 218 - RNA, 8 nt, Artificial Sequence
  Synthetic Construct
cacuagau

SEQ ID NO: 219 - RNA, 8 nt, Artificial Sequence
  Synthetic Construct
acuagauu

SEQ ID NO: 220 - RNA, 8 nt, Artificial Sequence
  Synthetic Construct
cuagauuu

SEQ ID NO: 221 - RNA, 8 nt, Artificial Sequence
  Synthetic Construct
uagauuua

SEQ ID NO: 222 - RNA, 8 nt, Artificial Sequence
  Synthetic Construct
agauuuau

SEQ ID NO: 223 - RNA, 8 nt, Artificial Sequence
  Synthetic Construct
gauuuaua

SEQ ID NO: 224 - RNA, 9 nt, Artificial Sequence
  Synthetic Construct
ccuaaaaau

SEQ ID NO: 225 - RNA, 9 nt, Artificial Sequence
  Synthetic Construct
cuaaaaaug

SEQ ID NO: 226 - RNA, 9 nt, Artificial Sequence
  Synthetic Construct
uaaaaaugu

SEQ ID NO: 227 - RNA, 9 nt, Artificial Sequence
  Synthetic Construct
aaaaaugua

SEQ ID NO: 228 - RNA, 9 nt, Artificial Sequence
  Synthetic Construct
aaaauguag

SEQ ID NO: 229 - RNA, 9 nt, Artificial Sequence
  Synthetic Construct
aaauguagc

SEQ ID NO: 230 - RNA, 9 nt, Artificial Sequence
  Synthetic Construct
aauguagca

SEQ ID NO: 231 - RNA, 9 nt, Artificial Sequence
  Synthetic Construct
auguagcag

SEQ ID NO: 232 - RNA, 9 nt, Artificial Sequence
  Synthetic Construct
uguagcaga

SEQ ID NO: 233 - RNA, 9 nt, Artificial Sequence
  Synthetic Construct
guagcagaa

SEQ ID NO: 234 - RNA, 9 nt, Artificial Sequence
  Synthetic Construct
uagcagaag

SEQ ID NO: 235 - RNA, 9 nt, Artificial Sequence
  Synthetic Construct
agcagaagc

SEQ ID NO: 236 - RNA, 9 nt, Artificial Sequence
  Synthetic Construct
gcagaagca

SEQ ID NO: 237 - RNA, 9 nt, Artificial Sequence
  Synthetic Construct
cagaagcau

SEQ ID NO: 238 - RNA, 9 nt, Artificial Sequence
  Synthetic Construct
agaagcauu

SEQ ID NO: 239 - RNA, 9 nt, Artificial Sequence
  Synthetic Construct
gaagcauuu

SEQ ID NO: 240 - RNA, 9 nt, Artificial Sequence
  Synthetic Construct
aagcauuuc

SEQ ID NO: 241 - RNA, 9 nt, Artificial Sequence
  Synthetic Construct
agcauuucc

SEQ ID NO: 242 - RNA, 9 nt, Artificial Sequence
  Synthetic Construct
gcauuuccg

SEQ ID NO: 243 - RNA, 9 nt, Artificial Sequence
  Synthetic Construct
cauuuccgc

SEQ ID NO: 244 - RNA, 9 nt, Artificial Sequence
  Synthetic Construct
auuuccgca

SEQ ID NO: 245 - RNA, 9 nt, Artificial Sequence
  Synthetic Construct
uuuccgcac

SEQ ID NO: 246 - RNA, 9 nt, Artificial Sequence
  Synthetic Construct
uuccgcaca

SEQ ID NO: 247 - RNA, 9 nt, Artificial Sequence
  Synthetic Construct
uccgcacac

SEQ ID NO: 248 - RNA, 9 nt, Artificial Sequence
  Synthetic Construct
ccgcacacu

SEQ ID NO: 249 - RNA, 9 nt, Artificial Sequence
  Synthetic Construct
cgcacacug

SEQ ID NO: 250 - RNA, 9 nt, Artificial Sequence
  Synthetic Construct
gcacacugg

SEQ ID NO: 251 - RNA, 9 nt, Artificial Sequence
  Synthetic Construct
cacacuggu

SEQ ID NO: 252 - RNA, 9 nt, Artificial Sequence
  Synthetic Construct
acacuggug

SEQ ID NO: 253 - RNA, 9 nt, Artificial Sequence
  Synthetic Construct
cacuggugu

SEQ ID NO: 254 - RNA, 9 nt, Artificial Sequence
  Synthetic Construct
acugguguc

SEQ ID NO: 255 - RNA, 9 nt, Artificial Sequence
  Synthetic Construct
cuggugucc

SEQ ID NO: 256 - RNA, 9 nt, Artificial Sequence
  Synthetic Construct
uggugucca

SEQ ID NO: 257 - RNA, 9 nt, Artificial Sequence
  Synthetic Construct
gguguccag

SEQ ID NO: 258 - RNA, 9 nt, Artificial Sequence
  Synthetic Construct
guguccaga

SEQ ID NO: 259 - RNA, 9 nt, Artificial Sequence
  Synthetic Construct
uguccagaa

SEQ ID NO: 260 - RNA, 9 nt, Artificial Sequence
  Synthetic Construct
guccagaau

SEQ ID NO: 261 - RNA, 9 nt, Artificial Sequence
  Synthetic Construct
uccagaauc

SEQ ID NO: 262 - RNA, 9 nt, Artificial Sequence
  Synthetic Construct
ccagaaucu

SEQ ID NO: 263 - RNA, 9 nt, Artificial Sequence
  Synthetic Construct
cagaaucua

SEQ ID NO: 264 - RNA, 9 nt, Artificial Sequence
  Synthetic Construct
agaaucuag

SEQ ID NO: 265 - RNA, 9 nt, Artificial Sequence
  Synthetic Construct
gaaucuagu

SEQ ID NO: 266 - RNA, 9 nt, Artificial Sequence
  Synthetic Construct
aaucuaguu

SEQ ID NO: 267 - RNA, 9 nt, Artificial Sequence
  Synthetic Construct
aucuaguuu

SEQ ID NO: 268 - RNA, 9 nt, Artificial Sequence
  Synthetic Construct
ucuaguuug

SEQ ID NO: 269 - RNA, 9 nt, Artificial Sequence
  Synthetic Construct
cuaguuugu

SEQ ID NO: 270 - RNA, 9 nt, Artificial Sequence
  Synthetic Construct
uaguuugug

SEQ ID NO: 271 - RNA, 9 nt, Artificial Sequence
  Synthetic Construct
aguuugugc

SEQ ID NO: 272 - RNA, 9 nt, Artificial Sequence
  Synthetic Construct
guuugugca

SEQ ID NO: 273 - RNA, 9 nt, Artificial Sequence
  Synthetic Construct
uuugugcag

SEQ ID NO: 274 - RNA, 9 nt, Artificial Sequence
  Synthetic Construct
uugugcaga

SEQ ID NO: 275 - RNA, 9 nt, Artificial Sequence
  Synthetic Construct
ugugcagaa

SEQ ID NO: 276 - RNA, 9 nt, Artificial Sequence
  Synthetic Construct
gugcagaaa

SEQ ID NO: 277 - RNA, 9 nt, Artificial Sequence
  Synthetic Construct
ugcagaaau

SEQ ID NO: 278 - RNA, 9 nt, Artificial Sequence
  Synthetic Construct
gcagaaaug

SEQ ID NO: 279 - RNA, 9 nt, Artificial Sequence
  Synthetic Construct
cagaaaugu

SEQ ID NO: 280 - RNA, 9 nt, Artificial Sequence
  Synthetic Construct
agaaauguu

SEQ ID NO: 281 - RNA, 9 nt, Artificial Sequence
  Synthetic Construct
gaaauguuu

SEQ ID NO: 282 - RNA, 9 nt, Artificial Sequence
  Synthetic Construct
aaauguuuc

SEQ ID NO: 283 - RNA, 9 nt, Artificial Sequence
  Synthetic Construct
aauguuucc

SEQ ID NO: 284 - RNA, 9 nt, Artificial Sequence
  Synthetic Construct
auguuucca

SEQ ID NO: 285 - RNA, 9 nt, Artificial Sequence
  Synthetic Construct
uguuuccac

SEQ ID NO: 286 - RNA, 9 nt, Artificial Sequence
  Synthetic Construct
guuuccacu

SEQ ID NO: 287 - RNA, 9 nt, Artificial Sequence
  Synthetic Construct
uuuccacua

SEQ ID NO: 288 - RNA, 9 nt, Artificial Sequence
  Synthetic Construct
uuccacuag

SEQ ID NO: 289 - RNA, 9 nt, Artificial Sequence
  Synthetic Construct
uccacuaga

SEQ ID NO: 290 - RNA, 9 nt, Artificial Sequence
  Synthetic Construct
ccacuagau

SEQ ID NO: 291 - RNA, 9 nt, Artificial Sequence
  Synthetic Construct
cacuagauu

SEQ ID NO: 292 - RNA, 9 nt, Artificial Sequence
  Synthetic Construct
acuagauuu

SEQ ID NO: 293 - RNA, 9 nt, Artificial Sequence
  Synthetic Construct
cuagauuua

SEQ ID NO: 294 - RNA, 9 nt, Artificial Sequence
  Synthetic Construct
uagauuuau

SEQ ID NO: 295 - RNA, 9 nt, Artificial Sequence
  Synthetic Construct
agauuuaua

SEQ ID NO: 296 - RNA, 10 nt, Artificial Sequence
  Synthetic Construct
ccuaaaaaug

SEQ ID NO: 297 - RNA, 10 nt, Artificial Sequence
  Synthetic Construct
ccuaaaaaug

SEQ ID NO: 298 - RNA, 10 nt, Artificial Sequence
  Synthetic Construct
cuaaaaaugu

SEQ ID NO: 299 - RNA, 10 nt, Artificial Sequence
  Synthetic Construct
uaaaaaugua

SEQ ID NO: 300 - RNA, 10 nt, Artificial Sequence
  Synthetic Construct
aaaaauguag

SEQ ID NO: 301 - RNA, 10 nt, Artificial Sequence
  Synthetic Construct
aaaauguagc

SEQ ID NO: 302 - RNA, 10 nt, Artificial Sequence
  Synthetic Construct
aaauguagca

SEQ ID NO: 303 - RNA, 10 nt, Artificial Sequence
  Synthetic Construct
aauguagcag

SEQ ID NO: 304 - RNA, 10 nt, Artificial Sequence
  Synthetic Construct
auguagcaga

SEQ ID NO: 305 - RNA, 10 nt, Artificial Sequence
  Synthetic Construct
uguagcagaa

SEQ ID NO: 306 - RNA, 10 nt, Artificial Sequence
  Synthetic Construct
guagcagaag

SEQ ID NO: 307 - RNA, 10 nt, Artificial Sequence
  Synthetic Construct
uagcagaagc

SEQ ID NO: 308 - RNA, 10 nt, Artificial Sequence
  Synthetic Construct
agcagaagca

SEQ ID NO: 309 - RNA, 10 nt, Artificial Sequence
  Synthetic Construct
gcagaagcau

SEQ ID NO: 310 - RNA, 10 nt, Artificial Sequence
  Synthetic Construct
cagaagcauu

SEQ ID NO: 311 - RNA, 10 nt, Artificial Sequence
  Synthetic Construct
agaagcauuu

SEQ ID NO: 312 - RNA, 10 nt, Artificial Sequence
  Synthetic Construct
gaagcauuuc

SEQ ID NO: 313 - RNA, 10 nt, Artificial Sequence
  Synthetic Construct
aagcauuucc

SEQ ID NO: 314 - RNA, 10 nt, Artificial Sequence
  Synthetic Construct
agcauuuccg

SEQ ID NO: 315 - RNA, 10 nt, Artificial Sequence
  Synthetic Construct
gcauuuccgc

SEQ ID NO: 316 - RNA, 10 nt, Artificial Sequence
  Synthetic Construct
cauuuccgca

SEQ ID NO: 317 - RNA, 10 nt, Artificial Sequence
  Synthetic Construct
auuuccgcac

SEQ ID NO: 318 - RNA, 10 nt, Artificial Sequence
  Synthetic Construct
uuuccgcaca

SEQ ID NO: 319 - RNA, 10 nt, Artificial Sequence
  Synthetic Construct
uuccgcacac

SEQ ID NO: 320 - RNA, 10 nt, Artificial Sequence
  Synthetic Construct
uccgcacacu

SEQ ID NO: 321 - RNA, 10 nt, Artificial Sequence
  Synthetic Construct
ccgcacacug

SEQ ID NO: 322 - RNA, 10 nt, Artificial Sequence
  Synthetic Construct
cgcacacugg

SEQ ID NO: 323 - RNA, 10 nt, Artificial Sequence
  Synthetic Construct
gcacacuggu

SEQ ID NO: 324 - RNA, 10 nt, Artificial Sequence
  Synthetic Construct
cacacuggug

SEQ ID NO: 325 - RNA, 10 nt, Artificial Sequence
  Synthetic Construct
acacuggugu

SEQ ID NO: 326 - RNA, 10 nt, Artificial Sequence
  Synthetic Construct
cacugguguc

SEQ ID NO: 327 - RNA, 10 nt, Artificial Sequence
  Synthetic Construct
acuggugucc

SEQ ID NO: 328 - RNA, 10 nt, Artificial Sequence
  Synthetic Construct
cuggugucca

SEQ ID NO: 329 - RNA, 10 nt, Artificial Sequence
  Synthetic Construct
ugguguccag

SEQ ID NO: 330 - RNA, 10 nt, Artificial Sequence
  Synthetic Construct
gguguccaga

SEQ ID NO: 331 - RNA, 10 nt, Artificial Sequence
  Synthetic Construct
guguccagaa

SEQ ID NO: 332 - RNA, 10 nt, Artificial Sequence
  Synthetic Construct
uguccagaau

SEQ ID NO: 333 - RNA, 10 nt, Artificial Sequence
  Synthetic Construct
guccagaauc

SEQ ID NO: 334 - RNA, 10 nt, Artificial Sequence
  Synthetic Construct
uccagaaucu

SEQ ID NO: 335 - RNA, 10 nt, Artificial Sequence
  Synthetic Construct
ccagaaucua

SEQ ID NO: 336 - RNA, 10 nt, Artificial Sequence
  Synthetic Construct
cagaaucuag

SEQ ID NO: 337 - RNA, 10 nt, Artificial Sequence
  Synthetic Construct
agaaucuagu

SEQ ID NO: 338 - RNA, 10 nt, Artificial Sequence
  Synthetic Construct
gaaucuaguu

SEQ ID NO: 339 - RNA, 10 nt, Artificial Sequence
  Synthetic Construct
aaucuaguuu

SEQ ID NO: 340 - RNA, 10 nt, Artificial Sequence
  Synthetic Construct
aucuaguuug

SEQ ID NO: 341 - RNA, 10 nt, Artificial Sequence
  Synthetic Construct
ucuaguuugu

SEQ ID NO: 342 - RNA, 10 nt, Artificial Sequence
  Synthetic Construct
cuaguuugug

SEQ ID NO: 343 - RNA, 10 nt, Artificial Sequence
  Synthetic Construct
uaguuugugc

SEQ ID NO: 344 - RNA, 10 nt, Artificial Sequence
  Synthetic Construct
aguuugugca

SEQ ID NO: 345 - RNA, 10 nt, Artificial Sequence
  Synthetic Construct
guuugugcag

SEQ ID NO: 346 - RNA, 10 nt, Artificial Sequence
  Synthetic Construct
uuugugcaga

SEQ ID NO: 347 - RNA, 10 nt, Artificial Sequence
  Synthetic Construct
uugugcagaa

SEQ ID NO: 348 - RNA, 10 nt, Artificial Sequence
  Synthetic Construct
ugugcagaaa

SEQ ID NO: 349 - RNA, 10 nt, Artificial Sequence
  Synthetic Construct
gugcagaaau

SEQ ID NO: 350 - RNA, 10 nt, Artificial Sequence
  Synthetic Construct
ugcagaaaug

SEQ ID NO: 351 - RNA, 10 nt, Artificial Sequence
  Synthetic Construct
gcagaaaugu

SEQ ID NO: 352 - RNA, 10 nt, Artificial Sequence
  Synthetic Construct
cagaaauguu

SEQ ID NO: 353 - RNA, 10 nt, Artificial Sequence
  Synthetic Construct
agaaauguuu

SEQ ID NO: 354 - RNA, 10 nt, Artificial Sequence
  Synthetic Construct
gaaauguuuc

SEQ ID NO: 355 - RNA, 10 nt, Artificial Sequence
  Synthetic Construct
aaauguuucc

SEQ ID NO: 356 - RNA, 10 nt, Artificial Sequence
  Synthetic Construct
aauguuucca

SEQ ID NO: 357 - RNA, 10 nt, Artificial Sequence
  Synthetic Construct
auguuuccac

SEQ ID NO: 358 - RNA, 10 nt, Artificial Sequence
  Synthetic Construct
uguuuccacu

SEQ ID NO: 359 - RNA, 10 nt, Artificial Sequence
  Synthetic Construct
guuuccacua

SEQ ID NO: 360 - RNA, 10 nt, Artificial Sequence
  Synthetic Construct
uuuccacuag

SEQ ID NO: 361 - RNA, 10 nt, Artificial Sequence
  Synthetic Construct
uuccacuaga

SEQ ID NO: 362 - RNA, 10 nt, Artificial Sequence
  Synthetic Construct
uccacuagau

SEQ ID NO: 363 - RNA, 10 nt, Artificial Sequence
  Synthetic Construct
ccacuagauu

SEQ ID NO: 364 - RNA, 10 nt, Artificial Sequence
  Synthetic Construct
cacuagauuu

SEQ ID NO: 365 - RNA, 10 nt, Artificial Sequence
  Synthetic Construct
acuagauuua

SEQ ID NO: 366 - RNA, 10 nt, Artificial Sequence
  Synthetic Construct
cuagauuuau

SEQ ID NO: 367 - RNA, 10 nt, Artificial Sequence
  Synthetic Construct
uagauuuaua

SEQ ID NO: 368 - RNA, 11 nt, Artificial Sequence
  Synthetic Construct
ccuaaaaaugu

SEQ ID NO: 369 - RNA, 11 nt, Artificial Sequence
  Synthetic Construct
cuaaaaaugua

SEQ ID NO: 370 - RNA, 11 nt, Artificial Sequence
  Synthetic Construct
uaaaaauguag

SEQ ID NO: 371 - RNA, 11 nt, Artificial Sequence
  Synthetic Construct
aaaaauguagc

SEQ ID NO: 372 - RNA, 11 nt, Artificial Sequence
  Synthetic Construct
aaaauguagca

SEQ ID NO: 373 - RNA, 11 nt, Artificial Sequence
  Synthetic Construct
aaauguagcag

SEQ ID NO: 374 - RNA, 11 nt, Artificial Sequence
  Synthetic Construct
aauguagcaga

SEQ ID NO: 375 - RNA, 11 nt, Artificial Sequence
  Synthetic Construct
auguagcagaa

SEQ ID NO: 376 - RNA, 11 nt, Artificial Sequence
  Synthetic Construct
uguagcagaag

SEQ ID NO: 377 - RNA, 11 nt, Artificial Sequence
  Synthetic Construct
guagcagaagc

SEQ ID NO: 378 - RNA, 11 nt, Artificial Sequence
  Synthetic Construct
uagcagaagca

SEQ ID NO: 379 - RNA, 11 nt, Artificial Sequence
  Synthetic Construct
agcagaagcau

SEQ ID NO: 380 - RNA, 11 nt, Artificial Sequence
  Synthetic Construct
gcagaagcauu

SEQ ID NO: 381 - RNA, 11 nt, Artificial Sequence
  Synthetic Construct
cagaagcauuu

SEQ ID NO: 382 - RNA, 11 nt, Artificial Sequence
  Synthetic Construct
agaagcauuuc

SEQ ID NO: 383 - RNA, 11 nt, Artificial Sequence
  Synthetic Construct
gaagcauuucc

SEQ ID NO: 384 - RNA, 11 nt, Artificial Sequence
  Synthetic Construct
aagcauuuccg

SEQ ID NO: 385 - RNA, 11 nt, Artificial Sequence
  Synthetic Construct
agcauuuccgc

SEQ ID NO: 386 - RNA, 11 nt, Artificial Sequence
  Synthetic Construct
gcauuuccgca

SEQ ID NO: 387 - RNA, 11 nt, Artificial Sequence
  Synthetic Construct
cauuuccgcac

SEQ ID NO: 388 - RNA, 11 nt, Artificial Sequence
  Synthetic Construct
auuuccgcaca

SEQ ID NO: 389 - RNA, 11 nt, Artificial Sequence
  Synthetic Construct
uuuccgcacac

SEQ ID NO: 390 - RNA, 11 nt, Artificial Sequence
  Synthetic Construct
uuccgcacacu

SEQ ID NO: 391 - RNA, 11 nt, Artificial Sequence
  Synthetic Construct
uccgcacacug

SEQ ID NO: 392 - RNA, 11 nt, Artificial Sequence
  Synthetic Construct
ccgcacacugg

SEQ ID NO: 393 - RNA, 11 nt, Artificial Sequence
  Synthetic Construct
cgcacacuggu

SEQ ID NO: 394 - RNA, 11 nt, Artificial Sequence
  Synthetic Construct
gcacacuggug

SEQ ID NO: 395 - RNA, 11 nt, Artificial Sequence
  Synthetic Construct
cacacuggugu

SEQ ID NO: 396 - RNA, 11 nt, Artificial Sequence
  Synthetic Construct
acacugguguc

SEQ ID NO: 397 - RNA, 11 nt, Artificial Sequence
  Synthetic Construct
cacuggugucc

SEQ ID NO: 398 - RNA, 11 nt, Artificial Sequence
  Synthetic Construct
acuggugucca

SEQ ID NO: 399 - RNA, 11 nt, Artificial Sequence
  Synthetic Construct
cugguguccag

SEQ ID NO: 400 - RNA, 11 nt, Artificial Sequence
  Synthetic Construct
ugguguccaga

SEQ ID NO: 401 - RNA, 11 nt, Artificial Sequence
  Synthetic Construct
gguguccagaa

SEQ ID NO: 402 - RNA, 11 nt, Artificial Sequence
  Synthetic Construct
guguccagaau

SEQ ID NO: 403 - RNA, 11 nt, Artificial Sequence
  Synthetic Construct
uguccagaauc

SEQ ID NO: 404 - RNA, 11 nt, Artificial Sequence
  Synthetic Construct
guccagaaucu

SEQ ID NO: 405 - RNA, 11 nt, Artificial Sequence
  Synthetic Construct
uccagaaucua

SEQ ID NO: 406 - RNA, 11 nt, Artificial Sequence
  Synthetic Construct
ccagaaucuag

SEQ ID NO: 407 - RNA, 11 nt, Artificial Sequence
  Synthetic Construct
cagaaucuagu

SEQ ID NO: 408 - RNA, 11 nt, Artificial Sequence
  Synthetic Construct
agaaucuaguu

SEQ ID NO: 409 - RNA, 11 nt, Artificial Sequence
  Synthetic Construct
gaaucuaguuu

SEQ ID NO: 410 - RNA, 11 nt, Artificial Sequence
  Synthetic Construct
aaucuaguuug

SEQ ID NO: 411 - RNA, 11 nt, Artificial Sequence
  Synthetic Construct
aucuaguuugu

SEQ ID NO: 412 - RNA, 11 nt, Artificial Sequence
  Synthetic Construct
ucuaguuugug

SEQ ID NO: 413 - RNA, 11 nt, Artificial Sequence
  Synthetic Construct
cuaguuugugc

SEQ ID NO: 414 - RNA, 11 nt, Artificial Sequence
  Synthetic Construct
uaguuugugca

SEQ ID NO: 415 - RNA, 11 nt, Artificial Sequence
  Synthetic Construct
aguuugugcag

SEQ ID NO: 416 - RNA, 11 nt, Artificial Sequence
  Synthetic Construct
guuugugcaga

SEQ ID NO: 417 - RNA, 11 nt, Artificial Sequence
  Synthetic Construct
uuugugcagaa

SEQ ID NO: 418 - RNA, 11 nt, Artificial Sequence
  Synthetic Construct
uugugcagaaa

SEQ ID NO: 419 - RNA, 11 nt, Artificial Sequence
  Synthetic Construct
ugugcagaaau

SEQ ID NO: 420 - RNA, 11 nt, Artificial Sequence
  Synthetic Construct
gugcagaaaug

SEQ ID NO: 421 - RNA, 11 nt, Artificial Sequence
  Synthetic Construct
ugcagaaaugu

SEQ ID NO: 422 - RNA, 11 nt, Artificial Sequence
  Synthetic Construct
gcagaaauguu

SEQ ID NO: 423 - RNA, 11 nt, Artificial Sequence
  Synthetic Construct
cagaaauguuu

SEQ ID NO: 424 - RNA, 11 nt, Artificial Sequence
  Synthetic Construct
agaaauguuuc

SEQ ID NO: 425 - RNA, 11 nt, Artificial Sequence
  Synthetic Construct
gaaauguuucc

SEQ ID NO: 426 - RNA, 11 nt, Artificial Sequence
  Synthetic Construct
aaauguuucca

SEQ ID NO: 427 - RNA, 11 nt, Artificial Sequence
  Synthetic Construct
aauguuuccac

SEQ ID NO: 428 - RNA, 11 nt, Artificial Sequence
  Synthetic Construct
auguuuccacu

SEQ ID NO: 429 - RNA, 11 nt, Artificial Sequence
  Synthetic Construct
uguuuccacua

SEQ ID NO: 430 - RNA, 11 nt, Artificial Sequence
  Synthetic Construct
guuuccacuag

SEQ ID NO: 431 - RNA, 11 nt, Artificial Sequence
  Synthetic Construct
uuuccacuaga

SEQ ID NO: 432 - RNA, 11 nt, Artificial Sequence
  Synthetic Construct
uuccacuagau

SEQ ID NO: 433 - RNA, 11 nt, Artificial Sequence
  Synthetic Construct
uccacuagauu

SEQ ID NO: 434 - RNA, 11 nt, Artificial Sequence
  Synthetic Construct
ccacuagauuu

SEQ ID NO: 435 - RNA, 11 nt, Artificial Sequence
  Synthetic Construct
cacuagauuua

SEQ ID NO: 436 - RNA, 11 nt, Artificial Sequence
  Synthetic Construct
acuagauuuau

SEQ ID NO: 437 - RNA, 11 nt, Artificial Sequence
  Synthetic Construct
cuagauuuaua

SEQ ID NO: 438 - RNA, 12 nt, Artificial Sequence
  Synthetic Construct
ccuaaaaaugua

SEQ ID NO: 439 - RNA, 12 nt, Artificial Sequence
  Synthetic Construct
cuaaaaauguag

SEQ ID NO: 440 - RNA, 12 nt, Artificial Sequence
  Synthetic Construct
uaaaaauguagc

SEQ ID NO: 441 - RNA, 12 nt, Artificial Sequence
  Synthetic Construct
aaaaauguagca

SEQ ID NO: 442 - RNA, 12 nt, Artificial Sequence
  Synthetic Construct
aaaauguagcag

SEQ ID NO: 443 - RNA, 12 nt, Artificial Sequence
  Synthetic Construct
aaauguagcaga

SEQ ID NO: 444 - RNA, 12 nt, Artificial Sequence
  Synthetic Construct
aauguagcagaa

SEQ ID NO: 445 - RNA, 12 nt, Artificial Sequence
  Synthetic Construct
auguagcagaag

SEQ ID NO: 446 - RNA, 12 nt, Artificial Sequence
  Synthetic Construct
uguagcagaagc

SEQ ID NO: 447 - RNA, 12 nt, Artificial Sequence
  Synthetic Construct
guagcagaagca

SEQ ID NO: 448 - RNA, 12 nt, Artificial Sequence
  Synthetic Construct
uagcagaagcau

SEQ ID NO: 449 - RNA, 12 nt, Artificial Sequence
  Synthetic Construct
agcagaagcauu

SEQ ID NO: 450 - RNA, 12 nt, Artificial Sequence
  Synthetic Construct
gcagaagcauuu

SEQ ID NO: 451 - RNA, 12 nt, Artificial Sequence
  Synthetic Construct
cagaagcauuuc

SEQ ID NO: 452 - RNA, 12 nt, Artificial Sequence
  Synthetic Construct
agaagcauuucc

SEQ ID NO: 453 - RNA, 12 nt, Artificial Sequence
  Synthetic Construct
gaagcauuuccg

SEQ ID NO: 454 - RNA, 12 nt, Artificial Sequence
  Synthetic Construct
aagcauuuccgc

SEQ ID NO: 455 - RNA, 12 nt, Artificial Sequence
  Synthetic Construct
agcauuuccgca

SEQ ID NO: 456 - RNA, 12 nt, Artificial Sequence
  Synthetic Construct
gcauuuccgcac

SEQ ID NO: 457 - RNA, 12 nt, Artificial Sequence
  Synthetic Construct
cauuuccgcaca

SEQ ID NO: 458 - RNA, 12 nt, Artificial Sequence
  Synthetic Construct
auuuccgcacac

SEQ ID NO: 459 - RNA, 12 nt, Artificial Sequence
  Synthetic Construct
uuuccgcacacu

SEQ ID NO: 460 - RNA, 12 nt, Artificial Sequence
  Synthetic Construct
uuccgcacacug

SEQ ID NO: 461 - RNA, 12 nt, Artificial Sequence
  Synthetic Construct
uccgcacacugg

SEQ ID NO: 462 - RNA, 12 nt, Artificial Sequence
  Synthetic Construct
ccgcacacuggu

SEQ ID NO: 463 - RNA, 12 nt, Artificial Sequence
  Synthetic Construct
cgcacacuggug

SEQ ID NO: 464 - RNA, 12 nt, Artificial Sequence
  Synthetic Construct
gcacacuggugu

SEQ ID NO: 465 - RNA, 12 nt, Artificial Sequence
  Synthetic Construct
cacacugguguc

SEQ ID NO: 466 - RNA, 12 nt, Artificial Sequence
  Synthetic Construct
acacuggugucc

SEQ ID NO: 467 - RNA, 12 nt, Artificial Sequence
  Synthetic Construct
cacuggugucca

SEQ ID NO: 468 - RNA, 12 nt, Artificial Sequence
  Synthetic Construct
acugguguccag

SEQ ID NO: 469 - RNA, 12 nt, Artificial Sequence
  Synthetic Construct
cugguguccaga

SEQ ID NO: 470 - RNA, 12 nt, Artificial Sequence
  Synthetic Construct
ugguguccagaa

SEQ ID NO: 471 - RNA, 12 nt, Artificial Sequence
  Synthetic Construct
gguguccagaau

SEQ ID NO: 472 - RNA, 12 nt, Artificial Sequence
  Synthetic Construct
guguccagaauc

SEQ ID NO: 473 - RNA, 12 nt, Artificial Sequence
  Synthetic Construct
uguccagaaucu

SEQ ID NO: 474 - RNA, 12 nt, Artificial Sequence
  Synthetic Construct
guccagaaucua

SEQ ID NO: 475 - RNA, 12 nt, Artificial Sequence
  Synthetic Construct
uccagaaucuag

SEQ ID NO: 476 - RNA, 12 nt, Artificial Sequence
  Synthetic Construct
ccagaaucuagu

SEQ ID NO: 477 - RNA, 12 nt, Artificial Sequence
  Synthetic Construct
cagaaucuaguu

SEQ ID NO: 478 - RNA, 12 nt, Artificial Sequence
  Synthetic Construct
agaaucuaguuu

SEQ ID NO: 479 - RNA, 12 nt, Artificial Sequence
  Synthetic Construct
gaaucuaguuug

SEQ ID NO: 480 - RNA, 12 nt, Artificial Sequence
  Synthetic Construct
aaucuaguuugu

SEQ ID NO: 481 - RNA, 12 nt, Artificial Sequence
  Synthetic Construct
aucuaguuugug

SEQ ID NO: 482 - RNA, 12 nt, Artificial Sequence
  Synthetic Construct
ucuaguuugugc

SEQ ID NO: 483 - RNA, 12 nt, Artificial Sequence
  Synthetic Construct
cuaguuugugca

SEQ ID NO: 484 - RNA, 12 nt, Artificial Sequence
  Synthetic Construct
uaguuugugcag

SEQ ID NO: 485 - RNA, 12 nt, Artificial Sequence
  Synthetic Construct
aguuugugcaga

SEQ ID NO: 486 - RNA, 12 nt, Artificial Sequence
  Synthetic Construct
guuugugcagaa

SEQ ID NO: 487 - RNA, 12 nt, Artificial Sequence
  Synthetic Construct
uuugugcagaaa

SEQ ID NO: 488 - RNA, 12 nt, Artificial Sequence
  Synthetic Construct
uugugcagaaau

SEQ ID NO: 489 - RNA, 12 nt, Artificial Sequence
  Synthetic Construct
ugugcagaaaug

SEQ ID NO: 490 - RNA, 12 nt, Artificial Sequence
  Synthetic Construct
gugcagaaaugu

SEQ ID NO: 491 - RNA, 12 nt, Artificial Sequence
  Synthetic Construct
ugcagaaauguu

SEQ ID NO: 492 - RNA, 12 nt, Artificial Sequence
  Synthetic Construct
gcagaaauguuu

SEQ ID NO: 493 - RNA, 12 nt, Artificial Sequence
  Synthetic Construct
cagaaauguuuc

SEQ ID NO: 494 - RNA, 12 nt, Artificial Sequence
  Synthetic Construct
agaaauguuucc

SEQ ID NO: 495 - RNA, 12 nt, Artificial Sequence
  Synthetic Construct
gaaauguuucca

SEQ ID NO: 496 - RNA, 12 nt, Artificial Sequence
  Synthetic Construct
aaauguuuccac

SEQ ID NO: 497 - RNA, 12 nt, Artificial Sequence
  Synthetic Construct
aauguuuccacu

SEQ ID NO: 498 - RNA, 12 nt, Artificial Sequence
  Synthetic Construct
auguuuccacua

SEQ ID NO: 499 - RNA, 12 nt, Artificial Sequence
  Synthetic Construct
uguuuccacuag

SEQ ID NO: 500 - RNA, 12 nt, Artificial Sequence
  Synthetic Construct
guuuccacuaga

SEQ ID NO: 501 - RNA, 12 nt, Artificial Sequence
  Synthetic Construct
uuuccacuagau

SEQ ID NO: 502 - RNA, 12 nt, Artificial Sequence
  Synthetic Construct
uuccacuagauu

SEQ ID NO: 503 - RNA, 12 nt, Artificial Sequence
  Synthetic Construct
uccacuagauuu

SEQ ID NO: 504 - RNA, 12 nt, Artificial Sequence
  Synthetic Construct
ccacuagauuua

SEQ ID NO: 505 - RNA, 12 nt, Artificial Sequence
  Synthetic Construct
cacuagauuuau

SEQ ID NO: 506 - RNA, 12 nt, Artificial Sequence
  Synthetic Construct
acuagauuuaua

SEQ ID NO: 507 - RNA, 15 nt, Artificial Sequence
  Synthetic Construct
ccuaaaaauguagca

SEQ ID NO: 508 - RNA, 15 nt, Artificial Sequence
  Synthetic Construct
cuaaaaauguagcag

SEQ ID NO: 509 - RNA, 15 nt, Artificial Sequence
  Synthetic Construct
uaaaaauguagcaga

SEQ ID NO: 510 - RNA, 15 nt, Artificial Sequence
  Synthetic Construct
aaaaauguagcagaa

SEQ ID NO: 511 - RNA, 15 nt, Artificial Sequence
  Synthetic Construct
aaaauguagcagaag

SEQ ID NO: 512 - RNA, 15 nt, Artificial Sequence
  Synthetic Construct
aaauguagcagaagc

SEQ ID NO: 513 - RNA, 15 nt, Artificial Sequence
  Synthetic Construct
aauguagcagaagca

SEQ ID NO: 514 - RNA, 15 nt, Artificial Sequence
  Synthetic Construct
auguagcagaagcau

SEQ ID NO: 515 - RNA, 15 nt, Artificial Sequence
  Synthetic Construct
uguagcagaagcauu

SEQ ID NO: 516 - RNA, 15 nt, Artificial Sequence
  Synthetic Construct
guagcagaagcauuu

SEQ ID NO: 517 - RNA, 15 nt, Artificial Sequence
  Synthetic Construct
uagcagaagcauuuc

SEQ ID NO: 518 - RNA, 15 nt, Artificial Sequence
  Synthetic Construct
agcagaagcauuucc

SEQ ID NO: 519 - RNA, 15 nt, Artificial Sequence
  Synthetic Construct
gcagaagcauuuccg

SEQ ID NO: 520 - RNA, 15 nt, Artificial Sequence
  Synthetic Construct
cagaagcauuuccgc

SEQ ID NO: 521 - RNA, 15 nt, Artificial Sequence
  Synthetic Construct
agaagcauuuccgca

SEQ ID NO: 522 - RNA, 15 nt, Artificial Sequence
  Synthetic Construct
gaagcauuuccgcac

SEQ ID NO: 523 - RNA, 15 nt, Artificial Sequence
  Synthetic Construct
aagcauuuccgcaca

SEQ ID NO: 524 - RNA, 15 nt, Artificial Sequence
  Synthetic Construct
agcauuuccgcacac

SEQ ID NO: 525 - RNA, 15 nt, Artificial Sequence
  Synthetic Construct
gcauuuccgcacacu

SEQ ID NO: 526 - RNA, 15 nt, Artificial Sequence
  Synthetic Construct
cauuuccgcacacug

SEQ ID NO: 527 - RNA, 15 nt, Artificial Sequence
  Synthetic Construct
auuuccgcacacugg

SEQ ID NO: 528 - RNA, 15 nt, Artificial Sequence
  Synthetic Construct
uuuccgcacacuggu

SEQ ID NO: 529 - RNA, 15 nt, Artificial Sequence
  Synthetic Construct
uuccgcacacuggug

SEQ ID NO: 530 - RNA, 15 nt, Artificial Sequence
  Synthetic Construct
uccgcacacuggugu

SEQ ID NO: 531 - RNA, 15 nt, Artificial Sequence
  Synthetic Construct
ccgcacacugguguc

SEQ ID NO: 532 - RNA, 15 nt, Artificial Sequence
  Synthetic Construct
cgcacacuggugucc

SEQ ID NO: 533 - RNA, 15 nt, Artificial Sequence
  Synthetic Construct
gcacacuggugucca

SEQ ID NO: 534 - RNA, 15 nt, Artificial Sequence
  Synthetic Construct
cacacugguguccag

SEQ ID NO: 535 - RNA, 15 nt, Artificial Sequence
  Synthetic Construct
acacugguguccaga

SEQ ID NO: 536 - RNA, 15 nt, Artificial Sequence
  Synthetic Construct
cacugguguccagaa

SEQ ID NO: 537 - RNA, 15 nt, Artificial Sequence
  Synthetic Construct
acugguguccagaau

SEQ ID NO: 538 - RNA, 15 nt, Artificial Sequence
  Synthetic Construct
cugguguccagaauc

SEQ ID NO: 539 - RNA, 15 nt, Artificial Sequence
  Synthetic Construct
ugguguccagaaucu

SEQ ID NO: 540 - RNA, 15 nt, Artificial Sequence
  Synthetic Construct
gguguccagaaucua

SEQ ID NO: 541 - RNA, 15 nt, Artificial Sequence
  Synthetic Construct
guguccagaaucuag

SEQ ID NO: 542 - RNA, 15 nt, Artificial Sequence
  Synthetic Construct
uguccagaaucuagu

SEQ ID NO: 543 - RNA, 15 nt, Artificial Sequence
  Synthetic Construct
guccagaaucuaguu

SEQ ID NO: 544 - RNA, 15 nt, Artificial Sequence
  Synthetic Construct
uccagaaucuaguuu

SEQ ID NO: 545 - RNA, 15 nt, Artificial Sequence
  Synthetic Construct
ccagaaucuaguuug

SEQ ID NO: 546 - RNA, 15 nt, Artificial Sequence
  Synthetic Construct
cagaaucuaguuugu

SEQ ID NO: 547 - RNA, 15 nt, Artificial Sequence
  Synthetic Construct
agaaucuaguuugug

SEQ ID NO: 548 - RNA, 15 nt, Artificial Sequence
  Synthetic Construct
gaaucuaguuugugc

SEQ ID NO: 549 - RNA, 15 nt, Artificial Sequence
  Synthetic Construct
aaucuaguuugugca

SEQ ID NO: 550 - RNA, 15 nt, Artificial Sequence
  Synthetic Construct
aucuaguuugugcag

SEQ ID NO: 551 - RNA, 15 nt, Artificial Sequence
  Synthetic Construct
ucuaguuugugcaga

SEQ ID NO: 552 - RNA, 15 nt, Artificial Sequence
  Synthetic Construct
cuaguuugugcagaa

SEQ ID NO: 553 - RNA, 15 nt, Artificial Sequence
  Synthetic Construct
uaguuugugcagaaa

SEQ ID NO: 554 - RNA, 15 nt, Artificial Sequence
  Synthetic Construct
aguuugugcagaaau

SEQ ID NO: 555 - RNA, 15 nt, Artificial Sequence
  Synthetic Construct
guuugugcagaaaug

SEQ ID NO: 556 - RNA, 15 nt, Artificial Sequence
  Synthetic Construct
uuugugcagaaaugu

SEQ ID NO: 557 - RNA, 15 nt, Artificial Sequence
  Synthetic Construct
uugugcagaaauguu

SEQ ID NO: 558 - RNA, 15 nt, Artificial Sequence
  Synthetic Construct
ugugcagaaauguuu

SEQ ID NO: 559 - RNA, 15 nt, Artificial Sequence
  Synthetic Construct
gugcagaaauguuuc

SEQ ID NO: 560 - RNA, 15 nt, Artificial Sequence
  Synthetic Construct
ugcagaaauguuucc

SEQ ID NO: 561 - RNA, 15 nt, Artificial Sequence
  Synthetic Construct
gcagaaauguuucca

SEQ ID NO: 562 - RNA, 15 nt, Artificial Sequence
  Synthetic Construct
cagaaauguuuccac

SEQ ID NO: 563 - RNA, 15 nt, Artificial Sequence
  Synthetic Construct
agaaauguuuccacu

SEQ ID NO: 564 - RNA, 15 nt, Artificial Sequence
  Synthetic Construct
gaaauguuuccacua

SEQ ID NO: 565 - RNA, 15 nt, Artificial Sequence
  Synthetic Construct
aaauguuuccacuag

SEQ ID NO: 566 - RNA, 15 nt, Artificial Sequence
  Synthetic Construct
aauguuuccacuaga

SEQ ID NO: 567 - RNA, 15 nt, Artificial Sequence
  Synthetic Construct
auguuuccacuagau

SEQ ID NO: 568 - RNA, 15 nt, Artificial Sequence
  Synthetic Construct
uguuuccacuagauu

SEQ ID NO: 569 - RNA, 15 nt, Artificial Sequence
  Synthetic Construct
guuuccacuagauuu

SEQ ID NO: 570 - RNA, 15 nt, Artificial Sequence
  Synthetic Construct
uuuccacuagauuua

SEQ ID NO: 571 - RNA, 15 nt, Artificial Sequence
  Synthetic Construct
uuccacuagauuuau

SEQ ID NO: 572 - RNA, 15 nt, Artificial Sequence
  Synthetic Construct
uccacuagauuuaua

SEQ ID NO: 573 - RNA, 20 nt, Artificial Sequence
  Synthetic Construct
ccuaaaaauguagcagaagc

SEQ ID NO: 574 - RNA, 20 nt, Artificial Sequence
  Synthetic Construct
cuaaaaauguagcagaagca

SEQ ID NO: 575 - RNA, 20 nt, Artificial Sequence
  Synthetic Construct
uaaaaauguagcagaagcau

SEQ ID NO: 576 - RNA, 20 nt, Artificial Sequence
  Synthetic Construct
aaaaauguagcagaagcauu

SEQ ID NO: 577 - RNA, 20 nt, Artificial Sequence
  Synthetic Construct
aaaauguagcagaagcauuu

SEQ ID NO: 578 - RNA, 20 nt, Artificial Sequence
  Synthetic Construct
aaauguagcagaagcauuuc

SEQ ID NO: 579 - RNA, 20 nt, Artificial Sequence
  Synthetic Construct
aauguagcagaagcauuucc

SEQ ID NO: 580 - RNA, 20 nt, Artificial Sequence
  Synthetic Construct
auguagcagaagcauuuccg

SEQ ID NO: 581 - RNA, 20 nt, Artificial Sequence
  Synthetic Construct
uguagcagaagcauuuccgc

SEQ ID NO: 582 - RNA, 20 nt, Artificial Sequence
  Synthetic Construct
guagcagaagcauuuccgca

SEQ ID NO: 583 - RNA, 20 nt, Artificial Sequence
  Synthetic Construct
uagcagaagcauuuccgcac

SEQ ID NO: 584 - RNA, 20 nt, Artificial Sequence
  Synthetic Construct
agcagaagcauuuccgcaca

SEQ ID NO: 585 - RNA, 20 nt, Artificial Sequence
  Synthetic Construct
gcagaagcauuuccgcacac

SEQ ID NO: 586 - RNA, 20 nt, Artificial Sequence
  Synthetic Construct
cagaagcauuuccgcacacu

SEQ ID NO: 587 - RNA, 20 nt, Artificial Sequence
  Synthetic Construct
agaagcauuuccgcacacug

SEQ ID NO: 588 - RNA, 20 nt, Artificial Sequence
  Synthetic Construct
gaagcauuuccgcacacugg

SEQ ID NO: 589 - RNA, 20 nt, Artificial Sequence
  Synthetic Construct
aagcauuuccgcacacuggu

SEQ ID NO: 590 - RNA, 20 nt, Artificial Sequence
  Synthetic Construct
agcauuuccgcacacuggug

SEQ ID NO: 591 - RNA, 20 nt, Artificial Sequence
  Synthetic Construct
gcauuuccgcacacuggugu

SEQ ID NO: 592 - RNA, 20 nt, Artificial Sequence
  Synthetic Construct
cauuuccgcacacugguguc

SEQ ID NO: 593 - RNA, 20 nt, Artificial Sequence
  Synthetic Construct
auuuccgcacacuggugucc

SEQ ID NO: 594 - RNA, 20 nt, Artificial Sequence
  Synthetic Construct
uuuccgcacacuggugucca

SEQ ID NO: 595 - RNA, 20 nt, Artificial Sequence
  Synthetic Construct
uuccgcacacugguguccag

SEQ ID NO: 596 - RNA, 20 nt, Artificial Sequence
  Synthetic Construct
uccgcacacugguguccaga

SEQ ID NO: 597 - RNA, 20 nt, Artificial Sequence
  Synthetic Construct
ccgcacacugguguccagaa

SEQ ID NO: 598 - RNA, 20 nt, Artificial Sequence
  Synthetic Construct
cgcacacugguguccagaau

SEQ ID NO: 599 - RNA, 20 nt, Artificial Sequence
  Synthetic Construct
gcacacugguguccagaauc

SEQ ID NO: 600 - RNA, 20 nt, Artificial Sequence
  Synthetic Construct
cacacugguguccagaaucu

SEQ ID NO: 601 - RNA, 20 nt, Artificial Sequence
  Synthetic Construct
acacugguguccagaaucua

SEQ ID NO: 602 - RNA, 20 nt, Artificial Sequence
  Synthetic Construct
cacugguguccagaaucuag

SEQ ID NO: 603 - RNA, 20 nt, Artificial Sequence
  Synthetic Construct
acugguguccagaaucuagu

SEQ ID NO: 604 - RNA, 20 nt, Artificial Sequence
  Synthetic Construct
cugguguccagaaucuaguu

SEQ ID NO: 605 - RNA, 20 nt, Artificial Sequence
  Synthetic Construct
ugguguccagaaucuaguuu

SEQ ID NO: 606 - RNA, 20 nt, Artificial Sequence
  Synthetic Construct
gguguccagaaucuaguuug

SEQ ID NO: 607 - RNA, 20 nt, Artificial Sequence
  Synthetic Construct
guguccagaaucuaguuugu

SEQ ID NO: 608 - RNA, 20 nt, Artificial Sequence
  Synthetic Construct
uguccagaaucuaguuugug

SEQ ID NO: 609 - RNA, 20 nt, Artificial Sequence
  Synthetic Construct
guccagaaucuaguuugugc

SEQ ID NO: 610 - RNA, 20 nt, Artificial Sequence
  Synthetic Construct
uccagaaucuaguuugugca

SEQ ID NO: 611 - RNA, 20 nt, Artificial Sequence
  Synthetic Construct
ccagaaucuaguuugugcag

SEQ ID NO: 612 - RNA, 20 nt, Artificial Sequence
  Synthetic Construct
cagaaucuaguuugugcaga

SEQ ID NO: 613 - RNA, 20 nt, Artificial Sequence
  Synthetic Construct
agaaucuaguuugugcagaa

SEQ ID NO: 614 - RNA, 20 nt, Artificial Sequence
  Synthetic Construct
gaaucuaguuugugcagaaa

SEQ ID NO: 615 - RNA, 20 nt, Artificial Sequence
  Synthetic Construct
aaucuaguuugugcagaaau

SEQ ID NO: 616 - RNA, 20 nt, Artificial Sequence
  Synthetic Construct
aucuaguuugugcagaaaug

SEQ ID NO: 617 - RNA, 20 nt, Artificial Sequence
  Synthetic Construct
ucuaguuugugcagaaaugu

SEQ ID NO: 618 - RNA, 20 nt, Artificial Sequence
  Synthetic Construct
cuaguuugugcagaaauguu

SEQ ID NO: 619 - RNA, 20 nt, Artificial Sequence
  Synthetic Construct
uaguuugugcagaaauguuu

SEQ ID NO: 620 - RNA, 20 nt, Artificial Sequence
  Synthetic Construct
aguuugugcagaaauguuuc

SEQ ID NO: 621 - RNA, 20 nt, Artificial Sequence
  Synthetic Construct
guuugugcagaaauguuucc

SEQ ID NO: 622 - RNA, 20 nt, Artificial Sequence
  Synthetic Construct
uuugugcagaaauguuucca

SEQ ID NO: 623 - RNA, 20 nt, Artificial Sequence
  Synthetic Construct
uugugcagaaauguuuccac

SEQ ID NO: 624 - RNA, 20 nt, Artificial Sequence
  Synthetic Construct
ugugcagaaauguuuccacu

SEQ ID NO: 625 - RNA, 20 nt, Artificial Sequence
  Synthetic Construct
gugcagaaauguuuccacua

SEQ ID NO: 626 - RNA, 20 nt, Artificial Sequence
  Synthetic Construct
ugcagaaauguuuccacuag

SEQ ID NO: 627 - RNA, 20 nt, Artificial Sequence
  Synthetic Construct
gcagaaauguuuccacuaga

SEQ ID NO: 628 - RNA, 20 nt, Artificial Sequence
  Synthetic Construct
cagaaauguuuccacuagau

SEQ ID NO: 629 - RNA, 20 nt, Artificial Sequence
  Synthetic Construct
agaaauguuuccacuagauu

SEQ ID NO: 630 - RNA, 20 nt, Artificial Sequence
  Synthetic Construct
gaaauguuuccacuagauuu

SEQ ID NO: 631 - RNA, 20 nt, Artificial Sequence
  Synthetic Construct
aaauguuuccacuagauuua

SEQ ID NO: 632 - RNA, 20 nt, Artificial Sequence
  Synthetic Construct
aauguuuccacuagauuuau

SEQ ID NO: 633 - RNA, 20 nt, Artificial Sequence
  Synthetic Construct
auguuuccacuagauuuaua

SEQ ID NO: 634 - RNA, 24 nt, Artificial Sequence
  Synthetic Construct
ccuaaaaauguagcagaagcauuu

SEQ ID NO: 635 - RNA, 24 nt, Artificial Sequence
  Synthetic Construct
cuaaaaauguagcagaagcauuuc

SEQ ID NO: 636 - RNA, 24 nt, Artificial Sequence
  Synthetic Construct
uaaaaauguagcagaagcauuucc

SEQ ID NO: 637 - RNA, 24 nt, Artificial Sequence
  Synthetic Construct
aaaaauguagcagaagcauuuccg

SEQ ID NO: 638 - RNA, 24 nt, Artificial Sequence
  Synthetic Construct
aaaauguagcagaagcauuuccgc

SEQ ID NO: 639 - RNA, 24 nt, Artificial Sequence
  Synthetic Construct
aaauguagcagaagcauuuccgca

SEQ ID NO: 640 - RNA, 24 nt, Artificial Sequence
  Synthetic Construct
aauguagcagaagcauuuccgcac

SEQ ID NO: 641 - RNA, 24 nt, Artificial Sequence
  Synthetic Construct
auguagcagaagcauuuccgcaca

SEQ ID NO: 642 - RNA, 24 nt, Artificial Sequence
  Synthetic Construct
uguagcagaagcauuuccgcacac

SEQ ID NO: 643 - RNA, 24 nt, Artificial Sequence
  Synthetic Construct
guagcagaagcauuuccgcacacu

SEQ ID NO: 644 - RNA, 24 nt, Artificial Sequence
  Synthetic Construct
uagcagaagcauuuccgcacacug

SEQ ID NO: 645 - RNA, 24 nt, Artificial Sequence
  Synthetic Construct
agcagaagcauuuccgcacacugg

SEQ ID NO: 646 - RNA, 24 nt, Artificial Sequence
  Synthetic Construct
gcagaagcauuuccgcacacuggu

SEQ ID NO: 647 - RNA, 24 nt, Artificial Sequence
  Synthetic Construct
cagaagcauuuccgcacacuggug

SEQ ID NO: 648 - RNA, 24 nt, Artificial Sequence
  Synthetic Construct
agaagcauuuccgcacacuggugu

SEQ ID NO: 649 - RNA, 24 nt, Artificial Sequence
  Synthetic Construct
gaagcauuuccgcacacugguguc

SEQ ID NO: 650 - RNA, 24 nt, Artificial Sequence
  Synthetic Construct
aagcauuuccgcacacuggugucc

SEQ ID NO: 651 - RNA, 24 nt, Artificial Sequence
  Synthetic Construct
agcauuuccgcacacuggugucca

SEQ ID NO: 652 - RNA, 24 nt, Artificial Sequence
  Synthetic Construct
gcauuuccgcacacugguguccag

SEQ ID NO: 653 - RNA, 24 nt, Artificial Sequence
  Synthetic Construct
cauuuccgcacacugguguccaga

SEQ ID NO: 654 - RNA, 24 nt, Artificial Sequence
  Synthetic Construct
auuuccgcacacugguguccagaa

SEQ ID NO: 655 - RNA, 24 nt, Artificial Sequence
  Synthetic Construct
uuuccgcacacugguguccagaau

SEQ ID NO: 656 - RNA, 24 nt, Artificial Sequence
  Synthetic Construct
uuccgcacacugguguccagaauc

SEQ ID NO: 657 - RNA, 24 nt, Artificial Sequence
  Synthetic Construct
uccgcacacugguguccagaaucu

SEQ ID NO: 658 - RNA, 24 nt, Artificial Sequence
  Synthetic Construct
ccgcacacugguguccagaaucua

SEQ ID NO: 659 - RNA, 24 nt, Artificial Sequence
  Synthetic Construct
cgcacacugguguccagaaucuag

SEQ ID NO: 660 - RNA, 24 nt, Artificial Sequence
  Synthetic Construct
gcacacugguguccagaaucuagu

SEQ ID NO: 661 - RNA, 24 nt, Artificial Sequence
  Synthetic Construct
cacacugguguccagaaucuaguu

SEQ ID NO: 662 - RNA, 24 nt, Artificial Sequence
  Synthetic Construct
acacugguguccagaaucuaguuu

SEQ ID NO: 663 - RNA, 24 nt, Artificial Sequence
  Synthetic Construct
cacugguguccagaaucuaguuug

SEQ ID NO: 664 - RNA, 24 nt, Artificial Sequence
  Synthetic Construct
acugguguccagaaucuaguuugu

SEQ ID NO: 665 - RNA, 24 nt, Artificial Sequence
  Synthetic Construct
cugguguccagaaucuaguuugug

SEQ ID NO: 666 - RNA, 24 nt, Artificial Sequence
  Synthetic Construct
ugguguccagaaucuaguuugugc

SEQ ID NO: 667 - RNA, 24 nt, Artificial Sequence
  Synthetic Construct
gguguccagaaucuaguuugugca

SEQ ID NO: 668 - RNA, 24 nt, Artificial Sequence
  Synthetic Construct
guguccagaaucuaguuugugcag

SEQ ID NO: 669 - RNA, 24 nt, Artificial Sequence
  Synthetic Construct
uguccagaaucuaguuugugcaga

SEQ ID NO: 670 - RNA, 24 nt, Artificial Sequence
  Synthetic Construct
guccagaaucuaguuugugcagaa

SEQ ID NO: 671 - RNA, 24 nt, Artificial Sequence
  Synthetic Construct
uccagaaucuaguuugugcagaaa

SEQ ID NO: 672 - RNA, 24 nt, Artificial Sequence
  Synthetic Construct
ccagaaucuaguuugugcagaaau

SEQ ID NO: 673 - RNA, 24 nt, Artificial Sequence
  Synthetic Construct
cagaaucuaguuugugcagaaaug

SEQ ID NO: 674 - RNA, 24 nt, Artificial Sequence
  Synthetic Construct
agaaucuaguuugugcagaaaugu

SEQ ID NO: 675 - RNA, 24 nt, Artificial Sequence
  Synthetic Construct
gaaucuaguuugugcagaaauguu

SEQ ID NO: 676 - RNA, 24 nt, Artificial Sequence
  Synthetic Construct
aaucuaguuugugcagaaauguuu

SEQ ID NO: 677 - RNA, 24 nt, Artificial Sequence
  Synthetic Construct
aucuaguuugugcagaaauguuuc

SEQ ID NO: 678 - RNA, 24 nt, Artificial Sequence
  Synthetic Construct
ucuaguuugugcagaaauguuucc

SEQ ID NO: 679 - RNA, 24 nt, Artificial Sequence
  Synthetic Construct
cuaguuugugcagaaauguuucca

SEQ ID NO: 680 - RNA, 24 nt, Artificial Sequence
  Synthetic Construct
uaguuugugcagaaauguuuccac

SEQ ID NO: 681 - RNA, 24 nt, Artificial Sequence
  Synthetic Construct
aguuugugcagaaauguuuccacu

SEQ ID NO: 682 - RNA, 24 nt, Artificial Sequence
  Synthetic Construct
guuugugcagaaauguuuccacua

SEQ ID NO: 683 - RNA, 24 nt, Artificial Sequence
  Synthetic Construct
uuugugcagaaauguuuccacuag

SEQ ID NO: 684 - RNA, 24 nt, Artificial Sequence
  Synthetic Construct
uugugcagaaauguuuccacuaga

SEQ ID NO: 685 - RNA, 24 nt, Artificial Sequence
  Synthetic Construct
ugugcagaaauguuuccacuagau

SEQ ID NO: 686 - RNA, 30 nt, Artificial Sequence
  Synthetic Construct
ccuaaaaauguagcagaagcauuuccgcac

SEQ ID NO: 687 - RNA, 30 nt, Artificial Sequence
  Synthetic Construct
cuaaaaauguagcagaagcauuuccgcaca

SEQ ID NO: 688 - RNA, 30 nt, Artificial Sequence
  Synthetic Construct
uaaaaauguagcagaagcauuuccgcacac

SEQ ID NO: 689 - RNA, 30 nt, Artificial Sequence
  Synthetic Construct
aaaaauguagcagaagcauuuccgcacacu

SEQ ID NO: 690 - RNA, 30 nt, Artificial Sequence
  Synthetic Construct
aaaauguagcagaagcauuuccgcacacug

SEQ ID NO: 691 - RNA, 30 nt, Artificial Sequence
  Synthetic Construct
aaauguagcagaagcauuuccgcacacugg

SEQ ID NO: 692 - RNA, 30 nt, Artificial Sequence
  Synthetic Construct
aauguagcagaagcauuuccgcacacuggu

SEQ ID NO: 693 - RNA, 30 nt, Artificial Sequence
  Synthetic Construct
auguagcagaagcauuuccgcacacuggug

SEQ ID NO: 694 - RNA, 30 nt, Artificial Sequence
  Synthetic Construct
uguagcagaagcauuuccgcacacuggugu

SEQ ID NO: 695 - RNA, 30 nt, Artificial Sequence
  Synthetic Construct
guagcagaagcauuuccgcacacugguguc

SEQ ID NO: 696 - RNA, 30 nt, Artificial Sequence
  Synthetic Construct
uagcagaagcauuuccgcacacuggugucc

SEQ ID NO: 697 - RNA, 30 nt, Artificial Sequence
  Synthetic Construct
agcagaagcauuuccgcacacuggugucca

SEQ ID NO: 698 - RNA, 30 nt, Artificial Sequence
  Synthetic Construct
gcagaagcauuuccgcacacugguguccag

SEQ ID NO: 699 - RNA, 30 nt, Artificial Sequence
  Synthetic Construct
cagaagcauuuccgcacacugguguccaga

SEQ ID NO: 700 - RNA, 30 nt, Artificial Sequence
  Synthetic Construct
agaagcauuuccgcacacugguguccagaa

SEQ ID NO: 701 - RNA, 30 nt, Artificial Sequence
  Synthetic Construct
gaagcauuuccgcacacugguguccagaau

SEQ ID NO: 702 - RNA, 30 nt, Artificial Sequence
  Synthetic Construct
aagcauuuccgcacacugguguccagaauc

SEQ ID NO: 703 - RNA, 30 nt, Artificial Sequence
  Synthetic Construct
agcauuuccgcacacugguguccagaaucu

SEQ ID NO: 704 - RNA, 30 nt, Artificial Sequence
  Synthetic Construct
gcauuuccgcacacugguguccagaaucua

SEQ ID NO: 705 - RNA, 30 nt, Artificial Sequence
  Synthetic Construct
cauuuccgcacacugguguccagaaucuag

SEQ ID NO: 706 - RNA, 30 nt, Artificial Sequence
  Synthetic Construct
auuuccgcacacugguguccagaaucuagu

SEQ ID NO: 707 - RNA, 30 nt, Artificial Sequence
  Synthetic Construct
uuuccgcacacugguguccagaaucuaguu

SEQ ID NO: 708 - RNA, 30 nt, Artificial Sequence
  Synthetic Construct
uuccgcacacugguguccagaaucuaguuu

SEQ ID NO: 709 - RNA, 30 nt, Artificial Sequence
  Synthetic Construct
uccgcacacugguguccagaaucuaguuug

SEQ ID NO: 710 - RNA, 30 nt, Artificial Sequence
  Synthetic Construct
ccgcacacugguguccagaaucuaguuugu

SEQ ID NO: 711 - RNA, 30 nt, Artificial Sequence
  Synthetic Construct
cgcacacugguguccagaaucuaguuugug

SEQ ID NO: 712 - RNA, 30 nt, Artificial Sequence
  Synthetic Construct
gcacacugguguccagaaucuaguuugugc

SEQ ID NO: 713 - RNA, 30 nt, Artificial Sequence
  Synthetic Construct
cacacugguguccagaaucuaguuugugca

SEQ ID NO: 714 - RNA, 30 nt, Artificial Sequence
  Synthetic Construct
acacugguguccagaaucuaguuugugcag

SEQ ID NO: 715 - RNA, 30 nt, Artificial Sequence
  Synthetic Construct
cacugguguccagaaucuaguuugugcaga

SEQ ID NO: 716 - RNA, 30 nt, Artificial Sequence
  Synthetic Construct
acugguguccagaaucuaguuugugcagaa

SEQ ID NO: 717 - RNA, 30 nt, Artificial Sequence
  Synthetic Construct
cugguguccagaaucuaguuugugcagaaa

SEQ ID NO: 718 - RNA, 30 nt, Artificial Sequence
  Synthetic Construct
ugguguccagaaucuaguuugugcagaaau

SEQ ID NO: 719 - RNA, 30 nt, Artificial Sequence
  Synthetic Construct
gguguccagaaucuaguuugugcagaaaug

SEQ ID NO: 720 - RNA, 30 nt, Artificial Sequence
  Synthetic Construct
guguccagaaucuaguuugugcagaaaugu

SEQ ID NO: 721 - RNA, 30 nt, Artificial Sequence
  Synthetic Construct
guccagaaucuaguuugugcagaaauguuu

SEQ ID NO: 722 - RNA, 30 nt, Artificial Sequence
  Synthetic Construct
uccagaaucuaguuugugcagaaauguuuc

SEQ ID NO: 723 - RNA, 30 nt, Artificial Sequence
  Synthetic Construct
ccagaaucuaguuugugcagaaauguuucc

SEQ ID NO: 724 - RNA, 30 nt, Artificial Sequence
  Synthetic Construct
cagaaucuaguuugugcagaaauguuucca

SEQ ID NO: 725 - RNA, 30 nt, Artificial Sequence
  Synthetic Construct
agaaucuaguuugugcagaaauguuuccac

SEQ ID NO: 726 - RNA, 30 nt, Artificial Sequence
  Synthetic Construct
gaaucuaguuugugcagaaauguuuccacu

SEQ ID NO: 727 - RNA, 30 nt, Artificial Sequence
  Synthetic Construct
aaucuaguuugugcagaaauguuuccacua

SEQ ID NO: 728 - RNA, 30 nt, Artificial Sequence
  Synthetic Construct
aucuaguuugugcagaaauguuuccacuag

SEQ ID NO: 729 - RNA, 30 nt, Artificial Sequence
  Synthetic Construct
ucuaguuugugcagaaauguuuccacuaga

SEQ ID NO: 730 - RNA, 30 nt, Artificial Sequence
  Synthetic Construct
cuaguuugugcagaaauguuuccacuagau

SEQ ID NO: 731 - RNA, 30 nt, Artificial Sequence
  Synthetic Construct
uaguuugugcagaaauguuuccacuagauu

SEQ ID NO: 732 - RNA, 30 nt, Artificial Sequence
  Synthetic Construct
aguuugugcagaaauguuuccacuagauuu

SEQ ID NO: 733 - RNA, 30 nt, Artificial Sequence
  Synthetic Construct
guuugugcagaaauguuuccacuagauuua

SEQ ID NO: 734 - RNA, 30 nt, Artificial Sequence
  Synthetic Construct
uuugugcagaaauguuuccacuagauuuau

SEQ ID NO: 735 - RNA, 30 nt, Artificial Sequence
  Synthetic Construct
uugugcagaaauguuuccacuagauuuaua

SEQ ID NO: 736 - RNA, 80 nt, Artificial Sequence
  Synthetic Construct
ccuaaaaauguagcagaagcauuuccgcacacugguguccagaaucuaguuugugcagaaauguuuccacuagauuuaua

SEQ ID NO: 737 - RNA, 6 nt, Artificial Sequence
  Synthetic Construct
gugugc

SEQ ID NO: 738 - RNA, 6 nt, Artificial Sequence
  Synthetic Construct
ugugcg

SEQ ID NO: 739 - RNA, 6 nt, Artificial Sequence
  Synthetic Construct
gugcgg

SEQ ID NO: 740 - RNA, 6 nt, Artificial Sequence
  Synthetic Construct
ugcgga

SEQ ID NO: 741 - RNA, 6 nt, Artificial Sequence
  Synthetic Construct
gcggaa

SEQ ID NO: 742 - RNA, 6 nt, Artificial Sequence
  Synthetic Construct
cggaaa

SEQ ID NO: 743 - RNA, 6 nt, Artificial Sequence
  Synthetic Construct
ggaaau

SEQ ID NO: 744 - RNA, 6 nt, Artificial Sequence
  Synthetic Construct
gaaaug

SEQ ID NO: 745 - RNA, 6 nt, Artificial Sequence
  Synthetic Construct
aaaugc

SEQ ID NO: 746 - RNA, 6 nt, Artificial Sequence
  Synthetic Construct
aaugcu

SEQ ID NO: 747 - RNA, 6 nt, Artificial Sequence
  Synthetic Construct
augcuu

SEQ ID NO: 748 - RNA, 6 nt, Artificial Sequence
  Synthetic Construct
ugcuuc

SEQ ID NO: 749 - RNA, 6 nt, Artificial Sequence
  Synthetic Construct
gcuucu

SEQ ID NO: 750 - RNA, 6 nt, Artificial Sequence
  Synthetic Construct
cuucug

SEQ ID NO: 751 - RNA, 6 nt, Artificial Sequence
  Synthetic Construct
uucugc

SEQ ID NO: 752 - RNA, 6 nt, Artificial Sequence
  Synthetic Construct
ucugcu

SEQ ID NO: 753 - RNA, 6 nt, Artificial Sequence
  Synthetic Construct
cugcua

SEQ ID NO: 754 - RNA, 7 nt, Artificial Sequence
  Synthetic Construct
gugugcg

SEQ ID NO: 755 - RNA, 7 nt, Artificial Sequence
  Synthetic Construct
ugugcgg

SEQ ID NO: 756 - RNA, 7 nt, Artificial Sequence
  Synthetic Construct
gugcgga

SEQ ID NO: 757 - RNA, 7 nt, Artificial Sequence
  Synthetic Construct
ugcggaa

SEQ ID NO: 758 - RNA, 7 nt, Artificial Sequence
  Synthetic Construct
gcggaaa

SEQ ID NO: 759 - RNA, 7 nt, Artificial Sequence
  Synthetic Construct
cggaaau

SEQ ID NO: 760 - RNA, 7 nt, Artificial Sequence
  Synthetic Construct
ggaaaug

SEQ ID NO: 761 - RNA, 7 nt, Artificial Sequence
  Synthetic Construct
gaaaugc

SEQ ID NO: 762 - RNA, 7 nt, Artificial Sequence
  Synthetic Construct
aaaugcu

SEQ ID NO: 763 - RNA, 7 nt, Artificial Sequence
  Synthetic Construct
aaugcuu

SEQ ID NO: 764 - RNA, 7 nt, Artificial Sequence
  Synthetic Construct
augcuuc

SEQ ID NO: 765 - RNA, 7 nt, Artificial Sequence
  Synthetic Construct
ugcuucu

SEQ ID NO: 766 - RNA, 7 nt, Artificial Sequence
  Synthetic Construct
gcuucug

SEQ ID NO: 767 - RNA, 7 nt, Artificial Sequence
  Synthetic Construct
cuucugc

SEQ ID NO: 768 - RNA, 7 nt, Artificial Sequence
  Synthetic Construct
uucugcu

SEQ ID NO: 769 - RNA, 7 nt, Artificial Sequence
  Synthetic Construct
ucugcua

SEQ ID NO: 770 - RNA, 8 nt, Artificial Sequence
  Synthetic Construct
gugugcgg

SEQ ID NO: 771 - RNA, 8 nt, Artificial Sequence
  Synthetic Construct
ugugcgga

SEQ ID NO: 772 - RNA, 8 nt, Artificial Sequence
  Synthetic Construct
gugcggaa

SEQ ID NO: 773 - RNA, 8 nt, Artificial Sequence
  Synthetic Construct
ugcggaaa

SEQ ID NO: 774 - RNA, 8 nt, Artificial Sequence
  Synthetic Construct
gcggaaau

SEQ ID NO: 775 - RNA, 8 nt, Artificial Sequence
  Synthetic Construct
cggaaaug

SEQ ID NO: 776 - RNA, 8 nt, Artificial Sequence
  Synthetic Construct
ggaaaugc

SEQ ID NO: 777 - RNA, 8 nt, Artificial Sequence
  Synthetic Construct
gaaaugcu

SEQ ID NO: 778 - RNA, 8 nt, Artificial Sequence
  Synthetic Construct
aaaugcuu

SEQ ID NO: 779 - RNA, 8 nt, Artificial Sequence
  Synthetic Construct
aaugcuuc

SEQ ID NO: 780 - RNA, 8 nt, Artificial Sequence
  Synthetic Construct
augcuucu

SEQ ID NO: 781 - RNA, 8 nt, Artificial Sequence
  Synthetic Construct
ugcuucug

SEQ ID NO: 782 - RNA, 8 nt, Artificial Sequence
  Synthetic Construct
gcuucugc

SEQ ID NO: 783 - RNA, 8 nt, Artificial Sequence
  Synthetic Construct
cuucugcu

SEQ ID NO: 784 - RNA, 8 nt, Artificial Sequence
  Synthetic Construct
uucugcua

SEQ ID NO: 785 - RNA, 9 nt, Artificial Sequence
  Synthetic Construct
gugugcgga

SEQ ID NO: 786 - RNA, 9 nt, Artificial Sequence
  Synthetic Construct
ugugcggaa

SEQ ID NO: 787 - RNA, 9 nt, Artificial Sequence
  Synthetic Construct
gugcggaaa

SEQ ID NO: 788 - RNA, 9 nt, Artificial Sequence
  Synthetic Construct
ugcggaaau

SEQ ID NO: 789 - RNA, 9 nt, Artificial Sequence
  Synthetic Construct
gcggaaaug

SEQ ID NO: 790 - RNA, 9 nt, Artificial Sequence
  Synthetic Construct
cggaaaugc

SEQ ID NO: 791 - RNA, 9 nt, Artificial Sequence
  Synthetic Construct
ggaaaugcu

SEQ ID NO: 792 - RNA, 9 nt, Artificial Sequence
  Synthetic Construct
gaaaugcuu

SEQ ID NO: 793 - RNA, 9 nt, Artificial Sequence
  Synthetic Construct
aaaugcuuc

SEQ ID NO: 794 - RNA, 9 nt, Artificial Sequence
  Synthetic Construct
aaugcuucu

SEQ ID NO: 795 - RNA, 9 nt, Artificial Sequence
  Synthetic Construct
augcuucug

SEQ ID NO: 796 - RNA, 9 nt, Artificial Sequence
  Synthetic Construct
ugcuucugc

SEQ ID NO: 797 - RNA, 9 nt, Artificial Sequence
  Synthetic Construct
gcuucugcu

SEQ ID NO: 798 - RNA, 9 nt, Artificial Sequence
  Synthetic Construct
cuucugcua

SEQ ID NO: 799 - RNA, 10 nt, Artificial Sequence
  Synthetic Construct
gugugcggaa

SEQ ID NO: 800 - RNA, 10 nt, Artificial Sequence
  Synthetic Construct
ugugcggaaa

SEQ ID NO: 801 - RNA, 10 nt, Artificial Sequence
  Synthetic Construct
gugcggaaau

SEQ ID NO: 802 - RNA, 10 nt, Artificial Sequence
  Synthetic Construct
ugcggaaaug

SEQ ID NO: 803 - RNA, 10 nt, Artificial Sequence
  Synthetic Construct
gcggaaaugc

SEQ ID NO: 804 - RNA, 10 nt, Artificial Sequence
  Synthetic Construct
cggaaaugcu

SEQ ID NO: 805 - RNA, 10 nt, Artificial Sequence
  Synthetic Construct
ggaaaugcuu

SEQ ID NO: 806 - RNA, 10 nt, Artificial Sequence
  Synthetic Construct
gaaaugcuuc

SEQ ID NO: 807 - RNA, 10 nt, Artificial Sequence
  Synthetic Construct
aaaugcuucu

SEQ ID NO: 808 - RNA, 10 nt, Artificial Sequence
  Synthetic Construct
aaugcuucug

SEQ ID NO: 809 - RNA, 10 nt, Artificial Sequence
  Synthetic Construct
augcuucugc

SEQ ID NO: 810 - RNA, 10 nt, Artificial Sequence
  Synthetic Construct
ugcuucugcu

SEQ ID NO: 811 - RNA, 10 nt, Artificial Sequence
  Synthetic Construct
gcuucugcua

SEQ ID NO: 812 - RNA, 11 nt, Artificial Sequence
  Synthetic Construct
gugugcggaaa

SEQ ID NO: 813 - RNA, 11 nt, Artificial Sequence
  Synthetic Construct
ugugcggaaau

SEQ ID NO: 814 - RNA, 11 nt, Artificial Sequence
  Synthetic Construct
gugcggaaaug

SEQ ID NO: 815 - RNA, 11 nt, Artificial Sequence
  Synthetic Construct
ugcggaaaugc

SEQ ID NO: 816 - RNA, 11 nt, Artificial Sequence
  Synthetic Construct
gcggaaaugcu

SEQ ID NO: 817 - RNA, 11 nt, Artificial Sequence
  Synthetic Construct
cggaaaugcuu

SEQ ID NO: 818 - RNA, 11 nt, Artificial Sequence
  Synthetic Construct
ggaaaugcuuc

SEQ ID NO: 819 - RNA, 11 nt, Artificial Sequence
  Synthetic Construct
gaaaugcuucu

SEQ ID NO: 820 - RNA, 11 nt, Artificial Sequence
  Synthetic Construct
aaaugcuucug

SEQ ID NO: 821 - RNA, 11 nt, Artificial Sequence
  Synthetic Construct
aaugcuucugc

SEQ ID NO: 822 - RNA, 11 nt, Artificial Sequence
  Synthetic Construct
augcuucugcu

SEQ ID NO: 823 - RNA, 11 nt, Artificial Sequence
  Synthetic Construct
ugcuucugcua

SEQ ID NO: 824 - RNA, 12 nt, Artificial Sequence
  Synthetic Construct
gugugcggaaau

SEQ ID NO: 825 - RNA, 12 nt, Artificial Sequence
  Synthetic Construct
ugugcggaaaug

SEQ ID NO: 826 - RNA, 12 nt, Artificial Sequence
  Synthetic Construct
gugcggaaaugc

SEQ ID NO: 827 - RNA, 12 nt, Artificial Sequence
  Synthetic Construct
ugcggaaaugcu

SEQ ID NO: 828 - RNA, 12 nt, Artificial Sequence
  Synthetic Construct
gcggaaaugcuu

SEQ ID NO: 829 - RNA, 12 nt, Artificial Sequence
  Synthetic Construct
cggaaaugcuuc

SEQ ID NO: 830 - RNA, 12 nt, Artificial Sequence
  Synthetic Construct
ggaaaugcuucu

SEQ ID NO: 831 - RNA, 12 nt, Artificial Sequence
  Synthetic Construct
gaaaugcuucug

SEQ ID NO: 832 - RNA, 12 nt, Artificial Sequence
  Synthetic Construct
aaaugcuucugc

SEQ ID NO: 833 - RNA, 12 nt, Artificial Sequence
  Synthetic Construct
aaugcuucugcu

SEQ ID NO: 834 - RNA, 12 nt, Artificial Sequence
  Synthetic Construct
augcuucugcua

SEQ ID NO: 835 - RNA, 13 nt, Artificial Sequence
  Synthetic Construct
gugugcggaaaug

SEQ ID NO: 836 - RNA, 13 nt, Artificial Sequence
  Synthetic Construct
ugugcggaaaugc

SEQ ID NO: 837 - RNA, 13 nt, Artificial Sequence
  Synthetic Construct
gugcggaaaugcu

SEQ ID NO: 838 - RNA, 13 nt, Artificial Sequence
  Synthetic Construct
ugcggaaaugcuu

SEQ ID NO: 839 - RNA, 13 nt, Artificial Sequence
  Synthetic Construct
gcggaaaugcuuc

SEQ ID NO: 840 - RNA, 13 nt, Artificial Sequence
  Synthetic Construct
cggaaaugcuucu

SEQ ID NO: 841 - RNA, 13 nt, Artificial Sequence
  Synthetic Construct
ggaaaugcuucug

SEQ ID NO: 842 - RNA, 13 nt, Artificial Sequence
  Synthetic Construct
gaaaugcuucugc

SEQ ID NO: 843 - RNA, 13 nt, Artificial Sequence
  Synthetic Construct
aaaugcuucugcu

SEQ ID NO: 844 - RNA, 13 nt, Artificial Sequence
  Synthetic Construct
aaugcuucugcua

SEQ ID NO: 845 - RNA, 14 nt, Artificial Sequence
  Synthetic Construct
gugugcggaaaugc

SEQ ID NO: 846 - RNA, 14 nt, Artificial Sequence
  Synthetic Construct
ugugcggaaaugcu

SEQ ID NO: 847 - RNA, 14 nt, Artificial Sequence
  Synthetic Construct
gugcggaaaugcuu

SEQ ID NO: 848 - RNA, 14 nt, Artificial Sequence
  Synthetic Construct
ugcggaaaugcuuc

SEQ ID NO: 849 - RNA, 14 nt, Artificial Sequence
  Synthetic Construct
gcggaaaugcuucu

SEQ ID NO: 850 - RNA, 14 nt, Artificial Sequence
  Synthetic Construct
cggaaaugcuucug

SEQ ID NO: 851 - RNA, 14 nt, Artificial Sequence
  Synthetic Construct
ggaaaugcuucugc

SEQ ID NO: 852 - RNA, 14 nt, Artificial Sequence
  Synthetic Construct
gaaaugcuucugcu

SEQ ID NO: 853 - RNA, 14 nt, Artificial Sequence
  Synthetic Construct
aaaugcuucugcua

SEQ ID NO: 854 - RNA, 15 nt, Artificial Sequence
  Synthetic Construct
gugugcggaaaugcu

SEQ ID NO: 855 - RNA, 15 nt, Artificial Sequence
  Synthetic Construct
ugugcggaaaugcuu

SEQ ID NO: 856 - RNA, 15 nt, Artificial Sequence
  Synthetic Construct
gugcggaaaugcuuc

SEQ ID NO: 857 - RNA, 15 nt, Artificial Sequence
  Synthetic Construct
ugcggaaaugcuucu

SEQ ID NO: 858 - RNA, 15 nt, Artificial Sequence
  Synthetic Construct
gcggaaaugcuucug

SEQ ID NO: 859 - RNA, 15 nt, Artificial Sequence
  Synthetic Construct
cggaaaugcuucugc

SEQ ID NO: 860 - RNA, 15 nt, Artificial Sequence
  Synthetic Construct
ggaaaugcuucugcu

SEQ ID NO: 861 - RNA, 15 nt, Artificial Sequence
  Synthetic Construct
gaaaugcuucugcua

SEQ ID NO: 862 - RNA, 16 nt, Artificial Sequence
  Synthetic Construct
gugugcggaaaugcuu

SEQ ID NO: 863 - RNA, 16 nt, Artificial Sequence
  Synthetic Construct
ugugcggaaaugcuuc

SEQ ID NO: 864 - RNA, 16 nt, Artificial Sequence
  Synthetic Construct
gugcggaaaugcuucu

SEQ ID NO: 865 - RNA, 16 nt, Artificial Sequence
  Synthetic Construct
ugcggaaaugcuucug

SEQ ID NO: 866 - RNA, 16 nt, Artificial Sequence
  Synthetic Construct
gcggaaaugcuucugc

SEQ ID NO: 867 - RNA, 16 nt, Artificial Sequence
  Synthetic Construct
cggaaaugcuucugcu

SEQ ID NO: 868 - RNA, 16 nt, Artificial Sequence
  Synthetic Construct
ggaaaugcuucugcua

SEQ ID NO: 869 - RNA, 17 nt, Artificial Sequence
  Synthetic Construct
gugugcggaaaugcuuc

SEQ ID NO: 870 - RNA, 17 nt, Artificial Sequence
  Synthetic Construct
ugugcggaaaugcuucu

SEQ ID NO: 871 - RNA, 17 nt, Artificial Sequence
  Synthetic Construct
gugcggaaaugcuucug

SEQ ID NO: 872 - RNA, 17 nt, Artificial Sequence
  Synthetic Construct
ugcggaaaugcuucugc

SEQ ID NO: 873 - RNA, 17 nt, Artificial Sequence
  Synthetic Construct
gcggaaaugcuucugcu

SEQ ID NO: 874 - RNA, 17 nt, Artificial Sequence
  Synthetic Construct
cggaaaugcuucugcua

SEQ ID NO: 875 - RNA, 18 nt, Artificial Sequence
  Synthetic Construct
gugugcggaaaugcuucu

SEQ ID NO: 876 - RNA, 18 nt, Artificial Sequence
  Synthetic Construct
ugugcggaaaugcuucug

SEQ ID NO: 877 - RNA, 18 nt, Artificial Sequence
  Synthetic Construct
gugcggaaaugcuucugc

SEQ ID NO: 878 - RNA, 18 nt, Artificial Sequence
  Synthetic Construct
ugcggaaaugcuucugcu

SEQ ID NO: 879 - RNA, 18 nt, Artificial Sequence
  Synthetic Construct
gcggaaaugcuucugcua

SEQ ID NO: 880 - RNA, 19 nt, Artificial Sequence
  Synthetic Construct
gugugcggaaaugcuucug

SEQ ID NO: 881 - RNA, 19 nt, Artificial Sequence
  Synthetic Construct
ugugcggaaaugcuucugc

SEQ ID NO: 882 - RNA, 19 nt, Artificial Sequence
  Synthetic Construct
gugcggaaaugcuucugcu

SEQ ID NO: 883 - RNA, 19 nt, Artificial Sequence
  Synthetic Construct
ugcggaaaugcuucugcua

SEQ ID NO: 884 - RNA, 20 nt, Artificial Sequence
  Synthetic Construct
gugugcggaaaugcuucugc

SEQ ID NO: 885 - RNA, 20 nt, Artificial Sequence
  Synthetic Construct
ugugcggaaaugcuucugcu

SEQ ID NO: 886 - RNA, 20 nt, Artificial Sequence
  Synthetic Construct
gugcggaaaugcuucugcua

SEQ ID NO: 887 - RNA, 21 nt, Artificial Sequence
  Synthetic Construct
gugugcggaaaugcuucugcu

SEQ ID NO: 888 - RNA, 21 nt, Artificial Sequence
  Synthetic Construct
ugugcggaaaugcuucugcua

SEQ ID NO: 889 - RNA, 22 nt, Artificial Sequence
  Synthetic Construct
gugugcggaaaugcuucugcua

SEQ ID NO: 890 - DNA, 20 bp, Artificial Sequence
  Synthetic Construct
agcagaagcatttccgcaca

SEQ ID NO: 891 - DNA, 19 bp, Artificial Sequence
  Synthetic Construct
taacacgtctatacgccca

SEQ ID NO: 892 - DNA, 29 bp, Artificial Sequence
  Synthetic Construct
agcttgctcatcagttgccacttccacat

SEQ ID NO: 893 - DNA, 29 bp, Artificial Sequence
  Synthetic Construct
aggccacattcacgtatatgataccaaca

SEQ ID NO: 894 - DNA, 29 bp, Artificial Sequence
  Synthetic Construct
tacgttgtgagtggtattattcagcacga

SEQ ID NO: 895 - DNA, 29 bp, Artificial Sequence
  Synthetic Construct
acaagaacctactgctaatgccaccacta

SEQ ID NO: 896 - DNA, 29 bp, Artificial Sequence
  Synthetic Construct
gtatgaagagcaagccttccaggacctga

SEQ ID NO: 897 - DNA, 29 bp, Artificial Sequence
  Synthetic Construct
agcactgcttcagtgccatgacaaacatc

SEQ ID NO: 898 - DNA, 29 bp, Artificial Sequence
  Synthetic Construct
cctggtggcagcacctcacatttgatgtg

SEQ ID NO: 899 - DNA, 29 bp, Artificial Sequence
  Synthetic Construct
ggctgtgtctgagaagagtaacttcctat

SEQ ID NO: 900 - DNA, 5 bp, Artificial Sequence
  Synthetic Construct
cgcac

SEQ ID NO: 901 - DNA, 5 bp, Artificial Sequence
  Synthetic Construct
gcgtg

SEQ ID NO: 902 - DNA, 20 bp, Artificial Sequence
  Synthetic Construct
agagtactctataaatctag

SEQ ID NO: 903 - DNA, 20 bp, Artificial Sequence
  Synthetic Construct
tttctgcacaaactagattc

SEQ ID NO: 904 - DNA, 20 bp, Artificial Sequence
  Synthetic Construct
agattctggacaccagtgtg

SEQ ID NO: 905 - DNA, 20 bp, Artificial Sequence
  Synthetic Construct
gcagaagcatttccgcacac

SEQ ID NO: 906 - RNA, 23 nt, Artificial Sequence
  Synthetic Construct
uuuguaacuugccauccgcacag

SEQ ID NO: 907 - DNA, 23 bp, Artificial Sequence
  Synthetic Construct
tttgtaacttgccatcgcgtgag

SEQ ID NO: 908 - DNA, 23 bp, Artificial Sequence
  Synthetic Construct
tttgtaacttgccatccgcacag

SEQ ID NO: 909 - RNA, 23 nt, Artificial Sequence
  Synthetic Construct
auaaauuguuauuuucgcacaau

SEQ ID NO: 910 - DNA, 23 bp, Artificial Sequence
  Synthetic Construct
ataaattgttattttgcgtgtat

SEQ ID NO: 911 - DNA, 23 bp, Artificial Sequence
  Synthetic Construct
ataaattgttattttcgcacaat

(19) United States
(12) Patent Application Publication
(10) Pub. No.: US 2022/0133767 A1
(43) Pub. Date: May 5, 2022

(54) TARGETING MICRORNAS TO OVERCOME DRUG TOLERANCE AND RESISTANCE

(71) Applicant: Beth Israel Deaconess Medical Center, Inc., Boston, MA (US)

(72) Inventors: Frank SLACK, Waban, MA (US); Wen Cai ZHANG, Brookline, MA (US)

(21) Appl. No.: 17/431,474

(22) PCT Filed: Feb. 19, 2020
(86) PCT No.: PCT/US2020/018826

§ 371 Date: Aug. 17, 2021

Related U.S. Application Data
(60) Provisional application No. 62/807,483, filed on Feb. 19, 2019.

(51) Int. Cl. A61K 31/7088; A61K 31/506; A61K 31/5377; A61K 45/06; C12N 15/113; C12N 5/09

(52) U.S. Cl. CPC A61K 31/7088; A61K 31/506; A61K 31/5377; A61K 45/06; C12N 15/113; C12N 5/0693; C12N 2310/113; C12N 2513/00; C12N 2501/06; C12N 2320/31; C12N 2310/3231; C12N 2310/20; C12N 2310/14; C12N 2503/02

(57) ABSTRACT

The invention provides methods and compositions for use in targeting micro RNAs (miRNAs), as well as methods and compositions for use in treating, reducing, inhibiting, or delaying resistance or tolerance to anti-cancer treatment, and methods and compositions for use in treating or preventing cancer.

### STATEMENT AS TO FEDERALLY FUNDED RESEARCH

The invention was made with government support under Grant No. CA196530 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Feb. 18, 2020 is named 01948-267WO2_Sequence_Listing_2.18.20_ST25 and is 176,514 bytes in size.

### FIELD OF THE INVENTION

This invention relates to methods and compositions for use in targeting micro RNAs (miRNAs), and methods of treating cancer.

### BACKGROUND

Relapsed disease following conventional treatments remains one of the central problems in cancer management, including epidermal growth factor receptor (EGFR)-based targeted therapy (Kobayashi et al., N. Engl. J. Med. 352(8):786-792, 2005; Paez et al., Science 304(5676):1497-1500, 2004). Tumor cells overcome anti-EGFR treatment by acquisition of drug binding-deficient mutations of EGFR and bypass through other protein tyrosine kinase signaling pathways (Niederst et al., Sci. Signal. 6(294):re6, 2013). For example, a majority of tumors from EGFR-mutant non-smal cell lung cancer (NSCLC) patients acquired resistance mutations such as EGFRT790M or EGFRC797S when the patients were treated with EGFR tyrosine kinase inhibitors (TKIs), gefitinib or erlotinib and osimertinib, respectively (Thress et al., Nat. Med. 21(6):560-562, 2015; Pao et al., PLoS Med. 2(3):e73, 2005). Recently, it has been found that EGFRT790M-positive drug-resistant cells can emerge from EGFRT790M-negative drug-tolerant cells that survive initial drug treatment (Hata et al., Nat. Med. 22(3):262-269, 2016; Ramirez et al., Nat. Commun. 7:10690, 2016). Thus, targeting drug-tolerant cells might be a new strategy to block drug resistance (Sharma et al., Cell 141(1):69-80, 2010; Smith et al., Cancer Cell 29(3):270-284, 2016). With success in applying osimertinib in the first-line treatment of EGFRT790M-positive NSCLC (Soria et al., N. Engl. J. Med. 378(2):113-125, 2018), it is therefore crucial to identify the changes driving drug-tolerance. However, the molecules driving drug-tolerance towards EGFR TKIs are not well studied.

Aberrantly regulated metabolic pathways lead to tumorigenesis and advantageous survival of tumor cells (Go et al., Biochemistry 53(5):947-956, 2014; Ward et al., Cancer Cell 21(3):297-308, 2012; Zhang et al., Cell 148(1-2):259-272, 2012; Jain et al., Science 336(6084):1040-1044, 2012; Vander Heiden et al., Science 324(5960):1029-1033, 2009). The tricarboxylic acid (TCA) cycle is a central pathway in the metabolism of sugars, lipids, and amino acids (Raimundo et al., Trends Mol. Med. 17(11):641-849, 2011). A dysfunctional TCA cycle induces oncogenesis by activating pseudohypoxia responses, which express hypoxia-associated proteins regardless of the oxygen status (Vyas et al., Cell 166(3):555-566, 2016; Sabharwal et al., Nat. Rev. Cancer 14(11):709-721, 2014; MacKenzie et al., Mol. Cell Biol. 27(9):3282-3289, 2007). For example, succinate accumulation caused by functional loss of the TCA cycle enzyme succinate dehydrogenase (SDH) stabilizes hypoxia-inducible factor 1alpha (HIF1alpha) via prolyl-hydroxylase (PHD) inhibition (Selak et al., Cancer Cell 7(1):77-85, 2005; Nowicki et al., FEBS J. 282(15):2796-2805, 2015). In addition, loss of function of Von Hippel-Lindau (VHL) also induces the pseudohypoxia response through decreased ubiquitination and proteasomal degradation of HIF1alpha (Kaelin, Nat. Rev. Cancer 2(9):673-682, 2002). Compared to other cancers, NSCLC is well vascularized and tumor cells depend on high levels of the iron-sulfur cluster biosynthetic enzymes to reduce oxidative damage due to exposure to high oxygen (Alvarez et al., Nature 551(7682):639-643, 2017). Most recently, it was shown that drug-tolerant persistent cancer cells were vulnerable to lipid hydroperoxidase GPX4 inhibition due to a disabled antioxidant program (Hangauer et al., Nature 551(7679):247-250, 2017). However, our understanding of changes conferring drug-tolerance remain limited.

There is a need for approaches to counteract cancer drug tolerance and resistance. Accordingly, we explored which signaling pathways initiate anticancer drug-tolerance and how this shapes cancer metabolism and tumor relapse.

### SUMMARY

The invention provides methods of treating, reducing, preventing, or delaying tolerance or resistance to anti-receptor tyrosine kinase (RTK) therapy in a subject (e.g., a human patient and/or a subject having cancer), the methods including administration of one or more miR-147b inhibitors to the subject. The invention also provides methods of treating or preventing cancer in a subject (e.g., a human patient and/or a subject having cancer), the methods including administering one or more miR-147b inhibitors to the subject.

In some embodiments, the RTK is selected from the group consisting of epidermal growth factor receptor (EGFR), human EGFR2 (HER2), HER3, anaplastic lymphoma kinase (ALK), ROS1, ERBB2/3/4, KIT, MET/hepatocyte growth factor receptor (HGFR), RON, platelet derived growth factor receptor (PDGFR), vascular endothelial cell growth factor receptor (VEGFR), VEGFR1, VEGFR2, fibroblast growth factor receptor (FGFR), insulin-like growth factor 1 receptor (IGF1R), and RET.

In some embodiments, the miR-147b inhibitor reduces a Von Hippel-Lindau (VHL)-pseudohypoxia response or counteracts metabolic changes in the tricarboxylic acid (TCA) cycle associated with drug tolerance in the subject.

In some embodiments, the subject has a cancer selected from the group consisting of kung cancer, non-small cell lung cancer, colorectal cancer, anal cancer, glioblastoma, squamous cell carcinoma, squamous cell carcinoma of the head and neck, pancreatic cancer, breast cancer, renal cell carcinoma, thyroid cancer, gastroesophageal adenocarcinoma, and gastric cancer, or one of the cancer types listed elsewhere herein.

In some embodiments, the methods further include administering an anti-RTK therapy to the subject. For example, an anti-EGFR therapy can be administered. In some embodiments, the anti-RTK (e.g., anti-EGFR) therapy includes a tyrosine kinase inhibitor (TKI). In some embodiment, the TKI is selected from the group consisting of gefitinib, erlotinib, afatinib, lapatinib, neratinib, osimertinib, vandetanib, crizotinib, dacomitinib, regorafenib, ponatinib, vismodegib, pazopanib, cabozantinib, bosutinib, axitinib, vemurafenib, ruxolitinib, nilotinib, dasatinib, imatinib, sunitinib, sorafenib, trametinib, cobimetanib, and dabrafenib. In some embodiments, the anti-EGFR therapy includes an anti-EGFR antibody or fragment thereof, or an anti-EGFR CAR T cell. In some embodiments, the anti-EGFR therapy includes an anti-EGFR antibody selected from the group consisting of cetuximab, necitumumab, panitumumab, nimotuzumab, futuximab, zatuximab, cetugex, and margetuximab. Other antibodies may also be administered, including those listed as follows. Anti-HER2 antibodies include trastuzumab, pertuzumab, trasgex, seribantumab, and patritumab. Antibodies against additional RTKs include the following: onartuzumab (HER3), namatumab (RON), ganitumab (RON), cixutumumab (RON), dalotuzumab (IGF1R), teprotumumab (IGF1R), icrucumab (VEGFR1), ramucirumab (VEGFR1), tanibirumab (VEGFR2), and olaratumab (PDGFR). In various embodiments, the one or more miR-147b inhibitors are administered before, at the same time as, or after the anti-RTK therapy.

In some embodiments, the subject has or is at risk of developing tolerance or resistance to anti-RTK therapy, e.g., an anti-EGFR therapy, an anti-AKL therapy, an anti-ROS1 therapy, an anti-ERBB2/3/4 therapy, an anti-KIT therapy, an anti-MET/hepatocyte growth factor receptor (HGFR) therapy, an anti-platelet derived growth factor receptor (PDGFR) therapy, an anti-vascular endothelial cell growth factor receptor (VEGFR) therapy, an anti-fibroblast growth factor receptor (FGFR) therapy, or an anti-RET therapy.

In some embodiments, the anti-RTK therapy to which the subject has or is at risk of developing tolerance or resistance includes a TKI, e.g., gefitinib, erlotinib, afatinib, lapatinib, neratinib, osimertinib, vandetanib, crizotinib, dacomitinib, regorafenib, ponatinib, vismodegib, pazopanib, cabozantinib, bosutinib, axitinib, vemurafenib, ruxolitinib, nilotinib, dasatinib, imatinib, sunitinib, sorafenib, trametinib, cobimetanib, or dabrafenib. In some embodiments, the subject has or is at risk of developing tolerance or resistance to an anti-EGFR therapy including an anti-EGFR antibody or fragment thereof, or an anti-EGFR CAR T cell. In some embodiments, the anti-EGFR therapy to which the subject has or is at risk of developing tolerance or resistance includes an anti-EGFR antibody selected from the group consisting of cetuximab, necitumumab, panitumumab, nimotuzumab, futuximab, zatuximab, cetugex, and margetuximab. Other antibodies may also be administered, including those listed as follows. Anti-HER2 antibodies include trastuzumab, pertuzumab, trasgex, seribantumab, and patritumab. Antibodies against additional RTKs include the following: onartuzumab (HER3), namatumab (RON), ganitumab (RON), cixutumumab (RON), dalotuzumab (IGF1R), teprotumumab (IGF1R), icrucumab (VEGFR1), ramucirumab (VEGFR1), tanibirumab (VEGFR2), and olaratumab (PDGFR).

In some embodiments, the one or more miR-147b inhibitors include one or more inhibitory molecule selected from the group consisting of an antisense oligonucleotide, an antagomir, an anti-miRNA sponge, a competitive inhibitor, a triplex-forming oligonucleotide, a double-stranded oligonucleotide, a short interfering RNA, an siRNA, an shRNA, a guide sequence for RNAse P, a small molecule, a catalytic RNA, and a ribozyme; or the inhibition is carried out by the use of a gene editing approach, such as CRISPR-cas9.

In some embodiments, the one or more miR-147b inhibitors are inhibitors of the production or activity of pri-miR-147b, pre-miR147b, or mature miR-147b.

The invention also provides single-stranded oligonucleotides including a total of 12 to 50 (or 10 to 60, or 8 to 75) interlinked nucleotides and having a nucleobase sequence including at least 6 contiguous nucleobases complementary to an equal-length portion of a miR-147b target nucleic acid.

In some embodiments, the oligonucleotide includes at least one modified nucleobase. In certain embodiments, the at least one modified nucleobase is selected from the group consisting of 5-methylcytosine, 7-deazaguanine, and 6-thioguanine.

In some embodiments, the oligonucleotide includes at least one modified internucleoside linkage. In certain embodiments, the modified internucleoside linkage is a phosphorothioate linkage. In some embodiments, the phosphorothioate linkage is a stereochemically enriched phosphorothioate linkage. In some embodiments, at least 50% or at least 70% of the internucleoside linkages in the oligonucleotide are each independently a modified internucleoside linkage.

In some embodiments, the oligonucleotide includes at least one modified sugar nucleoside. In certain embodiments, the at least one modified sugar nucleoside is a bridged nucleic acid. In some embodiments, the bridged nucleic acid is a locked nucleic acid (LNA), an ethylene-bridged nucleic acid (ENA), or a cEt nucleic acid. In some embodiments, the at least one modified sugar nucleoside is a 2′-modified sugar nucleoside, e.g., a sugar with a 2′-modification selected from the group consisting of 2′-fluoro, 2′-methoxy, and 2′-methoxyethoxy.

In some embodiments, the oligonucleotide includes deoxyribonucleotides. In some embodiments, the oligonucleotide includes ribonucleotides. In some embodiments, the oligonucleotide is a morpholino oligonucleotide. In some embodiments, the oligonucleotide is a peptide nucleic acid.

In further embodiments, the oligonucleotide includes a hydrophobic moiety covalently attached at its 5′-terminus, its 3′-terminus, or an internucleoside linkage of the oligonucleotide.

In additional embodiments, the oligonucleotide includes or consists of a sequence selected from the group consisting of SEQ ID NOs: 3 to 736 or a variant thereof (see, e.g., Tables 1 and 3), or the reverse complement thereof. The oligonucleotide may comprise deoxyribonucleotides, ribonucleotides, or a mixture thereof.

In some embodiments, the oligonucleotide includes at least 8 or at least 12 contiguous nucleobases complementary to an equal-length portion of a miR-147b target nucleic acid. In some embodiments, the oligonucleotide includes 20 or fewer contiguous nucleobases complementary to an equal-length portion of a miR-147b target nucleic acid. In some embodiments, the oligonucleotide includes a total of at least 12 interlinked nucleotides. In some embodiments, the oligonucleotide includes a total of 24 or fewer interlinked nucleotides.

In some embodiments, the oligonucleotide is a gapmer, headmer, tailmer, altmer, blockmer, skipmer, or unimer.

In some embodiments, the oligonucleotide targets a sequence comprising or consisting of nucleotides 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 46-51, 47-52, 48-53, 49-54, 50-55, 51-56, 52-57, 53-58, 54-59, 55-60, 56-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, or 75-80 of SEQ ID NO: 1. In some embodiments, the oligonucleotide targets said sequence and additionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 additional nucleotides of SEQ ID NO: 1, whether all on one side of the indicated fragment or wherein the fragment is between the one or more additional nucleotides. See below for additional, similar variants included in the invention.

The invention also provides double-stranded oligonucleotides including an oligonucleotide as described above hybridized to a complementary oligonucleotide.

Further, the invention provides double-stranded oligonucleotides including a passenger strand hybridized to a guide strand including a nucleobase sequence including at least 6 contiguous nucleobases complementary to an equal-length portion of a miR-147b target nucleic acid, wherein each of the passenger strand and the guide strand includes a total of 12 to 50 (or 10 to 60, or 8 to 75) interlinked nucleotides.

In some embodiments, the passenger strand and/or the guide strand includes at least one modified nucleobase, e.g., 5-methylcytosine, 7-deazaguanine, and 6-thioguanine.

In some embodiments, the passenger strand and/or the guide strand includes at least one modified internucleoside linkage, e.g., a phosphorothioate linkage (such as a stereochemically enriched phosphorothioate linkage).

In some embodiments, at least 50% or at least 70% of the internucleoside linkages in the passenger strand and/or the guide strand are each independently the modified internucleoside linkage.

In some embodiments, the passenger strand and/or the guide strand includes at least one modified sugar nucleoside, e.g., a bridged nucleic acid (such as, e.g., a locked nucleic acid (LNA), an ethylene-bridged nucleic acid (ENA), or a cEt nucleic acid). In some embodiments, the at least one modified sugar nucleoside is a 2′-modified sugar nucleoside, e.g., a sugar with a 2′-modification selected from the group consisting of 2′-fluoro, 2′-methoxy, and 2′-methoxyethoxy.

In some embodiments, the passenger strand and/or the guide strand includes deoxyribonucleotides. In some embodiments, the passenger strand and/or the guide strand includes ribonucleotides.

In some embodiments, the passenger strand and/or the guide strand includes a hydrophobic moiety covalently attached at a 5′-terminus, a 3′-terminus, or an internucleoside linkage of the passenger strand.

In some embodiments, the guide strand includes a sequence selected from the group consisting of SEQ ID NOs: 3 to 736 or a variant thereof (or the reverse complement thereof)(see, e.g., Tables 1 and 3). In some embodiments, the passenger strand includes a sequence selected from the group consisting of SEQ ID NOs: 3 to 736 or a variant thereof (or the reverse complement thereof)(see, e.g., Tables 1 and 3). The oligonucleotide may comprise deoxyribonucleotides, ribonucleotides, or a mixture thereof.

In some embodiments, the hybridized oligonucleotide includes at least one 3′-overhang (e.g., two 3′ overhangs). In some embodiments, the hybridized oligonucleotide includes a blunt end.

In some embodiments, the miR-147 target nucleic acid includes pri-miR-147b, pre-miR-147b, or mature miR-147b.

In some embodiments, the oligonucleotide targets a sequence comprising or consisting of nucleotides 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 48-51, 47-52, 48-53, 49-54, 50-55, 51-56, 52-57, 53-58, 54-59, 55-80, 56-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, or 75-80 of SEQ ID NO: 1. In some embodiments, the oligonucleotide targets said sequence and additionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 additional nucleotides of SEQ ID NO: 1, whether all on one side of the indicated fragment or wherein the fragment is between the one or more additional nucleotides. See below for additional, similar variants included in the invention.

The invention also includes oligonucleotides that compete with miR-147b for binding to a target mRNA or pre-mRNA sequence, thereby inhibiting or reducing the effects of miR-147b on the mRNA or pre-mRNA. In some embodiments, the oligonucleotides include or consists of a sequence selected from SEQ ID NOs: 1, 2, or 737 to 889 (or the reverse complement thereof)(see, e.g., Tables 2 and 4).

The invention further includes vectors including a sequence encoding an oligonucleotide as described herein, wherein the vector optionally further includes a promoter to direct transcription of the sequence. In some embodiments, the vector includes a sequence encoding multiple oligonucleotides, for example, the vector includes a sequence encoding 2, 3, 4, 5, 6, 7, 8, 9, or 10 oligonucleotides. In some embodiments, the vector is a virus, such as a lentivirus, an adenovirus, or an adeno-associated virus; or is a plasmid, a cosmid, or a phagemid.

The invention also provides pharmaceutical compositions including (i) an oligonucleotide as described herein, a vector as described herein, and/or a small molecule inhibitor of miR-147b, and (ii) a pharmaceutically acceptable excipient or carrier.

The invention additionally provides methods of treating a subject (e.g., a human patient and/or a subject having cancer) in need thereof, the methods including administering to the subject a therapeutically effective amount of an oligonucleotide as described herein, a vector as described herein, and/or a pharmaceutical composition as described herein.

In some embodiments, the methods further include administration of an additional anti-cancer agent, e.g., anti-RTK agent (see, e.g., those anti-RTK agents listed herein).

The invention also provides methods of determining whether tolerance or resistance of a cancer to anti-RTK therapy may be effectively treated, reduced, prevented, or delayed by anti-miR-147b therapy, the methods including determining the level of miR-147b in the cancer, wherein detection of an increased level of miR-147b, relative to a control, indicates that tolerance or resistance of the cancer to anti-RTK therapy may be effectively treated, reduced, prevented, or delayed with anti-miR-147b therapy, optionally in combination with anti-RTK therapy.

In these methods, the anti-miR-147 therapy can optionally be selected from an oligonucleotide as described herein, a vector as described herein, and/or a small molecule inhibitor of miR-147b, and/or the anti-RTK therapy can optionally be selected from a TKI, an anti-RTK antibody, and a CAR T cell directed against an RTK. Furthermore, in these methods, determination of the level of miR-147b in the cancer can be carried out by detection of the level of miR-147b in a sample from the subject (e.g., a human patient and/or a subject having cancer) having the cancer. Optionally, the sample includes tumor tissue, tissue swab, sputum, serum, or plasma. The methods further optionally include a step of administering an anti-miR147b therapy to a subject having the cancer (e.g., a human patient and/or a subject having cancer), if it is determined that tolerance or resistance of the cancer to anti-RTK therapy may be effectively treated, reduced, prevented, or delayed by anti-miR-147b therapy.

The invention further provides methods of determining whether a cancer may be effectively treated or prevented with an anti-miR-147b therapy, the methods including determining the level of miR-147b in the cancer, wherein detection of an increased level of miR-147b in the cancer, relative to a control, indicates that the cancer may effectively be treated or prevented with anti-miR-147b therapy, optionally in combination with anti-RTK therapy.

In these methods, the anti-miR-147 therapy can optionally be selected from an oligonucleotide as described herein, a vector as described herein, and/or a small molecule inhibitor of miR-147b, and/or the anti-RTK therapy can optionally be selected from a TKI, an anti-RTK antibody, and a CAR T cell directed against an RTK. Furthermore, in these methods, determination of the level of miR-147b in the cancer can be carried out by detection of the level of miR-147b in a sample from the subject (e.g., a human patient and/or a subject having cancer) having the cancer. Optionally, the sample includes tumor tissue, tissue swab, sputum, serum, or plasma. The methods further optionally include a step of administering an anti-miR147b therapy to a subject having the cancer (e.g., a human patient and/or a subject having cancer), if it is determined that the cancer may be effectively treated with anti-miR147b therapy.

The invention also provides methods of detecting a cancer cell in a sample, the methods including determining the level of miR-147b in the sample, wherein detection of an increased level of miR-147b in the sample, relative to a control, indicates the presence of a cancer cell in the sample.

The invention additionally provides methods of determining whether a cancer cell in a sample may be tolerant or resistant to anti-RTK therapy, the methods including determining the level of miR-147b in the sample, wherein detection of an increased level of miR-147b, relative to a control, indicates that the cancer cell may be tolerant or resistant to anti-RTK therapy.

In some embodiments of these methods, the anti-RTK therapy is anti-EGFR therapy (e.g., as described herein). In some embodiments, the sample includes tumor tissue, tissue swab, sputum, serum, or plasma.

Also provided by the invention are methods of making organoids including lung cells, the methods including the steps of: a. culturing lung cells in a medium including epidermal growth factor (EGF), fibroblast growth factor 2 (FGF2), and fibroblast growth factor 10 (FGF10); b. maintaining the cells in culture in a medium including Noggin and transforming growth factor-β (TGF-β); and c. differentiating the cells in a medium including fibroblast growth factor 7 (FGF7) and platelet-derived growth factor (PDGF).

In some embodiments, the lung cells are lung epithelial cells obtained from a sample of lung tissue of a subject. In some embodiments, the lung cells are immortalized lung epithelial cells. In some embodiments, the kung cells are cancerous. In some embodiments, the lung cells are non-cancerous. In some embodiments, the lung cells are tolerant or resistant to an anti-RTK agent. In some embodiments, the maintaining step is carried out on days 0-3 of the method, maintenance is carried out on days 4-6, and differentiation is carried out on days 7-24. In some embodiments, the organoids show ring-like structures upon treatment with an anti-RTK agent.

The invention further provides three-dimensional organoids including lung cells, wherein the organoid is optionally made by, or has features of organoids made using, the methods described above and elsewhere herein. In some embodiments, the lung cells include lung cancer cells. In some embodiments, the kung cells or lung cancer cells are primary cells, obtained or cultured from the cells of a subject (e.g., a human patient and/or a subject having cancer).

The invention also provides methods for identifying an agent that may be used (i) to treat, reduce, prevent, or delay tolerance or resistance to anti-RTK therapy, or (ii) in the treatment or prevention of cancer, the methods including contacting a cell with the agent and determining whether the agent decreases the level of miR-147b in the cell. In some embodiments, the cell is included within an organoid, such as an organoid as described herein. In some embodiments, the organoid includes lung cancer cells. In some embodiments, the organoid is an organoid as described herein and/or is made using a method as described herein. In some embodiments, the lung cancer cells are resistant to an anti-RTK therapy. In some embodiments, the cells are primary cells, obtained or cultured from the cells of a subject (e.g., a human patient and/or a subject having cancer). In some embodiments, the agent is a candidate compound, not previously known to be effective at treating, reducing, preventing, or delaying tolerance or resistance to anti-RTK therapy, or at treating or preventing cancer. In some embodiments, the method is carried out to determine an optimal approach to treat, reduce, prevent, or delay tolerance or resistance of a cancer to anti-RTK therapy in a subject, or to treat or prevent a cancer in a subject.

The invention additionally provides kits including one or more agents for detecting the level of miR-147b in a sample. In some embodiments, the agent includes an oligonucleotide, which is optionally an oligonucleotide as described herein. The invention further includes kits including one or more miR-147b inhibitors, which optionally is/are one or more oligonucleotides as described herein, and a second agent for treating cancer (e.g., as described herein).

The invention further provides compositions, as described herein, for use in the methods, as described herein, as well as use of the compositions described herein in the preparation of medicaments for the prevention or treatment of diseases or conditions (e.g., cancer), or for treating, reducing, preventing, or delaying tolerance or resistance to anti-receptor tyrosine kinase (RTK) therapy in a subject, as described herein.

### Definitions

The term “acyl,” as used herein, represents a chemical substituent of formula —C(O)—R, where R is alkyl, aryl, arylalkyl, cycloalkyl, heterocyclyl, heterocyclyl alkyl, heteroaryl, or heteroaryl alkyl. An optionally substituted acyl is an acyl that is optionally substituted as described herein for each group R.

The term “acyloxy,” as used herein, represents a chemical substituent of formula —OR, where R is acyl. An optionally substituted acyloxy is an acyloxy that is optionally substituted as described herein for acyl.

The term “alkanoyl,” as used herein, represents a chemical substituent of formula —C(O)—R, where R is alkyl. An optionally substituted alkanoyl is an alkanoyl that is optionally substituted as described herein for alkyl.

The term “alkoxy,” as used herein, represents a chemical substituent of formula —OR, where R is a C₁₋₆ alkyl group, unless otherwise specified. An optionally substituted alkoxy is an alkoxy group that is optionally substituted as defined herein for alkyl.

The term “alkyl,” as used herein, refers to an acyclic straight or branched chain saturated hydrocarbon group, which, when unsubstituted, has from 1 to 12 carbons, unless otherwise specified. In certain preferred embodiments, unsubstituted alkyl has from 1 to 6 carbons. Alkyl groups are exemplified by methyl; ethyl; n- and iso-propyl; n-, sec-, iso- and tert-butyl; neopentyl, and the like, and may be optionally substituted, valency permitting, with one, two, three, or, in the case of alkyl groups of two carbons or more, four or more substituents independently selected from the group consisting of: alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; and ═NR′, where R′ is H, alkyl, aryl, or heterocyclyl. In some embodiments, two substituents combine to form a group -L-CO—R, where L is a bond or optionally substituted C₁₋₁₁ alkylene, and R is hydroxyl or alkoxy. Each of the substituents may itself be unsubstituted or, valency permitting, substituted with unsubstituted substituent(s) defined herein for each respective group.

The term “alkylene,” as used herein, represents a divalent substituent that is an alkyl having one hydrogen atom replaced with a valency. An optionally substituted alkylene is an alkylene that is optionally substituted as described herein for alkyl.

The term “altmer,” as used herein, refers to an oligonucleotide having a pattern of structural features characterized by internucleoside linkages, in which no two consecutive internucleoside linkages have the same structural feature. In some embodiments, an altmer is designed such that it includes a repeating pattern. In some embodiments, an altmer is designed such that it does not include a repeating pattern. In instances, where the “same structural feature” refers to the stereochemical configuration of the internucleoside linkages, the altmer is a “stereoaltmer.”

The term “aryl,” as used herein, represents a mono-, bicyclic, or multicyclic carbocyclic ring system having one or two aromatic rings. Aryl group may include from 6 to 10 carbon atoms. All atoms within an unsubstituted carbocyclic aryl group are carbon atoms. Non-limiting examples of carbocyclic aryl groups include phenyl, naphthyl, 1,2-dihydronaphthyl, 1,2,3,4-tetrahydronaphthyl, fluorenyl, indanyl, indenyl, etc. The aryl group may be unsubstituted or substituted with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; and cyano. Each of the substituents may itself be unsubstituted or substituted with unsubstituted substituent(s) defined herein for each respective group.

The term “aryl alkyl,” as used herein, represents an alkyl group substituted with an aryl group. The aryl and alkyl portions may be optionally substituted as the individual groups as described herein.

The term “arylene,” as used herein, represents a divalent substituent that is an aryl having one hydrogen atom replaced with a valency. An optionally substituted arylene is an arylene that is optionally substituted as described herein for aryl.

The term “aryloxy,” as used herein, represents a group —OR, where R is aryl. Aryloxy may be an optionally substituted aryloxy. An optionally substituted aryloxy is aryloxy that is optionally substituted as described herein for aryl.

The term “bicyclic sugar moiety,” as used herein, represents a modified sugar moiety including two fused rings. In certain embodiments, the bicyclic sugar moiety includes a furanosyl ring.

The term “blockmer,” as used herein, refers to an oligonucleotide strand having a pattern of structural features characterized by the presence of at least two consecutive internucleoside linkages with the same structural feature. By same structural feature is meant the same stereochemistry at the internucleoside linkage phosphorus or the same modification at the linkage phosphorus. The two or more consecutive internucleoside linkages with the same structure feature are referred to as a “block.” In instances, where the “same structural feature” refers to the stereochemical configuration of the internucleoside linkages, the blockmer is a “stereoblockmer.”

The expression “C_{x-y},” as used herein, indicates that the group, the name of which immediately follows the expression, when unsubstituted, contains a total of from x to y carbon atoms. If the group is a composite group (e.g., aryl alkyl), C_{x-y} indicates that the portion, the name of which immediately follows the expression, when unsubstituted, contains a total of from x to y carbon atoms. For example, (C₅₋₁₀-aryl)-C₁₋₆-alkyl is a group, in which the aryl portion, when unsubstituted, contains a total of from 6 to 10 carbon atoms, and the alkyl portion, when unsubstituted, contains a total of from 1 to 6 carbon atoms.

The term “complementary,” as used herein in reference to a nucleobase sequence, refers to the nucleobase sequence having a pattern of contiguous nucleobases that permits an oligonucleotide having the nucleobase sequence to hybridize to another oligonucleotide or nucleic acid to form a duplex structure under physiological conditions. Complementary sequences include Watson-Crick base pairs formed from natural and/or modified nucleobases. Complementary sequences can also include non-Watson-Crick base pairs, such as wobble base pairs (guanosine-uracil, hypoxanthine-uracil, hypoxanthine-adenine, and hypoxanthine-cytosine), and Hoogsteen base pairs.

The term “contiguous,” as used herein in the context of an oligonucleotide, refers to nucleosides, nucleobases, sugar moieties, or internucleoside linkages that are immediately adjacent to each other. For example, “contiguous nucleobases” means nucleobases that are immediately adjacent to each other in a sequence.

The term “cycloalkyl,” as used herein, refers to a cyclic alkyl group having from three to ten carbons (e.g., a C₃-C₁₀ cycloalkyl), unless otherwise specified. Cycloalkyl groups may be monocyclic or bicyclic. Bicyclic cycloalkyl groups may be of bicyclo[p.q.0]alkyl type, in which each of p and q is, independently, 1, 2, 3, 4, 5, 6, or 7, provided that the sum of p and q is 2, 3, 4, 5, 6, 7, or 8. Alternatively, bicyclic cycloalkyl groups may include bridged cycloalkyl structures, e.g., bicyclo[p.q.r]alkyl, in which r is 1, 2, or 3, each of p and q is, independently, 1, 2, 3, 4, 5, or 6, provided that the sum of p, q, and r is 3, 4, 5, 6, 7, or 8. The cycloalkyl group may be a spirocyclic group, e.g., spiro[p.q]alkyl, in which each of p and q is, independently, 2, 3, 4, 5, 6, or 7, provided that the sum of p and q is 4, 5, 6, 7, 8, or 9. Non-limiting examples of cycloalkyl include cyclopropyl, cyclobutyl, cyclopentyl, cyclohexyl, cycloheptyl, 1-bicyclo[2.2.1.]heptyl, 2-bicyclo[2.2.1.]heptyl, 5-bicyclo[2.2.1.]heptyl, 7-bicyclo[2.2.1.]heptyl, and decalinyl. The cycloalkyl group may be unsubstituted or substituted (e.g., optionally substituted cycloalkyl) with one, two, three, four, or five substituents independently selected from the group consisting of: alkyl; alkoxy; acyloxy; amino; aryl; aryloxy; azido; cycloalkyl; cycloalkoxy; halo; heterocyclyl; heteroaryl; heterocyclylalkyl; heteroarylalkyl; heterocyclyloxy; heteroaryloxy; hydroxy; nitro; thiol; silyl; cyano; ═O; ═S; ═NR′, where R′ is H, alkyl, aryl, or heterocyclyl. Each of the substituents may itself be unsubstituted or substituted with unsubstituted substituent(s) defined herein for each respective group.

The term “cycloalkylene,” as used herein, represents a divalent substituent that is a cycloalkyl having one hydrogen atom replaced with a valency. An optionally substituted cycloalkylene is a cycloalkylene that is optionally substituted as described herein for cycloalkyl.

The term “cycloalkoxy,” as used herein, represents a group —OR, where R is cycloalkyl. Cycloalkoxy may be an optionally substituted cycloalkoxy. An optionally substituted cycloalkoxy is cycloalkoxy that is optionally substituted as described herein for cycloalkyl.

The term “duplex,” as used herein, represents two oligonucleotides that are paired through hybridization of complementary nucleobases.

The term “gapmer,” as used herein, refers to an oligonucleotide having an RNase H recruiting region (gap) flanked by a 5′ wing and 3′ wing, each of the wings including at least one affinity enhancing nucleoside (e.g., 1, 2, 3, or 4 affinity enhancing nucleosides).

The term “halo,” as used herein, represents a halogen selected from bromine, chlorine, iodine, and fluorine.

The term “headmer,” as used herein, refers to an oligonucleotide having an RNase H recruiting region (gap) flanked by a 5′ wing including at least one affinity enhancing nucleoside (e.g., 1, 2, 3, or 4 affinity enhancing nucleosides).

The term “heteroalkyl,” as used herein refers to an alkyl group interrupted one or more times by one or two heteroatoms each time. Each heteroatom is, independently, O, N, or S. None of the heteroalkyl groups includes two contiguous oxygen atoms. The heteroalkyl group may be unsubstituted or substituted (e.g., optionally substituted heteroalkyl). When heteroalkyl is substituted and the substituent is bonded to the heteroatom, the substituent is selected according to the nature and valency of the heteratom. Thus, the substituent bonded to the heteroatom, valency permitting, is selected from the group consisting of ═O, —N(R^{N2})₂, —SO₂OR^{N3}, —SO₂R^{N2}, —SOR^{N3}, —COOR^{N3}, an N protecting group, alkyl, aryl, cycloalkyl, heterocyclyl, or cyano, where each R^{N2} is independently H, alkyl, cycloalkyl, aryl, or heterocyclyl, and each R^{N3} is independently alkyl, cycloalkyl, aryl, or heterocyclyl. Each of these substituents may itself be unsubstituted or substituted with unsubstituted substituent(s) defined herein for each respective group. When heteroalkyl is substituted and the substituent is bonded to carbon, the substituent is selected from those described for alkyl, provided that the substituent on the carbon atom bonded to the heteroatom is not Cl, Br, or I. It is understood that carbon atoms are found at the termini of a heteroalkyl group. In some embodiments, heteroalkyl is PEG

The term “heteroalkylene,” as used herein, represents a divalent substituent that is a heteroalkyl having one hydrogen atom replaced with a valency. An optionally substituted heteroalkylene is a heteroalkylene that is optionally substituted as described herein for heteroalkyl.

The term “heteroaryl,” as used herein, represents a monocyclic 5-, 6-, 7-, or 8-membered ring system, or a fused or bridging bicyclic, tricyclic, or tetracyclic ring system; the ring system contains one, two, three, or four heteroatoms independently selected from the group consisting of nitrogen, oxygen, and sulfur; and at least one of the rings is an aromatic ring. Non-limiting examples of heteroaryl groups include benzimidazolyl, benzofuryl, benzothiazolyl, benzothienyl, benzoxazolyl, furyl, imidazolyl, indolyl, isoindazolyl, isoquinolinyl, isothiazolyl, isothiazolyl, isoxazolyl, oxadiazolyl, oxazolyl, purinyl, pyrrolyl, pyridinyl, pyrazinyl, pyrimidinyl, qunazolinyl, quinolinyl, thiadiazolyl (e.g., 1,3,4-thiadiazole), thiazolyl, thienyl, triazolyl, tetrazolyl, dihydroindolyl, tetrahydroquinolyl, tetrahydroisoquinolyl, etc. The term bicyclic, tricyclic, and tetracyclic heteroaryls include at least one ring having at least one heteroatom as described above and at least one aromatic ring. For example, a ring having at least one heteroatom may be fused to one, two, or three carbocyclic rings, e.g., an aryl ring, a cyclohexane ring, a cyclohexene ring, a cyclopentane ring, a cyclopentene ring, or another monocyclic heterocyclic ring. Examples of fused heteroaryls include 1,2,3,5,8,8a-hexahydroindolizine; 2,3-dihydrobenzofuran; 2,3-dihydroindole; and 2,3-dihydrobenzothiophene. Heteroaryl may be optionally substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl. Each of the substituents may itself be unsubstituted or substituted with unsubstituted substituent(s) defined herein for each respective group.

The term “heteroarylene,” as used herein, refers to a heteroaryl in which one hydrogen atom is replaced with a valency. An optionally substituted heteroaryle is a heteroarylene group that is optionally substituted as described herein for heteroaryl.

The term “heteroaryloxy,” as used herein, refers to a structure —OR, in which R is heteroaryl. Heteroaryloxy can be optionally substituted as defined for heteroaryl.

The term “heterocyclyl,” as used herein, represents a monocyclic, bicyclic, tricyclic, or tetracyclic ring system having fused or bridging 4-, 5-, 6-, 7-, or 8-membered rings, unless otherwise specified, the ring system containing one, two, three, or four heteroatoms independently selected from the group consisting of nitrogen, oxygen, and sulfur. Heterocyclyl may be aromatic or non-aromatic. An aromatic heterocyclyl is heteroaryl as described herein. Non-aromatic 5-membered heterocyclyl has zero or one double bonds, non-aromatic 6- and 7-membered heterocyclyl groups have zero to two double bonds, and non-aromatic 8-membered heterocyclyl groups have zero to two double bonds and/or zero or one carbon-carbon triple bond. Heterocyclyl groups have a carbon count of 1 to 16 carbon atoms unless otherwise specified. Certain heterocyclyl groups may have a carbon count up to 9 carbon atoms. Non-aromatic heterocyclyl groups include pyrrolinyl, pyrrolidinyl, pyrazolinyl, pyrazolidinyl, imidazolinyl, imidazolidinyl, piperidinyl, homopiperidinyl, piperazinyl, pyridazinyl, oxazolidinyl, isoxazolidiniyl, morpholinyl, thiomorpholinyl, thiazolidinyl, isothiazolidinyl, thiazolidinyl, tetrahydrofuranyl, dihydrofuranyl, tetrahydrothienyl, dihydrothienyl, pyranyl, dihydropyranyl, dithiazolyl, etc. The term “heterocyclyl” also represents a heterocyclic compound having a bridged multicyclic structure in which one or more carbons and/or heteroatoms bridges two non-adjacent members of a monocyclic ring, e.g., quinuclidine, tropanes, or diaza-bicyclo[2.2.2]octane. The term “heterocyclyl” includes bicyclic, tricyclic, and tetracyclic groups in which any of the above heterocyclic rings is fused to one, two, or three carbocyclic rings, e.g., a cyclohexane ring, a cyclohexene ring, a cyclopentane ring, a cyclopentene ring, or another heterocyclic ring. Examples of fused heterocyclyls include 1,2,3,5,8,8a-hexahydroindolizine; 2,3-dihydrobenzofuran; 2,3-dihydroindole; and 2,3-dihydrobenzothiophene. The heterocyclyl group may be unsubstituted or substituted with one, two, three, four, or five substituents independently selected from the group consisting of: alkyl; alkoxy; acyloxy; aryloxy; amino; arylalkoxy; cycloalkyl; cycloalkoxy; halogen; heterocyclyl; heterocyclyl alkyl; heteroaryl; heteroaryl alkyl; heterocyclyloxy; heteroaryloxy; hydroxyl; nitro; thiol; cyano; ═O; ═S; —NR₂, where each R is independently hydrogen, alkyl, acyl, aryl, arylalkyl, cycloalkyl, heterocyclyl, or heteroaryl; —COOR^{A}, where R^{A} is hydrogen, alkyl, aryl, arylalkyl, cycloalkyl, heterocyclyl, or heteroaryl; and —CON(R^{B})₂, where each R^{B} is independently hydrogen, alkyl, aryl, arylalkyl, cycloalkyl, heterocyclyl, or heteroaryl.

The term “heterocyclyl alkyl,” as used herein, represents an alkyl group substituted with a heterocyclyl group. The heterocyclyl and alkyl portions of an optionally substituted heterocyclyl alkyl are optionally substituted as described for heterocyclyl and alkyl, respectively.

The term “heterocyclylene,” as used herein, represents a divalent substituent that is a heterocyclyl having one hydrogen atom replaced with a valency. An optionally substituted heterocyclylene is a heterocyclylene that is optionally substituted as described herein for heterocyclyl.

The term “heterocyclyloxy,” as used herein, refers to a structure —OR, in which R is heterocyclyl. Heterocyclyloxy can be optionally substituted as described for heterocyclyl.

The terms “hydroxyl” and “hydroxy,” as used interchangeably herein, represent —OH.

The term “hydrophobic moiety,” as used herein, represents a monovalent group covalently linked to an oligonucleotide backbone, where the monovalent group is a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen. Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. A linker may optionally be used to connect the monovalent group to the oligonucleotide, and may be a linker consisting of 1, 2, 3, 4, 5, 6, 7, 8, 9, or 10 monomers independently selected from the group consisting of optionally substituted C₁₋₁₂ alkylene, optionally substituted C₂₋₁₂ heteroalkylene, optionally substituted C₆₋₁₀ arylene, optionally substituted C₃₋₈ cycloalkylene, optionally substituted C₁₋₉ heteroarylene, optionally substituted C₁₋₉ heterocyclylene, —O—, —S—S—, and —NR^{N}—, where each R^{N} is independently H or optionally substituted C₁₋₁₂ alkyl. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

The term “internucleoside linkage,” as used herein, represents a group or bond that forms a covalent linkage between adjacent nucleosides in an oligonucleotide. An internucleoside linkage is an unmodified internucleoside linkage or a modified internucleoside linkage. An “unmodified internucleoside linkage” is a phosphate (—O—P(O)(OH)—O—) internucleoside linkage (“phosphate phosphodiester”). A “modified internucleoside linkage” is an internucleoside linkage other than a phosphate phosphodiester.

The two main classes of modified internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH)—O—CH₂—), thiodiester (—O—C(O)—S—), thionocarbamate (—O—C(O)(NH)—S—), siloxane (—O—Si(H)₂—O—), and N,N′-dimethylhydrazine (—CH₂—N(CH₃)—N(CH₃)—). Phosphorothioate linkages are phosphodiester linkages and phosphotriester linkages in which one of the non-bridging oxygen atoms is replaced with a sulfur atom. In some embodiments, an internucleoside linkage is a group of the following structure:

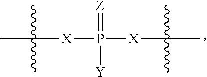

where

Z is O, S, or Se;

Y is —X-L-R¹;

each X is independently —O—, —S—, —N(-L-R¹)—, or L;

each L is independently a covalent bond or a linker (e.g., a linker consisting of 1, 2, 3, 4, 5, 6, 7, 8, 9, or 10 monomers independently selected from the group consisting of optionally substituted C₁₋₁₂ alkylene, optionally substituted C₂₋₁₂ heteroalkylene, optionally substituted C₆₋₁₀ arylene, optionally substituted C₃₋₈ cycloalkylene, optionally substituted C₁₋₉ heteroarylene, optionally substituted C₁₋₉ heterocyclylene, —O—, —S—S—, and —NR^{N}—, where each R^{N} is independently H or optionally substituted C₁₋₁₂ alkyl);

each R¹ is independently hydrogen, —S—S—R², —O—CO—R², —S—CO—R², optionally substituted C₁₋₉ heterocyclyl, or a hydrophobic moiety; and

each R² is independently optionally substituted C₁₋₁₀ alkyl, optionally substituted C₂₋₁₀ heteroalkyl, optionally substituted C₆₋₁₀ aryl, optionally substituted C₆₋₁₀ aryl C₁₋₆ alkyl, optionally substituted C₁₋₉ heterocyclyl, or optionally substituted C₁₋₉ heterocyclyl C₁₋₆ alkyl.

When L is a covalent bond, R¹ is hydrogen, Z is oxygen, and all X groups are —O—, the internucleoside group is known as a phosphate phosphodiester. When L is a covalent bond, R¹ is hydrogen, Z is sulfur, and all X groups are —O—, the internucleoside group is known as a phosphorothioate diester. When Z is oxygen, all X groups are —O—, and either (1) L is a linker or (2) R¹ is not a hydrogen, the internucleoside group is known as a phosphotriester. When Z is sulfur, all X groups are —O—, and either (1) L is a linker or (2) R¹ is not a hydrogen, the internucleoside group is known as a phosphorothioate triester. Non-limiting examples of phosphorothioate triester linkages and phosphotriester linkages are described in US 2017/0037399, the disclosure of which is incorporated herein by reference.

The term “morpholino,” as used herein in reference to a class of oligonucleotides, represents an oligomer of at least 10 morpholino monomer units interconnected by morpholino internucleoside linkages. A morpholino includes a 5′ group and a 3′ group. For example, a morpholino may be of the following structure:

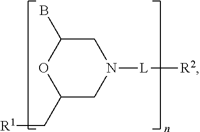

where

n is an integer of at least 10 (e.g., 12 to 30) indicating the number of morpholino units;

each B is independently a nucleobase;

R¹ is a 5′ group;

R² is a 3′ group; and

L is (i) a morpholino internucleoside linkage or, (ii) if L is attached to R², a covalent bond. A 5′ group in morpholino may be, e.g., hydroxyl, a hydrophobic moiety, phosphate, diphosphate, triphosphate, phosphorothioate, diphosphorothioate, triphosphorothioate, phosphorodithioate, disphorodihioate, triphosphorodithioate, phosphonate, phosphoramidate, a cell penetrating peptide, an endosomal escape moiety, or a neutral organic polymer. A 3′ group in morpholino may be, e.g., hydrogen, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer.

The term “morpholino internucleoside linkage,” as used herein, represents a divalent group of the following structure:

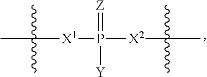

where

Z is O or S;

X¹ is a bond, —CH₂—, or —O—;

X² is a bond, —CH₂—O—, or —O—; and

Y is —NR₂, where each R is independently C₁₋₆ alkyl (e.g., methyl), or both R combine together with the nitrogen atom to which they are attached to form a C₂₋₉ heterocyclyl (e.g., N-piperazinyl); provided that both X¹ and X² are not simultaneously a bond.

The term “nucleobase,” as used herein, represents a nitrogen-containing heterocyclic ring found at the 1′ position of the ribofuranose/2′-deoxyribofuranose of a nucleoside. Nucleobases are unmodified or modified. As used herein, “unmodified” or“natural” nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and O-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e.g., 5-substituted pyrimidines; 6-azapyrimidines; N2-, N6-, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine, or 2-pyridone. Further nucleobases include those disclosed in Merigan et al., U.S. Pat. No. 3,687,808, those disclosed in The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

The term “nucleoside,” as used herein, represents sugar-nucleobase compounds and groups known in the art, as well as modified or unmodified 2′-deoxyribofuranose-nucleobase compounds and groups known in the art. The sugar may be ribofuranose. The sugar may be modified or unmodified. An unmodified ribofuranose-nucleobase is ribofuranose having an anomeric carbon bond to an unmodified nucleobase. Unmodified ribofuranose-nucleobases are adenosine, cytidine, guanosine, and uridine. Unmodified 2′-deoxyribofuranose-nucleobase compounds are 2′-deoxyadenosine, 2′-deoxycytidine, 2′-deoxyguanosine, and thymidine. The modified compounds and groups include one or more modifications selected from the group consisting of nucleobase modifications and sugar modifications described herein. A nucleobase modification is a replacement of an unmodified nucleobase with a modified nucleobase. A sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

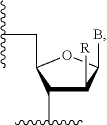

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

The term “nucleotide,” as used herein, represents a nucleoside bonded to an internucleoside linkage or a monovalent group of the following structure —X¹—P(X²)(R¹)₂, where X¹ is O, S, or NH, and X² is absent, ═O, or ═S, and each R¹ is independently —OH, —N(R²)₂, or —O—CH₂CH₂CN, where each R² is independently an optionally substituted alkyl, or both R² groups, together with the nitrogen atom to which they are attached, combine to form an optionally substituted heterocyclyl.

The term “oligonucleotide,” as used herein, represents a structure containing 10 or more contiguous nucleosides covalently bound together by internucleoside linkages. An oligonucleotide includes a 5′ end and a 3′ end. The 5′ end of an oligonucleotide may be, e.g., hydroxyl, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. The 3′ end of an oligonucleotide may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An oligonucleotide having a 5′-hydroxyl or 5′-phosphate has an unmodified 5′ terminus. An oligonucleotide having a 5′ terminus other than 5′-hydroxyl or 5′-phosphate has a modified 5′ terminus. An oligonucleotide having a 3′-hydroxyl or 3′-phosphate has an unmodified 3′ terminus. An oligonucleotide having a 3′ terminus other than 3′-hydroxyl or 3′-phosphate has a modified 3′ terminus. Oligonucleotides can be in double- or single-stranded form. Double-stranded oligonucleotide molecules can optionally include one or more single-stranded segments (e.g., overhangs).

The term “oxo,” as used herein, represents a divalent oxygen atom (e.g., the structure of oxo may be shown as ═O).

The term “pharmaceutically acceptable,” as used herein, refers to those compounds, materials, compositions, and/or dosage forms, which are suitable for contact with the tissues of an individual (e.g., a human), without excessive toxicity, irritation, allergic response, and other problem complications commensurate with a reasonable benefit/risk ratio.

The term “pharmaceutical composition,” as used herein, represents a composition containing an oligonucleotide described herein, formulated with a pharmaceutically acceptable excipient, diluent, or carrier, and manufactured or sold with the approval of a governmental regulatory agency as part of a therapeutic regimen for the treatment of disease in a subject.

The term “protecting group,” as used herein, represents a group intended to protect a functional group (e.g., a hydroxyl, an amino, or a carbonyl) from participating in one or more undesirable reactions during chemical synthesis. The term “O-protecting group,” as used herein, represents a group intended to protect an oxygen containing (e.g., phenol, hydroxyl or carbonyl) group from participating in one or more undesirable reactions during chemical synthesis. The term “N-protecting group,” as used herein, represents a group intended to protect a nitrogen containing (e.g., an amino or hydrazine) group from participating in one or more undesirable reactions during chemical synthesis. Commonly used O- and N-protecting groups are disclosed in Greene, “Protective Groups in Organic Synthesis,” 3^{rd} Edition (John Wiley & Sons, New York, 1999), which is incorporated herein by reference. Exemplary O- and N-protecting groups include alkanoyl, aryloyl, or carbamyl groups such as formyl, acetyl, propionyl, pivaloyl, t-butylacetyl, 2-chloroacetyl, 2-bromoacetyl, trifluoroacetyl, trichloroacetyl, phthalyl, o-nitrophenoxyacetyl, α-chlorobutyryl, benzoyl, 4-chlorobenzoyl, 4-bromobenzoyl, t-butyldimethylsilyl, tri-iso-propylsilyloxymethyl, 4,4′-dimethoxytrityl, isobutyryl, phenoxyacetyl, 4-isopropylpehenoxyacetyl, dimethylformamidino, and 4-nirobenzoyl.

Exemplary O-protecting groups for protecting carbonyl containing groups include, but are not limited to: acetals, acylals, 1,3-dithianes, 1,3-dioxanes, 1,3-dioxolanes, and 1,3-dithiolanes.

Other O-protecting groups include, but are not limited to: substituted alkyl, aryl, and arylalkyl ethers (e.g., trityl; methylthiomethyl; methoxymethyl; benzyloxymethyl; siloxymethyl; 2,2,2,-trichloroethoxymethyl; tetrahydropyranyl; tetrahydrofuranyl; ethoxyethyl; 1-[2-(trimethylsilyl)ethoxy]ethyl; 2-trimethylsilylethyl; t-butyl ether; p-chlorophenyl, p-methoxyphenyl, p-nitrophenyl, benzyl, p-methoxybenzyl, and nitrobenzyl); silyl ethers (e.g., trimethylsilyl; triethylsilyl; triisopropylsilyl; dimethylisopropylsilyl; t-butyldimethylsilyl; t-butyldiphenylsilyl; tribenzylsilyl; triphenylsilyl; and diphenymethylsilyl); carbonates (e.g., methyl, methoxymethyl, 9-fluorenylmethyl; ethyl; 2,2,2-trichloroethyl; 2-(trimethylsilyl)ethyl; vinyl, allyl, nitrophenyl; benzyl; methoxybenzyl; 3,4-dimethoxybenzyl; and nitrobenzyl).

Other N-protecting groups include, but are not limited to, chiral auxiliaries such as protected or unprotected D, L or D, L-amino acids such as alanine, leucine, phenylalanine, and the like; sulfonyl-containing groups such as benzenesulfonyl, p-toluenesulfonyl, and the like; carbamate forming groups such as benzyloxycarbonyl, p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyl oxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydroxy carbonyl, t-butyloxycarbonyl, diisopropylmethoxycarbonyl, isopropoxycarbonyl, ethoxycarbonyl, methoxycarbonyl, allyloxycarbonyl, 2,2,2-trichloroethoxycarbonyl, phenoxycarbonyl, 4-nitrophenoxy carbonyl, fluorenyl-9-methoxycarbonyl, cyclopentyloxycarbonyl, adamantyloxycarbonyl, cyclohexyloxycarbonyl, phenylthiocarbonyl, and the like, arylalkyl groups such as benzyl, triphenylmethyl, benzyloxymethyl, and the like and silyl groups such as trimethylsilyl, and the like.

The term “shRNA,” as used herein, refers to a double-stranded oligonucleotide of the invention having a passenger strand and a guide strand, where the passenger strand and the guide strand are covalently linked by a linker excisable through the action of the Dicer enzyme.

The term “siRNA,” as used herein, refers to a double-stranded oligonucleotide of the invention having a passenger strand and a guide strand, where the passenger strand and the guide strand are not covalently linked to each other.

The term “skipmer,” as used herein, refers a gapmer, in which alternating internucleoside linkages are phosphate phosphodiester linkages and intervening internucleoside linkages are modified internucleoside linkages.

The term “stereochemically enriched,” as used herein, refers to a local stereochemical preference for one enantiomer of the recited group over the opposite enantiomer of the same group. Thus, an oligonucleotide containing a stereochemically enriched internucleoside linkage is an oligonucleotide, in which a phosphorothioate of predetermined stereochemistry is present in preference to a phosphorothioate of stereochemistry that is opposite of the predetermined stereochemistry. This preference can be expressed numerically using a diastereomeric ratio for the phosphorothioate of the predetermined stereochemistry. The diastereomeric ratio for the phosphorothioate of the predetermined stereochemistry is the molar ratio of the diastereomers having the identified phosphorothioate with the predetermined stereochemistry relative to the diastereomers having the identified phosphorothioate with the stereochemistry that is opposite of the predetermined stereochemistry. The diastereomeric ratio for the phosphorothioate of the predetermined stereochemistry may be greater than or equal to 1.1 (e.g., greater than or equal to 4, greater than or equal to 9, greater than or equal to 19, or greater than or equal to 39).

The term “subject,” as used herein, refers to a human or non-human animal (e.g., a mammal) that is suffering from, or is at risk of, disease, disorder, or condition, as determined by a qualified professional (e.g., a physician or a nurse practitioner) with or without known in the art laboratory test(s) of sample(s) from the subject. The subject treated according to the methods of the invention may thus be a human patient, such as an adult patient or a pediatric patient. Non-limiting examples of diseases, disorders, and conditions include cancers. As one example, the cancer may be characterized by a mutant receptor tyrosine kinase (RTK; e.g., mutant epidermal growth factor receptor (EGFR), human EGFR2 (HER2), HER3, anaplastic lymphoma kinase (ALK), ROS1, ERBB2/3/4, KIT, MET/hepatocyte growth factor receptor (HGFR), RON, platelet derived growth factor receptor (PDGFR), vascular endothelial cell growth factor receptor (VEGFR), VEGFR1, VEGFR2, fibroblast growth factor receptor (FGFR), insulin-like growth factor 1 receptor (IGF1R), or RET). In various embodiments, the cancer may be tolerant or resistant to anti-RTK therapy, or at risk of such tolerance or resistance. Other examples of cancers that the subject may have or be at risk of developing are provided below. A subject treated according to the methods of the invention can optionally be at risk of developing cancer, diagnosed with cancer, in treatment for cancer, or in post-therapy recovery from cancer. The cancer treated according to the methods of the invention can optionally be a primary tumor, locally advanced, or metastatic.

A “sugar” or “sugar moiety” includes naturally occurring sugars having a furanose ring or a structure that is capable of replacing the furanose ring of a nucleoside. Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

The term “tailmer,” as used herein, refers to an oligonucleotide having an RNase H recruiting region (gap) flanked by a 3′ wing including at least one affinity enhancing nucleoside (e.g., 1, 2, 3, or 4 affinity enhancing nucleosides).

“Treatment” and “treating,” as used herein, refer to the medical management of a subject with the intent to improve, ameliorate, stabilize, prevent, or delay a disease, disorder, or condition (e.g., cancer, such as, for example, a cancer characterized by a mutant receptor tyrosine kinase (RTK), which is optionally resistant to RTK-targeted therapy). This term includes active treatment (treatment directed to improve the cancer, or to improve tolerance or resistance to treatment); causal treatment (treatment directed to the cause of the cancer, or to tolerance or resistance to treatment); palliative treatment (treatment designed for the relief of symptoms of the cancer, or for alleviating tolerance or resistance to treatment); preventative treatment (treatment directed to minimizing or partially or completely inhibiting the development of the cancer, or to minimizing or partially or completely inhibiting the development of resistance or tolerance to treatment); and supportive treatment (treatment employed to supplement another therapy).

The term “unimer,” as used herein, refers to an oligonucleotide having a pattern of structural features characterized by all of the internucleoside linkages having the same structural feature. By same structural feature is meant the same stereochemistry at the internucleoside linkage phosphorus or the same modification at the linkage phosphorus. In instances, where the “same structural feature” refers to the stereochemical configuration of the internucleoside linkages, the unimer is a “stereounimer.”

Enumeration of positions within oligonucleotides and nucleic acids, as used herein and unless specified otherwise, starts with the 5′-terminal nucleoside as 1 and proceeds in the 3′-direction.

The compounds described herein, unless otherwise noted, encompass isotopically enriched compounds (e.g., deuterated compounds), tautomers, and all stereoisomers and conformers (e.g. enantiomers, diastereomers, E/Z isomers, atropisomers, etc.), as well as racemates thereof and mixtures of different proportions of enantiomers or diastereomers, or mixtures of any of the foregoing forms as well as salts (e.g., pharmaceutically acceptable salts).

### BRIEF DESCRIPTION OF THE DRAWINGS

FIGS. 1a-1h. NSCLC cells adopt a tolerance strategy against EGFR-TKIs.

FIG. 1a: Representative phase contrast images of organoids from AALE cells cultured according to the protocol at top of the panel. Scale bar, 50 μm.

FIG. 1b: Top, the scenario of anti-EGFR tolerance and resistance in lung cancer. The tumor cells treated with the EGFR-TKI gefitinib or osimertinib enter a reversible drug-tolerant cycle (all arrows except for the two that are not curved, 1° Tolerant) with a brief therapy withdrawal (up to 21 days) followed by reinstatement of the 160 nM dose (2° Tolerant). Alternatively, the tumor cells treated continuously with gefitinib or osimertinib without therapy interruption undergo drug-tolerance briefly and go into a drug-resistance state in which cells do not respond to gefitinib (1° Resistant)/osimertinib (2° Resistant). Bottom, osimertinib treatment response on HCC827 organoids. Representative images of Parental cells, 1° Tolerant cells (derived from the Parental cells treated with 160 nM osimertinib for 11 days), Recovered cells (derived from the 1° Tolerant cells with a therapy withdrawal up to 21 days), and 2° Tolerant cells (derived from the Recovered cells by reinstatement of the 160 nM dose for 11 days). Scale bar, 200 μm.

FIG. 1c: Representative phase contrast microscopy (left panel) and H&E staining of HCC827 organoids derived from parental (top) and osimertinib-tolerant (bottom) cells. Images in dotted squares (middle panel) were amplified (right panel) and shown. Scale bar, 50 μm.

FIG. 1d: qRT-PCR analysis of SFTPC, HOPX, ID2, and CEACAM5 expression in single cell clone HCC827-derived organoids in the presence of osimertinib. Single cell clone derived cells were plated with geltrex and treated with 100 nM osimertinib (tolerant) or vehicle (parental) for 24 days. Gene expression for surviving organoids were analyzed. n=3 replicates.

FIG. 1e: Single cell clonogenicity of PC9 cells treated with gefitinib. A single cell was sorted by FACS into a 96-well plate and treated with 0.1, 0.4, and 2 μM gefitinib or the vehicle for 14 days. The frequency of colony formation was calculated as a ratio of the total number of colonies to the total number of wells plated with a single cell.

FIG. 1f: qRT-PCR analysis of top upregulated and downregulated genes in gefitinib-tolerant clones (n=2) compared with vehicle-treated parental single cell clone (n=1) in PC9. The gene expression in parental sensitive clone was calibrated as 1. ACTB was used as endogenous control. n=3 replicates.

FIG. 1g: Whole transcriptome and gene ontology analysis of gefitinib-tolerant clones (n=2) compared with the parental single cell clone (n=1) in PC9. The top bar in each set is “experimental” and the bottom bar in each set is “predicted.”

FIG. 1h: qRT-PCR analysis of genes in top regulated signaling pathways including Wnt planar cell polarity signaling, glutamine metabolic process, cellular response to hypoxia, and tricarboxylic acid cycle in gefitinib-tolerant clones (n=2) compared with parental the single cell clone (n=1) in PC9. The gene expression in parental sensitive clone was calibrated as 1. ACTB was used as endogenous control. n=3 replicates.

Data are mean±s.e.m. *P<0.05; **P<0.01; ***P<0.001; unpaired t test with Welch's correction (FIG. 1d); modified Fisher's exact test (FIG. 1g).

FIGS. 2a-2e. Gene expressions in lung organoids are comparable to clinical lung tissues.

FIG. 2a: Representative ZO-1 and Hoechst33342 whole-mount immunofluorescent staining on organoids from AALE cells. Z-stack confocal images were acquired with a 2-μm slice interval and 3-D projection was created. Scale bar, 50 μm.

FIG. 2b: Representative H&E staining on organoids from AALE cells. Three consecutive sections for H&E staining (1, 2, and 3) are shown. ★, the lumen in the same organoids. Scale bar, 50 μm.

FIG. 2c: Representative phase contrast microscopic images of organoids from AALE cells on day 24 at passages 2 and 15, respectively. Scale bar, 50 μm.

FIG. 2d: qRT-PCR analysis of ID2, SFTPC, HOPX, and NKX2.1 expression in AALE-derived organoids. Lung organoids established during culture on day 15 and 24 were analyzed. The relative gene expression in human adult lung was used as calibrated as 1. n=3 replicates. ***, P<0.001; ***, P<0.0001 (organoid d24 versus d15); unpaired two-tailed t test.

FIG. 2e: qRT-PCR analysis of CEACAM5, LIN28B, SFTPC, and HOPX expressions in organoids from lung adenocarcinoma patient-derived xenograft (PDX). Lung PDX tumor and PDX-derived organoids established during culture on day 15 and 24 were analyzed. The relative gene expression in human adult lung was calibrated as 1. n=3 replicates.

FIGS. 3a-3e. Lung tumor cells enter a reversible drug-tolerant state with EGFR-TKIs treatment.

FIG. 3a: Osimertinib treatment response on HCC827 cell monolayers and organoids for three days. The cell viability was measured on day 4. LD50, the median lethal dose. The monolayer curve is the straighter line.

FIG. 3b: Representative images of HCC827 parental (P) and tolerant (T) cells in cell monolayer and organoids. Parental cell monolayer (P) were derived from HCC827 cells plated at 300 single cells per 10-cm dish for 10 days followed by a treatment with 160 nM osimertinib for 12 days (T). The parental organoids (P) were derived by seeding 2000 single cells into 3D cultures in 96-well plate for 20 days followed by a treatment with 160 nM osimertinib for 21 days (T). The cell monolayer cultures were stained with Giemsa before images were taken. Scale bar, 1 mm.

FIG. 3c: Representative images of HCC827 drug-tolerant (T) organoids (middle) upon continuous treatments with the 160 nM dose (left arrow) and the increasing 480 nM dose of osimertinib (right arrow) for 9 days.

FIG. 3d: Representative images and treatment response of EGFR-TKI gefitinib on PC9 cell monolayers. Left, the tumor cells treated with the gefitinib for 6 days enter a reversible drug-tolerant cycle (all arrows, 1° Tolerant) with a brief therapy withdrawal (up to 16 days) followed by reinstatement of the 160 nM dose for 11 days (2° Tolerant). Right, the treatment response curve on the 1° Tolerant cells was shown. Scale bar, 200 μm. The “Tolerant PC9” is the top curve and the “Parental PC9” is the bottom curve.

FIG. 3e: Representative images and treatment response of osimertinib on H1975 cell monolayers. Top panel: (left) the tumor cells treated with the osimertinib for 12 days enter a reversible drug-tolerant cycle (all arrows except for the last one, 1° Tolerant) with a brief therapy withdrawal (up to 20 days) followed by reinstatement of the 160 nM dose for 12 days (2° Tolerant). (Right) The treatment response curve on the 1° Tolerant cells was shown. Bottom panel: 160 nM osimertinib was added into the confluent cells on day 0 and treated continuously across the periods as indicated. The fresh media was changed every three days. Scale bar, 200 μm. The “Tolerant H1975” is the top curve and the “Parental H1975” is the bottom curve.

Data are mean±s.e.m.

FIG. 4. Pyrosequencing for quantitative analysis of EGFR exon 19 and 20 sequence variations. Gefitinib-tolerant cells (top), parental cells (middle), and gefitinib-resistant cells (bottom) in PC9 were analyzed.

FIGS. 5a-6d. Frequency and gene expressions for drug-tolerance in single-cell derived clones from PC9 and HCC827.

FIGS. 5a and 5b: Frequency of drug-tolerant single cell clones in PC9 and HCC827 cells. Single cell-derived clones from PC9 (FIG. 5a) and HCC827 (FIG. 5b) were treated with gefitinib (2 μM) and osimertinib (2 μM), respectively. Following 14 days of treatment surviving drug-tolerant colonies were quantified. Single cell-derived clones from PC9 and HCC827 are designated single-cell clone 1, 2, 3, and 4.

FIGS. 5c and 5d: qRT-PCR analysis of genes in hypoxia signature (FIG. 5c) and TCA cycle (FIG. 5d) in osimertinib-tolerant single-cell clone 1 compared with parental PC9 clone. Each experiment was performed in triplicate.

FIGS. 6a-6g. MIR-147b initiates drug-tolerance.

FIG. 6a: A heat map showing top upregulated and downregulated miRNAs in two paired osimertinib-tolerant (OTR) and parental cells in PC9 and HCC827 by miRNA-seq analysis.

FIG. 6b: qRT-PCR analysis of miR-147b expressions in parental, recovered, primary, and secondary osimertinib-tolerant cells in PC9. The parental tumor cells treated with 160 nM EGFR-TKI osimertinib for 6 days enter a drug-tolerant state (primary tolerant cells) with a brief therapy withdrawal up to 18 days (recovered cells) followed by reinstatement of the 160 nM dose for 11 days (secondary tolerant cells). The relative miR-147b expression level in the parental cells were calibrated as 1. MiR-423 was used as endogenous control. n=3 replicates.

FIGS. 6c and 6d: Osimertinib (c) and gefitinib (d) treatment response on scrambled control (Scr) and miR-147b-overexpressing cells (147b) in HCC827 for 3 days. The top curve is 147b and the bottom curve is Scr in FIG. 6c and FIG. 6d. n=3 replicates.

FIG. 6e: Osimertinib (40 nM) and gefitinib (40 nM) treatment response on scrambled control and miR-147b-overexpressing cells in HCC827 by colony formation assay. 20, 40, and 80 cells were plated in 10-cm dish and the colonies were stained with Giemsa on day 10 and the total number of colonies were quantified. n=3 replicates. The left bar of each pair is “Scr” and the right bar of each pair is “147b.”

FIG. 6f-Osimertinib treatment response on H1975 cells with miR-147b knockdown (anti147b) and scrambled control (antictrl). The cell viability was measured on day 4. n=3 replicates. The top curve is “antictrl” and the bottom curve is “anti147b.”

FIG. 6g: Osimertinib (160 nM) treatment response on H1975 cells with miR-147b knockdown. Left, the monolayer colonies were treated for 10 days and stained with Giemsa. Right, the organoids were treated for 14 days. −, vehicle; +, osimertinib. Scale bar, 1000 μm. The left bar of each pair is “antictrl” and the right bar of each pair is “anti147b.”

Data are mean±s.e.m. *P<0.05; **P<0.01; ***p<0.001; one-way ANOVA (FIG. 6b); unpaired two-tailed t-test (FIGS. 6e and 6g).

All data are representative of two separate experiments.

FIGS. 7a-7h. MIR-147b expression levels increase in EGFR tyrosine kinase inhibitor-tolerant lung cancer cell line and patient-derived xenografts.

FIG. 7a: qRT-PCR analysis for miR-147b expressions in gefitinib and osimertinib tolerant cells compared with parental cells in PC9 and HCC827.

FIG. 7b: Osimertinib treatment response for three days on organoids from two representative EGFR mutant lung patient-derived xenografts (PDX_LU_10 and PDX_LU_11). The cell viability was measured on day 4. LD50, the median lethal dose. The top curves are the tolerant PDX samples and the bottom curves are the parental PDX samples.

FIGS. 7c and 7d: qRT-PCR analysis for miR-147b (FIG. 7c) and hypoxia genes (FIG. 7d) expression in osimertinib-tolerant organoids compared to parental organoids from lung PDXs (n=5).

FIGS. 7e and 7f-Representative phase contrast images of osimertinib-tolerant organoids derived from HCC827 single cell-derived organoids on day 1 (d1, FIG. 7e) and on day 24 (FIG. 7) with continuous osimertinib treatment at 100 nM for 21 days. Scale bar, 100 μm.

FIG. 7g: Relative expression for miR-147b and hypoxia genes in osimertinib-tolerant organoids from (FIG. 7e) and (FIG. 7f). The relative expression in organoids on day 1 treated with 100 nM osimertinib is calibrated as 1.

FIG. 7h: Relative expression for miR-147b and hypoxia genes in HCC827 single cells-derived organoids on day 2 (d2), d4, and d6 during cultures. The relative gene expression in organoids on d2 is calibrated as 1. n=3 replicate.

Data are mean±s.e.m. *P<0.05; **P<0.01; ***P<0.001; unpaired two-tailed t-test (FIGS. 7a, 7c, and 7d) and unpaired t test with Welch's correction (FIG. 7g) and two-way ANOVA analysis (FIG. 7h).

FIGS. 8a-8f. Upregulated mIR-147b expression is relevant to EGFR mutations in human lung cancer cell lines and tumor tissues.

FIG. 8a: Left, a heat map showing differential miRNA expression between EGFR mutant (mut, n=8) and RAS mutant (n=17) human lung adenocarcinoma cell lines. Right, scatter plot for differential miR-147b expression.

FIG. 8b: Fold change expression of miR-938, miR-141, miR-559, miR-200c, miR-136, miR-718, miR-548N, and miR-191 in EGFR mutant cell lines (n=8) compared to RAS mutant cell lines (n=17) in lung adenocarcinoma.

FIG. 8c: Real-time quantitative RT-PCR analysis for miR-147b expressions across human malignant lung cell lines with EGFR wild-type (WT, n=5), EGFR sensitizing mutations (n=4), and resistant mutations (n=3). The relative miR-147b expression in normal lung epithelial cell (AALE) was calibrated as 1.

FIG. 8d: Scatter plot with bar for miR-147b expression level in EGFR mutant lung cancer patient-derived xenografts (PDXs, n=5) relative to EGFR wild-type (WT, n=5) PDXs. The relative miR-147b expression level in human normal lung tissue was calibrated as 1. n=3 replicates.

FIG. 8e: Association between miR-147b expression levels and EGFR or KRAS mutations in lung adenocarcinoma tissues from the TCGA dataset (n=106).

FIG. 8f-miR-147b expression in EGFR and KRAS mutant lung adenocarcinoma tissues from the TCGA dataset. The cut-off value (horizontal axis crosses axis value) of low and high miR-147b expression level is the median value (0.84) across all tested 106 tissues. Read counts are reads per million miRNA mapped.

Data are mean±s.e.m. *P<0.05; **P<0.01; ***P<0.001; unpaired two-tailed t-test (a), Mann-Whitney test (FIGS. 8b, 8c, and 8d); unpaired two-tailed t-test with Welch's correction (FIG. 8e) and Fisher's exact test (FIG. 8f).

FIGS. 9a-9d. MIR-147b links osimertinib-tolerance to cancer stemness in H1975 cells.

FIG. 9a: Representative fluorescent image (top) and phase-contrast image (bottom) of H1975 tumor spheroids. Scale bar, 1000 μm.

FIG. 9b: Left, representative images of H1975 tumor spheroids infected with miR-147b inhibitor (anti147b) and scrambled control (antictrl). Right, spheroid quantification on day 7. n=3 replicates. Scale bar, 1000 μm. The first bar in each set is “antictrl” and the second bar in each set is “anti147b.”

FIG. 9c: Limiting dilution analysis and tumor spheroid formation assay for the frequency of tumor initiating cells (TICs) in H1975 cells with miR-147b knockdown or scrambled control. 1800, B00, and 300 single cells were plated into ultra-low attachment plates in serum-free media and the total number of spheroids was quantified on day 11. n=3 replicates.

FIG. 9d: Fold change for gene expression in H1975 cells with miR-147b knockdown relative to scrambled control by qRT-PCR analysis. n=3 replicates.

Data are mean±s.e.m. *P<0.05; **P<0.01; ***P<0.001; unpaired two-tailed t-test (FIGS. 9b, 9c, and 9d).

FIGS. 10a-10d. Depletion of mIR-147b with CRISPR reduces osimertinib tolerant state in H1975 cells.

FIG. 10a: qRT-PCR analysis of miR-147b expression levels in H1975 cells with CRISPR depletion. Cells were transfected with crRNA-147b 1 and 4 (1+4):tracrRNA. n=3 replicates.

FIGS. 10b and 10c: Cell viability of H1975 cells transfected with crRNA-147b 1 and 4:tracrRNA and negative control in monolayer culture (FIG. 10b) and organoids (FIG. 10c) for 3 days.

FIG. 10d: Osimertinib treatment response on H1975 organoids. The cells were transfected with crRNA-147b 1 and 4 (1+4):tracRNA and then treated with 100 nM osimertinib. The cell viability was measured after 72 hours. The relative cell viability in negative control cells treated with DMSO is calibrated as 1.

Data are mean±s.e.m. **, P<0.05; ***, P<0.001; ****, P<0.0001; two-tailed t-test (FIGS. 10a, 10b, and 10c), and two-way ANOVA (FIG. 10d).

FIGS. 11a-11e. MIR-147b-VHL axis mediates drug-tolerance through impaired VHL activity.

FIG. 11a: Left, gene candidates predicted for miR-147b by the TargetScan tool were shown in signaling pathways enriched for gefitinib-tolerance in PC9 single-cell clones in FIG. 1f. Right, qRT-PCR analysis for the predicted gene candidates for miR-147b in H1975 cells with miR-147b knockdown compared with scrambled control.

FIG. 11b: Left, computational prediction of RNA duplex formation between miR-147b (SEQ ID NO: 2) and the 3′UTR (untranslated region) of VHL mRNA (SEQ ID NO: 906). Mutations generated within the 3′UTR for the luciferase assay are shown by underlining (SEQ ID NOs: 907 and 908). Right, dual-luciferase reporter assay in miR-147b-overexpressing AALE cells. The Firefly luciferase and Renilla luciferase activities were measured 48 hours post co-transfection with miR-147b or control vector and wild-type (WT) or mutant (Mut) VHL 3′UTR. The first bar in each set is “Scr” and the second bar in each set is “147b.”

FIG. 11c: Western blot analysis and quantification of VHL in miR-147b-overexpressing AALE cells. β-Actin was used as loading control.

FIG. 11d: qRT-PCR analysis for fold change of hypoxia gene expression in AALE cells with miR-147b overexpression relative to scrambled control (147b/Scr) and cells with co-overexpression of miR-147b and VHL relative to scrambled control (147b+VHL/Scr). ACTB was used as endogenous control. n=3 replicates. The first bar in each set is “147b/Scr” and the second bar in each set is “147b+VHL/Scr.”

FIG. 11e: Fractional viability of HCC827 cells treated with vehicle, osimertinib (20 nM), miR-147b vector, VHL vector, or combinations. The cell viability was measured on day 3. The relative cell viability treated with vehicle on day 3 was calibrated as 1. n=3 replicates.

Data are mean±s.e.m. *P<0.05; **P<0.01; ***P<0.001; NS, not significant (P>0.05); unpaired two-tailed t-test (FIG. 11a-d); Kruskal-Wallis test (FIG. 11e). All data are representative of two separate experiments.

FIGS. 12a-12c. VHL and miRNA gene expression correlation reveals negative association between VHL and MIR147b.

FIG. 12a: Top candidate VHL-regulating miRNAs emerging from TargetScan tool with weighted context++ score.

FIGS. 12b and 12c: Scatterplots showing the expression of VHL on the x axis and the expressions of MIR147B (FIG. 12b) and other miRNA candidates (FIG. 12c) listed in (FIG. 12a) on the y axis in the human lung adenocarcinoma cell lines. n=80. Statistical significance was calculated using Spearman correlation test.

FIGS. 13a-13f. MIR-147b-SDH axis mediates drug tolerance through SDH enzyme activity in the TCA cycle.

FIG. 13a: Left, computational prediction of RNA duplex formation between miR-147b (SEQ ID NO: 2) and the 3′UTR of SDHD mRNA (SEQ ID NO: 909). Mutations generated within the 3′UTR for the luciferase assay are shown by underlining (SEQ ID NOs: 910 and 911). Right, dual-luciferase reporter assay in miR-147b-overexpressing AALE cells. The Firefly luciferase and Renilla luciferase activities were measured 48 hours post co-transfection with miR-147b or control vector and wild-type (WT) or mutant (Mut) SDHD 3′UTR. n=3 replicates.

FIG. 13b: Principal component analysis (PCA) of parental cells, osimertinib-tolerant cells (H1975OTR), and tolerant cells with miR-147b knockdown (H1975OTR-anti147b) in H1975 cell monolayers. The tolerant cells were derived from the parental cells treated with 100 nM osimertinib continuously for 21 days. n=5 replicates. The top set of points is H1975, the middle set of points is H1975OTR, and the third set of points is H1975OTR-anti147b.

FIG. 13c: A heat map showing top metabolites levels across cells of H1975, H1975OTR, and H1975OTR-anti147b. n=5 replicates.

FIG. 13d: Levels of succinate, 2-oxoglutarate, fumarate, and malate in cells of H1975, H1975OTR, and H1975OTR-anti147b. The relative levels in the parental H1975 cells were calibrated as 1. n=5 replicates.

FIG. 13e: Schematic of the interaction among miR-147b and SDH enzyme leading to dysregulated TCA cycle metabolites for drug-tolerance to EGFR tyrosine kinase inhibitors. Upregulated levels of oxoglutarate and succinate (in red) as well as downregulated levels of fumarate and malate (in green) in drug-tolerant cells are highlighted.

FIG. 13f: SDH inhibitor promotes drug-tolerance to osimertinib in H1975 cells. Vehicle or 100 nM osimertinib (osim)-treated cells were co-incubated with 0, 0.03, and 0.1 mM membrane-permeable dimethyl malonate (DMM) for 3 days. The cell viability was measured on day 4. Data are mean±s.e.m. *P<0.05; **P<0.01; ***P<0.001; ****P<0.0001; NS, not significant (P>0.05); unpaired two-tailed t-test (FIGS. 13a and 13); one-way ANOVA (FIG. 13d). The first bar of each set is 0 mM DMM, the second bar of each set is 0.03 mM DMM, and the third bar of each set is 0.1 mM DMM.

FIGS. 14a-14c. Metabolomics study in osimertinib-tolerant cell monolayers and organoids in H1975.

FIG. 14a: Levels of NAD+ and GSH across parental cells, osimertinib-tolerant cells, and osimertinib-tolerant cells with miR-147b knockdown (anti147b) in H1975 cells. n=5 replicates.

FIG. 14b: Partial-Least Squares Discriminant analysis (PLS-DA) of H1975 parental organoids, tolerant organoids, and tolerant organoids with miR-147b knockdown (anti147b). n=5 replicates. The first set of ponts is parental organoid, the second set of points is tolerant organoid, and the third set of points is tolerant organoid-anti147b.

FIG. 14c: Levels of fumarate, malate, and NAD+ across parental organoids, osimertinib-tolerant organoids, and osimertinib-tolerant organoids with miR-147b knockdown in H1975 cells. n=5 replicates.

Data are mean±s.e.m. *P<0.05; **P<0.01; ***P<0.001; ****P<0.0001; unpaired two-tailed t-test (FIGS. 14a and c).

FIGS. 15a-15g. Knocking down miR-147b by a LNA inhibitor inhibits tumor growth and potentiates osimertinib treatment in H1975 cells.

FIG. 15a: Subcutaneous xenograft tumor growth by H1975 cells with miR-147b knockdown (anti147b) compared to scrambled control (antictrl). 100,000 cells in serum-free medium and growth factor reduced Matrigel were inoculated into the flank of nude mice on day 0. The xenograft tumor formation was monitored by calipers twice a week. The recipient mice were euthanized on day 22. n=5 mice each group. The top curve is “antictrl” and the bottom curve is “anti147b.”

FIG. 15b: Images of xenograft tumors and quantification of tumor mass on day 22 post-transplantation. n=5 mice per group.

FIG. 15c: qRT-PCR analysis for miR-147b expression in H1975 cells with LNA miR-147b inhibitor upon 2 days post-transfection compared to scrambled LNA control. The fold change for miR-147b expression in scrambled control cells was calibrated as 1.

FIG. 15d: Osimertinib treatment response on H1975 organoids. The IC50 for osimertinib decreased 30-fold in cells with 120 nM LNA miR-147b inhibitor (anti147b) compared to scrambled control (antictrl). n=3 replicates. The tope curve is “LNA antictrl” and the bottom curve is “LNA anti147b.”

FIGS. 15e and 15f: qRT-PCR analysis for hypoxia gene expression in H1975 cells treated with 10 μM DMOG (e) and 30 μM R59949 (f) for three days. The relative gene expression in cells treated with vehicle was calibrated as 1 (dotted line). n=3 replicates.

FIG. 15g: qRT-PCR analysis for hypoxia gene expression in H1975 cells treated with 90 nM LNA miR-147b inhibitor, 30 μM R59949, or combinations for three days. The relative gene expression in scrambled control cells treated with vehicle was calibrated as 1 (dotted line). n=3 replicates. The first bar in each set is “LNA-anti147b+vehicle” and the second bar in each set is “LNA-anti147b+R9949.”

All figures show mean±s.e.m. *p<0.05; *p<0.01 and **p<0.001. unpaired two-tailed t-test (FIG. 15a, 15b, 15e, 15f, and 15g); one-way ANOVA (c).

FIGS. 16a-16i. Blocking miR-147b overcomes drug-tolerance.

FIG. 16a: Fractional viability of H1975 organoids treated with osimertinib (25 nM), LNA miR-147b inhibitor (LNA-anti147b, 90 nM), DMOG (10 μM), or combinations for 14 days.

FIG. 16b: qRT-PCR analysis for hypoxia gene expression in H1975 cells treated with 90 nM LNA miR-147b inhibitor (LNA-anti147b) and 10 μM DMOG or vehicle for three days. The relative gene expression in scrambled control cells treated with vehicle was calibrated as 1. n=3 replicates. The first bar in each set is “LNA-anti147b+vehicle” and the second bar in each set is “LNA-anti147b+DMOG.”

FIG. 16c: Fractional viability of H1975 organoids treated with 25 nM osimertinib, 90 nM LNA-anti147b, 30 μM R59949, or combinations for 14 days.

FIG. 16d: qRT-PCR analysis of HIF1A in H1975 cells with shRNAs against HIF1A. H1975 cells were transfected with shRNAs against HIF1A (shHIF1A-1 and -2) or scrambled control (shCtrl) and selected with 0.5 μg/ml puromycin. GAPDH was used as endogenous control.

FIG. 16e: Cell viability of H1975 cells with HIF1A knockdown treated with osimertinib. The cells with shRNAs against HIF1A (shHIF1A-1 and shHIF1A-2) and scrambled control cells (shCtrl) were treated with 100 nM osimertinib or vehicle for 3 days. The cell viability was analyzed on day 4. The first bar in each set is “DMSO” and the second bar in each set is “100 nM Osim.”

FIG. 16f: Cell viability of H1975 cells with constitutive active HIF1A mutant treated with osimertinib. The cells were transfected with HIF1A A588T and scrambled control cells (Scr) followed by 600 μg/ml neomycin selection. Then the cells were treated with 100 nM osimertinib or vehicle for 3 days. The cell viability was analyzed on day 4. The first bar in each set is “DMSO” and the second bar in each set is “100 nM Osim.”

FIG. 16g: Derivation and growth of organoids from lung PDX tumors. (top) Representative phase contrast microscopy for parental EGFR mutant lung PDX-derived organoids in PDX_LU_10 organoid. (Bottom) growth curve of PDX organoids. The organoids size was measured every two days. The media were replenished every three days till day 14. n=3 replicates. Scale bar, 50 μm.

FIG. 16h: Pretreatment response on lung PDX_LU_10 organoids with LNA miR-147b inhibitor (anti147b) and osimertinib. The organoids were established at medium size seven days after seeding 2000 single-cells into 3D cultures in 96-well plate. This time point was recorded as day 0. Then the organoids were administrated with LNA anti147b or antictrl (90 nM) on day 0 and day 2 or osimertinib (25 nM) on day 1 and day 4. The vehicle treated group did not receive treatments with LNA or osimertinib. The organoids size was measured every two days. The media were replenished every three days till day 14. n=3 replicates. The top curve is “LNA-antictrl+osimertinib” and the bottom curve is “LNA-anti147b+osimertinib.”

FIG. 16i: Schematic for miR-147b-driven drug-tolerance model. MiR-147b is enriched in a subpopulation of parental lung cancer cells entering drug-tolerant status when they are treated with EGFR-TKIs. MiR-147b mediates drug-tolerance through repressing activities of VHL and SDH leading to activated pseudohypoxia response. TKI, tyrosine kinase inhibitor; SDH, succinate dehydrogenase; TCA, tricarboxylic acid; PHD, prolyl-hydroxylase.

Data are mean±s.e.m. *P<0.05; **P<0.01; ***P<0.001; ****P<0.0001; NS, not significant; Kruskal-Wallis test (FIGS. 16a and 16c); unpaired two-tailed t-test (FIG. 16b, 16d, 16e, 16f, and 16h). All data are representative of two separate experiments.

FIGS. 17a and 17b. Drug-tolerance to osimertinib in H1975 cells is not dependent on EPAS1.

FIG. 17a: qRT-PCR analysis of EPAS1 in H1975 cells with shRNAs against EPAS1. H1975 cells were transfected with shRNAs against EPAS1 (shEPAS1-1 and shEPAS1-2) or scrambled control (shCtrl) and selected with 0.5 μg/ml puromycin for 9 days. GAPDH was used as endogenous control.

FIG. 17b: Cell viability of H1975 cells with EPAS1 knockdown treated with osimertinib. The cells with shRNAs against EPAS1 (shEPAS1-1 and shEPAS1-2) and scrambled control cells (shCtrl) were treated with 100 nM osimertinib or vehicle for 3 days. The cell viability was analyzed on day 4. The first bar in each set is “DMSO” and the second bar in each set is “100 nM Osim.”

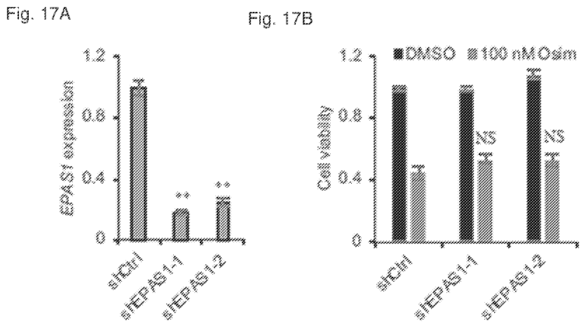

Data are mean±s.e.m. **P<0.01; NS, not significant; unpaired two-tailed t-test (FIGS. 17a and 17b).

### DETAILED DESCRIPTION

The invention is based, in part, on our discovery that miR-147b plays a role in tolerance and resistance of receptor tyrosine kinase (RTK) (e.g., epidermal growth factor (EGFR))-mutated cancer to RTK-targeted therapies, such as tyrosine kinase inhibitors (TKIs). We have also found that miR-147b inhibition can be used to treat cancer. Accordingly, the invention includes methods for treating, reducing, preventing, or delaying tolerance or resistance of cancer to RTK (e.g., EGFR)-targeted therapy by administration of one or more inhibitors of miR-147b, as well as methods of treating or preventing cancer using one or more of these inhibitors. As explained further below, these methods can optionally be carried out in combination with other therapies, such as anti-cancer therapies (e.g., TKIs or anti-RTK antibody therapy; also see below). The invention also provides miR-147b inhibitors, compositions including them (optionally in combination with other agents), diagnostic methods, and screening methods.

The invention is also based, in part, on our discovery of methods to prepare and use three-dimensional organoids including lung-derived cells, e.g., lung cancer cells. Accordingly, the invention also provides such organoids, as well as methods of their use.

The methods, inhibitors, compositions, and organoids of the invention are described further, as follows.

Micro RNAs—miR-147b

Micro RNAs (miRNAs) are small, non-coding RNA modulators of gene activity, which act primarily by base pairing to the 3′-untranslated regions of target RNAs (e.g., mRNAs and pre-mRNAs), leading to target RNA degradation or mRNA translation inhibition. MiRNAs are typically produced as follows. First, an initial transcript, pri-miRNA, is cleaved in the nucleus to generate pre-miRNA, which comprises a stem-loop structure. This molecule is then exported from the nucleus to the cytoplasm, where it is processed by Dicer to generate an miRNA duplex lacking a connecting loop. The reverse-complement of the mature miRNA sequence is then removed from the duplex, and the mature miRNA is incorporated into a multi-component RNA-induced silencing complex (RISC). The mature miRNA, in the context of RISC, can then act by base pairing to a target RNA, as noted above.

MiRNAs play critical roles in many biological processes, and their dysregulation accordingly plays roles in many different diseases. We have found that increased miR-147b levels are associated with tolerance and resistance to anti-RTK therapies, as described herein. We have also found that decreasing miR-147b levels is effective to counter these effects, and also to directly treat cancer. Accordingly, the present invention establishes miR-147b as a therapeutic target for treating, reducing, preventing, or delaying tolerance or resistance to anti-RTK therapy, as well as a target for anti-cancer treatment and prevention.

### Therapeutic Methods

MiR-147b inhibitors, such as those described herein, can be used in therapeutic methods, as noted above. In some examples, the inhibitors are used to treat, reduce, inhibit, or delay tolerance or resistance to an anti-cancer treatment. In particular, the inhibitors can be used in the context of tolerance or resistance of cancer to RTK-targeted therapies including, for example, TKIs and/or anti-RTK immunotherapies (e.g., antibody- or CAR T-based therapies). In other examples, the inhibitors are used to treat or prevent cancer directly.

Examples of RTKs, with respect to which a miR-147b inhibitor of the invention can be used to treat, reduce, inhibit, or delay tolerance or resistance to targeting thereof, include, e.g., epidermal growth factor receptor (EGFR), human EGFR2 (HER2), HER3, anaplastic lymphoma kinase (ALK), ROS1, ERBB2/3/4, KIT, MET/hepatocyte growth factor receptor (HGFR), RON, platelet derived growth factor receptor (PDGFR), vascular endothelial cell growth factor receptor (VEGFR), VEGFR1, VEGFR2, fibroblast growth factor receptor (FGFR), insulin-like growth factor 1 receptor (IGF1R), and RET.

The miR-147b inhibitors can be administered as sole therapeutic agents or, optionally, can be administered in combination with each other or one or more additional therapeutic agents (e.g., one or more anti-RTK therapy). MiR-147b inhibitors can be administered to a subject before, at the same time as, or after another therapeutic agent (e.g., an anti-RTK-targeted therapy), or after multiple rounds of another agent (e.g., an anti-RTK-targeted therapy), as determined to be appropriate by those of skill in the art. Accordingly, in some embodiments, the invention includes combination therapy methods, in which one or more miR-147b inhibitor is administered in combination with one or more other agents (e.g., anti-RTK therapy), and optionally one or more further anti-cancer treatments (see, e.g., below).

In addition to the above, miR-147b inhibitors can also be used to treat or prevent cancer, due to direct anti-cancer effects of the inhibitors. In these methods, the inhibitors can be used alone or in combination with each other or other anti-cancer treatments including (in addition to anti-RTK-targeted therapies), for example, the anti-cancer agents listed below, as well as other treatments (e.g., radiotherapy and surgery).

As noted above, examples of anti-RTK therapies include TKIs, anti-RTK antibodies, and anti-RTK CAR T cells. Examples of TKIs include gefitinib (Iressa®), erlotinib (Tarceva®), afatinib (Gilotrif®), lapatinib (Tykerb®), neratinib (Nertynx®), osimertinib (Tagrisso®), vandetanib (Caprelsa®), crizotinib (Xalcori®), dacomitinib (Vizimpro®), regorafenib (Stivarga®), ponatinib (Iclusig®), vismodegib (Erivedge®), pazopanib (Votrient®), cabozantinib (Cabozantinib®), bosutinib (Bosulif®), axitinib (Inlyta®), vemurafenib (Zelboraf®), ruxolitinib (Jakafi®), nilotinib (Tasigna®), dasatinib (Sprycel®), imatinib (Gleevec®), sunitinib (Sutent®), sorafenib (Nexavar®), trametinib (Mekinist®), cobimetanib (Cotellic®), and dabrafenib (Tafinlar®).

As is known in the art, TKIs such as these vary with respect to the RTKs that they target, and therefore also the cancer types targeted. Selection of a particular TKI for administration to a subject, in the context of a miR-147b inhibitor, can thus be carried out by those of skill in the art depending upon the particular cancer to be treated (see, e.g., Jeong et al., Curr. Probl. Cancer 37(3):110-144, 2013).

Examples of anti-RTK antibodies that can be used in the invention include anti-EGFR antibodies such as, for example, cetuximab (Erbitux®), nimotuzumab (TheraCIM®), necitumumab (Portrazza®), panitumumab (Vedibix®), futuximab, zatuximab, CetuGEX™, and margetuximab. Anti-HER2 antibodies include trastuzumab (Herceptin®), pertuzumab (Perjeta®), trasGEX™. seribantumab, and patritumab. Antibodies against additional RTKs include the following: onartuzumab (HER3), namatumab (RON), ganitumab (RON), cixutumumab (RON), dalotuzumab (IGF1R), teprotumumab (IGF1R), icrucumab (VEGFR1), ramucirumab (VEGFR1), tanibirumab (VEGFR2), and olaratumab (PDGFR) (Fauvel et al., Mabs 6(4):838-851, 2014). Accordingly, miR-147b inhibitors, such as those described herein, can be used to treat, reduce, inhibit, or delay tolerance or resistance to therapies such as these. They can also be administered with such therapies, in order to treat, reduce, inhibit, or delay tolerance, as well as to optionally provide a separate anti-cancer effect.

As noted above, the methods of the invention can also include administration of one or more additional anti-cancer agents. For example, agents such as antimetabolites (e.g., methotrexate, pemetrexed, purine antagonists (e.g., mercaptopurine, thioguanine, fludarabine phosphate, cladribine, or pentostatin), or pyrimidine antagonists (e.g., gemcitabine, capecitabine, fluoropyrimidines, fluorouracil, 5-fluorouracil, cytarabine, or azacitidine)), antibiotics (e.g., anthracyclines (e.g., doxorubicin, epirubicin, daunorubicin, or idarubicin), adriamycin, dactinomycin, idarubincin, plicamycin, mitomycin, bleomycin, or mitoxantrone), alkylating agents (e.g., cyciophosphamide, temozolomide, procarbazine, dacarbazine, altretamine, cisplatin, carboplatin, oxaliplatin, or nitrosoureas), plant alkaloids (e.g., vinblastine, vincristine, etoposide, teniposide, topotecan, irinotecan, paclitaxel, nab-paclitaxel, ABRAXA E® (protein-bound paclitaxel), or docetaxel), anti-tubulin agents (e.g., eribulin, ixabepilone, vinorelbine, or vincristine), anticoagulants (e.g., heparin or warfarin), biological agents (e.g., hormonal agents, cytokines, interleukins, interferons, granulocyte colony stimulating factor (G-CSF), macrophage colony stimulating factor (M-CSF), granulocyte macrophage colony stimulating factor (GM-CSF), or chemokines), and/or anti-angiogenic agents (e.g., angiostatin or endostatin) can be used.

The methods of the invention can further be carried out in combination with immunotherapeutic approaches to treating cancer. These include, for example, anti-CTLA-4 antagonist antibodies (e.g., ipilimumab, Yervoy®, BMS), anti-VEGF antibodies (e.g., bevacizumab, Avastin®), anti-OX40 agonist antibodies (e.g., Medi6469, MedImmune, and MOXR0916/RG7888, Roche), and PD-1 and/or PD-L1 targeted therapies (e.g., nivolumab (Opdivo®, BMS-936558, MDX-1106, and ONO-4538) and pembrolizumab (Keytruda®, MK-3475)). Further immunotherapeutic approaches include anti-TIGIT antagonist antibodies (e.g., BMS-986207, Bristol-Myers Squibb/Ono Pharmaceuticals), IDO inhibitors (see, e.g., US 2016/0060237 and US 2015/0352206; Indoximod, New Link Genetics), RORγ agonists (e.g., LYC-55716 (Lycera/Celgene) and INV-71 (Innovimmune)), and cancer vaccines (e.g., MAGE3 vaccine (e.g., for melanoma and bladder cancer), MUC1 vaccine (e.g., for breast cancer), EGFRv3 (such as Rindopepimut, e.g., for brain cancer, such as glioblastoma multiforme), or ALVAC-CEA (e.g., for CEA+ cancers)).

Further, as noted above, the miR-147b inhibitors of the invention can be used in the context of CAR T cell therapy, e.g., anti-RTK CAR T cell therapy. For example, CAR T cells directed against EGFR, which are useful against, e.g., gliomas and other EGFR⁺ solid tumors, can be used. In another example, CAR T cells directed against EGFRvIII, which are useful against, e.g., glioblastoma multiforme and gliomas, such as EGFRvIII+ gliomas, can be used.

Examples of cancers that can be treated according to the methods of the invention include lung cancer (e.g., adenocarcinoma of the lung; non-small cell lung cancer), colorectal cancer, anal cancer, glioblastoma, head and neck cancer (e.g., squamous cell carcinoma of the head and neck), pancreatic cancer, breast cancer, renal cell carcinoma, squamous cell carcinoma, thyroid cancer, gastroesophageal adenocarcinoma, and gastric cancer.

In further examples, the cancer can be selected from the group consisting of stomach cancer, colon cancer, liver cancer, biliary tract cancer, gallbladder cancer, rectal cancer, renal cancer, bladder cancer, endometrial cancer, ovarian cancer, cervical cancer, vulvar cancer, vaginal cancer, penile cancer, prostate cancer, testicular cancer, pelvic cancer, brain cancer, esophageal cancer, bronchus cancer, oral cancer, oropharyngeal cancer, larynx cancer, thyroid cancer, skin cancer, cancer of the central nervous system, cancer of the respiratory system, and cancer of the urinary system.

In still further examples, the cancer can be selected from the group consisting basal cell carcinoma, large cell carcinoma, small cell carcinoma, non-small cell lung carcinoma, renal carcinoma, hepatocarcinoma, gastric carcinoma, choriocarcinoma, adenocarcinoma, hepatocellular carcinoma, giant (or oat) cell carcinoma, adenosquamous carcinoma, anaplastic carcinoma, adrenocortical carcinoma, cholangiocarcinoma, Merkel cell carcinoma, ductal carcinoma in situ (DCIS), invasive ductal carcinoma, hepatoblastoma, medulloblastoma, nephroblastoma, neuroendocrine tumors, pheochromocytoma, neuroblastoma, pancreatoblastoma, pleuropulmonary blastoma, retinoblastoma, leukemia, B-cell leukemia, T-cell leukemia, acute myeloid leukemia (AML), chronic myeloid leukemia (CML), acute lymphocytic (lymphoblastic) leukemia (ALL), chronic lymphocytic leukemia (CLL), erythroleukemia, lymphoma, Hodgkin's lymphoma, non-Hodgkin's lymphoma, Burkitt lymphoma, follicular lymphoma, diffuse large B-cell lymphoma (DLBCL), thyoma, multiple myeloma, plasmacytoma, localized myeloma, extramedullary myeloma, melanoma, superficial spreading melanoma, nodular melanoma, lentigno maligna melanoma, acral lentiginous melanoma, amelanotic melanoma, ganglioneuroma, Pacinian neuroma, acoustic neuroma, astrocytoma, oligoastrocytoma, ependymoma, glioma, glioblastoma multiforme, brainstem glioma, optic nerve glioma, oligoastrocytoma, pheochromocytoma, meningioma, malignant mesothelioma, and a virally induced cancer.

In additional examples, the cancer is a sarcoma, for example, a sarcoma selected from the group consisting of angiosarcoma, hemangiosarcoma, chondrosarcoma, Ewing's sarcoma, fibrosarcoma, gastrointestinal stromal tumor, leiomyosarcoma, liposarcoma, malignant peripheral nerve sheath tumor, malignant fibrous cytoma, osteosarcoma, pleomorphic sarcoma, rhabdomyosarcoma, synovial sarcoma, vascular sarcoma, Kaposi's sarcoma, dermatofibrosarcoma, epithelioid sarcoma, leiomyosarcoma, and neurofibrosarcoma.

In further examples, the cancer is a breast cancer selected from the group consisting of triple-negative breast cancer, triple-positive breast cancer, HER2-negative breast cancer, HER2-positive breast cancer, estrogen receptor-positive breast cancer, estrogen receptor-negative breast cancer, progesterone receptor-positive breast cancer, progesterone receptor-negative breast cancer, ductal carcinoma in situ (DCIS), invasive ductal carcinoma, invasive lobular carcinoma, inflammatory breast cancer, Paget disease of the nipple, and phyllodes tumor.

Anti-cancer therapies, including miR-147b inhibitors and other anti-cancer therapies, such as those described above, are administered in the practice of the methods of the invention as is known in the art (e.g., according to FDA-approved regimens or other regimens determined to be appropriate by those of skill in the art). In some embodiments, anti-cancer therapies of the invention are administered in amounts effective to treat, reduce, inhibit, or delay resistance or tolerance to anti-RTK therapy, as described herein, or to treat or prevent cancer. The therapeutically effective amount is typically dependent upon the weight of the subject being treated, his or her physical or health condition, the extensiveness of the condition to be treated, the age of the subject being treated, pharmaceutical formulation methods, and/or administration methods (e.g., administration time and administration route).

In some embodiments, anti-cancer therapies such as those described above are administered by various mutes, including, but not limited to, oral, intravenous, intra-arterial, parenteral, intratumoral, intraperitoneal, and subcutaneous mutes. The appropriate formulation and mute of administration can be selected by those of skill in the art according to the intended application.

### MIR-147b Inhibitors

Inhibitors of miR-147b, according to the invention, can target the miRNA at any stage in the process of its production or action. Thus, for example, an inhibitor can block transcription of the pri-miRNA, formation of pre-miRNA, export of the pre-miRNA from the nucleus, Dicer cleavage to generate an miRNA duplex, formation of miRNA/RISC, or binding of miRNA/RISC to its target.

Several different types of molecules and approaches can be used to inhibit miR-147b, according to the invention. These include, for example, single-stranded antisense oligonucleotide (e.g., antagomir and anti-miR miRNA sponge), double-stranded oligonucleotide (e.g., short interfering RNA, such as siRNA and shRNA), small molecule, decoy, aptamer, catalytic RNA (e.g., ribozyme), and gene editing (e.g., CRISPR-cas9) based approaches. Descriptions of examples of molecules and approaches such as these, in the context of inhibiting miR-147b, are provided below.

### Antisense

In one approach, the invention provides antisense molecules that include sequences that are complementary to a target miR-147b sequence, which includes mature miR-147b or a precursor (i.e., pri-miR-147b or pre-miR-147b) or fragment thereof. These molecules are, in general, referred to herein as antisense molecules or antisense oligonucleotides. Specific examples of these types of molecules include antagomirs, miRNA sponges, and competitive inhibitors (see below).

Accordingly, the invention provides single-stranded oligonucleotides having nucleobase sequences with at least 6 contiguous nucleobases complementary to an equal-length portion within a miR-147b target sequence, as noted above (including pri-miR-147b, pre-miR-147b, mature mi-147b, as well as fragments thereof). This approach is typically referred to as an antisense approach, and the corresponding oligonucleotides of the invention are referred to as antisense oligonucleotides (ASO). Without wishing to be bound by theory, this approach involves hybridization of an oligonucleotide of the invention to a target miR-147b sequence, followed by ribonuclease H (RNase H) mediated cleavage of the target miR-147b nucleic acid. Alternatively, and without wishing to be bound by theory, this approach involves hybridization of an oligonucleotide of the invention to a target miR-147b sequence, thereby sterically blocking the target miR-147b nucleic acid from binding to its target mRNA or pre-mRNA. Alternatively, in some embodiments, the single-stranded oligonucleotide may be delivered to a patient as a double stranded oligonucleotide, where the oligonucleotide of the invention is hybridized to another oligonucleotide (e.g., an oligonucleotide having a total of 6 to 30 nucleotides).

An antisense oligonucleotide of the invention (e.g., a single-stranded oligonucleotide of the invention) includes a nucleobase sequence having at least 6 (e.g., at least 7, 8, 9, 10, 11, 12, 13, 14, 15, 16, 17, 18, 19, 20, 25, or 30) contiguous nucleobases complementary to, e.g., an equal-length portion within a miR-147b sequence. The equal-length portion may be disposed within the sequence at any position.

An antisense oligonucleotide of the invention (e.g., a single-stranded oligonucleotide of the invention) may be a gapmer, headmer, or tailmer. Gapmers are oligonucleotides having an RNase H recruiting region (gap) flanked by a 5′ wing and 3′ wing, each of the wings optionally including at least one affinity enhancing nucleoside (e.g., 1, 2, 3, or 4 affinity enhancing nucleosides). Headmers are oligonucleotides having an RNase H recruiting region (gap) flanked by a 5′ wing including at least one affinity enhancing nucleoside (e.g., 1, 2, 3, or 4 affinity enhancing nucleosides). Tailmers are oligonucleotides having an RNase H recruiting region (gap) flanked by a 3′ wing including at least one affinity enhancing nucleoside (e.g., 1, 2, 3, or 4 affinity enhancing nucleosides). In certain embodiments, each wing includes 1-5 nucleosides. In some embodiments, each nucleoside of each wing is a modified nucleoside. In particular embodiments, the gap includes 7-12 nucleosides. Typically, the gap region includes a plurality of contiguous, unmodified deoxyribonucleotides. For example, all nucleotides in the gap region are unmodified deoxyribonucleotides (2′-deoxyribofuranose-based nucleotides). In some embodiments, an antisense oligonucleotide of the invention (e.g., a single-stranded oligonucleotide of the invention) is a gapmer, headmer, or tailmer.

The 5′-wing may consist of, e.g., 1 to 8, 1 to 7, 1 to 6, 1 to 5, 2 to 5, 3 to 5, 4 or 5, 1 to 4, 1 to 3, 1 or 2, 2 to 4, 2 or 3, 3 or 4, 1, 2, 3, 4, 5, or 6 linked nucleosides. The 3′-wing may consists of, e.g., 1 to 8, 1 to 7, 1 to 6, 1 to 5, 2 to 5, 3 to 5, 4 or 5, 1 to 4, 1 to 3, 1 or 2, 2 to 4, 2 or 3, 3 or 4, 1, 2, 3, 4, 5, or 6 linked nucleosides.

The 5′-wing may include, e.g., at least 1, 2, 3, 4, 5, 6, 7, or 8 bridged nucleosides. The 5′-wing may include, e.g., at least 1, 2, 3, 4, 5, 6, 7, or 8 constrained ethyl (cEt) nucleosides. The 5′-wing may include, e.g., at least 1, 2, 3, 4, 5, 6, 7, or 8 LNA nucleosides. Each nucleoside of the 5′-wing may be, e.g., a bridged nucleoside. Each nucleoside of the 5′-wing may be, e.g., a constrained ethyl (cEt) nucleoside. Each nucleoside of the 5′-wing may be, e.g., a LNA nucleoside.

The 3′-wing may include, e.g., at least 1, 2, 3, 4, 5, 6, 7, or 8 bridged nucleosides. The 3′-wing may include, e.g., at least 1, 2, 3, 4, 5, 6, 7, or 8 constrained ethyl (cEt) nucleosides. The 3′-wing may include, e.g., at least 1, 2, 3, 4, 5, 6, 7, or 8 LNA nucleosides. Each nucleoside of the 3′-wing may be, e.g., a bridged nucleoside. Each nucleoside of the 3′-wing may be, e.g., a constrained ethyl (cEt) nucleoside. Each nucleoside of the 3′-wing may be, e.g., a LNA nucleoside.

The 5′-wing may include, e.g., at least 1, 2, 3, 4, 5, 6, 7, or 8 non-bicyclic modified nucleosides. The 5′-wing may include, e.g., at least 1, 2, 3, 4, 5, 6, 7, or 8 2′-substituted nucleosides. The 5′-wing may include, e.g., at least 1, 2, 3, 4, 5, 6, 7, or 8 2′-MOE nucleosides. The 5′-wing may include, e.g., at least 1, 2, 3, 4, 5, 6, 7, or 8 2′-OMe nucleosides. Each nucleoside of the 5′-wing may be, e.g., a non-bicyclic modified nucleoside. Each nucleoside of the 5′-wing may be, e.g., a 2′-substituted nucleoside. Each nucleoside of the 5′-wing may be, e.g., a 2′-MOE nucleoside. Each nucleoside of the 5′-wing may be, e.g., a 2′-OMe nucleoside.

The 3′-wing may include, e.g., at least 1, 2, 3, 4, 5, 6, 7, or 8 non-bicyclic modified nucleosides. The 3′-wing may include, e.g., at least 1, 2, 3, 4, 5, 6, 7, or 8 2′-substituted nucleosides. The 3′-wing may include, e.g., at least 1, 2, 3, 4, 5, 6, 7, or 8 2′-MOE nucleosides. The 3′-wing may include, e.g., at least 1, 2, 3, 4, 5, 6, 7, or 8 2′-OMe nucleosides. Each nucleoside of the 3′-wing may be, e.g., a non-bicyclic modified nucleoside. Each nucleoside of the 3′-wing may be, e.g., a 2′-substituted nucleoside. Each nucleoside of the 3′-wing may be, e.g., a 2′-MOE nucleoside. Each nucleoside of the 3′-wing may be, e.g., a 2′-OMe nucleoside.

The gap may consist of, e.g., 6 to 20 linked nucleosides. The gap may consist of, e.g., 6 to 15, 6 to 12, 6 to 10, 6 to 9, 6 to 8, 6 or 7, 7 to 10, 7 to 9, 7 or 8, 8 to 10, 8 or 9, 6, 7, 8, 9, 10, 11, or 12 linked nucleosides. Each nucleoside of the gap may be, e.g., a 2′-deoxynucleoside. The gap may include, e.g., one or more modified nucleosides. Each nucleoside of the gap may be, e.g., a 2′-deoxynucleoside or may be, e.g., a modified nucleoside that is “DNA-like.” In such embodiments, “DNA-like” means that the nucleoside has similar characteristics to DNA, such that a duplex including the gapmer and an RNA molecule is capable of activating RNase H. For example, under certain conditions, 2′-(ara)-F may support RNase H activation, and thus is DNA-like. In certain embodiments, one or more nucleosides of the gap is not a 2′-deoxynucleoside and is not DNA-like. In certain such embodiments, the gapmer nonetheless supports RNase H activation (e.g., by virtue of the number or placement of the non-DNA nucleosides).

In certain embodiments, gaps include a stretch of unmodified 2′-deoxynucleoside interrupted by one or more modified nucleosides, thus resulting in three sub-regions (two stretches of one or more 2′-deoxynucleosides and a stretch of one or more interrupting modified nucleosides). In certain embodiments, no stretch of unmodified 2′-deoxynucleosides is longer than 5, 6, or 7 nucleosides. In certain embodiments, such short stretches is achieved by using short gap regions. In certain embodiments, short stretches are achieved by interrupting a longer gap region.

The gap may include, e.g., one or more modified nucleosides. The gap may include, e.g., one or more modified nucleosides selected from among cEt, FHNA, LNA, and 2-thio-thymidine. The gap may include, e.g., one modified nucleoside. The gap may include, e.g., a 5′-substituted sugar moiety selected from the group consisting of 5′-Me and 5′-(R)-Me. The gap may include, e.g., two modified nucleosides. The gap may include, e.g., three modified nucleosides. The gap may include, e.g., four modified nucleosides. The gap may include, e.g., two or more modified nucleosides and each modified nucleoside is the same. The gap may include, e.g., two or more modified nucleosides and each modified nucleoside is different.

The gap may include, e.g., one or more modified internucleoside linkages. The gap may include, e.g., one or more methyl phosphonate linkages. In certain embodiments the gap may include, e.g., two or more modified internucleoside linkages. The gap may include, e.g., one or more modified linkages and one or more modified nucleosides. The gap may include, e.g., one modified linkage and one modified nucleoside. The gap may include, e.g., two modified linkages and two or more modified nucleosides.

An antisense oligonucleotide of the invention (e.g., a single-stranded oligonucleotide of the invention) may include one or more mismatches. For example, the mismatch may be specifically positioned within a gapmer, headmer, or tailmer. The mismatch may be, e.g., at position 1, 2, 3, 4, 5, 6, 7, or 8 (e.g., at position 1, 2, 3, or 4) from the 3′-end of the gap region. Alternatively, or additionally, the mismatch may be, e.g., at position 9, 8, 7, 6, 5, 4, 3, 2, or 1 (e.g., at position 4, 3, 2, or 1) from the 3′-end of the gap region. In some embodiments, the 5′ wing and/or 3′wing do not include mismatches.

An antisense oligonucleotide of the invention (e.g., a single-stranded oligonucleotide of the invention) may be a morpholino.

An antisense oligonucleotide of the invention (e.g., a single-stranded oligonucleotide of the invention) may include a total of X to Y interlinked nucleosides, where X represents the fewest number of nucleosides in the range and Y represents the largest number nucleosides in the range. In these embodiments, X and Y are each independently selected from the group consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, an oligonucleotide of the invention may include a total of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 interlinked nucleosides.

In some embodiments, an antisense oligonucleotide of the invention (e.g., a single-stranded oligonucleotide of the invention) includes at least one modified internucleoside linkage. A modified internucleoside linkage may be, e.g., a phosphorothioate internucleoside linkage (e.g., a phosphorothioate diester or phosphorothioate triester).

In some embodiments, an antisense oligonucleotide of the invention (e.g., a single-stranded oligonucleotide of the invention) includes at least one stereochemically enriched phosphorothioate-based internucleoside linkage. In some embodiments, an antisense oligonucleotide of the invention (e.g., a single-stranded oligonucleotide of the invention) includes a pattern of stereochemically enriched phosphorothioate internucleoside linkages described herein (e.g., a 5′-R_{P}S_{P}S_{P}-3′). These patterns may enhance target miR-147b cleavage by RNase H relative to a stereorandom corresponding oligonucleotide. In some embodiments, inclusion and/or location of particular stereochemically enriched linkages within an oligonucleotide may alter the cleavage pattern of a target nucleic acid, when such an oligonucleotide is utilized for cleaving the nucleic acid. For example, a pattern of internucleoside linkage P-stereogenic centers may increase cleavage efficiency of a target nucleic acid. A pattern of internucleoside linkage P-stereogenic centers may provide new cleavage sites in a target nucleic acid. A pattern of internucleoside linkage P-stereogenic centers may reduce the number of cleavage sites, for example, by blocking certain existing cleavage sites. Moreover, in some embodiments, a pattern of internucleoside linkage P-stereogenic centers may facilitate cleavage at only one site within the target sequence that is complementary to an oligonucleotide utilized for the cleavage. Cleavage efficiency may be increased by selecting a pattern of internucleoside linkage P-stereogenic centers that reduces the number of cleavage sites in a target nucleic acid.

Purity of an oligonucleotide may be expressed as the percentage of oligonucleotide molecules that are of the same oligonucleotide type within an oligonucleotide composition. At least about 10%, 20%, 30%, 40%, 50%, 60%, 70%, 80%, 90%, 92%, 94%, 95%, 96%, 97%, 98%, or 99% of the oligonucleotides may be, e.g., of the same oligonucleotide type.

An oligonucleotide may include 1, 2, 3, 4, 5, 6, 7, 8, 9, 10, 11, 12, 13, 14, 15, 16, 17, 18, 19, 20, 21, 22, 23, 24, 25, or more stereochemically enriched internucleoside linkages. An oligonucleotide may include at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched internucleoside linkages. Exemplary stereochemically enriched internucleoside linkages are described herein. An oligonucleotide may include at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched internucleoside linkages in the S_{P} configuration.

A stereochemically enriched internucleoside linkage may be, e.g., a stereochemically enriched phosphorothioate internucleoside linkage. A provided oligonucleotide may comprise at least 5%, 10%, 15%, 20%, 25%, 30%, 35%, 40%, 45%, 50%, 55%, 60%, 65%, 70%, 75%, 80%, 85%, 90%, 95%, or 100% stereochemically enriched phosphorothioate internucleoside linkages. All internucleoside linkages may be, e.g., stereochemically enriched phosphorothioate internucleoside linkages. In some embodiments, at least 10, 20, 30, 40, 50, 60, 70, 80, 90, or 95% stereochemically enriched phosphorothioate internucleoside linkages have the S_{P} stereochemical configuration. In some embodiments, less than 10, 20, 30, 40, 50, 60, 70, 80, 90, or 95% stereochemically enriched phosphorothioate internucleoside linkages have the S_{P} stereochemical configuration. In some embodiments, at least 10, 20, 30, 40, 50, 60, 70, 80, 90, or 95% stereochemically enriched phosphorothioate internucleoside linkages have the R_{P} stereochemical configuration. In some embodiments, less than 10, 20, 30, 40, 50, 60, 70, 80, 90, or 95% stereochemically enriched phosphorothioate internucleoside linkages have the R_{P} stereochemical configuration.

An oligonucleotide may have, e.g., only one R_{P} stereochemically enriched phosphorothioate internucleoside linkage. An oligonucleotide may have, e.g., multiple R_{P} stereochemically enriched phosphorothioate internucleoside linkages, where all internucleoside linkages are stereochemically enriched phosphorothioate internucleoside linkages. A stereochemically enriched phosphorothioate internucleoside linkage may be, e.g., a stereochemically enriched phosphorothioate diester linkage. In some embodiments, each stereochemically enriched phosphorothioate internucleoside linkage is independently a stereochemically enriched phosphorothioate diester linkage. In some embodiments, each internucleoside linkage is independently a stereochemically enriched phosphorothioate diester linkage. In some embodiments, each internucleoside linkage is independently a stereochemically enriched phosphorothioate diester linkage, and only one is R_{P}.

The gap region may include, e.g., a stereochemically enriched internucleoside linkage. The gap region may include, e.g., stereochemically enriched phosphorothioate internucleoside linkages. The gap region may have, e.g., a repeating pattern of internucleoside linkage stereochemistry. The gap region may have, e.g., a repeating pattern of internucleoside linkage stereochemistry. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7, or 8. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7, or 8. The gap region may have, e.g., a repeating pattern of internucleoside linkage stereochemistry, where the repeating pattern is (S_{P})ₘR_{P}, where m is 2, 3, 4, 5, 6, 7, or 8. The gap region may have, e.g., a repeating pattern of internucleoside linkage stereochemistry, where the repeating pattern is R_{P}(S_{P})ₘ, where m is 2, 3, 4, 5, 6, 7, or 8. The gap region may have, e.g., a repeating pattern of internucleoside linkage stereochemistry, where the repeating pattern is (S_{P})ₘR_{P} or R_{P}(S_{P})ₘ, where m is 2, 3, 4, 5, 6, 7, or 8. The gap region may have, e.g., a repeating pattern of internucleoside linkage stereochemistry, where the repeating pattern is a motif including at least 33% of internucleoside linkages with the S_{P} stereochemical identify. The gap region may have, e.g., a repeating pattern of internucleoside linkage stereochemistry, where the repeating pattern is a motif including at least 50% of internucleoside linkages with the S_{P} stereochemical identify. The gap region may have, e.g., a repeating pattern of internucleoside linkage stereochemistry, where the repeating pattern is a motif including at least 66% of internucleoside linkages with the S_{P} stereochemical identify. The gap region may have, e.g., a repeating pattern of internucleoside linkage stereochemistry, where the repeating pattern is a repeating triplet motif selected from R_{P}R_{P}S_{P} and S_{P}S_{P}R_{P}. The gap region may have, e.g., a repeating pattern of internucleoside linkage stereochemistry, where the repeating pattern is a repeating R_{P}R_{P}S_{P}. The gap region may have, e.g., a repeating pattern of internucleoside linkage stereochemistry, where the repeating pattern is a repeating S_{P}S_{P}R_{P}.

An oligonucleotide may include a pattern of internucleoside P-stereogenic centers in the gap region including (S_{P})ₘR_{P} or R_{P}(S_{P})ₘ. An oligonucleotide may include a pattern of internucleoside P-stereogenic centers in the gap region including R_{P}(S_{P})ₘ. An oligonucleotide may include a pattern of P-stereogenic centers in the gap region including (S_{P})ₘR_{P}. In some embodiments, m is 2. An oligonucleotide may include a pattern of internucleoside P-stereogenic centers in the gap region including R_{P}(S_{P})₂. An oligonucleotide may include a pattern of internucleoside P-stereogenic centers in the gap region including (S_{P})₂R_{P}(S_{P})₂. An oligonucleotide may include a pattern of internucleoside P-stereogenic centers in the gap region including (R_{P})₂R_{P}(S_{P})₂. An oligonucleotide may include a pattern of internucleoside P-stereogenic centers in the gap region including R_{P}S_{P}R_{P}(S_{P})₂. An oligonucleotide may include a pattern of internucleoside P-stereogenic centers in the gap region including S_{P}R_{P}R_{P}(S_{P})₂. An oligonucleotide may include a pattern of internucleoside P-stereogenic centers in the gap region including (S_{P})₂R_{P}.

An oligonucleotide may include a pattern of internucleoside P-stereogenic centers including (S_{P})ₘR_{P} or R_{P}(S_{P})ₘ. An oligonucleotide may include a pattern of internucleoside P-stereogenic centers including R_{P}(S_{P})ₘ. An oligonucleotide may include a pattern of internucleoside P-stereogenic centers including (S_{P})ₘR_{P}. In some embodiments, m is 2. An oligonucleotide may include a pattern of internucleoside P-stereogenic centers including R_{P}(S_{P})₂. An oligonucleotide may include a pattern of internucleoside P-stereogenic centers including (S_{P})₂R_{P}(S_{P})₂. An oligonucleotide may include a pattern of internucleoside P-stereogenic centers including (R_{P})₂R_{P}(S_{P})₂. An oligonucleotide may include a pattern of internucleoside P-stereogenic centers including R_{P}S_{P}R_{P}(S_{P})₂. An oligonucleotide may include a pattern of internucleoside P-stereogenic centers including S_{P}R_{P}R_{P}(S_{P})₂. An oligonucleotide may include a pattern of internucleoside P-stereogenic centers including (S_{P})₂R_{P}.

In the embodiments of internucleoside P-stereogenic center patterns, m is 2, 3, 4, 5, 6, 7, or 8, unless specified otherwise. In some embodiments of internucleoside P-stereogenic center patterns, m is 3, 4, 5, 6, 7, or 8. In some embodiments of internucleoside P-stereogenic center patterns, m is 4, 5, 6, 7, or 8. In some embodiments of internucleoside P-stereogenic center patterns, m is 5, 6, 7, or 8. In some embodiments of internucleoside P-stereogenic center patterns, m is 6, 7, or 8. In some embodiments of internucleoside P-stereogenic center patterns, m is 7 or 8. In some embodiments of internucleoside P-stereogenic center patterns, m is 2. In some embodiments of internucleoside P-stereogenic center patterns, m is 3. In some embodiments of internucleoside P-stereogenic center patterns, m is 4. In some embodiments of internucleoside P-stereogenic center patterns, m is 5. In some embodiments of internucleoside P-stereogenic center patterns, m is 6. In some embodiments of internucleoside P-stereogenic center patterns, m is 7. In some embodiments of internucleoside P-stereogenic center patterns, m is 8.

A repeating pattern may be, e.g., (S_{P})ₘ(R_{P})ₙ, where n is independently 1, 2, 3, 4, 5, 6, 7, or 8, and m is independently as described herein. An oligonucleotide may include a pattern of internucleoside P-stereogenic centers including (S_{P})ₘ(R_{P})ₙ. An oligonucleotide may include a pattern of internucleoside P-stereogenic centers including (Sₚ)ₘ(R_{P})ₙ. A repeating pattern may be, e.g., (R_{P})ₙ(S_{P})ₘ, where n is independently 1, 2, 3, 4, 5, 6, 7, or 8, and m is independently as described herein. An oligonucleotide may include a pattern of internucleoside P-stereogenic centers including (R_{P})ₙ(S_{P})ₘ. An oligonucleotide may include a pattern of internucleoside P-stereogenic centers in the gap region including (R_{P})ₙ(S_{P})ₘ. In some embodiments, (R_{P})ₙ(S_{P})ₘ is (R_{P})(S_{P})₂. In some embodiments, (S_{P})ₙ(R_{P})ₘ is (S_{P})₂(R_{P}).

A repeating pattern may be, e.g., (S_{P})ₘ(R_{P})ₙ(S_{P})ₜ, where each of n and t is independently 1, 2, 3, 4, 5, 6, 7, or 8, and m is as described herein. An oligonucleotide may include a pattern of internucleoside P-stereogenic centers including (S_{P})ₘ(R_{P})ₙ(S_{P})ₜ. An oligonucleotide may include a pattern of internucleoside P-stereogenic centers including (S_{P})ₘ(R_{P})ₙ(S_{P})ₜ. A repeating pattern may be, e.g., (S_{P})ₜ(R_{P})ₙ(S_{P})ₘ, where each of n and t is independently 1, 2, 3, 4, 5, 6, 7, or 8, and m is as described herein. An oligonucleotide may include a pattern of internucleoside P-stereogenic centers including (S_{P})ₜ(R_{P})ₙ(S_{P})ₘ. An oligonucleotide may include a pattern of internucleoside P-stereogenic centers in the gap region including (S_{P})ₜ(R_{P})ₙ(S_{P})ₘ.

A repeating pattern is (Np)ₜ(R_{P})ₙ(S_{P})ₘ, where each of n and t is independently 1, 2, 3, 4, 5, 6, 7, or 8, Np is independently R_{P} or S_{P}, and m is as described herein. An oligonucleotide may include a pattern of internucleoside P-stereogenic centers including (Np)ₜ(R_{P})ₙ(S_{P})ₘ. An oligonucleotide may include a pattern of internucleoside P-stereogenic centers in the gap region including (Np)ₜ(Rₚ)ₙ(S_{P})ₘ. A repeating pattern may be, e.g., (Np)ₜ(R_{P})ₙ(S_{P})ₘ, where each of n and t is independently 1, 2, 3, 4, 5, 6, 7, or 8, Np is independently R_{P} or S_{P}, and m is as described herein. An oligonucleotide may include a pattern of internucleoside P-stereogenic centers including (Np)ₜ(R_{P})ₙ(S_{P})ₘ. An oligonucleotide may include a pattern of internucleoside P-stereogenic centers in the gap region including (Np)ₜ(R_{P})ₙ(S_{P})ₘ. In some embodiments, Np is R_{P}. In some embodiments, Np is S_{P}. All Np may be, e.g., same. All Np may be, e.g., S_{P}. At least one Np may be, e.g., different from another Np. In some embodiments, t is 2.

In the embodiments of internucleoside P-stereogenic center patterns, n is 1, 2, 3, 4, 5, 6, 7, or 8. In some embodiments of internucleoside P-stereogenic center patterns, n is 2, 3, 4, 5, 6, 7, or 8. In some embodiments of internucleoside P-stereogenic center patterns, n is 3, 4, 5, 6, 7, or 8. In some embodiments of internucleoside P-stereogenic center patterns, n is 4, 5, 6, 7, or 8. In some embodiments of internucleoside P-stereogenic center patterns, n is 5, 6, 7, or 8. In some embodiments of internucleoside P-stereogenic center patterns, n is 6, 7, or 8. In some embodiments of internucleoside P-stereogenic center patterns, n is 7 or 8. In some embodiments of internucleoside P-stereogenic center patterns, n is 1. In some embodiments of internucleoside P-stereogenic center patterns, n is 2. In some embodiments of internucleoside P-stereogenic center patterns, n is 3. In some embodiments of internucleoside P-stereogenic center patterns, n is 4. In some embodiments of internucleoside P-stereogenic center patterns, n is 5. In some embodiments of internucleoside P-stereogenic center patterns, n is 6. In some embodiments of internucleoside P-stereogenic center patterns, n is 7. In some embodiments of internucleoside P-stereogenic center patterns, n is 8.

In the embodiments of internucleoside P-stereogenic center patterns, t is 1, 2, 3, 4, 5, 6, 7, or 8. In some embodiments of internucleoside P-stereogenic center patterns, t is 2, 3, 4, 5, 6, 7, or 8. In some embodiments of internucleoside P-stereogenic center patterns, t is 3, 4, 5, 6, 7, or 8. In some embodiments of internucleoside P-stereogenic center patterns, t is 4, 5, 6, 7, or 8. In some embodiments of internucleoside P-stereogenic center patterns, t is 5, 6, 7, or 8. In some embodiments of internucleoside P-stereogenic center patterns, t is 6, 7, or 8. In some embodiments of internucleoside P-stereogenic center patterns, t is 7 or 8. In some embodiments of internucleoside P-stereogenic center patterns, t is 1. In some embodiments of internucleoside P-stereogenic center patterns, t is 2. In some embodiments of internucleoside P-stereogenic center patterns, t is 3. In some embodiments of internucleoside P-stereogenic center patterns, t is 4. In some embodiments of internucleoside P-stereogenic center patterns, t is 5. In some embodiments of internucleoside P-stereogenic center patterns, t is 6. In some embodiments of internucleoside P-stereogenic center patterns, t is 7. In some embodiments of internucleoside P-stereogenic center patterns, t is 8.

At least one of m and t may be, e.g., greater than 2. At least one of m and t may be, e.g., greater than 3. At least one of m and t may be, e.g., greater than 4. At least one of m and t may be, e.g., greater than 5. At least one of m and t may be, e.g., greater than 6. At least one of m and t may be, e.g., greater than 7. In some embodiments, each of m and t is greater than 2. In some embodiments, each of m and t is greater than 3. In some embodiments, each of m and t is greater than 4. In some embodiments, each of m and t is greater than 5. In some embodiments, each of m and t is greater than 6. In some embodiments, each of m and t is greater than 7.

In some embodiments of internucleoside P-stereogenic center patterns, n is 1, and at least one of m and t is greater than 1. In some embodiments of internucleoside P-stereogenic center patterns, n is 1 and each of m and t is independent greater than 1. In some embodiments of internucleoside P-stereogenic center patterns, m>n and t>n. In some embodiments, (S_{P})ₘ(R_{P})ₙ(S_{P})ₜ is (S_{P})₂R_{P}(S_{P})₂. In some embodiments, (S_{P})ₜ(R_{P})ₙ(S_{P})ₘ is (S_{P})₂R_{P}(S_{P})₂. In some embodiments, (S_{P})ₜ(R_{P})ₙ(S_{P})ₘ is S_{P}R_{P}(S_{P})₂. In some embodiments, (Np)ₜ(R_{P})ₙ(S_{P})ₘ is (Np)ₜ(R_{P})ₙ(S_{P})ₘ. In some embodiments, (Np)ₜ(R_{P})ₙ(S_{P})ₘ is (Np)₂R_{P}(S_{P})ₘ. In some embodiments, (Np)ₜ(R_{P})ₙ(S_{P})ₘ is (R_{P})₂R_{P}(S_{P})ₘ. In some embodiments, (Np)ₜ(R_{P})ₙ(S_{P})ₘ is (S_{P})₂R_{P}(S_{P})ₘ. In some embodiments, (Np)ₜ(R_{P})ₙ(S_{P})ₘ is R_{P}S_{P}R_{P}(S_{P})ₘ. In some embodiments, (Np)ₜ(R_{P})ₙ(S_{P})ₘ is S_{P}R_{P}R_{P}(S_{P})ₘ.

In some embodiments, (S_{P})ₜ(R_{P})ₙ(S_{P})ₘ is S_{P}R_{P}S_{P}S_{P}. In some embodiments, (S_{P})ₜ(R_{P})ₙ(S_{P})ₘ is (S_{P})₂R_{P}(S_{P})₂. In some embodiments, (S_{P})ₜ(R_{P})ₙ(S_{P})ₘ is (S_{P})₃R_{P}(S_{P})₃. In some embodiments, (S_{P})ₜ(R_{P})ₙ(S_{P})ₘ is (S_{P})₄R_{P}(S_{P})₄. In some embodiments, (S_{P})ₜ(R_{P})ₙ(S_{P})ₘ is (S_{P})ₜR_{P}(S_{P})₅. In some embodiments, (S_{P})ₜ(R_{P})ₙ(S_{P})ₘ is S_{P}R_{P}(S_{P})₅. In some embodiments, (S_{P})ₜ(R_{P})ₙ(S_{P})ₘ is (S_{P})₂R_{P}(S_{P})₅. In some embodiments, (S_{P})ₜ(R_{P})ₙ(S_{P})ₘ is (S_{P})₃R_{P}(S_{P})₅. In some embodiments, (S_{P})ₜ(R_{P})ₙ(S_{P})ₘ is (S_{P})₄R_{P}(S_{P})₅. In some embodiments, (S_{P})ₜ(R_{P})ₙ(S_{P})ₘ is (S_{P})₅R_{P}(S_{P})₅.

In some embodiments, (S_{P})ₘ(R_{P})ₙ(S_{P})ₜ is (S_{P})₂R_{P}(S_{P})₂. In some embodiments, (S_{P})ₘ(R_{P})ₙ(S_{P})ₜ is (S_{P})₃R_{P}(S_{P})₃. In some embodiments, (S_{P})ₘ(R_{P})ₙ(S_{P})ₜ is (S_{P})₄R_{P}(S_{P})₄. In some embodiments, (S_{P})ₘ(R_{P})ₙ(S_{P})ₜ is (S_{P})ₘR_{P}(S_{P})₅. In some embodiments, (S_{P})ₘ(R_{P})ₙ(S_{P})ₜ is (S_{P})₂R_{P}(S_{P})₅. In some embodiments, (S_{P})ₘ(R_{P})ₙ(S_{P})ₜ is (S_{P})₃R_{P}(S_{P})₅. In some embodiments, (S_{P})ₘ(R_{P})ₙ(S_{P})ₜ is (S_{P})₄R_{P}(S_{P})₅. In some embodiments, (S_{P})ₘ(R_{P})ₙ(S_{P})ₜ is (S_{P})₅R_{P}(S_{P})₅.

The gap region may include, e.g., at least one R_{P} internucleoside linkage. The gap region may include, e.g., at least one R_{P} phosphorothioate internucleoside linkage. The gap region may include, e.g., at least two R_{P} internucleoside linkages. The gap region may include, e.g., at least two R_{P} phosphorothioate internucleoside linkages. The gap region may include, e.g., at least three R_{P} internucleoside linkages. The gap region may include, e.g., at least three R_{P} phosphorothioate internucleoside linkages. The gap region may include, e.g., at least 4, 5, 6, 7, 8, 9, or 10 R_{P} internucleoside linkages. The gap region may include, e.g., at least 4, 5, 6, 7, 8, 9, or 10 R_{P} phosphorothioate internucleoside linkages.

A gapmer may include a wing-gap-wing motif that is a 5-10-5 motif, where the nucleosides in each wing region are Z-MOE-modified nucleosides. A wing-gap-wing motif of a gapmer may be, e.g., a 5-10-5 motif where the nucleosides in the gap region are 2′-deoxyribonucleosides. A wing-gap-wing motif of a gapmer may be, e.g., a 5-10-5 motif, where all internucleoside linkages are phosphorothioate internucleoside linkages. A wing-gap-wing motif of a gapmer may be, e.g., a 5-10-5 motif, where all internucleoside linkages are stereochemically enriched phosphorothioate internucleoside linkages. A wing-gap-wing motif of a gapmer may be, e.g., a 5-10-5 motif, where the nucleosides in each wing region are Z-MOE-modified nucleosides, the nucleosides in the gap region are 2′-deoxyribonucleosides, and all internucleoside linkages are stereochemically enriched phosphorothioate internucleoside linkages.

In certain embodiments, a wing-gap-wing motif is a 5-10-5 motif where the residues at each wing region are not Z-MOE-modified residues. In certain embodiments, a wing-gap-wing motif is a 5-10-5 motif where the residues in the gap region are Z-deoxyribonucleotide residues. In certain embodiments, a wing-gap-wing motif is a 5-10-5 motif, where all internucleosidic linkages are phosphorothioate internucleosidic linkages. In certain embodiments, a wing-gap-wing motif is a 5-10-5 motif, where all internucleoside linkages are stereochemically enriched phosphorothioate internucleoside linkages. In certain embodiments, a wing-gap-wing motif is a 5-10-5 motif where the residues at each wing region are not Z-MOE-modified residues, the residues in the gap region are Z-deoxyribonucleotide, and all internucleoside linkages are stereochemically enriched phosphorothioate internucleoside linkages.

An oligonucleotide may include a region with at least one of the first, second, third, fifth, seventh, eighth, ninth, eighteenth, nineteenth, and twentieth internucleoside linkages being a P-stereogenic linkage (e.g., phosphorothioate phosphodiester or phosphorothioate phosphotriester). At least two of the first, second, third, fifth, seventh, eighth, ninth, eighteenth, nineteenth, and twentieth internucleoside linkages are stereogenic. At least three of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). At least four of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). At least five of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). At least six of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). At least seven of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). At least eight of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). At least nine of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). One of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). Two of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). Three of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). Four of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). Five of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). Six of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). Seven of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). Eight of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester). Nine of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotiester). Ten of the first, second, third, fifth, seventh, eighth, ninth, eighteenth, nineteenth, and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighteenth, nineteenth, and twentieth internucleoside linkages being P-stereogenic (e.g., phosphorothioate phosphodiester or phosphorothioate phosphotriester). At least two of the first, second, third, fifth, seventh, eighteenth, nineteenth, and twentieth internucleoside linkages may be, e.g., P-stereogenic (e.g., phosphorothioate phosphodiester or phosphorothioate phosphotiester). At least three of the first, second, third, fifth, seventh, eighteenth, nineteenth, and twentieth internucleoside linkages may be, e.g., P-stereogenic (e.g., phosphorothioate phosphodiester or phosphorothioate phosphotriester). At least four of the first, second, third, fifth, seventh, eighteenth, nineteenth, and twentieth internucleoside linkages may be, e.g., P-stereogenic (e.g., phosphorothioate phosphodiester or phosphorothioate phosphotiester). At least five of the first, second, third, fifth, seventh, eighteenth, nineteenth, and twentieth internucleoside linkages may be, e.g., P-stereogenic (e.g., phosphorothioate phosphodiester or phosphorothioate phosphotiester). At least six of the first, second, third, fifth, seventh, eighteenth, nineteenth, and twentieth internucleoside linkages may be, e.g., P-stereogenic (e.g., phosphorothioate phosphodiester or phosphorothioate phosphotriester). At least seven of the first, second, third, fifth, seventh, eighteenth, nineteenth, and twentieth internucleoside linkages may be, e.g., P-stereogenic (e.g., phosphorothioate phosphodiester or phosphorothioate phosphotiester). One of the first, second, third, fifth, seventh, eighteenth, nineteenth, and twentieth internucleoside may be, e.g., P-stereogenic (e.g., phosphorothioate phosphodiester or phosphorothioate phosphotiester). Two of the first, second, third, fifth, seventh, eighteenth, nineteenth, and twentieth internucleoside linkages may be, e.g., P-stereogenic (e.g., phosphorothioate phosphodiester or phosphorothioate phosphotiester). Three of the first, second, third, fifth, seventh, eighteenth, nineteenth, and twentieth internucleoside linkages may be, e.g., P-stereogenic (e.g., phosphorothioate phosphodiester or phosphorothioate phosphotriester). Four of the first, second, third, fifth, seventh, eighteenth, nineteenth, and twentieth internucleoside linkages may be, e.g., P-stereogenic (e.g., phosphorothioate phosphodiester or phosphorothioate phosphotiester). Five of the first, second, third, fifth, seventh, eighteenth, nineteenth, and twentieth internucleoside linkages may be, e.g., P-stereogenic (e.g., phosphorothioate phosphodiester or phosphorothioate phosphotiester). Six of the first, second, third, fifth, seventh, eighteenth, nineteenth, and twentieth internucleoside linkages may be, e.g., P-stereogenic (e.g., phosphorothioate phosphodiester or phosphorothioate phosphotriester). Seven of the first, second, third, fifth, seventh, eighteenth, nineteenth, and twentieth internucleoside linkages may be, e.g., P-stereogenic (e.g., phosphorothioate phosphodiester or phosphorothioate phosphotiester). Eight of the first, second, third, fifth, seventh, eighteenth, nineteenth, and twentieth internucleoside linkages may be, e.g., P-stereogenic (e.g., phosphorothioate phosphodiester or phosphorothioate phosphotiester).

An oligonucleotide may include a region with at least one of the first, second, third, fifth, seventh, eighth, ninth, eighteenth, nineteenth, and twentieth internucleoside linkages being P-stereogenic (e.g., phosphorothioate phosphodiester or phosphorothioate phosphotriester), and at least one internucleoside linkage being non-stereogenic. An oligonucleotide may include a region in which at least one of the first, second, third, fifth, seventh, eighteenth, nineteenth, and twentieth internucleoside linkages being P-stereogenic (e.g., phosphorothioate phosphodiester or phosphorothioate phosphotiester), and at least one internucleoside linkage being non-stereogenic. At least two internucleoside linkages may be, e.g., non-stereogenic. At least three internucleoside linkages may be, e.g., non-stereogenic. At least four internucleoside linkages may be, e.g., non-stereogenic. At least five internucleoside linkages may be, e.g., non-stereogenic. At least six internucleoside linkages may be, e.g., non-stereogenic. At least seven internucleoside linkages may be, e.g., non-stereogenic. At least eight internucleoside linkages may be, e.g., non-stereogenic. At least nine internucleoside linkages may be, e.g., non-stereogenic. At least 10 internucleoside linkages may be, e.g., non-stereogenic. At least 11 internucleoside linkages may be, e.g., non-stereogenic. At least 12 internucleoside linkages may be, e.g., non-stereogenic. At least 13 internucleoside linkages may be, e.g., non-stereogenic. At least 14 internucleoside linkages may be, e.g., non-stereogenic. At least 15 internucleoside linkages may be, e.g., non-stereogenic. At least 16 internucleoside linkages may be, e.g., non-stereogenic. At least 17 internucleoside linkages may be, e.g., non-stereogenic. At least 18 internucleoside linkages may be, e.g., non-stereogenic. At least 19 internucleoside linkages may be, e.g., non-stereogenic. At least 20 internucleoside linkages may be, e.g., non-stereogenic. In some embodiments, one internucleoside linkage is non-stereogenic. In some embodiments, two internucleoside linkages are non-stereogenic. In some embodiments, three internucleoside linkages are non-stereogenic. In some embodiments, four internucleoside linkages are non-stereogenic. In some embodiments, five internucleoside linkages are non-stereogenic. In some embodiments, six internucleoside linkages are non-stereogenic. In some embodiments, seven internucleoside linkages are non-stereogenic. In some embodiments, eight internucleoside linkages are non-stereogenic. In some embodiments, nine internucleoside linkages are non-stereogenic. In some embodiments, 10 internucleoside linkages are non-stereogenic. In some embodiments, 11 internucleoside linkages are non-stereogenic. In some embodiments, 12 internucleoside linkages are non-stereogenic. In some embodiments, 13 internucleoside linkages are non-stereogenic. In some embodiments, 14 internucleoside linkages are non-stereogenic. In some embodiments, 15 internucleoside linkages are non-stereogenic. In some embodiments, 16 internucleoside linkages are non-stereogenic. In some embodiments, 17 internucleoside linkages are non-stereogenic. In some embodiments, 18 internucleoside linkages are non-stereogenic. In some embodiments, 19 internucleoside linkages are non-stereogenic. In some embodiments, 20 internucleoside linkages are non-stereogenic. An oligonucleotide may include a region in which all internucleoside linkages, except at least one of the first, second, third, fifth, seventh, eighth, ninth, eighteenth, nineteenth, and twentieth internucleoside linkages which is P-stereogenic, are non-stereogenic.

An oligonucleotide may include a region with at least one of the first, second, third, fifth, seventh, eighth, ninth, eighteenth, nineteenth, and twentieth internucleoside linkages being P-stereogenic, and at least one internucleoside linkage being phosphate phosphodiester. An oligonucleotide may include a region with at least one of the first, second, third, fifth, seventh, eighteenth, nineteenth, and twentieth internucleoside linkages being P-stereogenic, and at least one internucleoside linkage being phosphate phosphodiester. At least two internucleoside linkages may be, e.g., phosphate phosphodiesters. At least three internucleoside linkages may be, e.g., phosphate phosphodiesters. At least four internucleoside linkages may be, e.g., phosphate phosphodiesters. At least five internucleoside linkages may be, e.g., phosphate phosphodiesters. At least six internucleoside linkages may be, e.g., phosphate phosphodiesters. At least seven internucleoside linkages may be, e.g., phosphate phosphodiesters. At least eight internucleoside linkages may be, e.g., phosphate phosphodiesters. At least nine internucleoside linkages may be, e.g., phosphate phosphodiesters. At least 10 internucleoside linkages may be, e.g., phosphate phosphodiesters. At least 11 internucleoside linkages may be, e.g., phosphate phosphodiesters. At least 12 internucleoside linkages may be, e.g., phosphate phosphodiesters. At least 13 internucleoside linkages may be, e.g., phosphate phosphodiesters. At least 14 internucleoside linkages may be, e.g., phosphate phosphodiesters. At least 15 internucleoside linkages may be, e.g., phosphate phosphodiesters. At least 16 internucleoside linkages may be, e.g., phosphate phosphodiesters. At least 17 internucleoside linkages may be, e.g., phosphate phosphodiesters. At least 18 internucleoside linkages may be, e.g., phosphate phosphodiesters. At least 19 internucleoside linkages may be, e.g., phosphate phosphodiesters. At least 20 internucleoside linkages may be, e.g., phosphate phosphodiesters. In some embodiments, one internucleoside linkage is phosphate phosphodiesters. In some embodiments, two internucleoside linkages are phosphate phosphodiesters.

In some embodiments, three internucleoside linkages are phosphate phosphodiesters. In some embodiments, four internucleoside linkages are phosphate phosphodiesters. In some embodiments, five internucleoside linkages are phosphate phosphodiesters. In some embodiments, six internucleoside linkages are phosphate phosphodiesters. In some embodiments, seven internucleoside linkages are phosphate phosphodiesters. In some embodiments, eight internucleoside linkages are phosphate phosphodiesters. In some embodiments, nine internucleoside linkages are phosphate phosphodiesters.

In some embodiments, 10 internucleoside linkages are phosphate phosphodiesters. In some embodiments, 11 internucleoside linkages are phosphate phosphodiesters. In some embodiments, 12 internucleoside linkages are phosphate phosphodiesters. In some embodiments, 13 internucleoside linkages are phosphate phosphodiesters. In some embodiments, 14 internucleoside linkages are phosphate phosphodiesters. In some embodiments, 15 internucleoside linkages are phosphate phosphodiesters. In some embodiments, 16 internucleoside linkages are phosphate phosphodiesters. In some embodiments, 17 internucleoside linkages are phosphate phosphodiesters. In some embodiments, 18 internucleoside linkages are phosphate phosphodiesters. In some embodiments, 19 internucleoside linkages are phosphate phosphodiesters. In some embodiments, 20 internucleoside linkages are phosphate phosphodiesters. An oligonucleotide may include a region with all internucleoside linkages, except at least one of the first, second, third, fifth, seventh, eighth, ninth, eighteenth, nineteenth, and twentieth internucleoside linkages being P-stereogenic, being phosphate phosphodiesters.

An oligonucleotide may include a region with at least one of the first, second, third, fifth, seventh, eighth, ninth, eighteenth, nineteenth, and twentieth internucleoside linkages being P-stereogenic, and at least 10% of all internucleoside linkages in the region being non-stereogenic. An oligonucleotide may include a region with at least one of the first, second, third, fifth, seventh, eighteenth, nineteenth, and twentieth internucleoside linkages being P-stereogenic, and at least 10% of all internucleoside linkages in the region being non-stereogenic. At least 20% of all the internucleoside linkages in the region may be, e.g., non-stereogenic. At least 30% of all the internucleoside linkages in the region may be, e.g., non-stereogenic. At least 40% of all the internucleoside linkages in the region may be, e.g., non-stereogenic.

At least 50% of all the internucleoside linkages in the region may be, e.g., non-stereogenic. At least 60% of all the internucleoside linkages in the region may be, e.g., non-stereogenic. At least 70% of all the internucleoside linkages in the region may be, e.g., non-stereogenic. At least 80% of all the internucleoside linkages in the region may be, e.g., non-stereogenic. At least 90% of all the internucleoside linkages in the region may be, e.g., non-stereogenic. At least 50% of all the internucleoside linkages in the region may be, e.g., non-stereogenic. A non-stereogenic internucleoside linkage may be, e.g., a phosphate phosphodiester. In some embodiments, each non-stereogenic internucleoside linkage is a phosphate phosphodiester.

The first internucleoside linkage of the region may be, e.g., an S_{P} internucleoside linkage. The first internucleoside linkage of the region may be, e.g., an R_{P} internucleoside linkage. The second internucleoside linkage of the region may be, e.g., an S_{P} internucleoside linkage. The second internucleoside linkage of the region may be, e.g., an R_{P} internucleoside linkage. The third internucleoside linkage of the region may be, e.g., an S_{P} internucleoside linkage. The third internucleoside linkage of the region may be, e.g., an R_{P} internucleoside linkage. The fifth internucleoside linkage of the region may be, e.g., an S_{P} internucleoside linkage. The fifth internucleoside linkage of the region may be, e.g., an R_{P} internucleoside linkage. The seventh internucleoside linkage of the region may be, e.g., an S_{P} internucleoside linkage. The seventh internucleoside linkage of the region may be, e.g., an R_{P} internucleoside linkage. The eighth internucleoside linkage of the region may be, e.g., an S_{P} internucleoside linkage. The eighth internucleoside linkage of the region may be, e.g., an R_{P} internucleoside linkage. The ninth internucleoside linkage of the region may be, e.g., an S_{P} internucleoside linkage. The ninth internucleoside linkage of the region may be, e.g., an R_{P} internucleoside linkage. The eighteenth internucleoside linkage of the region may be, e.g., an S_{P} internucleoside linkage. The eighteenth internucleoside linkage of the region may be, e.g., an R_{P} internucleoside linkage. The nineteenth internucleoside linkage of the region may be, e.g., an S_{P} internucleoside linkage. The nineteenth internucleoside linkage of the region may be, e.g., an R_{P} internucleoside linkage. The twentieth internucleoside linkage of the region may be, e.g., an S_{P} internucleoside linkage. The twentieth internucleoside linkage of the region may be, e.g., an R_{P} internucleoside linkage.

The region may have a length of, e.g., at least 21 bases. The region may have a length of, e.g., 21 bases.

In some embodiments, each stereochemically enriched internucleoside linkage in an oligonucleotide is a phosphorothioate phosphodiester.

An oligonucleotide may have, e.g., at least 25% of its internucleoside linkages in S_{P} configuration. An oligonucleotide may have, e.g., at least 30% of its internucleoside linkages in S_{P} configuration. An oligonucleotide may have, e.g., at least 35% of its internucleoside linkages in S_{P} configuration. An oligonucleotide may have, e.g., at least 40% of its internucleoside linkages in S_{P} configuration. An oligonucleotide may have, e.g., at least 45% of its internucleoside linkages in S_{P} configuration. An oligonucleotide may have, e.g., at least 50% of its internucleoside linkages in S_{P} configuration. An oligonucleotide may have, e.g., at least 55% of its internucleoside linkages in S_{P} configuration. An oligonucleotide may have, e.g., at least 60% of its internucleoside linkages in S_{P} configuration. An oligonucleotide may have, e.g., at least 65% of its internucleoside linkages in S_{P} configuration. An oligonucleotide may have, e.g., at least 70% of its internucleoside linkages in S_{P} configuration. An oligonucleotide may have, e.g., at least 75% of its internucleoside linkages in S_{P} configuration. An oligonucleotide may have, e.g., at least 80% of its internucleoside linkages in S_{P} configuration. An oligonucleotide may have, e.g., at least 85% of its internucleoside linkages in S_{P} configuration. An oligonucleotide may have, e.g., at least 90% of its internucleoside linkages in S_{P} configuration.

An oligonucleotide may include at least two internucleoside linkages having different stereochemical configuration and/or different P-modifications relative to one another. The oligonucleotide may have a structure represented by the following formula:

[S^{B}ₙ₁R^{B}ₙ₂S^{B}ₙ₃R^{B}ₙ₄ . . . S^{B}ₙₓR^{B}_{ny}]

where:

each R^{B} independently represents a block of nucleotide units having the R_{P} configuration at the internucleoside linkage phosphorus atom;

each S^{B} independently represents a block of nucleotide units having the S_{P} configuration at the internucleoside linkage phosphorus atom;

each of n1 to ny is zero or an integer, provided that at least one odd n and at least one even n must be non-zero so that the oligonucleotide includes at least two internucleoside linkages with different stereochemistry relative to one another; and

where the sum of n1 to ny is between 2 and 200.

In some embodiments, the sum of n1 to ny is between a lower limit selected from the group consisting of 2, 3, 4, 5, 6, 7, 8, 9, 10, 11, 12, 13, 14, 15, 16, 17, 18, 19, 20, 21, 22, 23, 24, 25, and more, and the upper limit selected from the group consisting of 5, 10, 15, 20, 25, 30, 35, 40, 45, 50, 55, 60, 65, 70, 75, 80, 85, 90, 95, 100, 110, 120, 130, 140, 150, 160, 170, 180, 190, and 200, the upper limit being greater than the lower limit. In some of these embodiments, each n has the same value. In some embodiments, each even n has the same value as each other even n. In some embodiments, each odd n has the same value each other odd n. At least two even n's may have, e.g., different values from one another. At least two odd n's may have, e.g., different values from one another.

At least two adjacent n's may be, e.g., equal to one another, so that an oligonucleotide includes adjacent blocks of S_{P} linkages and R_{P} linkages of equal lengths. In some embodiments, an oligonucleotide includes repeating blocks of S_{P} and R_{P} linkages of equal lengths. In some embodiments, an oligonucleotide includes repeating blocks of S_{P} and R_{P} linkages, where at least two such blocks are of different lengths from one another. In some such embodiments, each S_{P} block is of the same length and is of a different length from each R_{P} block, where all R_{P} blocks may optionally be of the same length as one another.

At least two skip-adjacent n's may be, e.g., equal to one another, so that a provided oligonucleotide includes at least two blocks of internucleoside linkages of a first stereochemistry that are equal in length to one another and are separated by a separating block of internucleoside linkages of the opposite stereochemistry, where the separating block may be of the same length or a different length from the blocks of first stereochemistry.

In some embodiments, n's associated with linkage blocks at the ends of an oligonucleotide are of the same length. In some embodiments, an oligonucleotide has terminal blocks of the same linkage stereochemistry. In some such embodiments, the terminal blocks are separated from one another by a middle block of the opposite linkage stereochemistry.

An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoblockmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoskipmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoaltmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a gapmer.

An oligonucleotide of formula [S^{B}ₙ₁R^{B}ₙ₂S^{B}ₙ₃R^{B}ₙ₄ . . . S^{B}ₙₓR^{B}_{ny}] may be, e.g., of any of the above described patterns and may further include, e.g., patterns of P-modifications. For instance, an oligonucleotide of formula [S^{B}ₙ₁R^{B}n₂S^{B}ₙ₃R^{B}ₙ₄ . . . S^{B}ₙₓR^{B}_{ny}] may be, e.g., a stereoskipmer and a P-modification skipmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoblockmer and a P-modification altmer. An oligonucleotide of formula [S^{B}ₙ₁R^{B}ₙ₂S^{B}ₙ₃R^{B}ₙ₄ . . . S^{B}ₙₓR^{B}_{ny}] may be, e.g., a stereoaltmer and a P-modification blockmer.

An oligonucleotide may include, e.g., at least one phosphate phosphodiester and at least two consecutive modified internucleoside linkages. An oligonucleotide may include, e.g., at least one phosphate phosphodiester and at least two consecutive phosphorothioate triesters.

An oligonucleotide may be, e.g., a blockmer. An oligonucleotide may be, e.g., a stereoblockmer. An oligonucleotide may be, e.g., a P-modification blockmer. An oligonucleotide may be, e.g., a linkage blockmer.

An oligonucleotide may be, e.g., an altmer. An oligonucleotide may be, e.g., a stereoaltmer. An oligonucleotide may be, e.g., a P-modification altmer. An oligonucleotide may be, e.g., a linkage altmer.

An oligonucleotide may be, e.g., a unimer. An oligonucleotide may be, e.g., a stereounimer. An oligonucleotide may be, e.g., a P-modification unimer. An oligonucleotide may be, e.g., a linkage unimer.

An oligonucleotide may be, e.g., a skipmer.

In addition to the above, an antisense oligonucleotide may be generated in vivo in a cell (e.g., in a cell of a subject, such as a cancer patient) expressing the oligonucleotide. Thus, for example, an miRNA sponge including multiple sequences that are antisense to a miR-147b sequence can be expressed in a cell. This can be achieved, for example, by introduction of a vector into the cell. Optionally, the vector includes a promoter to direct transcription of the oligonucleotide, which may include, e.g., 1, 2, 3, 4, 5, 6, 7, 8, 9, 10 or more sequences (e.g., tandem repeated sequences) that are antisense to, and thus soak up and deplete or reduce the miR-147b of the cell. The miRNA binding sites in such miRNA sponges can be either perfectly antisense or contain mismatches, e.g., in the middle positions. Thus, for example, sponges can include bulged nucleotides that are mispaired opposite miRNA positions, e.g., positions 9-12, as is known in the art. These miRNA binding sites can be placed, for example, in the 3′-UTR of a nontoxic gene expressed in the cell. An miRNA sponge can be used to achieve stable inhibition, as well as inducible or tissue-specific inhibition, of a target miRNA, as needed. In various examples, a vector, such as a viral vector, e.g., a lentivirus, an adenovirus, or an adeno-associated virus is used to achieve expression of the miRNA sponge. In other examples, the vector is a plasmid, a cosmid, a phagemid, or a P element. Expression of miRNA sponges can be transient or stable, as is known in the art. See, e.g., Ebert et al., Nat. Methods 4:721-726, 2007; Ebert et al., RNA 16:2043-2050, 2010; Chen et al., Oncol. Rep. 31:1573-1580, 2014, for additional information regarding miRNA sponges.

Antisense molecules can also be competitive inhibitors of miR-147b with respect to binding to miR-147b targets. Accordingly, such inhibitors hybridize to targets of miR-147b, thus blocking the binding of miR-147b to these targets. In some embodiments, such inhibitors do not facilite the activity of RNAse H. In some embodiments, the affinity of such inhibitors for the targets is sufficient to block the activity of miR-147b, but does not block functional processing of the target (e.g., translation of the target).

In addition to the antisense molecules described above, the invention includes peptide nucleic acids (PNAs), which are synthetic molecules having certain characteristics analogous to characteristics of typical naturally occurring nucleic acids. In particular, typical naturally occurring nucleic acids include a sugar-phosphate backbone, together with nitrogenous nucleobases. PNA molecules, by contrast, can include a pseudo-peptide backbone including N-(2-aminoethyl) glycine units (rather than, e.g., a sugar-phosphate backbone), together with nitrogenous nucleobases (as described, for example, in U.S. Pat. No. 9,193,758. See also Nielsen et al., Science 254: 1497-1500, 1991). In such PNA molecules, repeating N-(2-aminoethyl)-glycine units can be linked by amide bonds. The PNA pseudo-peptide backbone can be acyclic, achiral, and neutrally charged. Nucleobases can be attached to the PNA pseudo-peptide backbone through methylene carbonyl linkages. Due at least in part to their distinct, hybrid composition, PNAs are resistant to both nucleases and proteases. Accordingly, the invention includes PNA molecules targeted against miR-147b, as described herein.

### RNAi

In another approach, the invention provides a double-stranded oligonucleotide including a passenger strand hybridized to a guide strand having a nucleobase sequence with at least 6 contiguous nucleobases complementary to an equal-length portion within a target miR-147b sequence, which includes mature miR-147b or a precursor (i.e., pri-miR-147b or pre-miR-147b) or fragment thereof. This approach is typically referred to as an RNAi approach, and the corresponding oligonucleotides of the invention are referred to as siRNA. Without wishing to be bound by theory, this approach involves incorporation of the guide strand into an RNA-induced silencing complex (RISC), which can identify and hybridize to a miR-147b sequence in a cell through complementarity of a portion of the guide strand and the target nucleic acid. Upon identification (and hybridization), RISC may either remain on the target nucleic acid thereby sterically blocking translation or cleave the target nucleic acid.

A double-stranded oligonucleotide of the invention may be an siRNA of the invention. An siRNA of the invention includes a guide strand and a passenger strand that are not covalently linked to each other. Alternatively, a double-stranded oligonucleotide of the invention may be an shRNA. An shRNA of the invention includes a guide strand and a passenger strand that are covalently linked to each other by a linker. Without wishing to be bound by theory, shRNA is processed by the Dicer enzyme to remove the linker and produce a corresponding siRNA. A double-stranded oligonucleotide of the invention (e.g., an siRNA of the invention) includes a nucleobase sequence having at least 6 (e.g., at least 7, 8, 9, 10, 11, 12, 13, 14, 15, 16, 17, 18, 19, or 20) contiguous nucleobases complementary to an equal-length portion of a miR-147b target nucleic acid, as described herein.

Typically, a guide strand includes a seed region, a slicing site, and 5′- and 3′-terminal residues. The seed region—typically, a six nucleotide-long sequence from position 2 to position 7—are involved in the target nucleic acid recognition. The slicing site are the nucleotides (typically at positions 10 and 11) that are complementary to the target nucleosides linked by an internucleoside linkage that undergoes a RISC-mediated cleavage. The 5′- and 3′ terminal residues typically interact with or are blocked by the Ago2 component of RISC.

A double-stranded oligonucleotide of the invention (e.g., an siRNA of the invention) may include one or more mismatches. For example, the one or more mismatches may be included outside the seed region and the slicing site. Typically, the one or more mismatches may be included among the 5′- and/or 3′-terminal nucleosides.

A double-stranded oligonucleotide of the invention (e.g., an siRNA of the invention) may include a guide strand having total of X to Y interlinked nucleosides and a passenger strand having a total of X to Y interlinked nucleosides, where each X represents independently the fewest number of nucleosides in the range and each Y represents independently the largest number nucleosides in the range. In these embodiments, X and Y are each independently selected from the group consisting of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X<Y. For example, a strand (e.g., a guide strand or a passenger strand) in a double-stranded oligonucleotide of the invention may include a total of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 29, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 interlinked nucleosides.

### Complementarity

Oligonucleotides of the invention, such as antisense oligonucleotides and siRNA, can optionally be 100% complementary to a target sequence (e.g., miR-147b, or a precursor or fragment thereof, or a target of miR-147b). However, it is possible to introduce mismatch bases without eliminating activity. Accordingly an oligonucleotide of the invention may include (i) a nucleobase sequence having at least 6 contiguous nucleobases complementary to an equal-length portion within a target miR-147b sequence, which includes mature miR-147b or a precursor (i.e., pri-miR-147b or pre-miR-147b) or fragment thereof, and (ii) a nucleobase sequence having a plurality of nucleobases including one or more nucleobases complementary to a target miR-147b sequence (including mature miR-147b or a precursor (i.e., pri-miR-147b or pre-miR-147b) or fragment thereof) and one or more mismatches.

In some embodiments, oligonucleotides of the invention are complementary to a miR-147b target nucleic acid over the entire length of the oligonucleotide. In other embodiments, oligonucleotides can be variants that are at least 80%, 85%, 90%, 95%, 99%, or 100% complementary to the miR-147b target nucleic acid. In further embodiments, oligonucleotides are at least 80% complementary to the miR-147b target nucleic acid over the entire length of the oligonucleotide and include a nucleobase sequence that is fully complementary to a miR-147b target nucleic acid. The nucleobase sequence that is fully complementary may be, e.g., 6 to 20, 10 to 18, or 18 to 20 contiguous nucleobases in length.

An oligonucleotide of the invention may include one or more mismatched nucleobases relative to a target nucleic acid. In certain embodiments, an antisense or RNAi activity against the target is reduced by such mismatch, but activity against a non-target is reduced by a greater amount. Thus, the off-target selectivity of the oligonucleotides may be improved.

### Oligonucleotide Modifications

An oligonucleotide of the invention may be a modified oligonucleotide. A modified oligonucleotide of the invention includes one or more modifications, e.g., a nucleobase modification, a sugar modification, an internucleoside linkage modification, or a terminal modification.

Nucleobase Modifications

Oligonucleotides of the invention may include one or more modified nucleobases. Unmodified nucleobases include the purine bases adenine (A) and guanine (G), and the pyrimidine bases thymine (T), cytosine (C), and uracil (U). Modified nucleobases include 5-substituted pyrimidines, 6-azapyrimidines, alkyl or alkynyl substituted pyrimidines, alkyl substituted purines, and N-2, N-6 and O-6 substituted purines, as well as synthetic and natural nucleobases, e.g., 5-methylcytosine, 5-hydroxymethyl cytosine, xanthine, hypoxanthine, 2-aminoadenine, 6-alkyl (e.g., 6-methyl) adenine and guanine, 2-alkyl (e.g., 2-propyl) adenine and guanine, 2-thiouracil, 2-thiothymine, 2-thiocytosine, 5-halouracil, 5-halocytosine, 5-propynyl uracil, 5-propynyl cytosine, 5-trifluoromethyl uracil, 5-trifluoromethyl cytosine, 7-methyl guanine, 7-methyl adenine, 8-azaguanine, 8-azaadenine, 7-deazaguanine, 7-deazaadenine, 3-deazaguanine, 3-deazaadenine. Certain nucleobases are particularly useful for increasing the binding affinity of nucleic acids, e g., 5-substituted pyrimidines; 6-azapyrimidines; N2-, N6-, and/or 06-substituted purines. Nucleic acid duplex stability can be enhanced using, e.g., 5-methylcytosine. Non-limiting examples of nucleobases include: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH3) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example, 7-deazaadenine, 7-deazaguanine, 2-aminopyridine and 2-pyridone. Further nucleobases include those disclosed in Merigan et al., U.S. Pat. No. 3,687,808, those disclosed in The Concise Encyclopedia of Polymer Science and Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Sugar Modifications

Oligonucleotides of the invention may include one or more sugar modifications in nucleosides. Nucleosides having an unmodified sugar include a sugar moiety that is a furanose ring as found in ribonucleosides and 2′-deoxyribonucleosides.

Sugars included in the nucleosides of the invention may be non-furanose (or 4′-substituted furanose) rings or ring systems or open systems. Such structures include simple changes relative to the natural furanose ring (e.g., a six-membered ring). Alternative sugars may also include sugar surrogates wherein the furanose ring has been replaced with another ring system such as, e.g., a morpholino or hexitol ring system. Non-limiting examples of sugar moieties useful that may be included in the oligonucleotides of the invention include β-D-ribose, β-D-2′-deoxyribose, substituted sugars (e.g., 2′, 5′, and bis substituted sugars), 4′-S-sugars (e.g., 4′-S-ribose, 4′-S-2′-deoxyribose, and 4′-S-2′-substituted ribose), bicyclic sugar moieties (e.g., the 2′-O—CH₂-4′ or 2′-O—(CH₂)₂-4′ bridged ribose derived bicyclic sugars) and sugar surrogates (when the ribose ring has been replaced with a morpholino or a hexitol ring system).

Typically, a sugar modification may be, e.g., a 2′-substitution, locking, carbocyclization, or unlocking. A 2′-substitution is a replacement of 2′-hydroxyl in ribofuranose with 2′-fluoro, 2′-methoxy, or 2′-(2-methoxy)ethoxy. Alternatively, a 2′-substitution may be a 2′-(ara) substitution, which corresponds to the following structure:

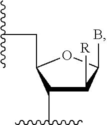

where B is a nucleobase, and R is a 2′-(ara) substituent (e.g., fluoro). 2′-(ara) substituents are known in the art and can be same as other 2′-substituents described herein. In some embodiments, 2′-(ara) substituent is a 2′-(ara)-F substituent (R is fluoro). A locking modification is an incorporation of a bridge between 4′-carbon atom and 2′-carbon atom of ribofuranose. Nucleosides having a sugar with a locking modification are known in the art as bridged nucleic acids, e.g., locked nucleic acids (LNA), ethylene-bridged nucleic acids (ENA), and cEt nucleic acids. The bridged nucleic acids are typically used as affinity enhancing nucleosides.

Internucleoside Linkage Modifications

Oligonucleotides of the invention may include one or more internucleoside linkage modifications. The two main classes of internucleoside linkages are defined by the presence or absence of a phosphorus atom. Non-limiting examples of phosphorus-containing internucleoside linkages include phosphodiester linkages, phosphotriester linkages, phosphorothioate diester linkages, phosphorothioate triester linkages, morpholino internucleoside linkages, methylphosphonates, and phosphoramidate. Non-limiting examples of non-phosphorus internucleoside linkages include methylenemethylimino (—CH₂—N(CH3)-O—CH2-), thiodiester (—O—C(O)—S—), thionocarbamate (—O—C(O)(NH)—S—), siloxane (—O—Si(H)2-O—), and N,N′-dimethylhydrazine (—CH2-N(CH3)-N(CH3)-). Modified linkages, compared to natural phosphodiester linkages, can be used to alter, typically increase, nuclease resistance of the oligonucleotide. Methods of preparation of phosphorous-containing and non-phosphorous-containing internucleoside linkages are known in the art.

Internucleoside linkages may be stereochemically enriched. For example, phosphorothioate-based internucleoside linkages (e.g., phosphorothioate diester or phosphorothioate triester) may be stereochemically enriched. The stereochemically enriched internucleoside linkages including a stereogenic phosphorus are typically designated S_{P} or R_{P} to identify the absolute stereochemistry of the phosphorus atom. Within an oligonucleotide, S_{P} phosphorothioate indicates the following structure:

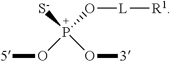

Within an oligonucleotide, R_{P} phosphorothioate indicates the following structure:

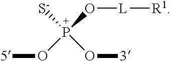

The oligonucleotides of the invention may include one or more neutral internucleoside linkages. Non-limiting examples of neutral internucleoside linkages include phosphotriesters, phosphorothioate triesters, methylphosphonates, methylenemethylimino (3′-CH₂—N(CH₃)—O-3′), amide-3 (3′-CH₂—C(═O)—N(H)-3′), amide-4 (3′-CH₂—N(H)—C(═O)-3′), formacetal (3′-O—CH₂—O-3′), and thioformacetal (3′-S—CH₂—O-3′). Further neutral internucleoside linkages include nonionic linkages including siloxane (dialkylsiloxane), carboxylate ester, carboxamide, sulfide, sulfonate ester, and amides (see, for example, Carbohydrate Modifications in Antisense Research; Y. S. Sanghvi and P. D. Cook, Eds., ACS Symposium Series 580; Chapters 3 and 4, 40-65).

Oligonucleotides may include, e.g., modified internucleoside linkages arranged along the oligonucleotide or region thereof in a defined pattern or modified internucleoside linkage motif. Oligonucleotides may include, e.g., a region having an alternating internucleoside linkage motif. In certain embodiments, oligonucleotides of the present disclosure include a region of uniformly modified internucleoside linkages. In certain such embodiments, the oligonucleotide may include, e.g., a region that is uniformly linked by phosphorothioate internucleoside linkages. The oligonucleotide may be, e.g., uniformly linked by phosphorothioate internucleoside linkages. Each internucleoside linkage of the oligonucleotide is selected from phosphodiester and phosphorothioate. Each internucleoside linkage of the oligonucleotide is selected from phosphodiester and phosphorothioate and at least one internucleoside linkage is phosphorothioate. The oligonucleotide may include, e.g., at least 6, 7, 8, 9, 10, 11, 12, 13, or 14 phosphorothioate internucleoside linkages.

The oligonucleotide may include, e.g., at least one block of at least 6 consecutive phosphorothioate internucleoside linkages. The oligonucleotide may include, e.g., at least one block of at least 7 consecutive phosphorothioate internucleoside linkages. The oligonucleotide may include, e.g., at least one block of at least 8 consecutive phosphorothioate internucleoside linkages. The oligonucleotide may include, e.g., at least one block of at least 9 consecutive phosphorothioate internucleoside linkages. The oligonucleotide may include, e.g., at least one block of at least 10 consecutive phosphorothioate internucleoside linkages. The oligonucleotide may include, e.g., at least one block of at least 12 consecutive phosphorothioate internucleoside linkages. In certain such embodiments, at least one such block is located at the 3′ end of the oligonucleotide. In certain such embodiments, at least one such block is located within 3 nucleosides of the 3′ end of the oligonucleotide. The oligonucleotide may include, e.g., fewer than 15 phosphorothioate internucleoside linkages. The oligonucleotide may include, e.g., fewer than 14 phosphorothioate internucleoside linkages. The oligonucleotide may include, e.g., fewer than 13 phosphorothioate internucleoside linkages. The oligonucleotide may include, e.g., fewer than 12 phosphorothioate internucleoside linkages. The oligonucleotide may include, e.g., fewer than 11 phosphorothioate internucleoside linkages. The oligonucleotide may include, e.g., fewer than 10 phosphorothioate internucleoside linkages. The oligonucleotide may include, e.g., fewer than 9 phosphorothioate internucleoside linkages. The oligonucleotide may include, e.g., fewer than 8 phosphorothioate internucleoside linkages. The oligonucleotide may include, e.g., fewer than 7 phosphorothioate internucleoside linkages. The oligonucleotide may include, e.g., fewer than 6 phosphorothioate internucleoside linkages. The oligonucleotide may include, e.g., fewer than 5 phosphorothioate internucleoside linkages. In some embodiments, at least one phosphorothioate internucleoside linkage is a phosphorothioate diester. In some embodiments, each phosphorothioate internucleoside linkage is a phosphorothioate diester. In some embodiments, at least one phosphorothioate internucleoside linkage is a phosphorothioate triester. In some embodiments, each phosphorothioate internucleoside linkage is a phosphorothioate triester. In some embodiments, each internucleoside linkage is independently a phosphodiester (e.g., phosphate phosphodiester or phosphorothioate diester).

An oligonucleotide may include a pattern of internucleoside P-stereogenic centers including (S_{P})ₘR_{P} or R_{P}(S_{P})ₘ. An oligonucleotide may include a pattern of internucleoside P-stereogenic centers including R_{P}(S_{P})ₘ. An oligonucleotide may include a pattern of internucleoside P-stereogenic centers including (S_{P})ₘR_{P}. In some embodiments, m is 2. An oligonucleotide may include a pattern of internucleoside P-stereogenic centers including R_{P}(S_{P})₂. An oligonucleotide may include a pattern of internucleoside P-stereogenic centers including (S_{P})₂R_{P}(S_{P})₂. An oligonucleotide may include a pattern of internucleoside P-stereogenic centers including (R_{P})₂R_{P}(S_{P})₂. An oligonucleotide may include a pattern of internucleoside P-stereogenic centers including R_{P}S_{P}R_{P}(S_{P})₂. An oligonucleotide may include a pattern of internucleoside P-stereogenic centers including S_{P}R_{P}R_{P}(S_{P})₂. An oligonucleotide may include a pattern of internucleoside P-stereogenic centers including (S_{P})₂R_{P}.

In the embodiments of internucleoside P-stereogenic center patterns, m is 2, 3, 4, 5, 6, 7, or 8, unless specified otherwise. In some embodiments of internucleoside P-stereogenic center patterns, m is 3, 4, 5, 6, 7, or 8. In some embodiments of internucleoside P-stereogenic center patterns, m is 4, 5, 6, 7, or 8. In some embodiments of internucleoside P-stereogenic center patterns, m is 5, 6, 7, or 8. In some embodiments of internucleoside P-stereogenic center patterns, m is 6, 7, or 8. In some embodiments of internucleoside P-stereogenic center patterns, m is 7 or 8. In some embodiments of internucleoside P-stereogenic center patterns, m is 2. In some embodiments of internucleoside P-stereogenic center patterns, m is 3. In some embodiments of internucleoside P-stereogenic center patterns, m is 4. In some embodiments of internucleoside P-stereogenic center patterns, m is 5. In some embodiments of internucleoside P-stereogenic center patterns, m is 6. In some embodiments of internucleoside P-stereogenic center patterns, m is 7. In some embodiments of internucleoside P-stereogenic center patterns, m is 8.

An oligonucleotide may include a region with at least one of the first, second, third, fifth, seventh, eighth, ninth, eighteenth, nineteenth, and twentieth internucleoside linkages being a P-stereogenic linkage (e.g., phosphorothioate phosphodiester or phosphorothioate phosphotriester). At least two of the first, second, third, fifth, seventh, eighth, ninth, eighteenth, nineteenth and twentieth internucleoside linkages are stereogenic. At least three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). At least nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Two of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Seven of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Nine of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester). Ten of the first, second, third, fifth, seventh, eighth, ninth, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighteenth, nineteenth and twentieth internucleoside linkages being P-stereogenic (e.g., phosphorothioate phosphodiester or phosphorothioate phosphotriester). At least two of the first, second, third, fifth, seventh, eighteenth, nineteenth and twentieth internucleoside linkages may be, e.g., P-stereogenic (e.g., phosphorothioate phosphodiester or phosphorothioate phosphotriester). At least three of the first, second, third, fifth, seventh, eighteenth, nineteenth and twentieth internucleoside linkages may be, e.g., P-stereogenic (e.g., phosphorothioate phosphodiester or phosphorothioate phosphotriester). At least four of the first, second, third, fifth, seventh, eighteenth, nineteenth and twentieth internucleoside linkages may be, e.g., P-stereogenic (e.g., phosphorothioate phosphodiester or phosphorothioate phosphotriester). At least five of the first, second, third, fifth, seventh, eighteenth, nineteenth and twentieth internucleoside linkages may be, e.g., P-stereogenic (e.g., phosphorothioate phosphodiester or phosphorothioate phosphotriester). At least six of the first, second, third, fifth, seventh, eighteenth, nineteenth and twentieth internucleoside linkages may be, e.g., P-stereogenic (e.g., phosphorothioate phosphodiester or phosphorothioate phosphotriester). At least seven of the first, second, third, fifth, seventh, eighteenth, nineteenth and twentieth internucleoside linkages may be, e.g., P-stereogenic (e.g., phosphorothioate phosphodiester or phosphorothioate phosphotriester). One of the first, second, third, fifth, seventh, eighteenth, nineteenth and twentieth internucleoside may be, e.g., P-stereogenic (e.g., phosphorothioate phosphodiester or phosphorothioate phosphotriester). Two of the first, second, third, fifth, seventh, eighteenth, nineteenth and twentieth internucleoside linkages may be, e.g., P-stereogenic (e.g., phosphorothioate phosphodiester or phosphorothioate phosphotriester). Three of the first, second, third, fifth, seventh, eighteenth, nineteenth and twentieth internucleoside linkages may be, e.g., P-stereogenic (e.g., phosphorothioate phosphodiester or phosphorothioate phosphotriester). Four of the first, second, third, fifth, seventh, eighteenth, nineteenth and twentieth internucleoside linkages may be, e.g., P-stereogenic (e.g., phosphorothioate phosphodiester or phosphorothioate phosphotriester). Five of the first, second, third, fifth, seventh, eighteenth, nineteenth and twentieth internucleoside linkages may be, e.g., P-stereogenic (e.g., phosphorothioate phosphodiester or phosphorothioate phosphotriester). Six of the first, second, third, fifth, seventh, eighteenth, nineteenth and twentieth internucleoside linkages may be, e.g., P-stereogenic (e.g., phosphorothioate phosphodiester or phosphorothioate phosphotriester).

Seven of the first, second, third, fifth, seventh, eighteenth, nineteenth and twentieth internucleoside linkages may be, e.g., P-stereogenic (e.g., phosphorothioate phosphodiester or phosphorothioate phosphotriester). Eight of the first, second, third, fifth, seventh, eighteenth, nineteenth and twentieth internucleoside linkages may be, e.g., P-stereogenic (e.g., phosphorothioate phosphodiester or phosphorothioate phosphotriester).

An oligonucleotide may include a region with at least one of the first, second, third, fifth, seventh, eighth, ninth, eighteenth, nineteenth, and twentieth internucleoside linkages being P-stereogenic (e.g., phosphorothioate phosphodiester or phosphorothioate phosphotriester), and at least one internucleoside linkage being non-stereogenic. An oligonucleotide may include a region in which at least one of the first, second, third, fifth, seventh, eighteenth, nineteenth, and twentieth internucleoside linkages being P-stereogenic (e.g., phosphorothioate phosphodiester or phosphorothioate phosphotriester), and at least one internucleoside linkage being non-stereogenic. At least two internucleoside linkages may be, e.g., non-stereogenic. At least three internucleoside linkages may be, e.g., non-stereogenic. At least four internucleoside linkages may be, e.g., non-stereogenic. At least five internucleoside linkages may be, e.g., non-stereogenic. At least six internucleoside linkages may be, e.g., non-stereogenic. At least seven internucleoside linkages may be, e.g., non-stereogenic. At least eight internucleoside linkages may be, e.g., non-stereogenic. At least nine internucleoside linkages may be, e.g., non-stereogenic. At least 10 internucleoside linkages may be, e.g., non-stereogenic. At least 11 internucleoside linkages may be, e.g., non-stereogenic. At least 12 internucleoside linkages may be, e.g., non-stereogenic. At least 13 internucleoside linkages may be, e.g., non-stereogenic. At least 14 internucleoside linkages may be, e.g., non-stereogenic. At least 15 internucleoside linkages may be, e.g., non-stereogenic. At least 16 internucleoside linkages may be, e.g., non-stereogenic. At least 17 internucleoside linkages may be, e.g., non-stereogenic. At least 18 internucleoside linkages may be, e.g., non-stereogenic. At least 19 internucleoside linkages may be, e.g., non-stereogenic. At least 20 internucleoside linkages may be, e.g., non-stereogenic. In some embodiments, one internucleoside linkage is non-stereogenic. In some embodiments, two internucleoside linkages are non-stereogenic. In some embodiments, three internucleoside linkages are non-stereogenic. In some embodiments, four internucleoside linkages are non-stereogenic. In some embodiments, five internucleoside linkages are non-stereogenic. In some embodiments, six internucleoside linkages are non-stereogenic. In some embodiments, seven internucleoside linkages are non-stereogenic. In some embodiments, eight internucleoside linkages are non-stereogenic. In some embodiments, nine internucleoside linkages are non-stereogenic. In some embodiments, 10 internucleoside linkages are non-stereogenic. In some embodiments, 11 internucleoside linkages are non-stereogenic. In some embodiments, 12 internucleoside linkages are non-stereogenic. In some embodiments, 13 internucleoside linkages are non-stereogenic. In some embodiments, 14 internucleoside linkages are non-stereogenic. In some embodiments, 15 internucleoside linkages are non-stereogenic. In some embodiments, 16 internucleoside linkages are non-stereogenic. In some embodiments, 17 internucleoside linkages are non-stereogenic. In some embodiments, 18 internucleoside linkages are non-stereogenic. In some embodiments, 19 internucleoside linkages are non-stereogenic. In some embodiments, 20 internucleoside linkages are non-stereogenic. An oligonucleotide may include a region in which all internucleoside linkages, except at least one of the first, second, third, fifth, seventh, eighth, ninth, eighteenth, nineteenth and twentieth internucleoside linkages which is P-stereogenic, are non-stereogenic.

An oligonucleotide may include a region with at least one of the first, second, third, fifth, seventh, eighth, ninth, eighteenth, nineteenth, and twentieth internucleoside linkages being P-stereogenic, and at least one internucleoside linkage being phosphate phosphodiester. An oligonucleotide may include a region with at least one of the first, second, third, fifth, seventh, eighteenth, nineteenth, and twentieth internucleoside linkages being P-stereogenic, and at least one internucleoside linkage being phosphate phosphodiester. At least two internucleoside linkages may be, e.g., phosphate phosphodiesters. At least three internucleoside linkages may be, e.g., phosphate phosphodiesters. At least four internucleoside linkages may be, e.g., phosphate phosphodiesters. At least five internucleoside linkages may be, e.g., phosphate phosphodiesters. At least six internucleoside linkages may be, e.g., phosphate phosphodiesters. At least seven internucleoside linkages may be, e.g., phosphate phosphodiesters. At least eight internucleoside linkages may be, e.g., phosphate phosphodiesters. At least nine internucleoside linkages may be, e.g., phosphate phosphodiesters. At least 10 internucleoside linkages may be, e.g., phosphate phosphodiesters. At least 11 internucleoside linkages may be, e.g., phosphate phosphodiesters. At least 12 internucleoside linkages may be, e.g., phosphate phosphodiesters. At least 13 internucleoside linkages may be, e.g., phosphate phosphodiesters. At least 14 internucleoside linkages may be, e.g., phosphate phosphodiesters. At least 15 internucleoside linkages may be, e.g., phosphate phosphodiesters. At least 16 internucleoside linkages may be, e.g., phosphate phosphodiesters. At least 17 internucleoside linkages may be, e.g., phosphate phosphodiesters. At least 18 internucleoside linkages may be, e.g., phosphate phosphodiesters. At least 19 internucleoside linkages may be, e.g., phosphate phosphodiesters. At least 20 internucleoside linkages may be, e.g., phosphate phosphodiesters. In some embodiments, one internucleoside linkage is phosphate phosphodiesters. In some embodiments, two internucleoside linkages are phosphate phosphodiesters.

In some embodiments, three internucleoside linkages are phosphate phosphodiesters. In some embodiments, four internucleoside linkages are phosphate phosphodiesters. In some embodiments, five internucleoside linkages are phosphate phosphodiesters. In some embodiments, six internucleoside linkages are phosphate phosphodiesters. In some embodiments, seven internucleoside linkages are phosphate phosphodiesters. In some embodiments, eight internucleoside linkages are phosphate phosphodiesters. In some embodiments, nine internucleoside linkages are phosphate phosphodiesters. In some embodiments, 10 internucleoside linkages are phosphate phosphodiesters. In some embodiments, 11 internucleoside linkages are phosphate phosphodiesters. In some embodiments, 12 internucleoside linkages are phosphate phosphodiesters. In some embodiments, 13 internucleoside linkages are phosphate phosphodiesters. In some embodiments, 14 internucleoside linkages are phosphate phosphodiesters. In some embodiments, 15 internucleoside linkages are phosphate phosphodiesters. In some embodiments, 16 internucleoside linkages are phosphate phosphodiesters. In some embodiments, 17 internucleoside linkages are phosphate phosphodiesters. In some embodiments, 18 internucleoside linkages are phosphate phosphodiesters. In some embodiments, 19 internucleoside linkages are phosphate phosphodiesters. In some embodiments, 20 internucleoside linkages are phosphate phosphodiesters. An oligonucleotide may include a region with all internucleoside linkages, except at least one of the first, second, third, fifth, seventh, eighth, ninth, eighteenth, nineteenth, and twentieth internucleoside linkages being P-stereogenic, being phosphate phosphodiesters.

An oligonucleotide may include a region with at least one of the first, second, third, fifth, seventh, eighth, ninth, eighteenth, nineteenth, and twentieth internucleoside linkages being P-stereogenic, and at least 10% of all internucleoside linkages in the region being non-stereogenic. An oligonucleotide may include a region with at least one of the first, second, third, fifth, seventh, eighteenth, nineteenth, and twentieth internucleoside linkages being P-stereogenic, and at least 10% of all internucleoside linkages in the region being non-stereogenic. At least 20% of all the internucleoside linkages in the region may be, e.g., non-stereogenic. At least 30% of al the internucleoside linkages in the region may be, e.g., non-stereogenic. At least 40% of all the internucleoside linkages in the region may be, e.g., non-stereogenic. At least 50% of all the internucleoside linkages in the region may be, e.g., non-stereogenic. At least 60% of all the internucleoside linkages in the region may be, e.g., non-stereogenic. At least 70% of all the internucleoside linkages in the region may be, e.g., non-stereogenic. At least 80% of all the internucleoside linkages in the region may be, e.g., non-stereogenic. At least 90% of all the internucleoside linkages in the region may be, e.g., non-stereogenic. At least 50% of all the internucleoside linkages in the region may be, e.g., non-stereogenic. A non-stereogenic internucleoside linkage may be, e.g., a phosphate phosphodiester. In some embodiments, each non-stereogenic internucleoside linkage is a phosphate phosphodiester.

The first internucleoside linkage of the region may be, e.g., an S_{P} internucleoside linkage. The first internucleoside linkage of the region may be, e.g., an R_{P} internucleoside linkage. The second internucleoside linkage of the region may be, e.g., an S_{P} internucleoside linkage. The second internucleoside linkage of the region may be, e.g., an R_{P} internucleoside linkage. The third internucleoside linkage of the region may be, e.g., an S_{P} internucleoside linkage. The third internucleoside linkage of the region may be, e.g., an R_{P} internucleoside linkage. The fifth internucleoside linkage of the region may be, e.g., an S_{P} internucleoside linkage. The fifth internucleoside linkage of the region may be, e.g., an R_{P} internucleoside linkage. The seventh internucleoside linkage of the region may be, e.g., an S_{P} internucleoside linkage. The seventh internucleoside linkage of the region may be, e.g., an R_{P} internucleoside linkage. The eighth internucleoside linkage of the region may be, e.g., an S_{P} internucleoside linkage. The eighth internucleoside linkage of the region may be, e.g., an R_{P} internucleoside linkage. The ninth internucleoside linkage of the region may be, e.g., an S_{P} internucleoside linkage. The ninth internucleoside linkage of the region may be, e.g., an R_{P} internucleoside linkage. The eighteenth internucleoside linkage of the region may be, e.g., an S_{P} internucleoside linkage. The eighteenth internucleoside linkage of the region may be, e.g., an R_{P} internucleoside linkage. The nineteenth internucleoside linkage of the region may be, e.g., an S_{P} internucleoside linkage. The nineteenth internucleoside linkage of the region may be, e.g., an R_{P} internucleoside linkage. The twentieth internucleoside linkage of the region may be, e.g., an S_{P} internucleoside linkage. The twentieth internucleoside linkage of the region may be, e.g., an R_{P} internucleoside linkage.

The region may have a length of, e.g., at least 21 bases. The region may have a length of, e.g., 21 bases.

In some embodiments, each stereochemically enriched internucleoside linkage in an oligonucleotide is a phosphorothioate phosphodiester.

An oligonucleotide may have, e.g., at least 25% of its internucleoside linkages in S_{P} configuration. An oligonucleotide may have, e.g., at least 30% of its internucleoside linkages in S_{P} configuration. An oligonucleotide may have, e.g., at least 35% of its internucleoside linkages in S_{P} configuration. An oligonucleotide may have, e.g., at least 40% of its internucleoside linkages in S_{P} configuration. An oligonucleotide may have, e.g., at least 45% of its internucleoside linkages in S_{P} configuration. An oligonucleotide may have, e.g., at least 50% of its internucleoside linkages in S_{P} configuration. An oligonucleotide may have, e.g., at least 55% of its internucleoside linkages in S_{P} configuration. An oligonucleotide may have, e.g., at least 60% of its internucleoside linkages in S_{P} configuration. An oligonucleotide may have, e.g., at least 65% of its internucleoside linkages in S_{P} configuration. An oligonucleotide may have, e.g., at least 70% of its internucleoside linkages in S_{P} configuration. An oligonucleotide may have, e.g., at least 75% of its internucleoside linkages in S_{P} configuration. An oligonucleotide may have, e.g., at least 80% of its internucleoside linkages in S_{P} configuration. An oligonucleotide may have, e.g., at least 85% of its internucleoside linkages in S_{P} configuration. An oligonucleotide may have, e.g., at least 90% of its internucleoside linkages in S_{P} configuration.

An oligonucleotide may include, e.g., at least one phosphate phosphodiester and at least two consecutive modified internucleoside linkages. An oligonucleotide may include, e.g., at least one phosphate phosphodiester and at least two consecutive phosphorothioate triesters.

An oligonucleotide may be, e.g., a blockmer. An oligonucleotide may be, e.g., a stereoblockmer. An oligonucleotide may be, e.g., a P-modification blockmer. An oligonucleotide may be, e.g., a linkage blockmer.

An oligonucleotide may be, e.g., an altmer. An oligonucleotide may be, e.g., a stereoaltmer. An oligonucleotide may be, e.g., a P-modification altmer. An oligonucleotide may be, e.g., a linkage altmer.

An oligonucleotide may be, e.g., a unimer. An oligonucleotide may be, e.g., a stereounimer. An oligonucleotide may be, e.g., a P-modification unimer. An oligonucleotide may be, e.g., a linkage unimer.

An oligonucleotide may be, e.g., a skipmer.

Terminal Modifications

Oligonucleotides of the invention may include a terminal modification. The terminal modification is a 5′-terminal modification or a 3′-terminal modification.

The 5 end of an oligonucleotide may be, e.g., hydroxyl, a hydrophobic moiety, 5′ cap, phosphate, diphosphate, triphosphate, phosphorothioate, diphosphorothioate, triphosphorothioate, phosphorodithioate, diphosphrodithioate, triphosphorodithioate, phosphonate, phosphoramidate, a cell penetrating peptide, an endosomal escape moiety, or a neutral organic polymer. An unmodified 5′-terminus is hydroxyl or phosphate. An oligonucleotide having a 5′ terminus other than 5′-hydroxyl or 5′-phosphate has a modified 5′ terminus.

The 3 end of an oligonucleotide may be, e.g., hydroxyl, a hydrophobic moiety, phosphate, diphosphate, triphosphate, phosphorothioate, diphosphorothioate, triphosphorothioate, phosphorodithioate, disphorodithioate, triphosphorodithioate, phosphonate, phosphoramidate, a cell penetrating peptide, an endosomal escape moiety, or a neutral organic polymer (e.g., polyethylene glycol). An unmodified 3′-terminus is hydroxyl or phosphate. An oligonucleotide having a 3′ terminus other than 3′-hydroxyl or 3′-phosphate has a modified 3′ terminus.

The terminal modification (e.g., 5′-terminal modification) may be, e.g., a hydrophobic moiety. Advantageously, an oligonucleotide including a hydrophobic moiety may exhibit superior cellular uptake, as compared to an oligonucleotide lacking the hydrophobic moiety. Oligonucleotides including a hydrophobic moiety may therefore be used in compositions that are substantially free of transfecting agents. A hydrophobic moiety is a monovalent group (e.g., a bile acid (e.g., cholic acid, taurocholic acid, deoxycholic acid, oleyl lithocholic acid, or oleoyl cholenic acid), glycolipid, phospholipid, sphingolipid, isoprenoid, vitamin, saturated fatty acid, unsaturated fatty acid, fatty acid ester, triglyceride, pyrene, porphyrine, texaphyrine, adamantine, acridine, biotin, coumarin, fluorescein, rhodamine, Texas-Red, digoxygenin, dimethoxytrityl, t-butydimethylsilyl, t-butyldiphenylsilyl, cyanine dye (e.g., Cy3 or Cy5), Hoechst 33258 dye, psoralen, or ibuprofen) covalently inked to the terminus of the oligonucleotide backbone (e.g., 5′-terminus). Non-limiting examples of the monovalent group include ergosterol, stigmasterol, β-sitosterol, campesterol, fucosterol, saringosterol, avenasterol, coprostanol, cholesterol, vitamin A, vitamin D, vitamin E, cardiolipin, and carotenoids. The linker connecting the monovalent group to the oligonucleotide may be a linker consisting of 1, 2, 3, 4, 5, 6, 7, 8, 9, or 10 monomers independently selected from the group consisting of optionally substituted C₁₋₁₂ alkylene, optionally substituted C₂₋₁₂ heteroalkylene, optionally substituted C₆₋₁₀ arylene, optionally substituted C₃₋₈ cycloalkylene, optionally substituted C₁₋₉ heteroarylene, optionally substituted C₁₋₉ heterocyclylene, —O—, —S—S—, and —NR^{N}—, where each R^{N} is independently H or optionally substituted C₁₋₁₂ alkyl. The linker may be bonded to an oligonucleotide through, e.g., an oxygen atom attached to a 5′-terminal carbon atom, a 3′-terminal carbon atom, a 5′-terminal phosphate or phosphorothioate, a 3′-terminal phosphate or phosphorothioate, or an internucleoside linkage.

### Preparation of Oligonucleotides

Oligonucleotides of the invention may be prepared using techniques and methods known in the art for the oligonucleotide synthesis. For example, oligonucleotides of the invention may be prepared using a phosphoramidite-based synthesis cycle. This synthesis cycle includes the steps of (1) de-blocking a 5-protected nucleotide to produce a 5′-deblocked nucleotide, (2) coupling the 5′-deblocked nucleotide with a 5-protected nucleoside phosphoramidite to produce nucleosides linked through a phosphite, (3) repeating steps (1) and (2) one or more times, as needed, (4) capping the 5′-terminus, and (5) oxidation or sulfurization of internucleoside phosphites. The reagents and reaction conditions useful for the oligonucleotide synthesis are known in the art.

The oligonucleotides disclosed herein may be linked to solid support as a result of solid-phase synthesis. Cleavable solid supports that may be used with the oligonucleotides are known in the art. Non-limiting examples of the solid support include, e.g., controlled pore glass or macroporous polystyrene bonded to a strand through a cleavable linker (e.g., succinate-based linker) known in the art (e.g., UnyLinker™). An oligonucleotide linked to solid support may be removed from the solid support by cleaving the linker connecting an oligonucleotide and solid support.

The oligonucleotides may further be synthesized such that they include any of the modifications described above and elsewhere herein including, e.g., 5′ and/or 3′ end modifications, or internucleoside modifications, used to facilitate targeting, delivery, and/or cell uptake. Also, as noted above, in certain instances, an oligonucleotide of the invention is synthesized in vivo. In such instances, an oligonucleotide (e.g., an miRNA sponge) may be generated from a vector (see above).

### Small Molecules and Other Inhibitors

As used herein, the term “smal molecule” refers to a molecule having a low molecular weight, typically less than 1000 Da. A small molecule may be naturally occurring or synthetic, and organic or inorganic. Smal molecule inhibitors of miR-147b can be identified, for example, using high throughput screening methods, which are optionally carried out in combination with bioinformatics-based analyses (see, e.g., Haga et al., Methods Mol. Biol. 1517:179-198, 2017). Furthermore, platforms for sequence-based design of small molecules targeting RNAs case be used (e.g., Infoma; Disney et al., ACS Chem. Biol. 11:1720-1728,2016). Also see, e.g., Xiao et al., Drug Target miRNA: Methods and Protocols, Schmidt, Ed., Springer, New York, N.Y., p. 169-178, 2017; and Vo et al., ACS Chem. Biol. 9:711-721, 2014; for additional information. Analyses of nucleic acid sequences, secondary structures, and effects of mutations, together with computer-aided drug design, can further be carried out to identify candidate small molecule inhibitors of miR-147b.

Small molecule inhibitors of the invention can act at any stage of miR-147b (or precursor) synthesis or affect its action, as described above. Thus, small molecule inhibitors can, for example, inhibit at the level of transcription pri-miR-147b, processing of pri-miR-147b to form pre-miR-147b, export of pre-miR-147b from the nucleus, processing of pre-miR-147b to form mature miR-147b, formation of miR-147b/RISC, and/or binding of miR-147b/RISC to its targets. Accordingly, small molecules can be screened for their activities at any one or more of these stages. In some embodiments, a small molecule inhibitor may target the narrow groove of the secondary structure of pre-miR-147b.

Other miR-147b inhibitors of the invention include, e.g., catalytic RNAs (e.g., ribozymes), aptamers, decoy oligonucleotides (see e.g., Wu et al., PlosOne 8(12):e82167, 2013; and Haraguchi et al., Nuc. Acids Res. 37:e43, 2009), and antibodies (e.g., antibodies that recognize RNA:RNA duplexes). In addition, gene editing approaches (e.g., CRISPR-cas9) can be used to knock-out miR147b or related molecules, as is known in the art. Small molecules and other miR-147b inhibitors identified using methods such as those described above can further be screened, for example, by use of organoids and related methods, such as those described herein.

### Pharmaceutical Compositions

An oligonucleotide, small molecule, decoy, or other miR-147b inhibitor of the invention (see, e.g., above) may be included in a pharmaceutical composition, optionally in combination with one or more additional miR-147b inhibitor or other therapeutic agent (see, e.g., above). A pharmaceutical composition typically includes a pharmaceutically acceptable diluent or carrier. A pharmaceutical composition may include (e.g., consist of), e.g., a sterile saline solution and an oligonucleotide of the invention. The sterile saline is typically a pharmaceutical grade saline. A pharmaceutical composition may include (e.g., consist of), e.g., sterile water and an oligonucleotide of the invention. The sterile water is typically a pharmaceutical grade water. A pharmaceutical composition may include (e.g., consist of), e.g., phosphate-buffered saline (PBS) and an oligonucleotide of the invention. The sterile PBS is typically a pharmaceutical grade PBS.

In certain embodiments, pharmaceutical compositions include one or more oligonucleotides and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose, and polyvinylpyrrolidone.

In certain embodiments, oligonucleotides may be admixed with pharmaceutically acceptable active and/or inert substances for the preparation of pharmaceutical compositions or formulations. Compositions and methods for the formulation of pharmaceutical compositions depend on a number of criteria, including, e.g., route of administration, extent of disease, or dose to be administered.

In certain embodiments, pharmaceutical compositions including an oligonucleotide encompass any pharmaceutically acceptable salts of the oligonucleotide, esters of the oligonucleotide, or salts of such esters. In certain embodiments, pharmaceutical compositions including an oligonucleotide, upon administration to a subject (e.g., a human), are capable of providing (directly or indirectly) the biologically active metabolite or residue thereof. Accordingly, for example, the disclosure is also drawn to pharmaceutically acceptable salts of oligonucleotides, prodrugs, pharmaceutically acceptable salts of such prodrugs, and other bioequivalents. Suitable pharmaceutically acceptable salts include, e.g., sodium and potassium salts. In certain embodiments, prodrugs include one or more conjugate group attached to an oligonucleotide, wherein the conjugate group is cleaved by endogenous nucleases within the body.

Lipid moieties have been used in nucleic acid therapies in a variety of methods. In certain such methods, the nucleic acid, such as an oligonucleotide, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions include a delivery system. Examples of delivery systems include, e.g., liposomes and emulsions. Certain delivery systems are useful for preparing certain pharmaceutical compositions including those including hydrophobic compounds. In certain embodiments, certain organic solvents such as dimethylsulfoxide are used.

In certain embodiments, pharmaceutical compositions include one or more tissue-specific delivery molecules designed to deliver the one or more pharmaceutical agents of the present invention to specific tissues or cell types. For example, in certain embodiments, pharmaceutical compositions include liposomes coated with a tissue-specific antibody.

In certain embodiments, pharmaceutical compositions include a co-solvent system. Certain of such co-solvent systems include, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol including 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal, intracerebroventricular, intracranial, intraocular etc.). In certain examples of such embodiments, a pharmaceutical composition includes a carrier and is formulated in aqueous solution, such as water or physiologically compatible buffers, such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions, or emulsions in oily or aqueous vehicles, and may contain formulatory agents such as suspending, stabilizing, and/or dispersing agents. Certain solvents suitable for use in pharmaceutical compositions for injection include, e.g., lipophilic solvents and fatty oils, such as sesame oil, synthetic fatty acid esters, such as ethyl oleate or triglycerides, and liposomes.

### Organoids

Conventional two-dimensional (2D) monolayer cell culture has been widely applied in vitro for screening small molecules targeting oncogenic signaling in cancers, including of EGFR TKIs against EGFR in lung cancer. However, cell lines typically grown in a 2D monolayer fail to represent the native architecture and cellular heterogeneity observed in the tumors from which these lines are derived. In recent years, evidence has accumulated pointing to the existence of a new dimension of intratumor heterogeneity and a hitherto-unappreciated subclass of neoplastic cells within tumors, termed tumor-initiating cells (TICs). The concept of TICs has significant clinical implications in that TICs are more resistant to current therapeutics including chemotherapy and radiotherapy. Thus, the current 2D monolayer cell culture might not be the ideal model to find new vulnerabilities for treatment resistance.

The present invention provides organoids, which are three-dimensional (3D) collections of organ-specific cell types that develop from stem cells or organ progenitors and self-organize through cell sorting and spatially restricted lineage commitment in a manner similar to that seen in vivo. The organoids of the present invention, which are based on lung cells (including, e.g., lung cancer cells) are designed to represent the native architecture of patient-derived tumors and treatment response towards current therapeutics. Accordingly, the invention provides methods for culturing lung cells (including lung cancer cells) as organoids from both primary tissues and cell lines. In one embodiment, the present invention provides methods for culturing lung tissue that maintains the differentiated state of the alveolar epithelial cells of the lung, or recapitulates the phenotype of lung tumors.

In some embodiments, methods for obtaining organoids according to the invention include the following steps: (a) obtaining a sample of lung tissue from a subject; (b) dissociating the sample of lung tissue; (c) isolating dissociated lung epithelial cells from the sample of lung tissue; and (d) culturing the dissociated lung epithelial cells. In some embodiments, the lung tissue is non-cancerous. In other embodiments, the lung tissue is cancerous. In further embodiments, the organoids are used as lung cancer xenografts in animal models, e.g., patient-derived xenograft (PDX)-containing mice.

In more detail, a stepwise method to establish lung organoids ex vivo, according to the invention, mimics the dynamic process of benign and malignant lung tissues formation, and includes stages of initiation (days 0-3), maintenance (days 4-6), and differentiation (days 7-24). The protocol first uses factors such as, e.g., EGF, FGF2, FGF10, and other niche factors to promote self-renewal of stem-like cells in the lung organoid. Then, factors such as FGF7 and PDGF are used during the differentiation stage to induce the differentiation of stem-like cells. Details of specific methods that can be used to generate organoids are found below in the Examples.

### Diagnostic and Screening Methods

The invention provides diagnostic methods that can be used to determine whether a subject has a cancer that may be (or be at risk of becoming) tolerant or resistant to anti-RTK therapy (e.g., anti-EGFR therapy; also see above) and, if so, if the resistance or tolerance may effectively be treated, reduced, prevented, or delayed by administration of a miR-147b inhibitor, as described herein. The invention also includes diagnostic methods that can be used to determine whether a subject has a cancer that may be effectively treated by administration of a miR-147b inhibitor, as described herein.

According to these methods, a sample from a subject (e.g., a human patient) is obtained and the sample is assayed for the presence of miR-147b (or a precursor or fragment thereof). Samples that can be used in these methods include, e.g., tumor tissues, tissue swabs, sputum, or blood samples (e.g., serum or plasma). Detection of miR-147b (or a precursor or fragment thereof) can carried out using standard methods including, e.g., hybridization assays, RNA-Seq, RT-PCR, and microarray-based assays. In both methods, detection of an increased level of miR-147b (or a precursor or fragment thereof), relative to a control (e.g., cells from a tissue-matched cancer that is not anti-RTK-therapy resistant or normal tissue-matched cells, as determined to be appropriate by those of skill in the art), indicates that miR-147b-targeted treatment may be effective. The level of increase that is diagnostic can be determined by those of skill in the art and may be, e.g., an increase of 25%, 50%, 100%, 150%, 200%, 300%, 500%, or more. Optionally, these diagnostic methods can also include a step of administering a miR-147b inhibitor to a subject identified as potentially benefiting from such treatment.

The invention further provides screening methods, which can be used to identify or characterize new miR-147b inhibitors, and also to select treatment that may be effective for a particular subject (e.g., a human patient having cancer). In these methods, a cell expressing miR-147b is contacted with a candidate inhibitor and the effects of the inhibitor on miR-147b expression or activity is determined (e.g., by RNA-Seq, etc.). A candidate inhibitor that is found to decrease the expression level or activity of miR-147b, relative to a control, can be considered as a potential miR-147b inhibitor that can be subject to further analysis, as needed. According to theses methods, the cells can be cultured cells (e.g., lung cancer-derived cell lines or primary cells) or the cells can be present in animal models (e.g., PDX-animal models, such as mice). Advantageously, the cells are lung cells (e.g., lung cancer cells) that are cultured to form organoids, as described above. As explained above, these structures model certain aspects of lung structure in vivo, and thus can provide for more accurate characterization of a candidate therapeutic agent (e.g., a miR-147b inhibitor). Moreover, if an organoid is derived from cells of a particular patient (e.g., cancer cells from a particular patient), the organoid can advantageously be used to test various treatments (e.g., miR-147b inhibitors, anti-RTK therapies, and/or other treatments), in order to identify a treatment protocol and regimen that may be particularly well-suited to the patient from whom the cells are derived. In addition to the above, the screening methods can be used to test combinations of therapies, e.g., combinations of miR-147b inhibitors of the invention with each other and other agents, such as other agents and treatments listed herein (e.g., carboplatin-base chemotherapy, radiotherapy, EGFR-based targeted therapy, and PD-1/PD-L1 based immunotherapy).

### Kits

The invention also provides kits for use in carrying out the methods of the invention. In some embodiments, a kit of the invention includes one or more agents (e.g., antisense oligonucleotides) for use in detecting the level of miR-147b (or a precursor or fragment thereof) in a sample (e.g., a patient sample, such as tumor tissue, tissue swab, sputum, or blood (e.g., serum or plasma)). In some embodiments, a kit of the invention includes multiple miR-147b inhibitors, as described herein, optionally in combination with one or more other therapeutic agent (e.g., a TKI, such as a TKI as described herein). In other, related embodiments, the kits include a miR-147b inhibitor in combination with one or more other therapeutic agent (e.g., a TKI, such as a TKI as described herein).

### Sequences

The sequence of pri-miR-147b is as follows: UAUAAAUCUAGUGGAAACAUUUCUGCACAAACUAGAUUCUGGACACCAGUGUGCGGAAAUGCUUC UGCUACAUUUUUAGG (SEQ ID NO: 1), while the sequence of mature miR-147b is: GUGUGCGGAAAUGCUUCUGCUA (SEQ ID NO: 2). Sequences that are antisense to these molecules can be used in the invention. Examples of such sequences, which can be used to target miR-147b (or a precursor or fragment thereof), according to the invention, include those comprising or consisting of the sequences in Tables 1 and 3 (e.g., SEQ ID NOs: 3-735). These sequences are various fragments of the reverse complement of SEQ ID NO: 1 (CCUAAAAAUGUAGCAGAAGCAUUUCCGCACACUGGUGUCCAGAAU CUAGUUUGUGCAGAAAUGUUUCCACUAGAUUUAUA; SEQ ID NO: 736). The sequences can comprise or be components of, e.g., antisense molecules described herein, or fragments thereof (e.g., a gap, 5′-wing, or 3′-wing). The sequences can further be present in molecules in single-stranded form or in double-stranded form. Furthermore, the sequences can be encoded in vectors, as described herein, for in vivo expression. As explained above, such sequences can optionally be present for expression as tandem multimers.

Sequences that can be used as competitive inhibitors, to compete with miR-147b for binding to an mRNA or pre-mRNA target, include the mature miR-147b sequence itself (SEQ ID NO: 2), or fragments or variants thereof. Examples of sequences that can be included in molecules that target miR-147b binding sites, according to the invention, include those comprising or consisting of the sequences in Tables 2 and 4 (e.g., SEQ ID NOs: 737-889).

For all the sequences listed herein, it is to be understood that U's are replaced with T's in the context of deoxyribonucleic acid molecules, and T's are to be replaced with U's in the context of ribonucleic acid molecules. Accordingly, even if a sequence is listed herein including U's, the sequence can be considered as including T's in their place, if appropriate in the context of the type of molecule under consideration. Similarly, if a sequence is listed herein including T's, the sequence can be considered as including U's in their place, if appropriate under the circumstances. Accordingly, if reference is made to a sequence identification number herein, then whether a T or U is to be considered in the sequence, regardless of the indicator in the sequence identifier, is based on the type of molecule intended. Mixed sequences, including both U's and T's are also included in the invention. Such molecules may include, e.g., T's in the gap region of an antisense molecule and then T's and/or U's in the wing(s). Such mixed sequences are included in the invention based on, e.g., the sequences listed in Tables 1-4, wherein one or more (e.g., all) U's are replaced with one or more T's. In addition, those of skill in the art can readily determine the sequence of a reference strand to utilize, relative to the miRNA sequences described herein, in the various contexts described herein. Furthermore, as is understood in the art, the length of each of these sequences may vary by the addition or deletion of 1 or more (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more) nucleotides on either or both ends. Also, as described above, additional sequences included in the invention are variants having sequence identity to these sequences (e.g., at least 70%, 75%, 80%, 85%, 90%, 95%, 99%, or 100%). Variants having one or more (e.g., 1, 2, 3, 4, 5, 6, 7, 8, 9, 10, 11, 12, 13, 14, 15, 16, 17, 18, 19, 20, or more) deletions or substations are also included in the invention. All sequences listed herein are in 5 to 3 orientation, unless otherwise indicated. In Tables 1-4, sequence identifiers are listed in one column, with the corresponding sequence in the next column. Sequences such as those listed in the Tables below, as well as in the Examples, below, can be included within the context of various molecules described above and elsewhere herein (e.g., antisense and siRNA molecules).

In some embodiments, the methods of the invention include targeting of sequences of or within SEQ ID NO: 1, e.g., sequences comprising or consisting of nucleotides 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 48-51, 47-52, 48-53, 49-54, 50-55, 51-56, 52-57, 53-58, 54-59, 55-80, 58-61, 57-62, 58-63, 59-64, 60-65, 61-66, 62-67, 63-68, 64-69, 65-70, 68-71, 67-72, 68-73, 69-74, 70-75, 71-76, 72-77, 73-78, 74-79, or 75-80 of SEQ ID NO: 1. In some embodiments, the methods of the invention include targeting sequences that consist of one of the sequence fragments listed immediately above and 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 additional nucleotides of SEQ ID NO: 1, whether all on one side of the indicated fragment or wherein the fragment is between the one or more additional nucleotides. In some embodiments the sequence targeted consists of or comprises SEQ ID NO: 2.

Accordingly, for example, sequences comprising or consisting of nucleotides 1-7, 2-8, 3-9, 4-10, 5-11, 6-12, 7-13, 8-14, 9-15, 10-16, 11-17, 12-18, 13-19, 14-20, 15-21, 16-22, 17-23, 18-24, 19-25, 20-26, 21-27, 22-28, 23-29, 24-30, 25-31, 26-32, 27-33, 28-34, 29-35, 30-36, 31-37, 32-38, 33-39, 34-40, 35-41, 36-42, 37-43, 38-44, 39-45, 40-46, 41-47, 42-48, 43-49, 44-50, 45-51, 48-52, 47-53,48-54, 49-55, 50-56, 51-57, 52-58, 53-59, 54-60, 55-61, 56-62, 57-63, 58-84, 59-65, 60-66, 61-67, 62-68, 63-69, 64-70, 65-71, 66-72, 67-73, 68-74, 69-75, 70-76, 71-77, 72-78, 73-79, or 74-80 of SEQ ID NO: 1, optionally having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, or 73 additional nucleotides of SEQ ID NO: 1, whether all on one side of the indicated fragment or wherein the fragment is between the one or more additional nucleotides, can be targeted. In some embodiments the sequence targeted consists of or comprises SEQ ID NO: 2.

In other embodiments, for example, sequences comprising or consisting of nucleotides 1-8, 2-9, 3-10, 4-11, 5-12, 6-13, 7-14, 8-15, 9-16, 10-17, 11-18, 12-19, 13-20, 14-21, 15-22, 16-23, 17-24, 18-25, 19-26, 20-27, 21-28, 22-29, 23-30, 24-31, 25-32, 26-33, 27-34, 28-35, 29-36, 30-37, 31-38, 32-39, 33-40, 34-41, 35-42, 36-43, 37-44, 38-45, 39-46, 40-47, 41-48, 42-49, 43-50, 44-51, 45-52, 46-53, 47-54, 48-55, 49-56, 50-57, 51-58, 52-59, 53-60, 54-61, 55-62, 56-63, 57-64, 58-65, 59-66, 60-67, 61-68, 62-69, 63-70, 64-71, 65-72, 66-73, 67-74, 68-75, 69-76, 70-77, 71-78, 72-79, or 73-80 of SEQ ID NO: 1, optionally having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 additional nucleotides of SEQ ID NO: 1, whether all on one side of the indicated fragment or wherein the fragment is between the one or more additional nucleotides, can be targeted. In some embodiments the sequence targeted consists of or comprises SEQ ID NO: 2.

In other embodiments, for example, sequences comprising or consisting of nucleotides 1-10, 2-11, 3-12, 4-13, 5-14, 6-15, 7-16, 8-17, 9-18, 10-19, 11-20, 12-21, 13-22, 14-23, 15-24, 16-25, 17-26, 18-27, 19-28, 20-29, 21-30, 22-31, 23-32, 24-33, 25-34, 26-35, 27-36, 28-37, 29-38, 30-39, 31-40, 32-41, 33-42, 34-43, 35-44, 36-45, 37-46, 38-47, 39-48, 40-49, 41-50, 42-51, 43-52, 44-53, 45-54, 48-55, 47-56, 48-57, 49-58, 50-59, 51-40, 52-61, 53-62, 54-63, 55-64, 58-65, 57-66, 58-67, 59-88, 60-69, or 61-70 of SEQ ID NO: 1, optionally having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, or 70 additional nucleotides of SEQ ID NO: 1, whether all on one side of the indicated fragment or wherein the fragment is between the one or more additional nucleotides, can be targeted. In some embodiments the sequence targeted consists of or comprises SEQ ID NO: 2.

In other embodiments, for example, sequences comprising or consisting of nucleotides 1-12, 2-13, 3-14, 4-15, 5-16, 6-17, 7-18, 8-19, 9-20, 10-21, 11-22, 12-23, 13-24, 14-25, 15-26, 16-27, 17-28, 18-29, 19-30, 20-31, 21-32, 22-33, 23-34, 24-35, 25-36, 26-37, 27-38, 28-39, 29-40, 30-41, 31-42, 32-43, 33-44, 34-45, 35-46, 36-47, 37-48, 38-49, 39-50, 40-51, 41-52, 42-53, 43-54, 44-55, 45-56, 48-57, 47-58, 48-59, 49-80, 50-61, 51-62, 52-63, 53-64, 54-65, 55-66, 58-67, 57-88, 58-69, or 59-70 of SEQ ID NO: 1, optionally having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, or 68 additional nucleotides of SEQ ID NO: 1, whether all on one side of the indicated fragment or wherein the fragment is between the one or more additional nucleotides, can be targeted. In some embodiments the sequence targeted consists of or comprises SEQ ID NO: 2.

In other embodiments, for example, sequences comprising or consisting of nucleotides 1-15, 2-16, 3-17, 4-18, 5-19, 6-20, 7-21, 8-22, 9-23, 10-24, 11-25, 12-26, 13-27, 14-28, 15-29, 16-30, 17-31, 18-32, 19-33, 20-34, 21-35, 22-36, 23-37, 24-38, 25-39, 26-40, 27-41, 28-42, 29-43, 30-44, 31-45, 32-46, 33-47, 34-48, 35-49, 36-50, 37-51, 38-52, 39-53, 40-54, 41-55, 42-56, 43-57, 44-58, 45-59, 48-80, 47-61, 48-62, 49-63, 50-84, 51-65, 52-66, 53-67, 54-68, 55-69, 58-70, 57-71, 58-72, 59-73, 60-74, 61-75, 62-76, 63-77, 64-78, 65-79, or 66-80 of SEQ ID NO: 1, optionally having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, or 65 additional nucleotides of SEQ ID NO: 1, whether all on one side of the indicated fragment or wherein the fragment is between the one or more additional nucleotides, can be targeted. In some embodiments the sequence targeted consists of or comprises SEQ ID NO: 2.

In other embodiments, for example, sequences comprising or consisting of nucleotides 1-18, 2-19, 3-20, 4-21, 5-22, 6-23, 7-24, 8-25, 9-26, 10-27, 11-28, 12-29, 13-30, 14-31, 15-32, 16-33, 17-34, 18-35, 19-36, 20-37, 21-38, 22-39, 23-40, 24-41, 25-42, 26-43, 27-44, 28-45, 29-46, 30-47, 31-48, 32-49, 33-50, 34-51, 35-52, 36-53, 37-54, 38-55, 39-56, 40-57, 41-58, 42-59, 43-60, 44-61, 45-62, 48-63, 47-64, 48-65, 49-66, 50-67, 51-68, 52-69, 53-70, 54-71, 55-72, 58-73, 57-74, 58-75, 59-76, 60-77, 61-78, 62-79, or 63-80 of SEQ ID NO: 1, optionally having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, or 62 additional nucleotides of SEQ ID NO: 1, whether all on one side of the indicated fragment or wherein the fragment is between the one or more additional nucleotides, can be targeted. In some embodiments the sequence targeted consists of or comprises SEQ ID NO: 2.

In other embodiments, for example, sequences comprising or consisting of nucleotides 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 48-65, 47-66, 48-67, 49-68, 50-69, 51-70, 52-71, 53-72, 54-73, 55-74, 58-75, 57-76, 58-77, 59-78, 60-79, or 61-80 of SEQ ID NO: 1, optionally having 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 additional nucleotides of SEQ ID NO: 1, whether all on one side of the indicated fragment or wherein the fragment is between the one or more additional nucleotides, can be targeted. In some embodiments the sequence targeted consists of or comprises SEQ ID NO: 2.

As noted above, the sequences of Tables 1 and 2, below, can each be considered to include one or more T's in place of one or more noted U, depending upon the use. Accordingly, the following tables describe the specifically listed sequences, as well as variants in which one or more U is replaced with a T. Furthermore, the sequences of Tables 1 and 2 can be considered as DNA, RNA, mixed DNA and RNA, or modifications thereof, and each of these different types of molecules is thus described herein. Tables 3 and 4 include the same sequences as Tables 1 and 2, respectively, but with U's replaced with T's. The same sequence identifiers are used to show the corresponding sequences.

TABLE 1

| | | | | | | | |
|---|---|---|---|---|---|---|---|
| 3 | CCUAAA | 52 | UUUGUG | 101 | UCCGCAC | 150 | AUUUAUA |
| 4 | CUAAAA | 53 | UUGUGC | 102 | CCGCACA | 151 | CCUAAAAA |
| 5 | UAAAAA | 54 | UGUGCA | 103 | CGCACAC | 152 | CUAAAAAU |
| 6 | AAAAAU | 55 | GUGCAG | 104 | GCACACU | 153 | UAAAAAUG |
| 7 | AAAAUG | 56 | UGCAGA | 105 | CACACUG | 154 | AAAAAUGU |
| 8 | AAAUGU | 57 | GCAGAA | 106 | ACACUGG | 155 | AAAAUGUA |
| 9 | AAUGUA | 58 | CAGAAA | 107 | CACUGGU | 156 | AAAUGUAG |
| 10 | AUGUAG | 59 | AGAAAU | 108 | ACUGGUG | 157 | AAUGUAGC |
| 11 | UGUAGC | 60 | GAAAUG | 109 | CUGGUGU | 158 | AUGUAGCA |
| 12 | GUAGCA | 61 | AAAUGU | 110 | UGGUGUC | 159 | UGUAGCAG |
| 13 | UAGCAG | 62 | AAUGUU | 111 | GGUGUCC | 160 | GUAGCAGA |
| 14 | AGCAGA | 63 | AUGUUU | 112 | GUGUCCA | 161 | UAGCAGAA |
| 15 | GCAGAA | 64 | UGUUUC | 113 | UGUCCAG | 162 | AGCAGAAG |
| 16 | CAGAAG | 65 | GUUUCC | 114 | GUCCAGA | 163 | GCAGAAGC |
| 17 | AGAAGC | 66 | UUUCCA | 115 | UCCAGAA | 164 | CAGAAGCA |
| 18 | GAAGCA | 67 | UUCCAC | 116 | CCAGAAU | 165 | AGAAGCAU |
| 19 | AAGCAU | 68 | UCCACU | 117 | CAGAAUC | 166 | GAAGCAUU |
| 20 | AGCAUU | 69 | CCACUA | 118 | AGAAUCU | 167 | AAGCAUUU |
| 21 | GCAUUU | 70 | CACUAG | 119 | GAAUCUA | 168 | AGCAUUUC |
| 22 | CAUUUC | 71 | ACUAGA | 120 | AAUCUAG | 169 | GCAUUUCC |
| 23 | AUUUCC | 72 | CUAGAU | 121 | AUCUAGU | 170 | CAUUUCCG |
| 24 | UUUCCG | 73 | UAGAUU | 122 | UCUAGUU | 171 | AUUUCCGC |
| 25 | UUCCGC | 74 | AGAUUU | 123 | CUAGUUU | 172 | UUUCCGCA |
| 26 | UCCGCA | 75 | GAUUUA | 124 | UAGUUUG | 173 | UUCCGCAC |
| 27 | CCGCAC | 76 | AUUUAU | 125 | AGUUUGU | 174 | UCCGCACA |
| 28 | CGCACA | 77 | UUUAUA | 126 | GUUUGUG | 175 | CCGCACAC |
| 29 | GCACAC | 78 | CCUAAAA | 127 | UUUGUGC | 176 | CGCACACU |
| 30 | CACACU | 79 | CUAAAAA | 128 | UUGUGCA | 177 | GCACACUG |
| 31 | ACACUG | 80 | UAAAAAU | 129 | UGUGCAG | 178 | CACACUGG |
| 32 | CACUGG | 81 | AAAAAUG | 130 | GUGCAGA | 179 | ACACUGGU |
| 33 | ACUGGU | 82 | AAAAUGU | 131 | UGCAGAA | 180 | CACUGGUG |
| 34 | CUGGUG | 83 | AAAUGUA | 132 | GCAGAAA | 181 | ACUGGUGU |
| 35 | UGGUGU | 84 | AAUGUAG | 133 | CAGAAAU | 182 | CUGGUGUC |
| 36 | GGUGUC | 85 | AUGUAGC | 134 | AGAAAUG | 183 | UGGUGUCC |
| 37 | GUGUCC | 86 | UGUAGCA | 135 | GAAAUGU | 184 | GGUGUCCA |
| 38 | UGUCCA | 87 | GUAGCAG | 136 | AAAUGUU | 185 | GUGUCCAG |
| 39 | GUCCAG | 88 | UAGCAGA | 137 | AAUGUUU | 186 | UGUCCAGA |
| 40 | UCCAGA | 89 | AGCAGAA | 138 | AUGUUUC | 187 | GUCCAGAA |
| 41 | CCAGAA | 90 | GCAGAAG | 139 | UGUUUCC | 188 | UCCAGAAU |
| 42 | CAGAAU | 91 | CAGAAGC | 140 | GUUUCCA | 189 | CCAGAAUC |
| 43 | AGAAUC | 92 | AGAAGCA | 141 | UUUCCAC | 190 | CAGAAUCU |
| 44 | GAAUCU | 93 | GAAGCAU | 142 | UUCCACU | 191 | AGAAUCUA |
| 45 | AAUCUA | 94 | AAGCAUU | 143 | UCCACUA | 192 | GAAUCUAG |
| 46 | AUCUAG | 95 | AGCAUUU | 144 | CCACUAG | 193 | AAUCUAGU |
| 47 | UCUAGU | 96 | GCAUUUC | 145 | CACUAGA | 194 | AUCUAGUU |
| 48 | CUAGUU | 97 | CAUUUCC | 146 | ACUAGAU | 195 | UCUAGUUU |
| 49 | UAGUUU | 98 | AUUUCCG | 147 | CUAGAUU | 196 | CUAGUUUG |
| 50 | AGUUUG | 99 | UUUCCGC | 148 | UAGAUUU | 197 | UAGUUUGU |
| 51 | GUUUGU | 100 | UUCCGCA | 149 | AGAUUUA | 198 | AGUUUGUG |
| 199 | GUUUGUGC | 248 | CCGCACACU | 297 | CCUAAAAAUG | 346 | UUUGUGCAGA |
| 200 | UUUGUGCA | 249 | CGCACACUG | 298 | CUAAAAAUGU | 347 | UUGUGCAGAA |
| 201 | UUGUGCAG | 250 | GCACACUGG | 299 | UAAAAAUGUA | 348 | UGUGCAGAAA |
| 202 | UGUGCAGA | 251 | CACACUGGU | 300 | AAAAAUGUAG | 349 | GUGCAGAAAU |
| 203 | GUGCAGAA | 252 | ACACUGGUG | 301 | AAAAUGUAGC | 350 | UGCAGAAAUG |
| 204 | UGCAGAAA | 253 | CACUGGUGU | 302 | AAAUGUAGCA | 351 | GCAGAAAUGU |
| 205 | GCAGAAAU | 254 | ACUGGUGUC | 303 | AAUGUAGCAG | 352 | CAGAAAUGUU |
| 206 | CAGAAAUG | 255 | CUGGUGUCC | 304 | AUGUAGCAGA | 353 | AGAAAUGUUU |
| 207 | AGAAAUGU | 256 | UGGUGUCCA | 305 | UGUAGCAGAA | 354 | GAAAUGUUUC |
| 208 | GAAAUGUU | 257 | GGUGUCCAG | 306 | GUAGCAGAAG | 355 | AAAUGUUUCC |
| 209 | AAAUGUUU | 258 | GUGUCCAGA | 307 | UAGCAGAAGC | 356 | AAUGUUUCCA |
| 210 | AAUGUUUC | 259 | UGUCCAGAA | 308 | AGCAGAAGCA | 357 | AUGUUUCCAC |
| 211 | AUGUUUCC | 260 | GUCCAGAAU | 309 | GCAGAAGCAU | 358 | UGUUUCCACU |
| 212 | UGUUUCCA | 261 | UCCAGAAUC | 310 | CAGAAGCAUU | 359 | GUUUCCACUA |
| 213 | GUUUCCAC | 262 | CCAGAAUCU | 311 | AGAAGCAUUU | 360 | UUUCCACUAG |
| 214 | UUUCCACU | 263 | CAGAAUCUA | 312 | GAAGCAUUUC | 361 | UUCCACUAGA |
| 215 | UUCCACUA | 264 | AGAAUCUAG | 313 | AAGCAUUUCC | 362 | UCCACUAGAU |
| 216 | UCCACUAG | 265 | GAAUCUAGU | 314 | AGCAUUUCCG | 363 | CCACUAGAUU |
| 217 | CCACUAGA | 266 | AAUCUAGUU | 315 | GCAUUUCCGC | 364 | CACUAGAUUU |
| 218 | CACUAGAU | 267 | AUCUAGUUU | 316 | CAUUUCCGCA | 365 | ACUAGAUUUA |
| 219 | ACUAGAUU | 268 | UCUAGUUUG | 317 | AUUUCCGCAC | 366 | CUAGAUUUAU |
| 220 | CUAGAUUU | 269 | CUAGUUUGU | 318 | UUUCCGCACA | 367 | UAGAUUUAUA |
| 221 | UAGAUUUA | 270 | UAGUUUGUG | 319 | UUCCGCACAC | 368 | CCUAAAAAUGU |
| 222 | AGAUUUAU | 271 | AGUUUGUGC | 320 | UCCGCACACU | 369 | CUAAAAAUGUA |
| 223 | GAUUUAUA | 272 | GUUUGUGCA | 321 | CCGCACACUG | 370 | UAAAAAUGUAG |
| 224 | CCUAAAAAU | 273 | UUUGUGCAG | 322 | CGCACACUGG | 371 | AAAAAUGUAGC |
| 225 | CUAAAAAUG | 274 | UUGUGCAGA | 323 | GCACACUGGU | 372 | AAAAUGUAGCA |
| 226 | UAAAAAUGU | 275 | UGUGCAGAA | 324 | CACACUGGUG | 373 | AAAUGUAGCAG |
| 227 | AAAAAUGUA | 276 | GUGCAGAAA | 325 | ACACUGGUGU | 374 | AAUGUAGCAGA |
| 228 | AAAAUGUAG | 277 | UGCAGAAAU | 326 | CACUGGUGUC | 375 | AUGUAGCAGAA |
| 229 | AAAUGUAGC | 278 | GCAGAAAUG | 327 | ACUGGUGUCC | 376 | UGUAGCAGAAG |
| 230 | AAUGUAGCA | 279 | CAGAAAUGU | 328 | CUGGUGUCCA | 377 | GUAGCAGAAGC |
| 231 | AUGUAGCAG | 280 | AGAAAUGUU | 329 | UGGUGUCCAG | 378 | UAGCAGAAGCA |
| 232 | UGUAGCAGA | 281 | GAAAUGUUU | 330 | GGUGUCCAGA | 379 | AGCAGAAGCAU |
| 233 | GUAGCAGAA | 282 | AAAUGUUUC | 331 | GUGUCCAGAA | 380 | GCAGAAGCAUU |
| 234 | UAGCAGAAG | 283 | AAUGUUUCC | 332 | UGUCCAGAAU | 381 | CAGAAGCAUUU |
| 235 | AGCAGAAGC | 284 | AUGUUUCCA | 333 | GUCCAGAAUC | 382 | AGAAGCAUUUC |
| 236 | GCAGAAGCA | 285 | UGUUUCCAC | 334 | UCCAGAAUCU | 383 | GAAGCAUUUCC |
| 237 | CAGAAGCAU | 286 | GUUUCCACU | 335 | CCAGAAUCUA | 384 | AAGCAUUUCCG |
| 238 | AGAAGCAUU | 287 | UUUCCACUA | 336 | CAGAAUCUAG | 385 | AGCAUUUCCGC |
| 239 | GAAGCAUUU | 288 | UUCCACUAG | 337 | AGAAUCUAGU | 386 | GCAUUUCCGCA |
| 240 | AAGCAUUUC | 289 | UCCACUAGA | 338 | GAAUCUAGUU | 387 | CAUUUCCGCAC |
| 241 | AGCAUUUCC | 290 | CCACUAGAU | 339 | AAUCUAGUUU | 388 | AUUUCCGCACA |
| 242 | GCAUUUCCG | 291 | CACUAGAUU | 340 | AUCUAGUUUG | 389 | UUUCCGCACAC |
| 243 | CAUUUCCGC | 292 | ACUAGAUUU | 341 | UCUAGUUUGU | 390 | UUCCGCACACU |
| 244 | AUUUCCGCA | 293 | CUAGAUUUA | 342 | CUAGUUUGUG | 391 | UCCGCACACUG |
| 245 | UUUCCGCAC | 294 | UAGAUUUAU | 343 | UAGUUUGUGC | 392 | CCGCACACUGG |
| 246 | UUCCGCACA | 295 | AGAUUUAUA | 344 | AGUUUGUGCA | 393 | CGCACACUGGU |
| 247 | UCCGCACAC | 296 | CCUAAAAAUG | 345 | GUUUGUGCAG | 394 | GCACACUGGUG |

| | | | | | |
|---|---|---|---|---|---|
| 395 | CACACUGGUGU | 443 | AAAUGUAGCAGA | 491 | UGCAGAAAUGUU |
| 396 | ACACUGGUGUC | 444 | AAUGUAGCAGAA | 492 | GCAGAAAUGUUU |
| 397 | CACUGGUGUCC | 445 | AUGUAGCAGAAG | 493 | CAGAAAUGUUUC |
| 398 | ACUGGUGUCCA | 446 | UGUAGCAGAAGC | 494 | AGAAAUGUUUCC |
| 399 | CUGGUGUCCAG | 447 | GUAGCAGAAGCA | 495 | GAAAUGUUUCCA |
| 400 | UGGUGUCCAGA | 448 | UAGCAGAAGCAU | 496 | AAAUGUUUCCAC |
| 401 | GGUGUCCAGAA | 449 | AGCAGAAGCAUU | 497 | AAUGUUUCCACU |
| 402 | GUGUCCAGAAU | 450 | GCAGAAGCAUUU | 498 | AUGUUUCCACUA |
| 403 | UGUCCAGAAUC | 451 | CAGAAGCAUUUC | 499 | UGUUUCCACUAG |
| 404 | GUCCAGAAUCU | 452 | AGAAGCAUUUCC | 500 | GUUUCCACUAGA |
| 405 | UCCAGAAUCUA | 453 | GAAGCAUUUCCG | 501 | UUUCCACUAGAU |
| 406 | CCAGAAUCUAG | 454 | AAGCAUUUCCGC | 502 | UUCCACUAGAUU |
| 407 | CAGAAUCUAGU | 455 | AGCAUUUCCGCA | 503 | UCCACUAGAUUU |
| 408 | AGAAUCUAGUU | 456 | GCAUUUCCGCAC | 504 | CCACUAGAUUUA |
| 409 | GAAUCUAGUUU | 457 | CAUUUCCGCACA | 505 | CACUAGAUUUAU |
| 410 | AAUCUAGUUUG | 458 | AUUUCCGCACAC | 506 | ACUAGAUUUAUA |
| 411 | AUCUAGUUUGU | 459 | UUUCCGCACACU | 507 | CCUAAAAAUGUAGCA |
| 412 | UCUAGUUUGUG | 460 | UUCCGCACACUG | 508 | CUAAAAAUGUAGCAG |
| 413 | CUAGUUUGUGC | 461 | UCCGCACACUGG | 509 | UAAAAAUGUAGCAGA |
| 414 | UAGUUUGUGCA | 462 | CCGCACACUGGU | 510 | AAAAAUGUAGCAGAA |
| 415 | AGUUUGUGCAG | 463 | CGCACACUGGUG | 511 | AAAAUGUAGCAGAAG |
| 416 | GUUUGUGCAGA | 464 | GCACACUGGUGU | 512 | AAAUGUAGCAGAAGC |
| 417 | UUUGUGCAGAA | 465 | CACACUGGUGUC | 513 | AAUGUAGCAGAAGCA |
| 418 | UUGUGCAGAAA | 466 | ACACUGGUGUCC | 514 | AUGUAGCAGAAGCAU |
| 419 | UGUGCAGAAAU | 467 | CACUGGUGUCCA | 515 | UGUAGCAGAAGCAUU |
| 420 | GUGCAGAAAUG | 468 | ACUGGUGUCCAG | 516 | GUAGCAGAAGCAUUU |
| 421 | UGCAGAAAUGU | 469 | CUGGUGUCCAGA | 517 | UAGCAGAAGCAUUUC |
| 422 | GCAGAAAUGUU | 470 | UGGUGUCCAGAA | 518 | AGCAGAAGCAUUUCC |
| 423 | CAGAAAUGUUU | 471 | GGUGUCCAGAAU | 519 | GCAGAAGCAUUUCCG |
| 424 | AGAAAUGUUUC | 472 | GUGUCCAGAAUC | 520 | CAGAAGCAUUUCCGC |
| 425 | GAAAUGUUUCC | 473 | UGUCCAGAAUCU | 521 | AGAAGCAUUUCCGCA |
| 426 | AAAUGUUUCCA | 474 | GUCCAGAAUCUA | 522 | GAAGCAUUUCCGCAC |
| 427 | AAUGUUUCCAC | 475 | UCCAGAAUCUAG | 523 | AAGCAUUUCCGCACA |
| 428 | AUGUUUCCACU | 476 | CCAGAAUCUAGU | 524 | AGCAUUUCCGCACAC |
| 429 | UGUUUCCACUA | 477 | CAGAAUCUAGUU | 525 | GCAUUUCCGCACACU |
| 430 | GUUUCCACUAG | 478 | AGAAUCUAGUUU | 526 | CAUUUCCGCACACUG |
| 431 | UUUCCACUAGA | 479 | GAAUCUAGUUUG | 527 | AUUUCCGCACACUGG |
| 432 | UUCCACUAGAU | 480 | AAUCUAGUUUGU | 528 | UUUCCGCACACUGGU |
| 433 | UCCACUAGAUU | 481 | AUCUAGUUUGUG | 529 | UUCCGCACACUGGUG |
| 434 | CCACUAGAUUU | 482 | UCUAGUUUGUGC | 530 | UCCGCACACUGGUGU |
| 435 | CACUAGAUUUA | 483 | CUAGUUUGUGCA | 531 | CCGCACACUGGUGUC |
| 436 | ACUAGAUUUAU | 484 | UAGUUUGUGCAG | 532 | CGCACACUGGUGUCC |
| 437 | CUAGAUUUAUA | 485 | AGUUUGUGCAGA | 533 | GCACACUGGUGUCCA |
| 438 | CCUAAAAAUGUA | 486 | GUUUGUGCAGAA | 534 | CACACUGGUGUCCAG |
| 439 | CUAAAAAUGUAG | 487 | UUUGUGCAGAAA | 535 | ACACUGGUGUCCAGA |
| 440 | UAAAAAUGUAGC | 488 | UUGUGCAGAAAU | 536 | CACUGGUGUCCAGAA |
| 441 | AAAAAUGUAGCA | 489 | UGUGCAGAAAUG | 537 | ACUGGUGUCCAGAAU |
| 442 | AAAAUGUAGCAG | 490 | GUGCAGAAAUGU | 538 | CUGGUGUCCAGAAUC |

| | | | |
|---|---|---|---|
| 539 | UGGUGUCCAGAAUCU | 587 | AGAAGCAUUUCCGCACACUG |
| 540 | GGUGUCCAGAAUCUA | 588 | GAAGCAUUUCCGCACACUGG |
| 541 | GUGUCCAGAAUCUAG | 589 | AAGCAUUUCCGCACACUGGU |
| 542 | UGUCCAGAAUCUAGU | 590 | AGCAUUUCCGCACACUGGUG |
| 543 | GUCCAGAAUCUAGUU | 591 | GCAUUUCCGCACACUGGUGU |
| 544 | UCCAGAAUCUAGUUU | 592 | CAUUUCCGCACACUGGUGUC |
| 545 | CCAGAAUCUAGUUUG | 593 | AUUUCCGCACACUGGUGUCC |
| 546 | CAGAAUCUAGUUUGU | 594 | UUUCCGCACACUGGUGUCCA |
| 547 | AGAAUCUAGUUUGUG | 595 | UUCCGCACACUGGUGUCCAG |
| 548 | GAAUCUAGUUUGUGC | 596 | UCCGCACACUGGUGUCCAGA |
| 549 | AAUCUAGUUUGUGCA | 597 | CCGCACACUGGUGUCCAGAA |
| 550 | AUCUAGUUUGUGCAG | 598 | CGCACACUGGUGUCCAGAAU |
| 551 | UCUAGUUUGUGCAGA | 599 | GCACACUGGUGUCCAGAAUC |
| 552 | CUAGUUUGUGCAGAA | 600 | CACACUGGUGUCCAGAAUCU |
| 553 | UAGUUUGUGCAGAAA | 601 | ACACUGGUGUCCAGAAUCUA |
| 554 | AGUUUGUGCAGAAAU | 602 | CACUGGUGUCCAGAAUCUAG |
| 555 | GUUUGUGCAGAAAUG | 603 | ACUGGUGUCCAGAAUCUAGU |
| 556 | UUUGUGCAGAAAUGU | 604 | CUGGUGUCCAGAAUCUAGUU |
| 557 | UUGUGCAGAAAUGUU | 605 | UGGUGUCCAGAAUCUAGUUU |
| 558 | UGUGCAGAAAUGUUU | 606 | GGUGUCCAGAAUCUAGUUUG |
| 559 | GUGCAGAAAUGUUUC | 607 | GUGUCCAGAAUCUAGUUUGU |
| 560 | UGCAGAAAUGUUUCC | 608 | UGUCCAGAAUCUAGUUUGUG |
| 561 | GCAGAAAUGUUUCCA | 609 | GUCCAGAAUCUAGUUUGUGC |
| 562 | CAGAAAUGUUUCCAC | 610 | UCCAGAAUCUAGUUUGUGCA |
| 563 | AGAAAUGUUUCCACU | 611 | CCAGAAUCUAGUUUGUGCAG |
| 564 | GAAAUGUUUCCACUA | 612 | CAGAAUCUAGUUUGUGCAGA |
| 565 | AAAUGUUUCCACUAG | 613 | AGAAUCUAGUUUGUGCAGAA |
| 566 | AAUGUUUCCACUAGA | 614 | GAAUCUAGUUUGUGCAGAAA |
| 567 | AUGUUUCCACUAGAU | 615 | AAUCUAGUUUGUGCAGAAAU |
| 568 | UGUUUCCACUAGAUU | 616 | AUCUAGUUUGUGCAGAAAUG |
| 569 | GUUUCCACUAGAUUU | 617 | UCUAGUUUGUGCAGAAAUGU |
| 570 | UUUCCACUAGAUUUA | 618 | CUAGUUUGUGCAGAAAUGUU |
| 571 | UUCCACUAGAUUUAU | 619 | UAGUUUGUGCAGAAAUGUUU |
| 572 | UCCACUAGAUUUAUA | 620 | AGUUUGUGCAGAAAUGUUUC |
| 573 | CCUAAAAAUGUAGCAGAAGC | 621 | GUUUGUGCAGAAAUGUUUCC |
| 574 | CUAAAAAUGUAGCAGAAGCA | 622 | UUUGUGCAGAAAUGUUUCCA |
| 575 | UAAAAAUGUAGCAGAAGCAU | 623 | UUGUGCAGAAAUGUUUCCAC |
| 576 | AAAAAUGUAGCAGAAGCAUU | 624 | UGUGCAGAAAUGUUUCCACU |
| 577 | AAAAUGUAGCAGAAGCAUUU | 625 | GUGCAGAAAUGUUUCCACUA |
| 578 | AAAUGUAGCAGAAGCAUUUC | 626 | UGCAGAAAUGUUUCCACUAG |
| 579 | AAUGUAGCAGAAGCAUUUCC | 627 | GCAGAAAUGUUUCCACUAGA |
| 580 | AUGUAGCAGAAGCAUUUCCG | 628 | CAGAAAUGUUUCCACUAGAU |
| 581 | UGUAGCAGAAGCAUUUCCGC | 629 | AGAAAUGUUUCCACUAGAUU |
| 582 | GUAGCAGAAGCAUUUCCGCA | 630 | GAAAUGUUUCCACUAGAUUU |
| 583 | UAGCAGAAGCAUUUCCGCAC | 631 | AAAUGUUUCCACUAGAUUUA |
| 584 | AGCAGAAGCAUUUCCGCACA | 632 | AAUGUUUCCACUAGAUUUAU |
| 585 | GCAGAAGCAUUUCCGCACAC | 633 | AUGUUUCCACUAGAUUUAUA |
| 586 | CAGAAGCAUUUCCGCACACU | 634 | CCUAAAAAUGUAGCAGAAGCAUUU |
| 635 | CUAAAAAUGUAGCAGAAGCAUUUC | 678 | UCUAGUUUGUGCAGAAAUGUUUCC |
| 636 | UAAAAAUGUAGCAGAAGCAUUUCC | 679 | CUAGUUUGUGCAGAAAUGUUUCCA |
| 637 | AAAAAUGUAGCAGAAGCAUUUCCG | 680 | UAGUUUGUGCAGAAAUGUUUCCAC |
| 638 | AAAAUGUAGCAGAAGCAUUUCCGC | 681 | AGUUUGUGCAGAAAUGUUUCCACU |
| 639 | AAAUGUAGCAGAAGCAUUUCCGCA | 682 | GUUUGUGCAGAAAUGUUUCCACUA |
| 640 | AAUGUAGCAGAAGCAUUUCCGCAC | 683 | UUUGUGCAGAAAUGUUUCCACUAG |
| 641 | AUGUAGCAGAAGCAUUUCCGCACA | 684 | UUGUGCAGAAAUGUUUCCACUAGA |
| 642 | UGUAGCAGAAGCAUUUCCGCACAC | 685 | UGUGCAGAAAUGUUUCCACUAGAU |
| 643 | GUAGCAGAAGCAUUUCCGCACACU | 686 | CCUAAAAAUGUAGCAGAAGCAUUUCCGCAC |
| 644 | UAGCAGAAGCAUUUCCGCACACUG | 687 | CUAAAAAUGUAGCAGAAGCAUUUCCGCACA |
| 645 | AGCAGAAGCAUUUCCGCACACUGG | 688 | UAAAAAUGUAGCAGAAGCAUUUCCGCACAC |
| 646 | GCAGAAGCAUUUCCGCACACUGGU | 689 | AAAAAUGUAGCAGAAGCAUUUCCGCACACU |
| 647 | CAGAAGCAUUUCCGCACACUGGUG | 690 | AAAAUGUAGCAGAAGCAUUUCCGCACACUG |
| 648 | AGAAGCAUUUCCGCACACUGGUGU | 691 | AAAUGUAGCAGAAGCAUUUCCGCACACUGG |
| 649 | GAAGCAUUUCCGCACACUGGUGUC | 692 | AAUGUAGCAGAAGCAUUUCCGCACACUGGU |
| 650 | AAGCAUUUCCGCACACUGGUGUCC | 693 | AUGUAGCAGAAGCAUUUCCGCACACUGGUG |
| 651 | AGCAUUUCCGCACACUGGUGUCCA | 694 | UGUAGCAGAAGCAUUUCCGCACACUGGUGU |
| 652 | GCAUUUCCGCACACUGGUGUCCAG | 695 | GUAGCAGAAGCAUUUCCGCACACUGGUGUC |
| 653 | CAUUUCCGCACACUGGUGUCCAGA | 696 | UAGCAGAAGCAUUUCCGCACACUGGUGUCC |
| 654 | AUUUCCGCACACUGGUGUCCAGAA | 697 | AGCAGAAGCAUUUCCGCACACUGGUGUCCA |
| 655 | UUUCCGCACACUGGUGUCCAGAAU | 698 | GCAGAAGCAUUUCCGCACACUGGUGUCCAG |
| 656 | UUCCGCACACUGGUGUCCAGAAUC | 699 | CAGAAGCAUUUCCGCACACUGGUGUCCAGA |
| 657 | UCCGCACACUGGUGUCCAGAAUCU | 700 | AGAAGCAUUUCCGCACACUGGUGUCCAGAA |
| 658 | CCGCACACUGGUGUCCAGAAUCUA | 701 | GAAGCAUUUCCGCACACUGGUGUCCAGAAU |
| 659 | CGCACACUGGUGUCCAGAAUCUAG | 702 | AAGCAUUUCCGCACACUGGUGUCCAGAAUC |
| 660 | GCACACUGGUGUCCAGAAUCUAGU | 703 | AGCAUUUCCGCACACUGGUGUCCAGAAUCU |
| 661 | CACACUGGUGUCCAGAAUCUAGUU | 704 | GCAUUUCCGCACACUGGUGUCCAGAAUCUA |
| 662 | ACACUGGUGUCCAGAAUCUAGUUU | 705 | CAUUUCCGCACACUGGUGUCCAGAAUCUAG |
| 663 | CACUGGUGUCCAGAAUCUAGUUUG | 706 | AUUUCCGCACACUGGUGUCCAGAAUCUAGU |
| 664 | ACUGGUGUCCAGAAUCUAGUUUGU | 707 | UUUCCGCACACUGGUGUCCAGAAUCUAGUU |
| 665 | CUGGUGUCCAGAAUCUAGUUUGUG | 708 | UUCCGCACACUGGUGUCCAGAAUCUAGUUU |
| 666 | UGGUGUCCAGAAUCUAGUUUGUGC | 709 | UCCGCACACUGGUGUCCAGAAUCUAGUUUG |
| 667 | GGUGUCCAGAAUCUAGUUUGUGCA | 710 | CCGCACACUGGUGUCCAGAAUCUAGUUUGU |
| 668 | GUGUCCAGAAUCUAGUUUGUGCAG | 711 | CGCACACUGGUGUCCAGAAUCUAGUUUGUG |
| 669 | UGUCCAGAAUCUAGUUUGUGCAGA | 712 | GCACACUGGUGUCCAGAAUCUAGUUUGUGC |
| 670 | GUCCAGAAUCUAGUUUGUGCAGAA | 713 | CACACUGGUGUCCAGAAUCUAGUUUGUGCA |
| 671 | UCCAGAAUCUAGUUUGUGCAGAAA | 714 | ACACUGGUGUCCAGAAUCUAGUUUGUGCAG |
| 672 | CCAGAAUCUAGUUUGUGCAGAAAU | 715 | CACUGGUGUCCAGAAUCUAGUUUGUGCAGA |
| 673 | CAGAAUCUAGUUUGUGCAGAAAUG | 716 | ACUGGUGUCCAGAAUCUAGUUUGUGCAGAA |
| 674 | AGAAUCUAGUUUGUGCAGAAAUGU | 717 | CUGGUGUCCAGAAUCUAGUUUGUGCAGAAA |
| 675 | GAAUCUAGUUUGUGCAGAAAUGUU | 718 | UGGUGUCCAGAAUCUAGUUUGUGCAGAAAU |
| 676 | AAUCUAGUUUGUGCAGAAAUGUUU | 719 | GGUGUCCAGAAUCUAGUUUGUGCAGAAAUG |
| 677 | AUCUAGUUUGUGCAGAAAUGUUUC | 720 | GUGUCCAGAAUCUAGUUUGUGCAGAAAUGU |

| | |
|---|---|
| 721 | GUCCAGAAUCUAGUUUGUGCAGAAAUGUUU |
| 722 | UCCAGAAUCUAGUUUGUGCAGAAAUGUUUC |
| 723 | CCAGAAUCUAGUUUGUGCAGAAAUGUUUCC |
| 724 | CAGAAUCUAGUUUGUGCAGAAAUGUUUCCA |
| 725 | AGAAUCUAGUUUGUGCAGAAAUGUUUCCAC |
| 726 | GAAUCUAGUUUGUGCAGAAAUGUUUCCACU |
| 727 | AAUCUAGUUUGUGCAGAAAUGUUUCCACUA |
| 728 | AUCUAGUUUGUGCAGAAAUGUUUCCACUAG |
| 729 | UCUAGUUUGUGCAGAAAUGUUUCCACUAGA |
| 730 | CUAGUUUGUGCAGAAAUGUUUCCACUAGAU |
| 731 | UAGUUUGUGCAGAAAUGUUUCCACUAGAUU |
| 732 | AGUUUGUGCAGAAAUGUUUCCACUAGAUUU |
| 733 | GUUUGUGCAGAAAUGUUUCCACUAGAUUUA |
| 734 | UUUGUGCAGAAAUGUUUCCACUAGAUUUAU |
| 735 | UUGUGCAGAAAUGUUUCCACUAGAUUUAUA |

TABLE 2

| | | | | | |
|---|---|---|---|---|---|
| 737 | GUGUGC | 785 | GUGUGCGGA | 833 | AAUGCUUCUGCU |
| 738 | UGUGCG | 786 | UGUGCGGAA | 834 | AUGCUUCUGCUA |
| 739 | GUGCGG | 787 | GUGCGGAAA | 835 | GUGUGCGGAAAUG |
| 740 | UGCGGA | 788 | UGCGGAAAU | 836 | UGUGCGGAAAUGC |
| 741 | GCGGAA | 789 | GCGGAAAUG | 837 | GUGCGGAAAUGCU |
| 742 | CGGAAA | 790 | CGGAAAUGC | 838 | UGCGGAAAUGCUU |
| 743 | GGAAAU | 791 | GGAAAUGCU | 839 | GCGGAAAUGCUUC |
| 744 | GAAAUG | 792 | GAAAUGCUU | 840 | CGGAAAUGCUUCU |
| 745 | AAAUGC | 793 | AAAUGCUUC | 841 | GGAAAUGCUUCUG |
| 746 | AAUGCU | 794 | AAUGCUUCU | 842 | GAAAUGCUUCUGC |
| 747 | AUGCUU | 795 | AUGCUUCUG | 843 | AAAUGCUUCUGCU |
| 748 | UGCUUC | 796 | UGCUUCUGC | 844 | AAUGCUUCUGCUA |
| 749 | GCUUCU | 797 | GCUUCUGCU | 845 | GUGUGCGGAAAUGC |
| 750 | CUUCUG | 798 | CUUCUGCUA | 846 | UGUGCGGAAAUGCU |
| 751 | UUCUGC | 799 | GUGUGCGGAA | 847 | GUGCGGAAAUGCUU |
| 752 | UCUGCU | 800 | UGUGCGGAAA | 848 | UGCGGAAAUGCUUC |
| 753 | CUGCUA | 801 | GUGCGGAAAU | 849 | GCGGAAAUGCUUCU |
| 754 | GUGUGCG | 802 | UGCGGAAAUG | 850 | CGGAAAUGCUUCUG |
| 755 | UGUGCGG | 803 | GCGGAAAUGC | 851 | GGAAAUGCUUCUGC |
| 756 | GUGCGGA | 804 | CGGAAAUGCU | 852 | GAAAUGCUUCUGCU |
| 757 | UGCGGAA | 805 | GGAAAUGCUU | 853 | AAAUGCUUCUGCUA |
| 758 | GCGGAAA | 806 | GAAAUGCUUC | 854 | GUGUGCGGAAAUGCU |
| 759 | CGGAAAU | 807 | AAAUGCUUCU | 855 | UGUGCGGAAAUGCUU |
| 760 | GGAAAUG | 808 | AAUGCUUCUG | 856 | GUGCGGAAAUGCUUC |
| 761 | GAAAUGC | 809 | AUGCUUCUGC | 857 | UGCGGAAAUGCUUCU |
| 762 | AAAUGCU | 810 | UGCUUCUGCU | 858 | GCGGAAAUGCUUCUG |
| 763 | AAUGCUU | 811 | GCUUCUGCUA | 859 | CGGAAAUGCUUCUGC |
| 764 | AUGCUUC | 812 | GUGUGCGGAAA | 860 | GGAAAUGCUUCUGCU |
| 765 | UGCUUCU | 813 | UGUGCGGAAAU | 861 | GAAAUGCUUCUGCUA |
| 766 | GCUUCUG | 814 | GUGCGGAAAUG | 862 | GUGUGCGGAAAUGCUU |
| 767 | CUUCUGC | 815 | UGCGGAAAUGC | 863 | UGUGCGGAAAUGCUUC |
| 768 | UUCUGCU | 816 | GCGGAAAUGCU | 864 | GUGCGGAAAUGCUUCU |
| 769 | UCUGCUA | 817 | CGGAAAUGCUU | 865 | UGCGGAAAUGCUUCUG |
| 770 | GUGUGCGG | 818 | GGAAAUGCUUC | 866 | GCGGAAAUGCUUCUGC |
| 771 | UGUGCGGA | 819 | GAAAUGCUUCU | 867 | CGGAAAUGCUUCUGCU |
| 772 | GUGCGGAA | 820 | AAAUGCUUCUG | 868 | GGAAAUGCUUCUGCUA |
| 773 | UGCGGAAA | 821 | AAUGCUUCUGC | 869 | GUGUGCGGAAAUGCUU |
| | | | | | C |
| 774 | GCGGAAAU | 822 | AUGCUUCUGCU | 870 | UGUGCGGAAAUGCUUC |
| | | | | | U |
| 775 | CGGAAAUG | 823 | UGCUUCUGCUA | 871 | GUGCGGAAAUGCUUCU |
| | | | | | G |
| 776 | GGAAAUGC | 824 | GUGUGCGGAAA | 872 | UGCGGAAAUGCUUCUG |
| | | | | | C |
| 777 | GAAAUGCU | 825 | UGUGCGGAAAU | 873 | GCGGAAAUGCUUCUGC |
| | | | G | | U |
| 778 | AAAUGCUU | 826 | GUGCGGAAAUG | 874 | CGGAAAUGCUUCUGCU |
| | | | C | | A |
| 779 | AAUGCUUC | 827 | UGCGGAAAUGC | 875 | GUGUGCGGAAAUGCUU |
| | | | U | | CU |
| 780 | AUGCUUCU | 828 | GCGGAAAUGCU | 876 | UGUGCGGAAAUGCUUC |
| | | | U | | UG |
| 781 | UGCUUCUG | 829 | CGGAAAUGCUU | 877 | GUGCGGAAAUGCUUCU |
| | | | C | | GC |
| 782 | GCUUCUGC | 830 | GGAAAUGCUUC | 878 | UGCGGAAAUGCUUCUG |
| | | | U | | CU |
| 783 | CUUCUGCU | 831 | GAAAUGCUUCU | 879 | GCGGAAAUGCUUCUGC |
| | | | G | | UA |
| 784 | UUCUGCUA | 832 | AAAUGCUUCUG | 880 | GUGUGCGGAAAUGCUU |
| | | | C | | CUG |

| | |
|---|---|
| 881 | UGUGCGGAAAUGCUUCUGC |
| 882 | GUGCGGAAAUGCUUCUGCU |
| 883 | UGCGGAAAUGCUUCUGCUA |
| 884 | GUGUGCGGAAAUGCUUCUGC |
| 885 | UGUGCGGAAAUGCUUCUGCU |
| 886 | GUGCGGAAAUGCUUCUGCUA |
| 887 | GUGUGCGGAAAUGCUUCUGCU |
| 888 | UGUGCGGAAAUGCUUCUGCUA |
| 889 | GUGUGCGGAAAUGCUUCUGCUA |

TABLE 3

| | | | | | | | |
|---|---|---|---|---|---|---|---|
| 3 | CCTAAA | 52 | TTTGTG | 101 | TCCGCAC | 150 | ATTTATA |
| 4 | CTAAAA | 53 | TTGTGC | 102 | CCGCACA | 151 | CCTAAAAA |
| 5 | TAAAAA | 54 | TGTGCA | 103 | CGCACAC | 152 | CTAAAAAT |
| 6 | AAAAAT | 55 | GTGCAG | 104 | GCACACT | 153 | TAAAAATG |
| 7 | AAAATG | 56 | TGCAGA | 105 | CACACTG | 154 | AAAAATGT |
| 8 | AAATGT | 57 | GCAGAA | 106 | ACACTGG | 155 | AAAATGTA |
| 9 | AATGTA | 58 | CAGAAA | 107 | CACTGGT | 156 | AAATGTAG |
| 10 | ATGTAG | 59 | AGAAAT | 108 | ACTGGTG | 157 | AATGTAGC |
| 11 | TGTAGC | 60 | GAAATG | 109 | CTGGTGT | 158 | ATGTAGCA |
| 12 | GTAGCA | 61 | AAATGT | 110 | TGGTGTC | 159 | TGTAGCAG |
| 13 | TAGCAG | 62 | AATGTT | 111 | GGTGTCC | 160 | GTAGCAGA |
| 14 | AGCAGA | 63 | ATGTTT | 112 | GTGTCCA | 161 | TAGCAGAA |
| 15 | GCAGAA | 64 | TGTTTC | 113 | TGTCCAG | 162 | AGCAGAAG |
| 16 | CAGAAG | 65 | GTTTCC | 114 | GTCCAGA | 163 | GCAGAAGC |
| 17 | AGAAGC | 66 | TTTCCA | 115 | TCCAGAA | 164 | CAGAAGCA |
| 18 | GAAGCA | 67 | TTCCAC | 116 | CCAGAAT | 165 | AGAAGCAT |
| 19 | AAGCAT | 68 | TCCACT | 117 | CAGAATC | 166 | GAAGCATT |
| 20 | AGCATT | 69 | CCACTA | 118 | AGAATCT | 167 | AAGCATTT |
| 21 | GCATTT | 70 | CACTAG | 119 | GAATCTA | 168 | AGCATTTC |
| 22 | CATTTC | 71 | ACTAGA | 120 | AATCTAG | 169 | GCATTTCC |
| 23 | ATTTCC | 72 | CTAGAT | 121 | ATCTAGT | 170 | CATTTCCG |
| 24 | TTTCCG | 73 | TAGATT | 122 | TCTAGTT | 171 | ATTTCCGC |
| 25 | TTCCGC | 74 | AGATTT | 123 | CTAGTTT | 172 | TTTCCGCA |
| 26 | TCCGCA | 75 | GATTTA | 124 | TAGTTTG | 173 | TTCCGCAC |
| 27 | CCGCAC | 76 | ATTTAT | 125 | AGTTTGT | 174 | TCCGCACA |
| 28 | CGCACA | 77 | TTTATA | 126 | GTTTGTG | 175 | CCGCACAC |
| 29 | GCACAC | 78 | CCTAAAA | 127 | TTTGTGC | 176 | CGCACACT |
| 30 | CACACT | 79 | CTAAAAA | 128 | TTGTGCA | 177 | GCACACTG |
| 31 | ACACTG | 80 | TAAAAAT | 129 | TGTGCAG | 178 | CACACTGG |
| 32 | CACTGG | 81 | AAAAATG | 130 | GTGCAGA | 179 | ACACTGGT |
| 33 | ACTGGT | 82 | AAAATGT | 131 | TGCAGAA | 180 | CACTGGTG |
| 34 | CTGGTG | 83 | AAATGTA | 132 | GCAGAAA | 181 | ACTGGTGT |
| 35 | TGGTGT | 84 | AATGTAG | 133 | CAGAAAT | 182 | CTGGTGTC |
| 36 | GGTGTC | 85 | ATGTAGC | 134 | AGAAATG | 183 | TGGTGTCC |
| 37 | GTGTCC | 86 | TGTAGCA | 135 | GAAATGT | 184 | GGTGTCCA |
| 38 | TGTCCA | 87 | GTAGCAG | 136 | AAATGTT | 185 | GTGTCCAG |
| 39 | GTCCAG | 88 | TAGCAGA | 137 | AATGTTT | 186 | TGTCCAGA |
| 40 | TCCAGA | 89 | AGCAGAA | 138 | ATGTTTC | 187 | GTCCAGAA |
| 41 | CCAGAA | 90 | GCAGAAG | 139 | TGTTTCC | 188 | TCCAGAAT |
| 42 | CAGAAT | 91 | CAGAAGC | 140 | GTTTCCA | 189 | CCAGAATC |
| 43 | AGAATC | 92 | AGAAGCA | 141 | TTTCCAC | 190 | CAGAATCT |
| 44 | GAATCT | 93 | GAAGCAT | 142 | TTCCACT | 191 | AGAATCTA |
| 45 | AATCTA | 94 | AAGCATT | 143 | TCCACTA | 192 | GAATCTAG |
| 46 | ATCTAG | 95 | AGCATTT | 144 | CCACTAG | 193 | AATCTAGT |
| 47 | TCTAGT | 96 | GCATTTC | 145 | CACTAGA | 194 | ATCTAGTT |
| 48 | CTAGTT | 97 | CATTTCC | 146 | ACTAGAT | 195 | TCTAGTTT |
| 49 | TAGTTT | 98 | ATTTCCG | 147 | CTAGATT | 196 | CTAGTTTG |
| 50 | AGTTTG | 99 | TTTCCGC | 148 | TAGATTT | 197 | TAGTTTGT |
| 51 | GTTTGT | 100 | TTCCGCA | 149 | AGATTTA | 198 | AGTTTGTG |
| 199 | GTTTGTGC | 248 | CCGCACACT | 297 | CCTAAAAATG | 346 | TTTGTGCAGA |
| 200 | TTTGTGCA | 249 | CGCACACTG | 298 | CTAAAAATGT | 347 | TTGTGCAGAA |
| 201 | TTGTGCAG | 250 | GCACACTGG | 299 | TAAAAATGTA | 348 | TGTGCAGAAA |
| 202 | TGTGCAGA | 251 | CACACTGGT | 300 | AAAAATGTAG | 349 | GTGCAGAAAT |
| 203 | GTGCAGAA | 252 | ACACTGGTG | 301 | AAAATGTAGC | 350 | TGCAGAAATG |
| 204 | TGCAGAAA | 253 | CACTGGTGT | 302 | AAATGTAGCA | 351 | GCAGAAATGT |
| 205 | GCAGAAAT | 254 | ACTGGTGTC | 303 | AATGTAGCAG | 352 | CAGAAATGTT |
| 206 | CAGAAATG | 255 | CTGGTGTCC | 304 | ATGTAGCAGA | 353 | AGAAATGTTT |
| 207 | AGAAATGT | 256 | TGGTGTCCA | 305 | TGTAGCAGAA | 354 | GAAATGTTTC |
| 208 | GAAATGTT | 257 | GGTGTCCAG | 306 | GTAGCAGAAG | 355 | AAATGTTTCC |
| 209 | AAATGTTT | 258 | GTGTCCAGA | 307 | TAGCAGAAGC | 356 | AATGTTTCCA |
| 210 | AATGTTTC | 259 | TGTCCAGAA | 308 | AGCAGAAGCA | 357 | ATGTTTCCAC |
| 211 | ATGTTTCC | 260 | GTCCAGAAT | 309 | GCAGAAGCAT | 358 | TGTTTCCACT |
| 212 | TGTTTCCA | 261 | TCCAGAATC | 310 | CAGAAGCATT | 359 | GTTTCCACTA |
| 213 | GTTTCCAC | 262 | CCAGAATCT | 311 | AGAAGCATTT | 360 | TTTCCACTAG |
| 214 | TTTCCACT | 263 | CAGAATCTA | 312 | GAAGCATTTC | 361 | TTCCACTAGA |
| 215 | TTCCACTA | 264 | AGAATCTAG | 313 | AAGCATTTCC | 362 | TCCACTAGAT |
| 216 | TCCACTAG | 265 | GAATCTAGT | 314 | AGCATTTCCG | 363 | CCACTAGATT |
| 217 | CCACTAGA | 266 | AATCTAGTT | 315 | GCATTTCCGC | 364 | CACTAGATTT |
| 218 | CACTAGAT | 267 | ATCTAGTTT | 316 | CATTTCCGCA | 365 | ACTAGATTTA |
| 219 | ACTAGATT | 268 | TCTAGTTTG | 317 | ATTTCCGCAC | 366 | CTAGATTTAT |
| 220 | CTAGATTT | 269 | CTAGTTTGT | 318 | TTTCCGCACA | 367 | TAGATTTATA |
| 221 | TAGATTTA | 270 | TAGTTTGTG | 319 | TTCCGCACAC | 368 | CCTAAAAATGT |
| 222 | AGATTTAT | 271 | AGTTTGTGC | 320 | TCCGCACACT | 369 | CTAAAAATGTA |
| 223 | GATTTATA | 272 | GTTTGTGCA | 321 | CCGCACACTG | 370 | TAAAAATGTAG |
| 224 | CCTAAAAAT | 273 | TTTGTGCAG | 322 | CGCACACTGG | 371 | AAAAATGTAGC |
| 225 | CTAAAAATG | 274 | TTGTGCAGA | 323 | GCACACTGGT | 372 | AAAATGTAGCA |
| 226 | TAAAAATGT | 275 | TGTGCAGAA | 324 | CACACTGGTG | 373 | AAATGTAGCAG |
| 227 | AAAAATGTA | 276 | GTGCAGAAA | 325 | ACACTGGTGT | 374 | AATGTAGCAGA |
| 228 | AAAATGTAG | 277 | TGCAGAAAT | 326 | CACTGGTGTC | 375 | ATGTAGCAGAA |
| 229 | AAATGTAGC | 278 | GCAGAAATG | 327 | ACTGGTGTCC | 376 | TGTAGCAGAAG |
| 230 | AATGTAGCA | 279 | CAGAAATGT | 328 | CTGGTGTCCA | 377 | GTAGCAGAAGC |
| 231 | ATGTAGCAG | 280 | AGAAATGTT | 329 | TGGTGTCCAG | 378 | TAGCAGAAGCA |
| 232 | TGTAGCAGA | 281 | GAAATGTTT | 330 | GGTGTCCAGA | 379 | AGCAGAAGCAT |
| 233 | GTAGCAGAA | 282 | AAATGTTTC | 331 | GTGTCCAGAA | 380 | GCAGAAGCATT |
| 234 | TAGCAGAAG | 283 | AATGTTTCC | 332 | TGTCCAGAAT | 381 | CAGAAGCATTT |
| 235 | AGCAGAAGC | 284 | ATGTTTCCA | 333 | GTCCAGAATC | 382 | AGAAGCATTTC |
| 236 | GCAGAAGCA | 285 | TGTTTCCAC | 334 | TCCAGAATCT | 383 | GAAGCATTTCC |
| 237 | CAGAAGCAT | 286 | GTTTCCACT | 335 | CCAGAATCTA | 384 | AAGCATTTCCG |
| 238 | AGAAGCATT | 287 | TTTCCACTA | 336 | CAGAATCTAG | 385 | AGCATTTCCGC |
| 239 | GAAGCATTT | 288 | TTCCACTAG | 337 | AGAATCTAGT | 386 | GCATTTCCGCA |
| 240 | AAGCATTTC | 289 | TCCACTAGA | 338 | GAATCTAGTT | 387 | CATTTCCGCAC |
| 241 | AGCATTTCC | 290 | CCACTAGAT | 339 | AATCTAGTTT | 388 | ATTTCCGCACA |
| 242 | GCATTTCCG | 291 | CACTAGATT | 340 | ATCTAGTTTG | 389 | TTTCCGCACAC |
| 243 | CATTTCCGC | 292 | ACTAGATTT | 341 | TCTAGTTTGT | 390 | TTCCGCACACT |
| 244 | ATTTCCGCA | 293 | CTAGATTTA | 342 | CTAGTTTGTG | 391 | TCCGCACACTG |
| 245 | TTTCCGCAC | 294 | TAGATTTAT | 343 | TAGTTTGTGC | 392 | CCGCACACTGG |
| 246 | TTCCGCACA | 295 | AGATTTATA | 344 | AGTTTGTGCA | 393 | CGCACACTGGT |
| 247 | TCCGCACAC | 296 | CCTAAAAATG | 345 | GTTTGTGCAG | 394 | GCACACTGGTG |

| | | | | | |
|---|---|---|---|---|---|
| 395 | CACACTGGTGT | 443 | AAATGTAGCAGA | 491 | TGCAGAAATGTT |
| 396 | ACACTGGTGTC | 444 | AATGTAGCAGAA | 492 | GCAGAAATGTTT |
| 397 | CACTGGTGTCC | 445 | ATGTAGCAGAAG | 493 | CAGAAATGTTTC |
| 398 | ACTGGTGTCCA | 446 | TGTAGCAGAAGC | 494 | AGAAATGTTTCC |
| 399 | CTGGTGTCCAG | 447 | GTAGCAGAAGCA | 495 | GAAATGTTTCCA |
| 400 | TGGTGTCCAGA | 448 | TAGCAGAAGCAT | 496 | AAATGTTTCCAC |
| 401 | GGTGTCCAGAA | 449 | AGCAGAAGCATT | 497 | AATGTTTCCACT |
| 402 | GTGTCCAGAAT | 450 | GCAGAAGCATTT | 498 | ATGTTTCCACTA |
| 403 | TGTCCAGAATC | 451 | CAGAAGCATTTC | 499 | TGTTTCCACTAG |
| 404 | GTCCAGAATCT | 452 | AGAAGCATTTCC | 500 | GTTTCCACTAGA |
| 405 | TCCAGAATCTA | 453 | GAAGCATTTCCG | 501 | TTTCCACTAGAT |
| 406 | CCAGAATCTAG | 454 | AAGCATTTCCGC | 502 | TTCCACTAGATT |
| 407 | CAGAATCTAGT | 455 | AGCATTTCCGCA | 503 | TCCACTAGATTT |
| 408 | AGAATCTAGTT | 456 | GCATTTCCGCAC | 504 | CCACTAGATTTA |
| 409 | GAATCTAGTTT | 457 | CATTTCCGCACA | 505 | CACTAGATTTAT |
| 410 | AATCTAGTTTG | 458 | ATTTCCGCACAC | 506 | ACTAGATTTATA |
| 411 | ATCTAGTTTGT | 459 | TTTCCGCACACT | 507 | CCTAAAAATGTAGCA |
| 412 | TCTAGTTTGTG | 460 | TTCCGCACACTG | 508 | CTAAAAATGTAGCAG |
| 413 | CTAGTTTGTGC | 461 | TCCGCACACTGG | 509 | TAAAAATGTAGCAGA |
| 414 | TAGTTTGTGCA | 462 | CCGCACACTGGT | 510 | AAAAATGTAGCAGAA |
| 415 | AGTTTGTGCAG | 463 | CGCACACTGGTG | 511 | AAAATGTAGCAGAAG |
| 416 | GTTTGTGCAGA | 464 | GCACACTGGTGT | 512 | AAATGTAGCAGAAGC |
| 417 | TTTGTGCAGAA | 465 | CACACTGGTGTC | 513 | AATGTAGCAGAAGCA |
| 418 | TTGTGCAGAAA | 466 | ACACTGGTGTCC | 514 | ATGTAGCAGAAGCAT |
| 419 | TGTGCAGAAAT | 467 | CACTGGTGTCCA | 515 | TGTAGCAGAAGCATT |
| 420 | GTGCAGAAATG | 468 | ACTGGTGTCCAG | 516 | GTAGCAGAAGCATTT |
| 421 | TGCAGAAATGT | 469 | CTGGTGTCCAGA | 517 | TAGCAGAAGCATTTC |
| 422 | GCAGAAATGTT | 470 | TGGTGTCCAGAA | 518 | AGCAGAAGCATTTCC |
| 423 | CAGAAATGTTT | 471 | GGTGTCCAGAAT | 519 | GCAGAAGCATTTCCG |
| 424 | AGAAATGTTTC | 472 | GTGTCCAGAATC | 520 | CAGAAGCATTTCCGC |
| 425 | GAAATGTTTCC | 473 | TGTCCAGAATCT | 521 | AGAAGCATTTCCGCA |
| 426 | AAATGTTTCCA | 474 | GTCCAGAATCTA | 522 | GAAGCATTTCCGCAC |
| 427 | AATGTTTCCAC | 475 | TCCAGAATCTAG | 523 | AAGCATTTCCGCACA |
| 428 | ATGTTTCCACT | 476 | CCAGAATCTAGT | 524 | AGCATTTCCGCACAC |
| 429 | TGTTTCCACTA | 477 | CAGAATCTAGTT | 525 | GCATTTCCGCACACT |
| 430 | GTTTCCACTAG | 478 | AGAATCTAGTTT | 526 | CATTTCCGCACACTG |
| 431 | TTTCCACTAGA | 479 | GAATCTAGTTTG | 527 | ATTTCCGCACACTGG |
| 432 | TTCCACTAGAT | 480 | AATCTAGTTTGT | 528 | TTTCCGCACACTGGT |
| 433 | TCCACTAGATT | 481 | ATCTAGTTTGTG | 529 | TTCCGCACACTGGTG |
| 434 | CCACTAGATTT | 482 | TCTAGTTTGTGC | 530 | TCCGCACACTGGTGT |
| 435 | CACTAGATTTA | 483 | CTAGTTTGTGCA | 531 | CCGCACACTGGTGTC |
| 436 | ACTAGATTTAT | 484 | TAGTTTGTGCAG | 532 | CGCACACTGGTGTCC |
| 437 | CTAGATTTATA | 485 | AGTTTGTGCAGA | 533 | GCACACTGGTGTCCA |
| 438 | CCTAAAAATGTA | 486 | GTTTGTGCAGAA | 534 | CACACTGGTGTCCAG |
| 439 | CTAAAAATGTAG | 487 | TTTGTGCAGAAA | 535 | ACACTGGTGTCCAGA |
| 440 | TAAAAATGTAGC | 488 | TTGTGCAGAAAT | 536 | CACTGGTGTCCAGAA |
| 441 | AAAAATGTAGCA | 489 | TGTGCAGAAATG | 537 | ACTGGTGTCCAGAAT |
| 442 | AAAATGTAGCAG | 490 | GTGCAGAAATGT | 538 | CTGGTGTCCAGAATC |

| | | | |
|---|---|---|---|
| 539 | TGGTGTCCAGAATCT | 587 | AGAAGCATTTCCGCACACTG |
| 540 | GGTGTCCAGAATCTA | 588 | GAAGCATTTCCGCACACTGG |
| 541 | GTGTCCAGAATCTAG | 589 | AAGCATTTCCGCACACTGGT |
| 542 | TGTCCAGAATCTAGT | 590 | AGCATTTCCGCACACTGGTG |
| 543 | GTCCAGAATCTAGTT | 591 | GCATTTCCGCACACTGGTGT |
| 544 | TCCAGAATCTAGTTT | 592 | CATTTCCGCACACTGGTGTC |
| 545 | CCAGAATCTAGTTTG | 593 | ATTTCCGCACACTGGTGTCC |
| 546 | CAGAATCTAGTTTGT | 594 | TTTCCGCACACTGGTGTCCA |
| 547 | AGAATCTAGTTTGTG | 595 | TTCCGCACACTGGTGTCCAG |
| 548 | GAATCTAGTTTGTGC | 596 | TCCGCACACTGGTGTCCAGA |
| 549 | AATCTAGTTTGTGCA | 597 | CCGCACACTGGTGTCCAGAA |
| 550 | ATCTAGTTTGTGCAG | 598 | CGCACACTGGTGTCCAGAAT |
| 551 | TCTAGTTTGTGCAGA | 599 | GCACACTGGTGTCCAGAATC |
| 552 | CTAGTTTGTGCAGAA | 600 | CACACTGGTGTCCAGAATCT |
| 553 | TAGTTTGTGCAGAAA | 601 | ACACTGGTGTCCAGAATCTA |
| 554 | AGTTTGTGCAGAAAT | 602 | CACTGGTGTCCAGAATCTAG |
| 555 | GTTTGTGCAGAAATG | 603 | ACTGGTGTCCAGAATCTAGT |
| 556 | TTTGTGCAGAAATGT | 604 | CTGGTGTCCAGAATCTAGTT |
| 557 | TTGTGCAGAAATGTT | 605 | TGGTGTCCAGAATCTAGTTT |
| 558 | TGTGCAGAAATGTTT | 606 | GGTGTCCAGAATCTAGTTTG |
| 559 | GTGCAGAAATGTTTC | 607 | GTGTCCAGAATCTAGTTTGT |
| 560 | TGCAGAAATGTTTCC | 608 | TGTCCAGAATCTAGTTTGTG |
| 561 | GCAGAAATGTTTCCA | 609 | GTCCAGAATCTAGTTTGTGC |
| 562 | CAGAAATGTTTCCAC | 610 | TCCAGAATCTAGTTTGTGCA |
| 563 | AGAAATGTTTCCACT | 611 | CCAGAATCTAGTTTGTGCAG |
| 564 | GAAATGTTTCCACTA | 612 | CAGAATCTAGTTTGTGCAGA |
| 565 | AAATGTTTCCACTAG | 613 | AGAATCTAGTTTGTGCAGAA |
| 566 | AATGTTTCCACTAGA | 614 | GAATCTAGTTTGTGCAGAAA |
| 567 | ATGTTTCCACTAGAT | 615 | AATCTAGTTTGTGCAGAAAT |
| 568 | TGTTTCCACTAGATT | 616 | ATCTAGTTTGTGCAGAAATG |
| 569 | GTTTCCACTAGATTT | 617 | TCTAGTTTGTGCAGAAATGT |
| 570 | TTTCCACTAGATTTA | 618 | CTAGTTTGTGCAGAAATGTT |
| 571 | TTCCACTAGATTTAT | 619 | TAGTTTGTGCAGAAATGTTT |
| 572 | TCCACTAGATTTATA | 620 | AGTTTGTGCAGAAATGTTTC |
| 573 | CCTAAAAATGTAGCAGAAGC | 621 | GTTTGTGCAGAAATGTTTCC |
| 574 | CTAAAAATGTAGCAGAAGCA | 622 | TTTGTGCAGAAATGTTTCCA |
| 575 | TAAAAATGTAGCAGAAGCAT | 623 | TTGTGCAGAAATGTTTCCAC |
| 576 | AAAAATGTAGCAGAAGCATT | 624 | TGTGCAGAAATGTTTCCACT |
| 577 | AAAATGTAGCAGAAGCATTT | 625 | GTGCAGAAATGTTTCCACTA |
| 578 | AAATGTAGCAGAAGCATTTC | 626 | TGCAGAAATGTTTCCACTAG |
| 579 | AATGTAGCAGAAGCATTTCC | 627 | GCAGAAATGTTTCCACTAGA |
| 580 | ATGTAGCAGAAGCATTTCCG | 628 | CAGAAATGTTTCCACTAGAT |
| 581 | TGTAGCAGAAGCATTTCCGC | 629 | AGAAATGTTTCCACTAGATT |
| 582 | GTAGCAGAAGCATTTCCGCA | 630 | GAAATGTTTCCACTAGATTT |
| 583 | TAGCAGAAGCATTTCCGCAC | 631 | AAATGTTTCCACTAGATTTA |
| 584 | AGCAGAAGCATTTCCGCACA | 632 | AATGTTTCCACTAGATTTAT |
| 585 | GCAGAAGCATTTCCGCACAC | 633 | ATGTTTCCACTAGATTTATA |
| 586 | CAGAAGCATTTCCGCACACT | 634 | CCTAAAAATGTAGCAGAAGCATTT |
| 635 | CTAAAAATGTAGCAGAAGCATTTC | 678 | TCTAGTTTGTGCAGAAATGTTTCC |
| 636 | TAAAAATGTAGCAGAAGCATTTCC | 679 | CTAGTTTGTGCAGAAATGTTTCCA |
| 637 | AAAAATGTAGCAGAAGCATTTCCG | 680 | TAGTTTGTGCAGAAATGTTTCCAC |
| 638 | AAAATGTAGCAGAAGCATTTCCGC | 681 | AGTTTGTGCAGAAATGTTTCCACT |
| 639 | AAATGTAGCAGAAGCATTTCCGCA | 682 | GTTTGTGCAGAAATGTTTCCACTA |
| 640 | AATGTAGCAGAAGCATTTCCGCAC | 683 | TTTGTGCAGAAATGTTTCCACTAG |
| 641 | ATGTAGCAGAAGCATTTCCGCACA | 684 | TTGTGCAGAAATGTTTCCACTAGA |
| 642 | TGTAGCAGAAGCATTTCCGCACAC | 685 | TGTGCAGAAATGTTTCCACTAGAT |
| 643 | GTAGCAGAAGCATTTCCGCACACT | 686 | CCTAAAAATGTAGCAGAAGCATTTCCGCAC |
| 644 | TAGCAGAAGCATTTCCGCACACTG | 687 | CTAAAAATGTAGCAGAAGCATTTCCGCACA |
| 645 | AGCAGAAGCATTTCCGCACACTGG | 688 | TAAAAATGTAGCAGAAGCATTTCCGCACAC |
| 646 | GCAGAAGCATTTCCGCACACTGGT | 689 | AAAAATGTAGCAGAAGCATTTCCGCACACT |
| 647 | CAGAAGCATTTCCGCACACTGGTG | 690 | AAAATGTAGCAGAAGCATTTCCGCACACTG |
| 648 | AGAAGCATTTCCGCACACTGGTGT | 691 | AAATGTAGCAGAAGCATTTCCGCACACTGG |
| 649 | GAAGCATTTCCGCACACTGGTGTC | 692 | AATGTAGCAGAAGCATTTCCGCACACTGGT |
| 650 | AAGCATTTCCGCACACTGGTGTCC | 693 | ATGTAGCAGAAGCATTTCCGCACACTGGTG |
| 651 | AGCATTTCCGCACACTGGTGTCCA | 694 | TGTAGCAGAAGCATTTCCGCACACTGGTGT |
| 652 | GCATTTCCGCACACTGGTGTCCAG | 695 | GTAGCAGAAGCATTTCCGCACACTGGTGTC |
| 653 | CATTTCCGCACACTGGTGTCCAGA | 696 | TAGCAGAAGCATTTCCGCACACTGGTGTCC |
| 654 | ATTTCCGCACACTGGTGTCCAGAA | 697 | AGCAGAAGCATTTCCGCACACTGGTGTCCA |
| 655 | TTTCCGCACACTGGTGTCCAGAAT | 698 | GCAGAAGCATTTCCGCACACTGGTGTCCAG |
| 656 | TTCCGCACACTGGTGTCCAGAATC | 699 | CAGAAGCATTTCCGCACACTGGTGTCCAGA |
| 657 | TCCGCACACTGGTGTCCAGAATCT | 700 | AGAAGCATTTCCGCACACTGGTGTCCAGAA |
| 658 | CCGCACACTGGTGTCCAGAATCTA | 701 | GAAGCATTTCCGCACACTGGTGTCCAGAAT |
| 659 | CGCACACTGGTGTCCAGAATCTAG | 702 | AAGCATTTCCGCACACTGGTGTCCAGAATC |
| 660 | GCACACTGGTGTCCAGAATCTAGT | 703 | AGCATTTCCGCACACTGGTGTCCAGAATCT |
| 661 | CACACTGGTGTCCAGAATCTAGTT | 704 | GCATTTCCGCACACTGGTGTCCAGAATCTA |
| 662 | ACACTGGTGTCCAGAATCTAGTTT | 705 | CATTTCCGCACACTGGTGTCCAGAATCTAG |
| 663 | CACTGGTGTCCAGAATCTAGTTTG | 706 | ATTTCCGCACACTGGTGTCCAGAATCTAGT |
| 664 | ACTGGTGTCCAGAATCTAGTTTGT | 707 | TTTCCGCACACTGGTGTCCAGAATCTAGTT |
| 665 | CTGGTGTCCAGAATCTAGTTTGTG | 708 | TTCCGCACACTGGTGTCCAGAATCTAGTTT |
| 666 | TGGTGTCCAGAATCTAGTTTGTGC | 709 | TCCGCACACTGGTGTCCAGAATCTAGTTTG |
| 667 | GGTGTCCAGAATCTAGTTTGTGCA | 710 | CCGCACACTGGTGTCCAGAATCTAGTTTGT |
| 668 | GTGTCCAGAATCTAGTTTGTGCAG | 711 | CGCACACTGGTGTCCAGAATCTAGTTTGTG |
| 669 | TGTCCAGAATCTAGTTTGTGCAGA | 712 | GCACACTGGTGTCCAGAATCTAGTTTGTGC |
| 670 | GTCCAGAATCTAGTTTGTGCAGAA | 713 | CACACTGGTGTCCAGAATCTAGTTTGTGCA |
| 671 | TCCAGAATCTAGTTTGTGCAGAAA | 714 | ACACTGGTGTCCAGAATCTAGTTTGTGCAG |
| 672 | CCAGAATCTAGTTTGTGCAGAAAT | 715 | CACTGGTGTCCAGAATCTAGTTTGTGCAGA |
| 673 | CAGAATCTAGTTTGTGCAGAAATG | 716 | ACTGGTGTCCAGAATCTAGTTTGTGCAGAA |
| 674 | AGAATCTAGTTTGTGCAGAAATGT | 717 | CTGGTGTCCAGAATCTAGTTTGTGCAGAAA |
| 675 | GAATCTAGTTTGTGCAGAAATGTT | 718 | TGGTGTCCAGAATCTAGTTTGTGCAGAAAT |
| 676 | AATCTAGTTTGTGCAGAAATGTTT | 719 | GGTGTCCAGAATCTAGTTTGTGCAGAAATG |
| 677 | ATCTAGTTTGTGCAGAAATGTTTC | 720 | GTGTCCAGAATCTAGTTTGTGCAGAAATGT |

| | |
|---|---|
| 721 | GTCCAGAATCTAGTTTGTGCAGAAATGTTT |
| 722 | TCCAGAATCTAGTTTGTGCAGAAATGTTTC |
| 723 | CCAGAATCTAGTTTGTGCAGAAATGTTTCC |
| 724 | CAGAATCTAGTTTGTGCAGAAATGTTTCCA |
| 725 | AGAATCTAGTTTGTGCAGAAATGTTTCCAC |
| 726 | GAATCTAGTTTGTGCAGAAATGTTTCCACT |
| 727 | AATCTAGTTTGTGCAGAAATGTTTCCACTA |
| 728 | ATCTAGTTTGTGCAGAAATGTTTCCACTAG |
| 729 | TCTAGTTTGTGCAGAAATGTTTCCACTAGA |
| 730 | CTAGTTTGTGCAGAAATGTTTCCACTAGAT |
| 731 | TAGTTTGTGCAGAAATGTTTCCACTAGATT |
| 732 | AGTTTGTGCAGAAATGTTTCCACTAGATTT |
| 733 | GTTTGTGCAGAAATGTTTCCACTAGATTTA |
| 734 | TTTGTGCAGAAATGTTTCCACTAGATTTAT |
| 735 | TTGTGCAGAAATGTTTCCACTAGATTTATA |

TABLE 4

| | | | | | |
|---|---|---|---|---|---|
| 737 | GTGTGC | 785 | GTGTGCGGA | 833 | AATGCTTCTGCT |
| 738 | TGTGCG | 786 | TGTGCGGAA | 834 | ATGCTTCTGCTA |
| 739 | GTGCGG | 787 | GTGCGGAAA | 835 | GTGTGCGGAAATG |
| 740 | TGCGGA | 788 | TGCGGAAAT | 836 | TGTGCGGAAATGC |
| 741 | GCGGAA | 789 | GCGGAAATG | 837 | GTGCGGAAATGCT |
| 742 | CGGAAA | 790 | CGGAAATGC | 838 | TGCGGAAATGCTT |
| 743 | GGAAAT | 791 | GGAAATGCT | 839 | GCGGAAATGCTTC |
| 744 | GAAATG | 792 | GAAATGCTT | 840 | CGGAAATGCTTCT |
| 745 | AAATGC | 793 | AAATGCTTC | 841 | GGAAATGCTTCTG |
| 746 | AATGCT | 794 | AATGCTTCT | 842 | GAAATGCTTCTGC |
| 747 | ATGCTT | 795 | ATGCTTCTG | 843 | AAATGCTTCTGCT |
| 748 | TGCTTC | 796 | TGCTTCTGC | 844 | AATGCTTCTGCTA |
| 749 | GCTTCT | 797 | GCTTCTGCT | 845 | GTGTGCGGAAATGC |
| 750 | CTTCTG | 798 | CTTCTGCTA | 846 | TGTGCGGAAATGCT |
| 751 | TTCTGC | 799 | GTGTGCGGAA | 847 | GTGCGGAAATGCTT |
| 752 | TCTGCT | 800 | TGTGCGGAAA | 848 | TGCGGAAATGCTTC |
| 753 | CTGCTA | 801 | GTGCGGAAAT | 849 | GCGGAAATGCTTCT |
| 754 | GTGTGCG | 802 | TGCGGAAATG | 850 | CGGAAATGCTTCTG |
| 755 | TGTGCGG | 803 | GCGGAAATGC | 851 | GGAAATGCTTCTGC |
| 756 | GTGCGGA | 804 | CGGAAATGCT | 852 | GAAATGCTTCTGCT |
| 757 | TGCGGAA | 805 | GGAAATGCTT | 853 | AAATGCTTCTGCTA |
| 758 | GCGGAAA | 806 | GAAATGCTTC | 854 | GTGTGCGGAAATGCT |
| 759 | CGGAAAT | 807 | AAATGCTTCT | 855 | TGTGCGGAAATGCTT |
| 760 | GGAAATG | 808 | AATGCTTCTG | 856 | GTGCGGAAATGCTTC |
| 761 | GAAATGC | 809 | ATGCTTCTGC | 857 | TGCGGAAATGCTTCT |
| 762 | AAATGCT | 810 | TGCTTCTGCT | 858 | GCGGAAATGCTTCTG |
| 763 | AATGCTT | 811 | GCTTCTGCTA | 859 | CGGAAATGCTTCTGC |
| 764 | ATGCTTC | 812 | GTGTGCGGAAA | 860 | GGAAATGCTTCTGCT |
| 765 | TGCTTCT | 813 | TGTGCGGAAAT | 861 | GAAATGCTTCTGCTA |
| 766 | GCTTCTG | 814 | GTGCGGAAATG | 862 | GTGTGCGGAAATGCTT |
| 767 | CTTCTGC | 815 | TGCGGAAATGC | 863 | TGTGCGGAAATGCTTC |
| 768 | TTCTGCT | 816 | GCGGAAATGCT | 864 | GTGCGGAAATGCTTCT |
| 769 | TCTGCTA | 817 | CGGAAATGCTT | 865 | TGCGGAAATGCTTCTG |
| 770 | GTGTGCGG | 818 | GGAAATGCTTC | 866 | GCGGAAATGCTTCTGC |
| 771 | TGTGCGGA | 819 | GAAATGCTTCT | 867 | CGGAAATGCTTCTGCT |
| 772 | GTGCGGAA | 820 | AAATGCTTCTG | 868 | GGAAATGCTTCTGCTA |
| 773 | TGCGGAAA | 821 | AATGCTTCTGC | 869 | GTGTGCGGAAATGCTT |
| | | | | | T |
| 774 | GCGGAAAT | 822 | ATGCTTCTGCT | 870 | TGTGCGGAAATGCTTC |
| | | | | | T |
| 775 | CGGAAATG | 823 | TGCTTCTGCTA | 871 | GTGCGGAAATGCTTCT |
| | | | | | G |
| 776 | GGAAATGC | 824 | GTGTGCGGAAA | 872 | TGCGGAAATGCTTCTG |
| | | | T | | C |
| 777 | GAAATGCT | 825 | TGTGCGGAAAT | 873 | GCGGAAATGCTTCTGC |
| | | | G | | T |
| 778 | AAATGCTT | 826 | GTGCGGAAATG | 874 | CGGAAATGCTTCTGCT |
| | | | C | | A |
| 779 | AATGCTTC | 827 | TGCGGAAATGC | 875 | GTGTGCGGAAATGCTT |
| | | | T | | CT |
| 780 | ATGCTTCT | 828 | GCGGAAATGCT | 876 | TGTGCGGAAATGCTTC |
| | | | T | | TG |
| 781 | TGCTTCTG | 829 | CGGAAATGCTT | 877 | GTGCGGAAATGCTTCT |
| | | | C | | GC |
| 782 | GCTTCTGC | 830 | GGAAATGCTTC | 878 | TGCGGAAATGCTTCTG |
| | | | T | | CT |
| 783 | CTTCTGCT | 831 | GAAATGCTTCT | 879 | GCGGAAATGCTTCTGC |
| | | | G | | TA |
| 784 | TTCTGCTA | 832 | AAATGCTTCTG | 880 | GTGTGCGGAAATGCTT |
| | | | C | | CTG |

| | |
|---|---|
| 881 | TGTGCGGAAATGCTTCTGC |
| 882 | GTGCGGAAATGCTTCTGCT |
| 883 | TGCGGAAATGCTTCTGCTA |
| 884 | GTGTGCGGAAATGCTTCTGC |
| 885 | TGTGCGGAAATGCTTCTGCT |
| 886 | GTGCGGAAATGCTTCTGCTA |
| 887 | GTGTGCGGAAATGCTTCTGCT |
| 888 | TGTGCGGAAATGCTTCTGCTA |
| 889 | GTGTGCGGAAATGCTTCTGCTA |

The following examples are meant to illustrate the invention. They are not meant to limit the invention in anyway.

### EXAMPLES

Drug-tolerance is an acute defense response prior to a fully drug-resistant state and tumor relapse. There are few therapeutic agents targeting drug-tolerance in the clinic. Here we show that miR-147b initiates a reversible tolerant-state to the EGFR inhibitor osimertinib in non-small cell lung cancer. MiR-147b was the most upregulated non-coding RNA in osimertinib-tolerant and EGFR mutated lung cancer cells by miRNA-seq analysis. Whole transcriptome analysis of single-cell derived clones revealed a link between osimertinib-tolerance and pseudohypoxia responses irrespective of oxygen levels. Further metabolomics and genetic studies demonstrated that osimertinib-tolerance is driven by miR-147b repression of VHL and succinate dehydrogenase linked to the tricarboxylic acid cycle and pseudohypoxia pathways. Locked nucleic acid miR-147b inhibitor pretreatment delayed osimertinib-associated drug tolerance in patient-derived organoids. The link between miR-147b and tricarboxylic acid cycle may provide promising targets for preventing tumor relapse.

### MiRNA Expression in Lung Cancer Cell Lines

We analyzed a publicly available RNA sequencing dataset in an unbiased way for 122 human lung cancer cell lines. Eight of the cell lines contained EGFR mutations (sensitive and resistant to TKI); 72 of the cell lines were wild type EGFR (EGFR^{wt}). In a cohort 1, which included frequently studied EGFRmut and EGFR^{wt} lung cancer cell lines (n=15), we found the top six-upregulated miRNAs in a comparison of EGFRmut versus EGFR^{wt} include miR-147b, miR-936, miR-614, miR-222, miR-433, and miR-127 (p<0.05). Several miRNAs in the set up upregulated miRNAs were reported previously to be associated with the EGFR signaling pathway, including miR-222 and miR-127. We focused our study on miR-147b because miR-147b is the most upregulated miRNA in EGFRmut lung cancer cells from our analysis and because the function of miR-147b is not well known.

In addition to acquiring additional EGFR mutations such as T790M or C797S, lung cancer cells also activate alternative RTKs via bypass mechanisms to promote cancer cell survival and proliferation. To understand whether miR-147b is associated with mutations in other RTKs, we analyzed miR-147b expression in cancer cells of cohort 2 with mutations in other RTKs, including BRAF, ALK, ROS1, and ERBB2/3/4. As expected, cancer cells with those RTKs mutations also expressed higher levels of miR-147b compared with EGFR^{wt} cancer cells. Then we asked whether miR-147b expression is linked to tolerance and resistance to EGFR inhibition. To address this question, we derived a number of gefitinib-resistant lung cancer cell lines upon continuous gefitinib treatment in parental sensitive cancer cells in vitro. Consistent with RNA seq analysis, our qPCR results showed that miR-147b was expressed ˜20 fold higher in EGFRmut lung cancer cell lines (n=7) compared with EGFR^{wt} cell lines (n=5). Moreover, the expression levels of miR-147b in gefitinib-resistant cancer cells (PC9ER, H1975, and HCC827GR, n=3) were up to three-fold higher than gefitinib-sensitive cancer cells (H1650, PC9, H3255, and HCC827). These results indicate that upregulation of miR-147b correlates with an activated EGFR signaling pathway and increased resistance to EGFR TKIs.

In addition, we tested whether miR-147b expression could distinguish tumor cells from normal cells. To address this question, we included one immortalize lung epithelial cell line AALE in our study. Our results demonstrated that expression levels of miR-147b were up to 700-fold up-regulated in a comparison of EGFRmut cancer cells versus normal cells. Analysis of a separate qRT-PCR dataset showed that the expression level of miR-147b was 2.4-fold higher in EGFRmut lung cancer tissues than normal lung tissues. Thus, we have found a new miRNA, miR-147b, which is linked to tumorigenesis and increased resistance to current EGFR-based targeted therapy.

### Lung Cancer Cells Adopt a Tolerance Strategy to EGFR Inhibitors

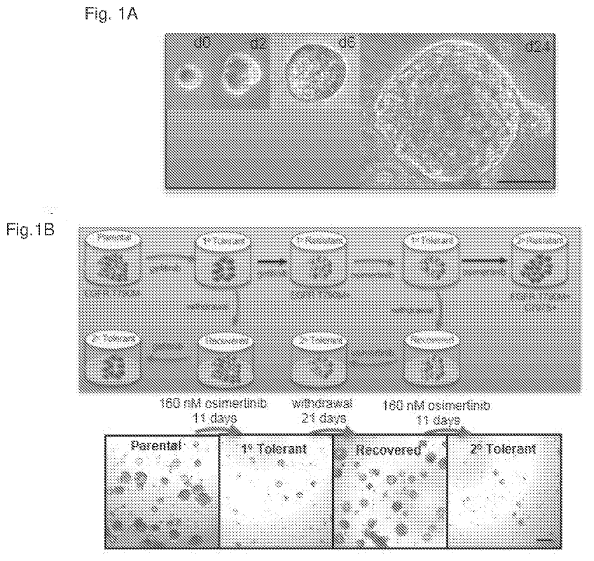

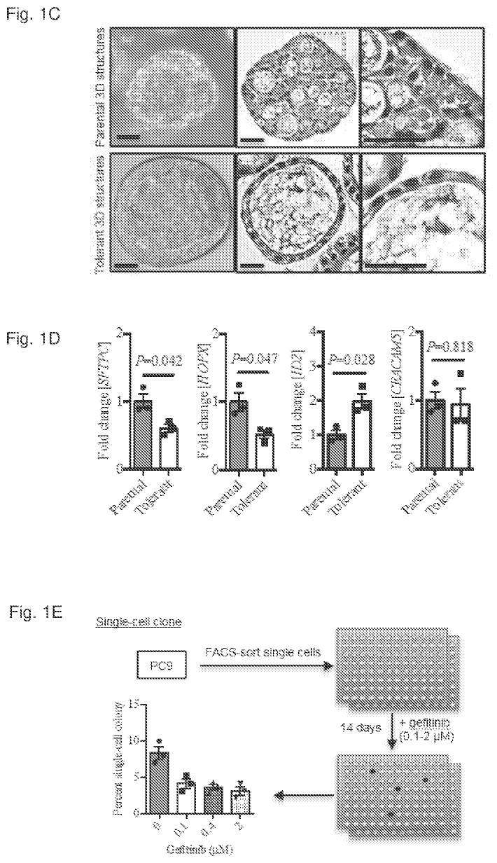

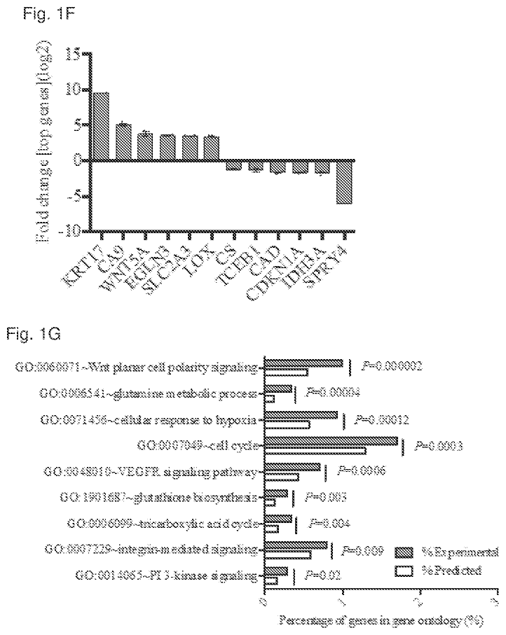

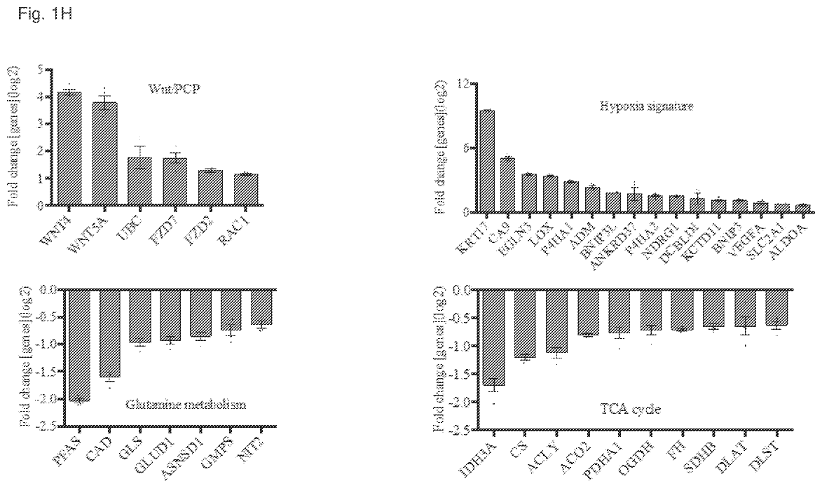

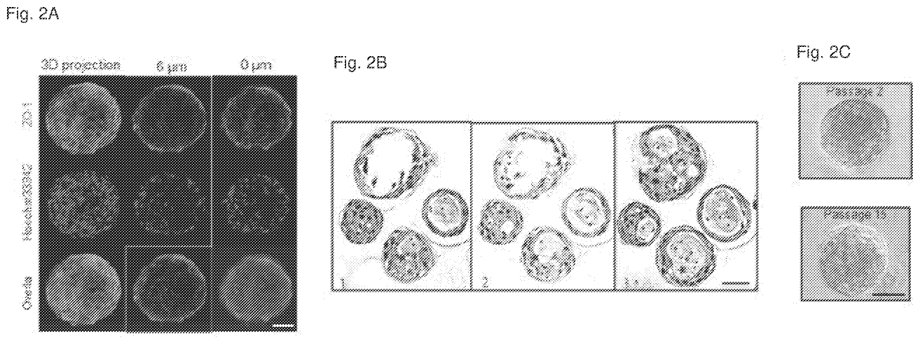

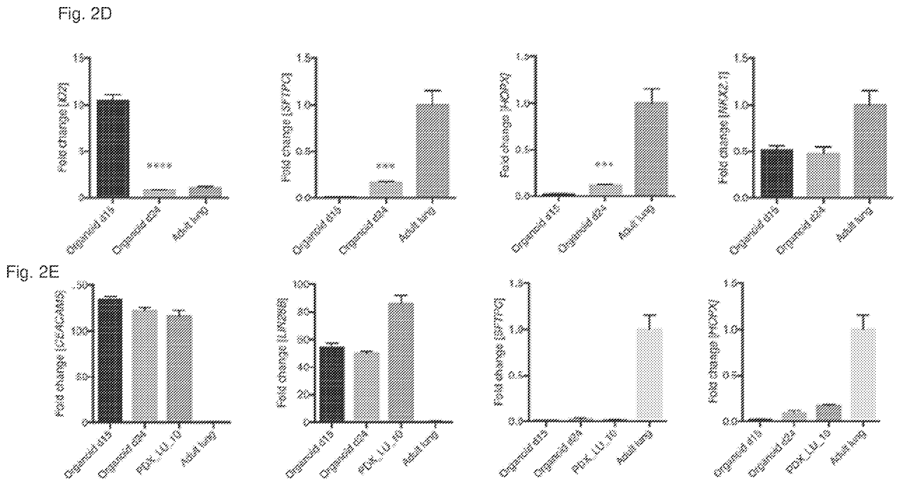

Due to an advantage for visualizing in vivo-like structures in organoids, we established 3D lung organoids in immortalized tracheobronchial epithelial AALE cells and EGFR mutated lung cancer HCC827 cells (FIG. 1a-c and FIG. 2a-c). Compared with adult lung tissues, AALE-derived lung organoids express higher levels of lung progenitor cell gene inhibitor of DNA binding 2 (ID2) on day 15 followed by decreased expression on day 24 by qRT-PCR analysis (FIG. 2d). In contrast, the organoids from AALE express lower levels of type I and II pneumocyte markers including surfactant protein C (SFTPC), HOP homeobox (HOPX), and NK2 homeobox 1 (NKX2.1) (transcription termination factor 1, TTF-1) on day 15 followed by increasing expressions on day 24 (FIG. 2d). The gene expression levels of ID2, SFTPC, HOPX, and NKX2.1 in lung organoids are comparable to those in adult lung tissues, which is consistent with a previous finding of lung organoids differentiated from pluripotent stem cells (Dye et al., Elife 4:e05098, 2015). Similarly, organoids from lung adenocarcinoma patient-derived xenograft tumor (PDX_LU_10) on day 25 express tumor and lung-relevant genes including carcinoembryonic antigen related cell adhesion molecule 5 (CEACAM5), Lin-28 homolog B (LIN28B), SFTPC, and HOPX, which are comparable to those in the parental tumor (FIG. 2e). Collectively, our data suggest that lung organoids cultured over time are relevant to clinical tissues.

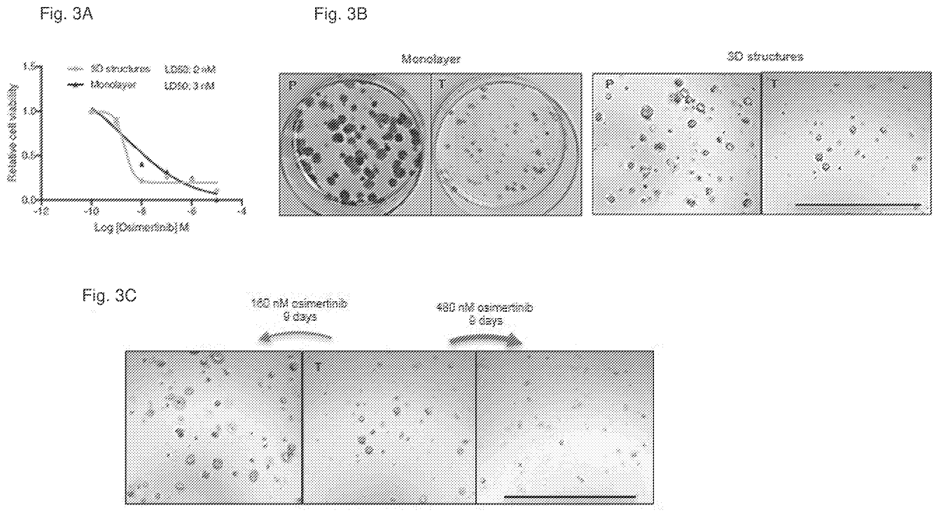

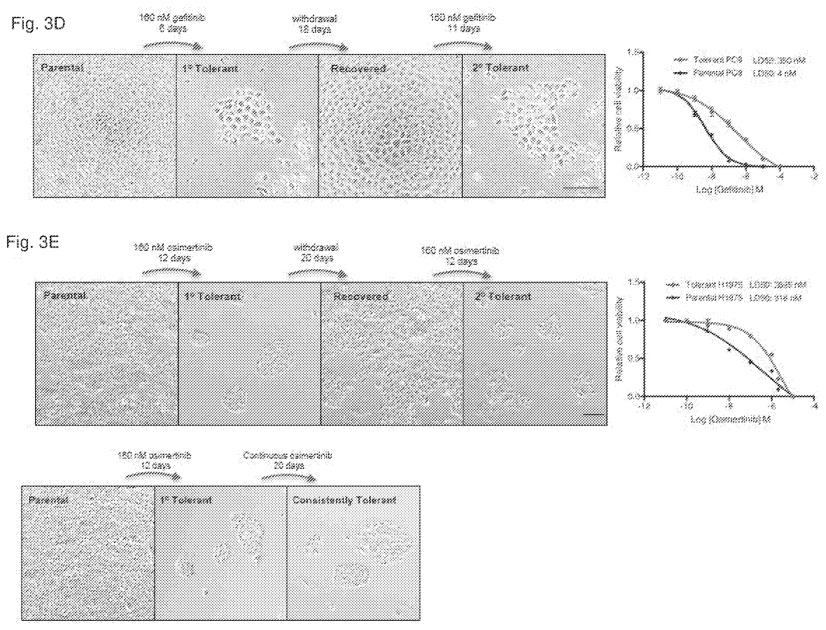

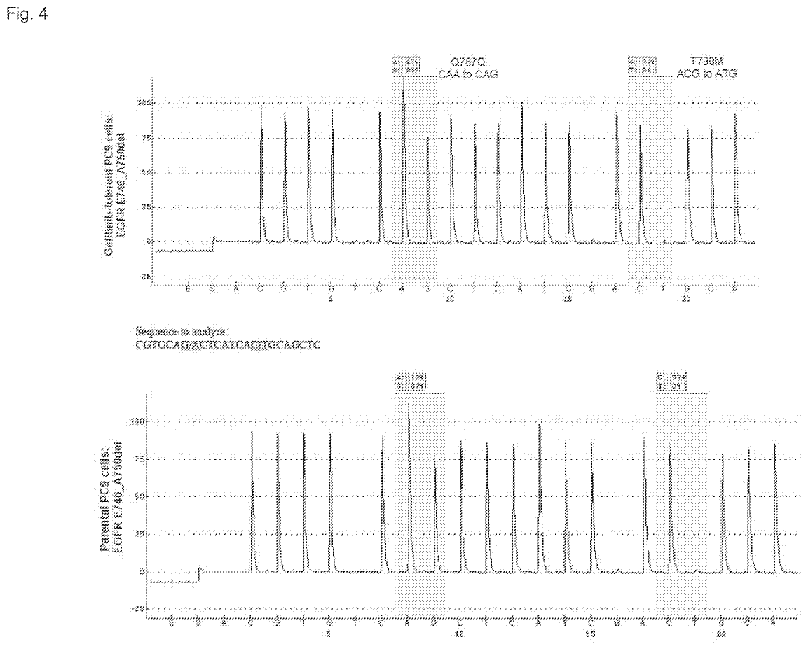

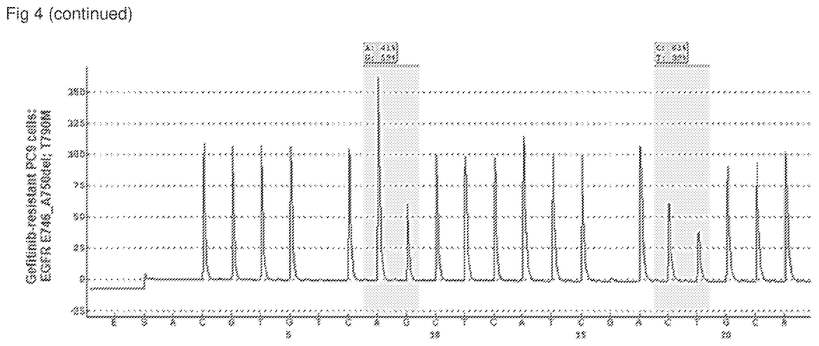

Using both organoid and monolayer cultures, we treated HCC827 cells with serially diluted osimertinib for three days to observe their acute treatment responses. We found that a subpopulation of tumor cells survived cytotoxic doses (0.01-2 μM) of osimertinib treatment initially (FIG. 3a). Surprisingly, a small percentage of cells could survive longer than 2-3 weeks in both monolayer and organoid models when they were treated with 160 nM of osimertinib continuously (FIG. 3b). However, different from drug-resistant cells, most of those surviving organoids disappeared when they were treated with three-fold higher concentration of osimertinib for additional 9 days (FIG. 3c). This indicates that some tumor cells adopt a new strategy different from that applied for drug-resistance to protect themselves during the early-stage of treatment response to anti-EGFR therapy. To further understand the protective strategy applied by some tumor cells, after 11-days treatment, we withdrew osimertinib on HCC827 organoids and found that the initially surviving organoids recovered with increasing size within the following 21 days. Those recovered organoids remained similarly sensitive to osimertinib when they were exposed to the same dose of osimertinib again (FIG. 1b). Another two lung cancer cell lines with EGFR mutations, PC9 and H1975 cells, entered a similar “tolerance cycle” when gefitinib/osimertinib treatments alternated with treatment withdrawal (FIG. 3d-e). This suggests that a subpopulation of tumor cells enter a reversible tolerant state to defend against EGFR-TKIs at the early stages of anti-EGFR treatment. To understand whether the above tolerance is conferred by acquisition of EGFR T790M mutation, we performed pyrosequencing for quantitative analysis of EGFR exon 19 and 20 sequence variations (FIG. 4). We found that the drug-tolerant cells demonstrated comparable EGFR exon 19 and 20 sequence to the parental cells in PC9 rather than EGFRT790M-positive gefitinib-resistant PC9 cells. This indicates that the tolerant strategy adopted by tumor cells against EGFR-TKI might be mediated by mechanisms different from EGFRT790M mutation. Furthermore, single HCC827 cells mixed with geltrex were plated in 96-well plate and divided into two groups. After 24-hours, one half of the cells was treated with 100 nM osimertinib for 21 days (tolerant organoids) and the other half of cells were treated with DMSO as control (parental organoids) (FIG. 1c). Then we looked at the microscopic structures of organoids from parental cells and osimertinib-tolerant cells in HCC827 by H&E staining in histology. The parental cell-derived organoids showed an adenocarcinoma-like structure. Unexpectedly, a “ring-like” structure was found in the osimertinib-tolerant organoids (FIG. 1c). To understand the gene expression in those structures, we performed qRT-PCR analysis on the parental organoids and tolerant organoids derived from single HCC827 cells. Both parental and osimertinib-tolerant organoids express comparable levels of CEACAM5 (FIG. 1d). However, osimertinib-tolerant organoids expressed two-fold lower levels of SFTPC and HOPX but up to two-fold higher levels of ID2 (FIG. 1d), suggesting that osimertinib-tolerant organoids are enriched for stemness relevant genes.

To better understand the transcriptomic changes and tumor heterogeneity conferring osimertinib or gefitinib tolerance in lung cancer, we developed single cell-derived clones in PC9 (FIG. 1e). A single cell was sorted into a 96-well plate at one cell per well by fluorescence-activated cell sorting (FACS). On the following day, the cells were treated with 0.1, 0.4, and 2 μM gefitinib or the vehicle for 14 days (n=192 wells per group). The frequency of colony formation was 8.3%±0.7% and 3.6%±0.3% in the vehicle-treated and all three gefitinib-treated groups, respectively (FIG. 1e). One parental single cell-derived clone treated with vehicle that was sensitive to gefitinib and two drug-tolerant single cell-derived clones treated with 0.4 μM gefitinib were randomly selected and applied for the following whole transcriptome analysis by microarray. We found the top changed genes included upregulated expression of KRT17 (keratin 17), CA9 (carbonic anhydrase 9), WNT5A (Wnt family member 5A), EGLN3 (Egl-9 family hypoxia inducible factor 3), SLC2A3 (solute carrier family 2 member 3), and LOX (lysyl oxidase), as well as downregulated expression of SPRY4 (sprouty RTK signaling antagonist 4) and IDH3A (isocitrate dehydrogenase 3 (NAD(+)) alpha) (FIG. 1f). Gene ontology analysis demonstrated the top differentially-expressed signaling pathways in the gefitinib-tolerant single-cell clones, including Wnt planar cell polarity (Wnt/PCP) signaling, glutamine metabolic process, cellular response to hypoxia, cell cycle, VEGFR signaling pathway, glutathione derivative biosynthesis, tricarboxylic acid (TCA) cycle, integrin-mediated signaling and PI 3-kinase signaling (FIG. 1g). The gene signatures for activated Wnt/PCP signaling and the hypoxia response as well as inactivated glutamine metabolic process and the TCA cycle were validated by qRT-PCR (FIG. 1h). An activated Wnt/PCP signaling pathway has been linked to drug resistance in many studies (Zhan et al., Oncogene 36(11):1461-1473, 2017). It was unexpected that activated hypoxia responses, as well as inactivated metabolic processes, such as glutamine process and the TCA cycle, are among the top signaling pathways relevant to drug-tolerance. Our data suggests that these pathways might cooperatively maintain a “tolerance signature” in EGFR mutant lung cancer cells when they were exposed to EGFR-TKIs.

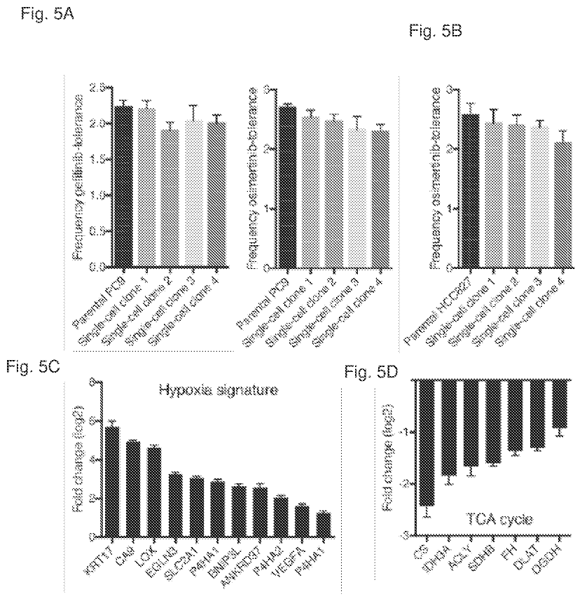

To exclude the possibility that pre-existing cellular heterogeneity could be responsible for this tolerance, we made single cell clones first followed by exposure to 2 μM gefitinib. In parallel, as in the previous experiment, PC9 cells were cloned in the same concentration of gefitinib (parental clones) as control. All tested single cell-derived clones generate gefitinib-tolerant clones at a frequency of 1.9˜2.1% (n=4 clones), which is comparable to that in parental PC9 clones (2.2 t 0.1%) (FIG. 5a). A similar frequency of osimertinib-tolerance was found between single-cell clones and parental clones in PC9 cells (FIG. 5a). Consistently, both single-cell clones from HCC827 and parental clones demonstrated a comparable frequency of osimertinib-tolerance (FIG. 5b). All of our data strongly suggest that drug-tolerance is spontaneously acquired rather than a reflection of pre-existing cellular heterogeneity, which is consistent with previous findings (Sharma et al., Cell 141(1):69-80, 2010; Smith et al., Cancer Cell 29(3):270-284, 2016). In addition, compared with PC9 cells tolerant to gefitinib (FIG. 1h), the cells tolerant to osimertinib express similar genes in hypoxia pathway and the TCA cycle (FIG. 5c-d). This suggests that lung cancer cells utilize similar strategies to protect themselves from drug-induced cytotoxicity when the cells are treated with either gefitinib or osimertinib. Collectively, our data has demonstrated that drug-tolerance is acquired spontaneously by a small population of lung cancer cells.

### MicroRNA-147b Initiates Anticancer Drug Tolerance

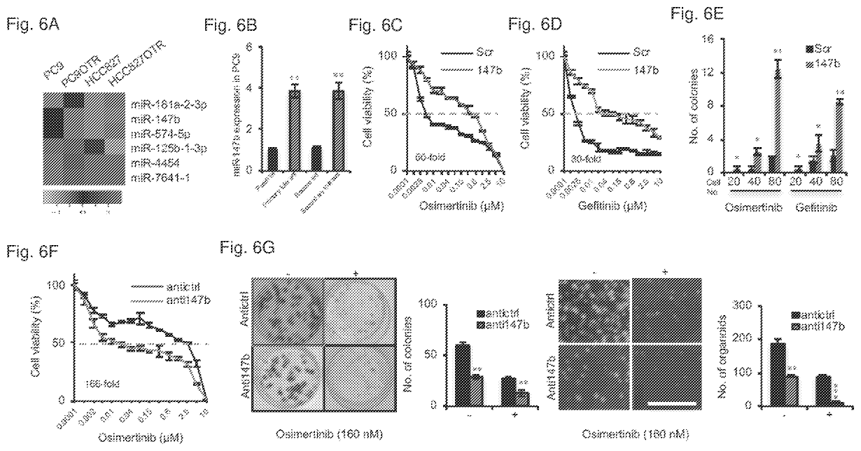

To test which microRNAs (miRNAs) are linked to osimertinib-tolerance, we performed miRNA-seq analysis in two paired osimertinib-tolerant cells and osimertinib-sensitive parental cells from HCC827 and PC9. A list of differentially expressed miRNAs (n=45) relevant to osimertinib-tolerance was derived from this analysis. The top upregulated miRNAs included miR-181a-2-3p, miR-147b, miR-574-5p and the top downregulated miRNAs included miR-7641-1, miR-4454, and miR-125b-1-3p (FIG. 6a). It has been reported that overexpression of miR-181a and miR-574 confers chemoresistance in lung and other cancers (Li et al., Int. J. Oncol. 47(4):1379-1392, 2015; Sun et al., Eur. Rev. Med. Pharmacol. Sci. 22(5):1342-1350, 2018; Galluzzi et al., Cancer Res. 70(5):1793-1803, 2010). However, miR-147b is an miRNA that has not been well studied in drug tolerance. Thus, we focused on miR-147b in our following drug-tolerance study.

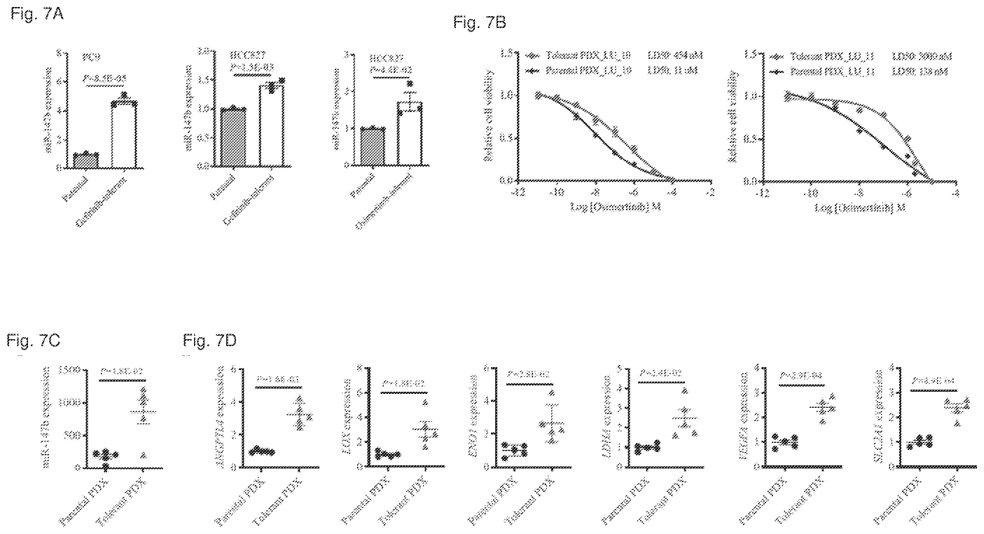

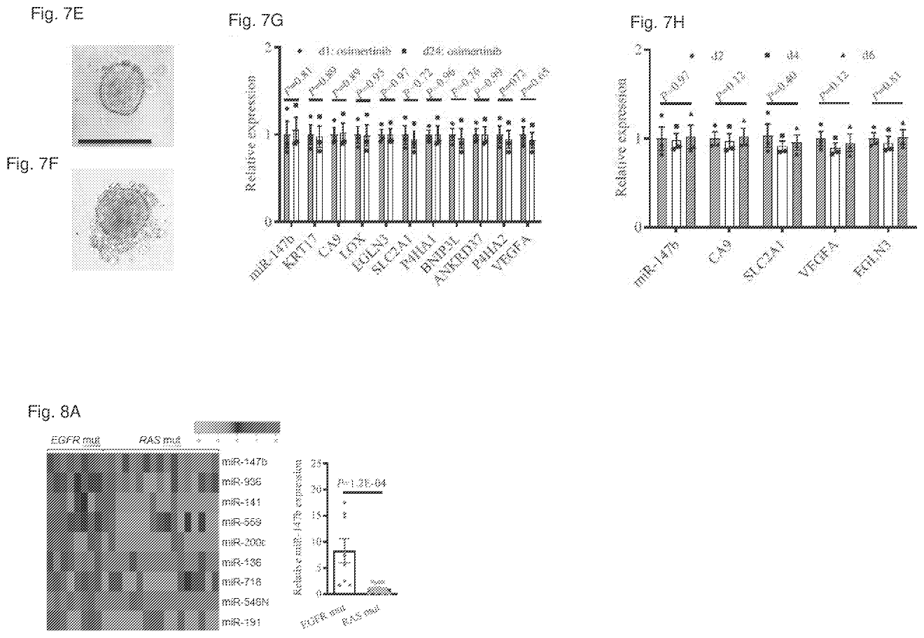

As expected, our qRT-PCR analysis validated the up to five-fold upregulation of miR-147b expression in gefitinib- and osimertinib-tolerant cells compared with parental cells in both PC9 and HCC827 (FIG. 6b and FIG. 7a). Furthermore, the expression levels of miR-147b decreased in the recovered primary drug-tolerant cells in PC9 cells upon osimertinib withdrawal for 18 days. MiR-147b expression levels rose in the recovered cells when osimertinib was administered again after 11 days (FIG. 6b). Next, we established parental organoids from PDX lung tumors harboring EGFR mutations and created osimertinib-tolerant organoids (FIG. 7b) by continuous treatment with 100 nM osimertinib for 21 days. Consistently, miR-147b expression levels in osimertinib-tolerant PDX organoids showed up to five-fold increase compared with parental PDX organoids (n=5) (FIG. 7c). In addition, hypoxia genes including ANGPTL4 (angiopoietin like 4), LOX, ENO1, LDHA (lactate dehydrogenase A), VEGFA (vascular endothelial growth factor A), and SLC2A1 (solute carrier family 2 member 1) were also upregulated in osimertinib-tolerant PDX organoids (FIG. 7d). To understand effects of organoid culture stages on outcome of drug-tolerance, we made established organoids (grown for 24 days) first followed by osimertinib treatment for additional 21 days. Our data showed that drug-tolerant cells derived from organoids on day 24 form comparable structures and express similar levels of miR-147b and pseudohypoxia genes compared to those derived from organoids on day 1 (FIG. 7e-g). Thus, our data indicate that the organoid culture stage does not affect the outcome of drug-tolerance. Then we asked whether heterogeneity existed in the initial organoids with respect to expressions for miR-147b and pseudohypoxia genes. To answer the question, using initial organoids established from single cell-derived HCC827 organoids on days 2, 4, and 6, we performed qRT-PCR analysis on miR-147b and pseudohypoxia gene expression. Our data demonstrated that there is no significant difference regarding to miR-147b and pseudohypoxia genes expressions in the initial organoids (FIG. 7h). Collectively, our data suggests that miR-147b expression levels are relevant to the reversible drug-tolerance.

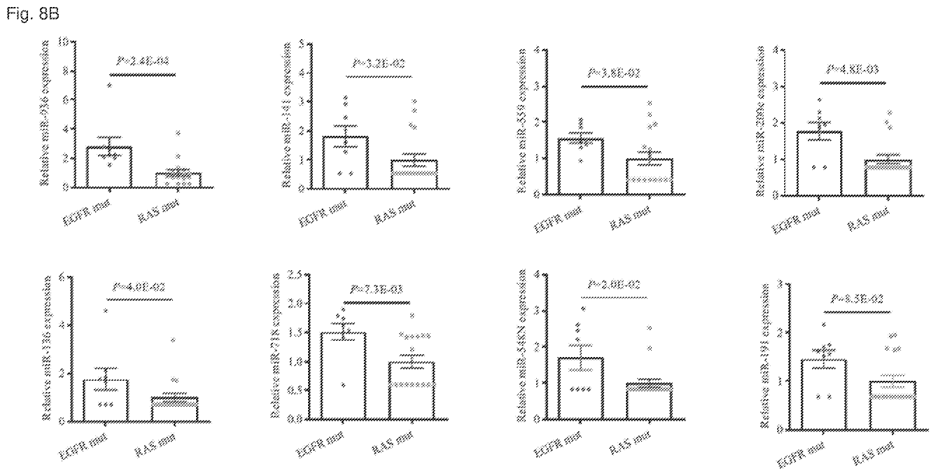

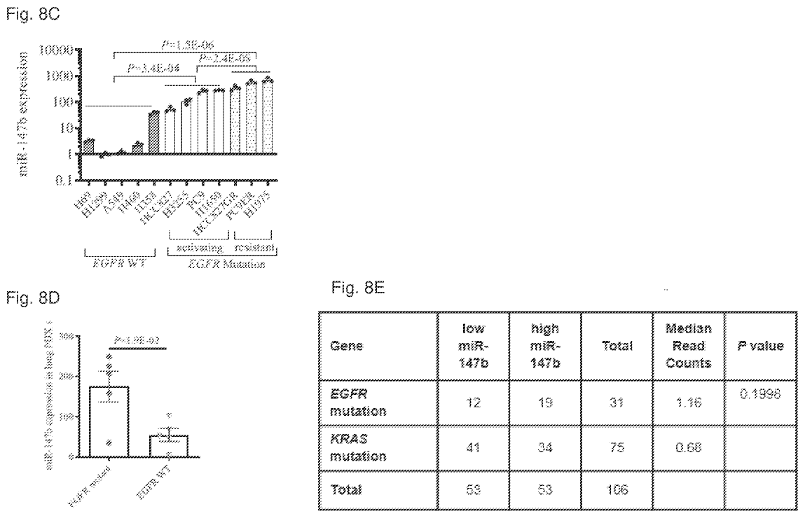

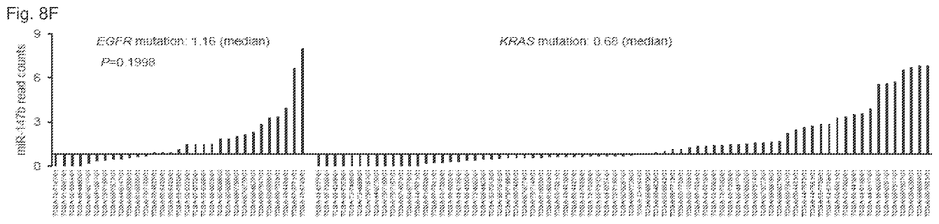

EGFR and KRAS mutations are widely known as mutually exclusive in lung cancer patients, and mutations in KRAS are associated with a lack of sensitivity to gefitinib (Pao et al., PloS Med. 2(1):e17, 2005). EGFR-TKI tolerant cells still respond to EGFR inhibitors at higher concentrations (FIG. 3c), because they harbor the same EGFR activating mutation as their parental cells (FIG. 4), thus we hypothesized that miR-147b expression might be distinguishable in patients with mutated EGFR rather than mutated RAS. To validate this hypothesis, we performed whole transcriptome RNA-seq analysis on a cohort of lung adenocarcinoma cell lines for miRNA profiles relevant to EGFR mutations using a public dataset (Klijn et al., Nat. Biotechnol. 33(3):306-312, 2015). We found that the top upregulated miRNAs include miR-147b, miR-936, miR-141, miR-559, and miR-200c in EGFR mutant cell lines (n=8) compared with RAS mutant cell lines (n=17) (FIG. 8a-b). Consistently, qRT-PCR analysis demonstrated that miR-147b expression levels in lung cancer cell lines with TKI sensitizing or resistant EGFR mutations (n=7) were higher than those in EGFR wild-type lung cancer cell lines (n=5) (FIG. 8c). Interestingly, the miR-147b expression levels in cancer cells (HCC827GR, PC9ER, and H1975) with EGFR^{T790M} were even higher than those (HCC827, H3255, PC9, and H1650) with EGFR sensitizing mutations (FIG. 8c). Next, analysis of lung adenocarcinoma patient-derived xenografts (PDXs) showed that miR-147b expression levels in EGFR mutant PDX tumors (176 t 38) were up to four-fold higher than those in the EGFR wild-type lung cancers (54 t 16) (P<0.05) (FIG. 8d). This is consistent with our data for human lung cancer cell lines (FIG. 8a). Further analysis of lung adenocarcinoma tissues in The Cancer Genome Atlas (TCGA) dataset (Cancer Genome Atlas Research Network, Nature 511(7511):543-550, 2014; Anaya et al., Peer J. Preprints. 4:e2574v1, 2016) showed that the median read counts of miR-147b in EGFR mutant tumors (median=1.16, n=31) are 1.7-fold higher than those in KRAS mutant tumors (median=0.68, n=75) (P=0.2) (FIG. 8e-t). The above data suggests that miR-147b might be a potent marker in EGFR mutant lung cancers.

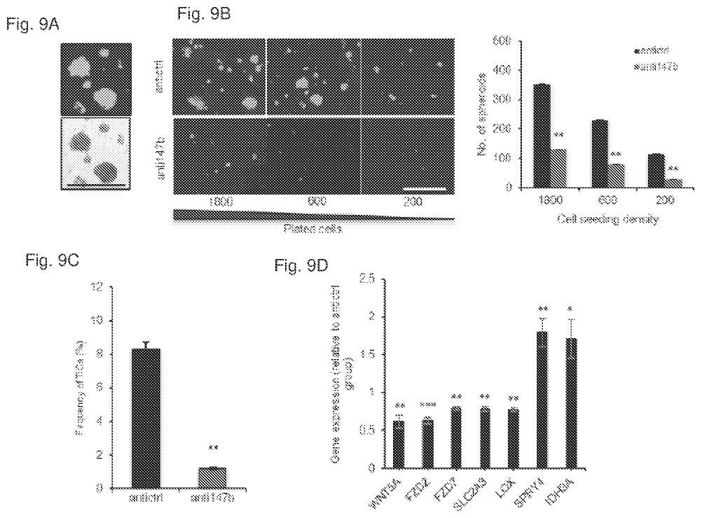

Furthermore, to study the functional roles of miR-147b in regulating drug-tolerance, we overexpressed lentiviral miR-147b in HCC827 cells. We found that the enforced overexpression of miR-147b enhanced drug-tolerance by 60-fold and 30-fold at the half-maximum inhibitory concentration (IC50) of osimertinib and gefitinib, respectively (FIG. 6c-d). As expected, miR-147b overexpression in HCC827 cells rescued decreased colony-formation induced by treatments with osimertinib or gefitinib (FIG. 8e). Conversely, knocking down miR-147b by lentiviral infection on H1975 cells increased their sensitivity towards osimertinib by 166-fold at the IC50 (FIG. 6f). As expected, miR-147b knockdown almost abolished all the drug-tolerant colonies and organoids in the presence of osimertinib within 12-21 days (FIG. 6g). This suggests that miR-147b is critical for regulating drug-tolerance. Furthermore, a spheroid-formation assay and limiting-dilution analysis showed that knocking down miR-147b decreased the frequency of tumor-initiating cell (TIC) by seven-fold from 1/11.8 (8.5%) to 1/83.1 (1.2%) (FIG. 9a-c). Consistently, miR-147b knockdown decreased expression levels of stemness-related genes in Wnt/PCP signaling pathway by qRT-PCR analysis, including WNT5A, FZD2, and FZD7 (Asad et al., Cell Death Dis. 5:e1346, 2014)(FIG. 9d). In addition, miR-147b knockdown also downregulated expression levels for SLC2A3 and LOX, as well as upregulated expression levels for SPRY4 and IDH3A (FIG. 9d). The dysregulated gene profile is consistent to those dysregulated in drug-tolerant cells (FIG. 1f). Furthermore, using a CRISPR (clustered regularly interspaced short palindromic repeats)-Cas9 approach, we knocked out miR-147b in H1975 cells (FIG. 10a) and demonstrated that miR-147b knockout could consistently reduce cell viability in organoids and decrease osimertinib-tolerance in H1975 cells (FIG. 10b-d). Thus, EGFR-TKIs tolerance is conferred by miR-147b and cancer-stemness.

miR-147b-VHL Axis Confers Drug-Tolerance

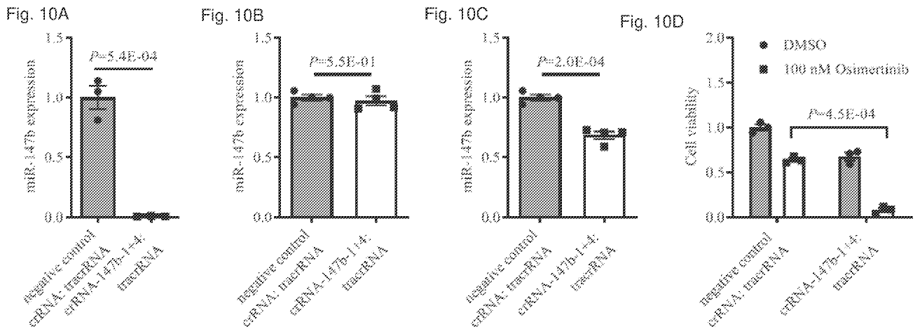

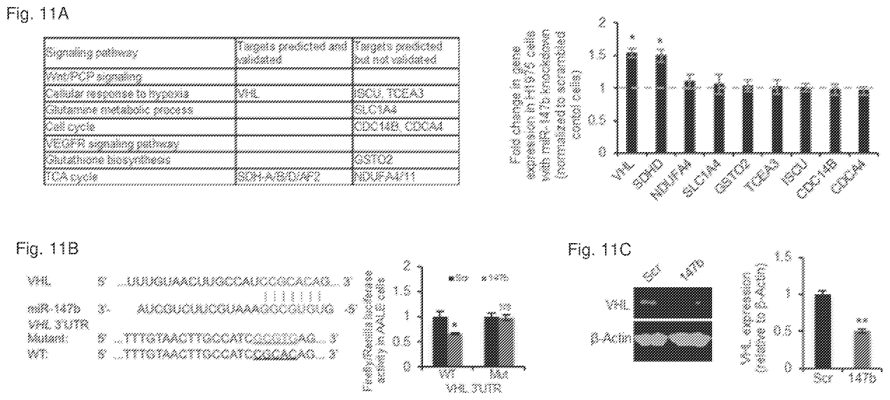

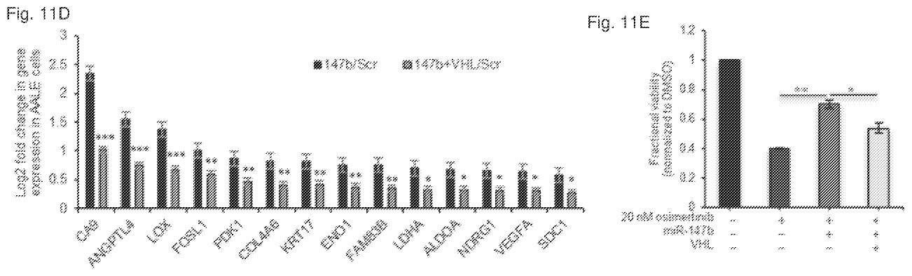

To study which genes are repressed by miR-147b directly, we performed sequence-based target prediction using the TargetScan tool. The predicted targets were then analyzed to match the signaling pathways for drug-tolerance (FIG. 11a). Our data had shown that VHL and SDHD are the top two most upregulated targets upon miR-147b knockdown in H1975 cells in the list of predicted targets for miR-147b (FIG. 11a). They are matched to the signaling pathways, cellular response to hypoxia and the TCA cycle, respectively (FIG. 11a). However, expression levels for other predicted targets relevant to a “tolerance gene signature” including ISCU (iron-sulfur cluster assembly enzyme) and TCEA3 (transcription elongation factor A3) (involved in cellular response to hypoxia) as well as NDUFA4 (NADH dehydrogenase (ubiquinone) 1 alpha subcomplex, 4, 9 kDa) (involved in the TCA cycle) were not upregulated significantly in cells with miR-147b knockdown (FIG. 11a). This indicated that VHL and SDH are potential targets of miR-147b in the context of drug-tolerance.

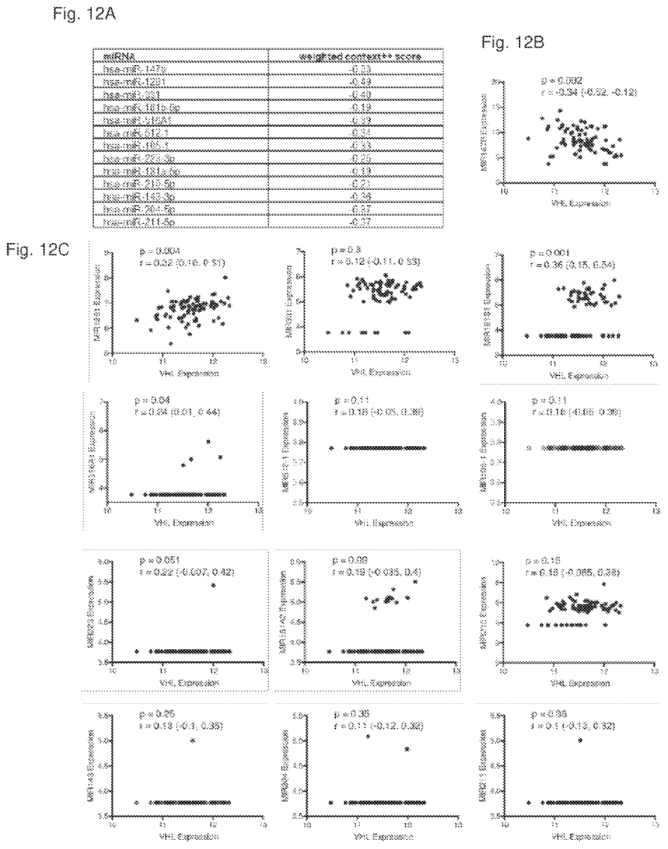

Next, we designed a dual-luciferase assay based on the VHL 3′UTR, wild-type and mutant in those predicted 3′UTR miR-147b binding sites (FIG. 11b). We found that the 3′UTR luciferase activity of VHL was downregulated when miR-147b was overexpressed in AALE cells. However, the luciferase activity for 3′UTR mutant VHL did not change upon overexpression of miR-147b (FIG. 11b). Then we asked whether miR-147b is more likely to be experimentally validated among the top candidate VHL-regulating miRNAs emerging from the TargetScan tool (FIG. 12a). We performed a correlation analysis for VHL and non-coding gene expression in 60 human lung adenocarcinoma cell lines using RNA-seq data (Klijn et al., Nat. Biotechnol. 33(3):306-312, 2015). Our results demonstrated that miR-147b is the most negatively correlating miRNA, supporting our findings that miR-147b can regulate VHL negatively (r=−0.34, P=0.002) (FIG. 12b-c).

Furthermore, we checked the VHL protein level in miR-147b overexpressing cells in AALE. The VHL protein levels decreased only two-fold when miR-147b was overexpressed in AALE cells (FIG. 11c). In the cytoplasm, an E3 ubiquitin ligase containing the VHL tumor suppressor protein targets HIF1alpha for destruction in the presence of oxygen (Ivan et al., Science 292(5516):464-468, 2001). Loss of VHL function leads to the alteration of numerous direct HIF1alpha-mediated transcriptional programs that alter cellular metabolism and induces angiogenesis independent of oxygen levels (Frew et al., Sci. Signal. 1(24):pe30, 2008). Thus, we hypothesize that the changes required for miR-147b induced pseudohypoxia depend on the activity of VHL. To test this hypothesis, we overexpressed VHL in miR-147b overexpressing cells on AALE. As expected, gain-of-function of VHL decreased expression levels of pseudohypoxia genes induced by miR-147b. Those perturbed genes included CA9, ANGPTL4, LOX, FOSL1 (FOS like 1, AP-1 transcription factor subunit), PDK1 (pyruvate dehydrogenase kinase 1), COL4A6 (collagen type IV alpha 6 chain), ENO1 (enolase 1), FAM83B (family with sequence similarity 83 member B), LDHA, ALDOA (aldolase, fructose-bisphosphate A), NDRG1 (n-Myc downstream regulated 1), VEGFA and SDC1 (syndecan 1) (FIG. 11d). Further, functional assay showed that the enhanced osimertinib-tolerance induced by miR-147b-overexpression was reduced upon VHL overexpression on HCC827 cells (FIG. 11e). Taken together, these data indicate that the activity of VHL for repressing “pseudohypoxia gene signature” mediates drug-tolerance initiated by miR-147b.

Tricarboxylic Acid Pathways Mediate Drug Tolerance and Depend on miR-147b

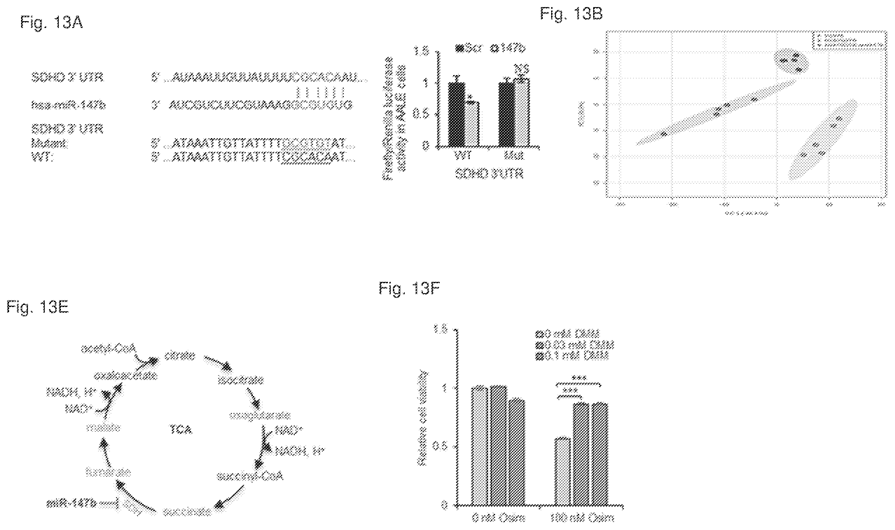

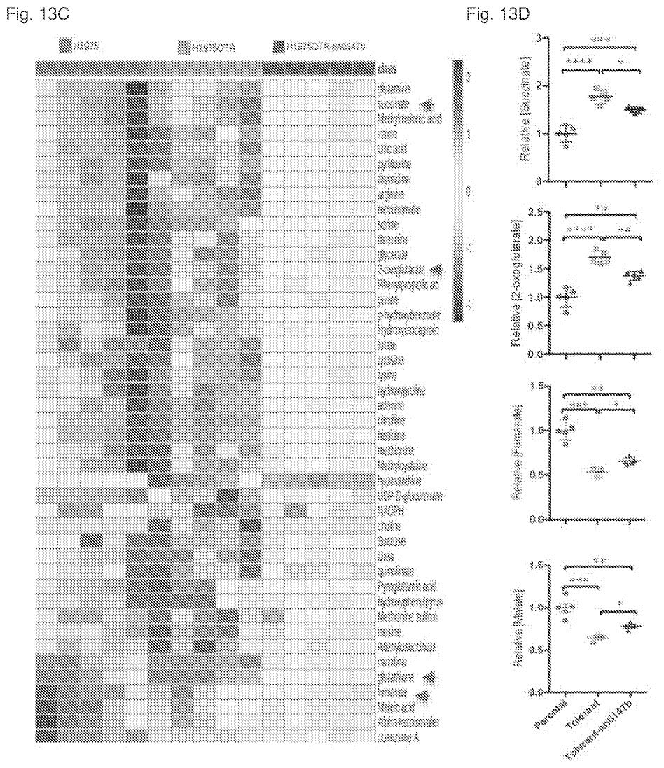

In addition to the functional roles of VHL-mediated “pseudohypoxia gene signature” in drug-tolerance, we hypothesized that another predicted target of miR-147b, SDH might also mediate drug-tolerance induced by miR-147b through its impact on the TCA cycle. To test this hypothesis, we first designed a dual-luciferase assay based on the SDHD 3′UTR, wild-type and mutant in the predicted miR-147b 3′UTR binding sites (FIG. 13a). We found that luciferase activity of 3′UTR SDHD wild type rather than mutant SDHD was downregulated upon overexpression of miR-147b on AALE cells (FIG. 13a). This strongly suggests that SDHD is a direct target repressed by miR-147b.

SDHD, one of the subunits of SDH complex, catalyzes the conversion of succinate to fumarate and regulates both the TCA cycle and the ETC. We asked whether miR-147b-SDHD axis mediated drug-tolerance could impact on the metabolite changes in metabolic pathways. To answer this question, the human lung adenocarcinoma cell line H1975 harboring with EGFR T790M; L858R mutations was used for a metabolomics study. Cells with either EGFR L858R or EGFR T790M are sensitive to osimertinib. The osimertinib-tolerant cells (H1975OTR) were derived from parental H1975 treated with 100 nM osimertinib for 21 days in monolayer cultures. H1975OTR cells are stable and continue to proliferate even in the presence of 100 nM osimertinib. As a control, H1975 cells were treated with vehicle for 21 days. Then we performed a LC/MS metabolomics study using the paired H1975 and H1975OTR cells (FIG. 13b). Analysis of metabolite levels demonstrated that the metabolites in both the TCA cycle and the electron transport chain (ETC) related redox reactions were perturbed in drug-tolerant cells. We observed up to a two-fold rise of succinate and 2-oxoglutarate levels but up to a two-fold decrease of fumarate and malate levels in drug-tolerant cells (FIG. 13c-d and FIG. 14a). In addition, the levels of oxidized nicotinamide adenine dinucleotide (NAD+) decreased 26% in drug-tolerant cells compared to the parental cells (FIG. 14a). This is consistent with a previous finding showing that decreased NAD+ levels induced a pseudohypoxia state in aging (Gomes et al., Cell 155(7):1624-1638, 2013). Furthermore, reduced glutathione (GSH), the master antioxidant, decreased 86% in drug-tolerant cells compared with the parental cells (FIG. 13c). Our data suggest that the metabolic changes in the TCA cycle might be important in regulating drug-tolerance.

Then we asked whether the perturbed metabolite changes could be rescued by blocking miR-147b in drug-tolerant cells. To address this question, we knocked down miR-147b in drug-tolerant cells in H1975 and analyzed the metabolic changes with LC/MS tool. As expected, the increased levels of succinate and 2-oxoglutarate, as well as the decreased levels of metabolites such as fumarate, malate, NAD+, and GSH, were partially rescued by knocking down miR-147b on osimertinib-tolerant cells (FIG. 13c-e and FIG. 14a). These data confirmed our hypothesis that one role of miR-147b is through repressing the enzyme activity of SDH.

Then we asked whether the metabolomic changes in the monolayer cultures are reproducible in the 3D organoid models. To address this question, we established drug-tolerant organoids and parental organoids by continuous treatments with 100 nM osimertinib or vehicle for 21 days on H1975 cells and performed a LC/MS metabolomics study. Consistently, the levels of fumarate, malate, and NAD+ were reduced in osimertinib-tolerant organoids. Knockdown of miR-147b rescued the decreased levels of the above metabolites in those tolerant organoids (FIG. 14b-c). Our data suggest that the metabolic changes due to the depression of SDH by miR-147b might regulate drug-tolerance (FIG. 13e). To further confirm the functional roles of SDH activity in mediating drug-tolerance, we treated H1975 cells with membrane-permeable dimethyl malonate (DMM), one of the inhibitors of SDH in the presence of 100 nM osimertinib. Our results demonstrated that DMM effectively rescued the decreased drug-tolerance to osimertinib (FIG. 13). Collectively, our data have demonstrated that repressed SDH activity by miR-147b mediates osimertinib-tolerance in lung cancer.

Blocking miR-147b Overcomes Drug Tolerance

To understand the functional roles of miR-147b in driving EGFR-TKI tolerance and resistance, we perturbed miR-147b expression using lentiviral inhibitors against miR-147b in H1975 organoids that are partially sensitive to osimertinib. We found that knocking down miR-147b alone or osimertinib administration alone decreased the total number of organoids by two-fold (p<0.01). Following co-treatment with osimertinib and miR-147b inhibition, the number of organoid was decreased by up to 18-fold compared with the control group (p<0.01). This suggests that miR-147b inhibition is synergistic with osimertinib in overcoming TKI-tolerance. Then we treated H1975 cells with serially diluted doses of osimertinib. Our data demonstrated that the IC50 value decreased 166-fold in H1975 cells with miR-147b knockdown compared with the control group. This shows that blocking miR-147b sensitizes H1975 cells towards osimertinib. Unexpectedly, miR-147b knockdown did not decrease 2D-monolayer cell proliferation at high cell-density except at clonal cell-density in H1975 cells. Clonal tumor initiation capacity and clonal long-term repopulation are the principal properties of TICs in cancers.

We hypothesized that functions of miR-147b in driving EGFR-TKI tolerance and resistance are conferred by cancer sternness and TICs. To test this hypothesis, we applied cancer-stemness assays including spheroid-formation assay and limiting-dilution analysis here. Knocking down miR-147b decreased the TICs frequency by seven-fold from 1/11.8 (8.5%) to 1/83.1 (1.2%) (p<0.01). As expected, combinational therapy with miR-147b inhibition and osimertinib almost abolished all those osimertinib-tolerant tumor spheroids and tumor colonies. Consistently, RNA-seq analysis demonstrated that miR-147b knockdown decreased expression levels of stemness-related genes, including activated leukocyte cell adhesion molecule (ALCAM), glycine decarboxylase (GLDC), thyroid transcription factor 1 (TTF1), and AXL receptor tyrosine kinase (AXL). Thus, the osimertinib-tolerance is conferred by miR-147b and cancer-stemness in lung cancer.

Furthermore, we found that mir-147b overexpression enhances malignant transformation and EGFR-TKI tolerance and resistance. First, to understand whether overexpression of miR-147b is linked to lung cancer patient survival, we performed prognosis analysis using the TCGA data portal (http://cancergenome.nih.gov/) and oncoLnc resource. The hazard ratio of miR-147b-high/low was 1.5 (95% confidence interval 1.1-2.2) (p<0.05). Next, we asked whether overexpression of miR-147b would make TKI-sensitive cells more tolerant towards TKIs. To address this question, we used lentiviral vectors with miR-147b to enforce the overexpression of miR-147b. The expression level of miR-147b increased 15-fold in HCC827 cells by qRT-PCR analysis. Using a colony-formation assay, we found that miR-147b overexpression increased colony formation by three-fold. As expected, the frequency of stem-like cells in HCC827 cells increased three-fold from 1/12.69 to 1/4.67 by limiting-dilution assay. To quantify the potential effects of miR-147b overexpression on tolerance to osimertinib treatment, we performed an IC50 assay and found that enforced expression of miR-147b enhanced osimertinib-tolerance in H1975 cells. Further, a clonogenicity assay with osimertinib or gefitinib treatment demonstrated that miR-147b overexpression rescued the osimertinib/gefitinib-induced reduction of colony formation. Thus, it suggests that miR-147b overexpression is important for enhanced osimertinib-tolerance via enhancing cancer sternness.

Next, to understand the potential oncogenic roles of miR-147b, we utilized lung patient-derived xenograft (PDX) tumors directly to analyze the expression of miR-147b. We demonstrated that the miR-147b expression levels in PDX tumors were up to 160-fold higher than normal lung tissues. Then we overexpressed miR-147b using a lentiviral vector in an immortalized human normal lung epithelial cell AALE with undetectable expression level of miR-147b. Overexpression of miR-147b was seen at 43-fold, and enhanced AALE cell proliferation by 1.4-fold on day six. Measuring DNA synthesis is the most precise way to detect changes in cell proliferation. An image-based proliferation assay demonstrated that EdU-positive cells were up two-fold in miR-147b-overexpressing cells compared with scrambled control cells. More interestingly, AALE cells with miR-147b overexpression that were grown for three consecutive passages grew in spherical colonies containing approximately 100 cells spontaneously, while cells expressing the scrambled control grew in flat monolayers. This indicates that miR-147b may enhance the anchorage-independent growth in AALE cells. Then we hypothesized that miR-147b might also decrease the dependence on growth factors for cell growth. EGF is the crucial growth factor for epithelial cells development and growth. To support this hypothesis, we starved the cells in EGF-free media overnight and then tested the growth of cells in media containing various concentrations of EGF. Our data demonstrated that the growth of miR-147-overexpressing cells was less dependent on EGF (R²=0.46) compared with scrambled cells (R²=0.72) significantly (p<0.05). It suggests that miR-147b overexpression could rescue EGF-withdrawal-induced proliferation reduction. Consistently, RNA sequencing analysis showed that miR-147b overexpression increased proliferation-promoting genes including EGFR, MYC, ID1, and NOTCH1 and decreased proliferation-inhibitory genes such as BMP4. The apoptosis-inhibitory genes such as RIPK3 were elevated and the apoptosis-promoting genes such as CD40 were decreased in cells with miR-147b overexpression compared with control cells.

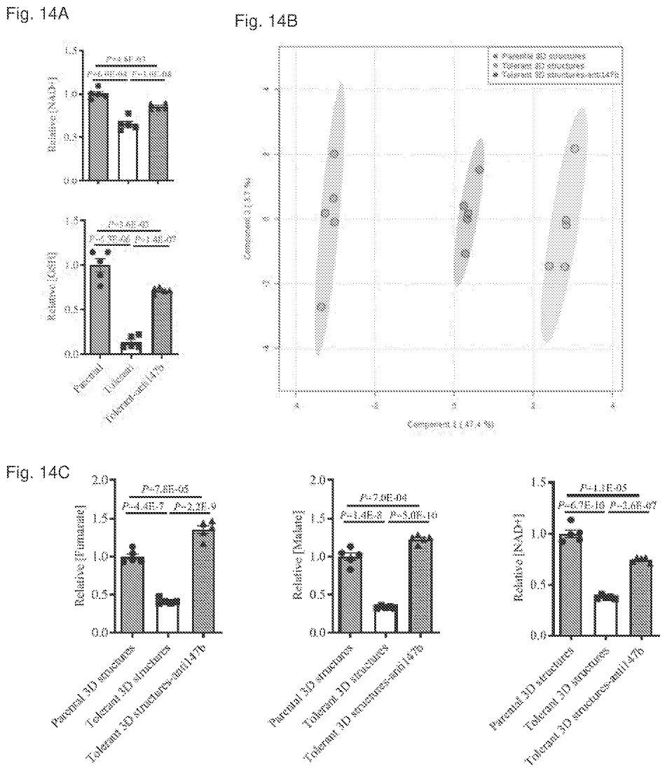

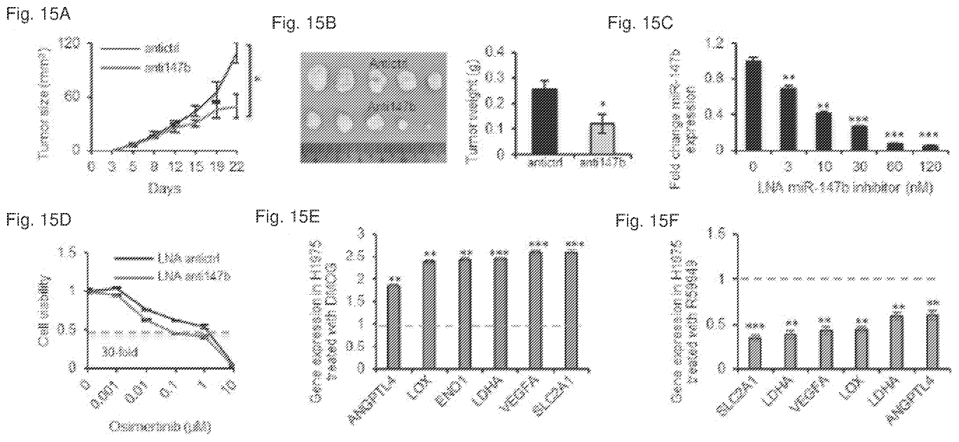

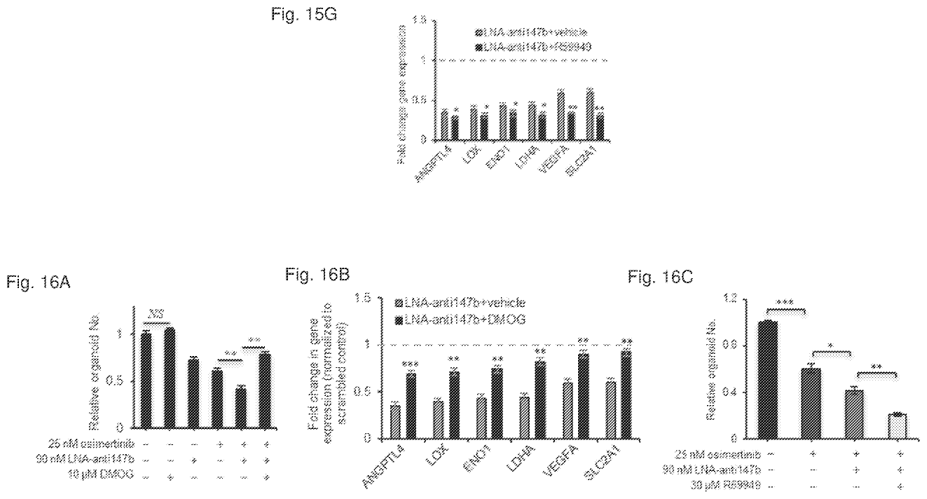

We asked whether miR-147b is a druggable target in lung cancer. First, we knocked down miR-147b with a lentiviral miRNA inhibitor in H1975 cells and transplanted those cells into nude mice. The tumor growth in the cohort with miR-147b knockdown was up to two-fold slower compared with that in the control group (FIG. 15a-b). This indicates that blocking miR-147b inhibits tumor growth in vivo.

Additionally, analysis of GTEx (https://www.gtexportal.org/home/) RNA-seq in 53 tissues from 570 human healthy donors demonstrated that cells and tissues expressing the highest levels of miR-147b are transverse colon, small intestine, and esophagus. The remaining tissues, including lung tissue, express low levels of miR-147b. VHL is moderately expressed in normal lung tissues and other normal tissues indicating that miR-147b-VHL axis might be therapeutic targets that are crucial for tumor initiation and maintenance.

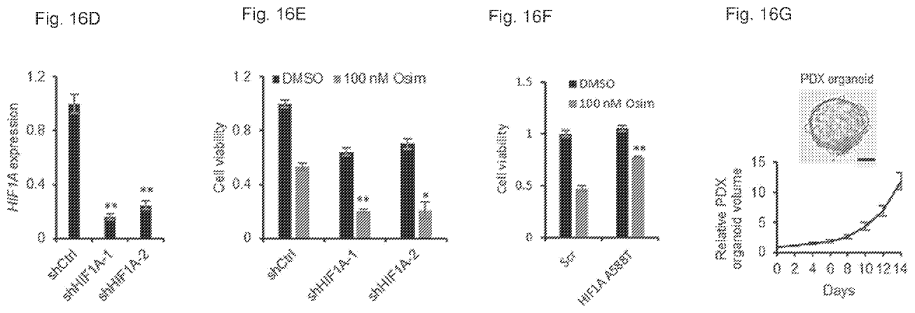

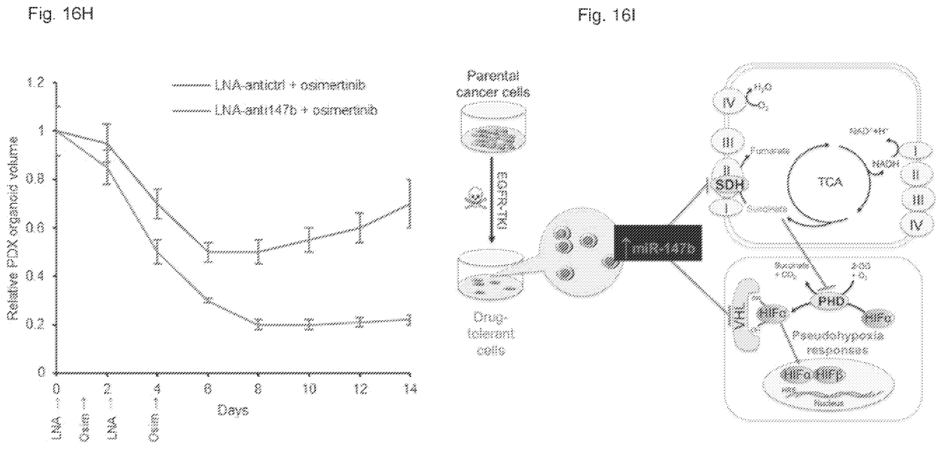

Furthermore, to understand functional roles of miR-147b in regulating drug-tolerance via regulation of a pseudohypoxia signaling pathway, we blocked miR-147b by administration of locked nucleic acid (LNA) miRNA inhibitors, as well as perturbing pseudohypoxia signaling with small molecule activators and inhibitors. First, LNA-miR-147b inhibitor treatment increased the sensitivity of drug-tolerant organoids to osimertinib by 30-fold compared with the control group in H1975 (FIG. 15c-d). The small molecule dimethyloxaloylglycine (DMOG) was reported to activate a pseudohypoxia response through repressing its negative regulator PHD2. As expected, treatment with a single dose of 10 μM DMOG induced upregulated expression of pseudohypoxia genes in H1975 (FIG. 15e). Further functional assays demonstrated that co-treatment with DMOG rescued reduced osimertinib-tolerance caused by LNA-miR-147b inhibitor (FIG. 16a). Consistent with the functional rescue experiment, the reduced levels of “pseudohypoxia genes” induced by miR-147b knockdown were rescued significantly by co-treatments with DMOG in H1975 organoids (FIG. 16b). Then we hypothesized that blocking the pseudohypoxia signaling pathway with small molecules might further enhance drug sensitivity induced by miR-147b inhibitor. To address this question, we applied another small molecule R59949 to this study due to its role in inhibiting pseudohypoxia response through activation of PHD2. We confirmed that treatment with a single dose of 30 μM R59949 induced downregulation of pseudohypoxia genes compared with vehicle treated H1975 cells (FIG. 15f). And co-administration of R59949 and LNA-miR-147b inhibitor showed stronger inhibition on drug-tolerance to EGFR-TKI compared with a single agent of LNA-miR-147b inhibitor (FIG. 16c). Consistently, the reduced expression levels of“pseudohypoxia genes” induced by LNA-miR-147b inhibitor were further inhibited by co-treatment with R59949 in H1975 cells (FIG. 15g). This strongly supports our idea that miR-147b and miR-147b-induced pseudohypoxia signaling pathway are druggable targets to overcome osimertinib-tolerance in lung cancer.

To understand roles of HIF-1 or HIF-2 in the osimertinib tolerant state, we knocked down HIF1A and HIF2A/EPAS1 (endothelial PAS domain protein 1) using lentiviral shRNAs in H1975 cells and investigated their effect on osimertinib response. Our results showed that HIF1A knockdown increased cell sensitivity up to 2.6-fold towards osimertinib (FIG. 16d-e). However, HIF2A knockdown did not change drug sensitivity towards osimertinib significantly (FIG. 17a-b). Furthermore, to better understand whether gain of HIF-1 is sufficient to induce a tolerant state, we overexpressed constitutive active HIF1A using mutant HIF1A A588T in H1975 cells. As expected, overexpression of HIF1A A588T increased drug-tolerance towards osimertinib by up to two-fold (FIG. 16t). Thus, our results have now demonstrated that HIF1A rather than HIF2A is sufficient to induce an osimertinib tolerant state.

Lastly, we asked whether we could delay drug-tolerance to EGFR-TKIs by targeting miR-147b. To address this question, organoids obtained from PDX lung tumors were tested. Among these PDX lung tumors, one EGFR T790M mutated PDX tumor-derived organoid (PDX_LU_10) at passage two was tested in the following functional study (FIG. 16g). We established PDX organoids at medium size one week after seeding single-cells into 3D cultures. We recorded this time point as day 0 before the administration of LNAs or osimertinib. As expected, the PDX organoids increased their volumes up to ten-fold within 14 days in the vehicle-treated group (FIG. 16g). With the administration of 25 nM osimertinib to PDX organoids on day 1 and 4, the size of the tumor organoids decreased 50% on day 6 and then started to recover gradually with a 40% increase on day 14. To test whether early perturbation of miR-147b could delay drug-tolerance to osimertinib, we pretreated the organoids with 90 nM LNA miR-147b inhibitor on day 0 and repeated the treatment on day 2. These pretreatments with LNA miR-147b inhibitor further decreased osimertinib-tolerance on day 8 by 80% compared with control cells. Furthermore, the PDX organoids volume increased no more than 10% of that in control cells with the single agent of osimertinib from day 8 to day 14 (FIG. 16h). Our data suggests that early treatment of EGFR mutant lung cancer with miR-147b inhibitor might delay drug-tolerance to EGFR-TKIs compared with single EGFR-TKI treatment.

### Methods

Cell Culture. Human lung EGFR-wild type cell lines H358, H460, A549, H1299, and H69 (ATCC) as well as EGFR-mutant cell lines H1650, H1975, HCC827, PC9, PC9ER, and H3255 (provided by S.K.) were cultured in DMEM (high glucose) (GIBCO) with 10% FBS, 2 mM L-glutamine and 1% penicillin-streptomycin. Immortalized tracheobronchial epithelial AALE cells (provided by W.C.H.) were derived as previously described (Lundberg et al., Oncogene 21(29):4577-4586, 2002) and maintained in SAGM media (Lonza). Each cell line was maintained in a 5% CO₂ atmosphere at 37° C. Cell line identities were confirmed by STR fingerprinting and all were found negative for mycoplasma using the MycoAler Kit (Lonza).

Mice. All research involving animals complied with protocols approved by the BIDMC Biological Resource Center Institutional Animal Care and Use Committee. 4-6 weeks old female nude immunodeficient mice (Jackson Laboratory) were used for subcutaneous injections. For subcutaneous xenograft tumor assay, 100,000 cells in serum-free medium and growth factor reduced Matrigel (BD) (1:1) were inoculated into the flank of nude mice. The xenograft tumor formation was monitored by calipers twice a week. The recipient mice were monitored and euthanized when the tumors reached 1 cm in diameter.

Patient-derived Xenograft Tumor Specimens. Tumor samples from patient-derived xenografts (PDXs) were generated at The Jackson Laboratory and the Yale Cancer Center by subcutaneous implantation of previously passaged tumors in up to 5 female NSG mice. When tumor samples reached 1000 mm³ they were shipped to the laboratory in frozen media of DMEM with 90% FBS and 10% DMSO in dry ice. Samples were washed with cold phosphate buffer saline (PBS) with antibiotics (Sigma-Aldrich, St. Louis, Mo.) three times, chopped with a sterile blade, and incubated in 0.001% deoxyribonuclease (DNase) (Sigma-Aldrich, St. Louis, Mo.), 1 mg/ml collagenase/dispase (Roche, Indianapolis, Ind.), 200 U/ml penicillin, 200 μg/ml streptomycin, 0.5 μg/ml amphotericin B (2% antibiotics, Sigma) in DMEM/F12 medium (GIBCO, Grandlsland, N.Y.) at 37° C. water bath for 3 hours with intermittent shaking. After incubation, the suspensions were repeatedly triturated, passed through 70 μm and 40 μm cell-strainers (BD Falcon, San Jose, Calif.), and centrifuged at 122 g for 5 minutes at 4° C. Cells were resuspended in red blood cell lysis buffer (eBioscience, San Diego, Calif.) for 4 minutes at room temperature with intermittent shaking, before resuspension in serum-free medium. After lysis, cell viability was evaluated by trypan blue dye exclusion. Live single cells accounted for 90% of the whole population and dead cells accounted for less than 10%. Each tumor sample yielded ˜1×10⁵ to 1×10⁸ cells, depending on the sample size.

Antibodies. For immunofluorescence staining, primary mouse anti-human ZO-1 (1:100, cat #339100) was from Thermo Fisher Scientific. Secondary goat anti-mouse IgG conjugated with Alexa Fluor 488 (1:500, cat #A-11055) was from Invitrogen. For western blot, primary rabbit anti-VHL antibody (1:100, Cat #PA5-27322) was from Thermo Fisher Scientific. Mouse anti-β-actin (1:5,000, clone C4, Santa Cruz, sc-47778) was used as loading control. IRDye 680RD goat anti-rabbit (1:20,000, LI-COR926-68171, LI-COR Biosciences) and IRDye 800CW goat-anti-mouse (1:20,000, LI-COR827-08364, LI-COR Biosciences) were used as secondary antibodies.

3D Spheroids and Organoids. For 3D spheroid formation, single-cell suspensions (10,000 cells/well) were plated in 6-well ultra-low attachment (Corning) or non-treated cell culture plates (Nunc) in DMEM/F12 medium containing 2 mM L-glutamine, 15 mM HEPES, 1 mg/ml NaHCO₃, 0.6% Glucose, 1% NEAA, 4 mg/nl BSA (Sigma), ITS (0.05 mg/ml insulin/transferrin/selenous acid, BD Biosciences), 1% antibiotics (Sigma), 50 ng/ml EGF, and 20 ng/ml FGF2 (Invitrogen). Fresh medium was replenished every 3 days. Spheroids were cultured for 10-14 days and then quantified. For passaging, spheroids were digested by accutase (Chemicon) into single cells and re-plated into the above plates. For limiting dilution assays, 200, 600, and 1800 cells were plated to assess spheroid formation.

For 3D organoid formation, single-cell suspensions (2000 cells/well/20 μl) were co-plated with geltrex (25 μl) in 96-well non-treated clear plates (Corning, cat #08-772-53). The plate was incubated for 20 minutes at 37° C. followed by adding 100 μl complete growth media. The complete growth media was advanced DMEM/F12 with glutamax (1×), HEPES (1×), 1.25 mM N-Acetylcysteine, 10 mM Nicotinamide, 10 μM Forskolin, B27 (1×), 5 ng/ml Noggin, 100 ng/ml FGF10, 20 ng/ml FGF2, 50 ng/ml EGF, 10 ng/ml PDGFA, 10 ng/ml FGF7, 1% penicillin-streptomycin, and 10 μM Y-27632. Y-27632 was used only for the initial three days because Y27632 is a rock inhibitor preventing apoptosis of single cells (Watanabe et al., Nat. Biotechnol. 25(6):681-686, 2007). PDGFA and FGF7 were not used until day 7 in organoid cultures because they are important for alveolarization during late lung development (Padela et al., Pediatr. Res. 63(3):232-8, 2008; Bostrom et al., Cell 85(6):863-873, 1996). FGF10 is essential for maintenance of lung progenitor cells and branching morphogenesis as well as tissue homeostasis in the adult lung (Sekine et al., Nat. Genet. 21(1):138-141, 1999). EGF and FGF2 are mitogens for growth of epithelial cells and used for maintaining lung tumor-initiating cells previously by us (Zhang et al., Cell 148(1-2):259-272, 2012). Noggin binds and inactivates bone morphogenetic protein-4 and is involved in the development of the lungs (Krause et al., Int. J. Biochem. Cell Biol. 43(4):478-481, 2011). The media was changed every three days in 24 days. The organoids were photographed with a microscope (Evos Fla., Life Technology) and their size was measured by ImageJ.

Colony Formation Assay in Plate. Single cells were plated in 10 cm dish in triplicates with 20, 40, 80, or 300 cells per dish. Fresh medium was replenished every 3 days. The cells were incubated for 10-12 days followed by Giemsa (Sigma) staining. The plates were air-dried, photos taken, and the total number of colonies was analyzed by openCFU (opencfu.sourceforge.net).

Single Cell-Derived Clones of PC9 and HCC827 Cells. In PC9 and HCC827 cells, a single cell was sorted into a 96-well plate at one cell per well by fluorescence-activated cell sorting (FACS) using a FACSAria (BD). The single cell in each well was confirmed under a microscope 12 hours after sorting. Gefitinib or osimertinib were administrated to both parental clones and single-cell clones. In parental clones, the single cells were treated immediately with 0.1, 0.4, and 2 μM gefitinib, osimertinib, or vehicle for 14 days on the second day (n=192 wells per group). In single-cell clones, clones were made first, and then exposed to 0.1-2 μM gefitinib, osimertinib, or vehicle for 14 days. Drug responses of the surviving clones were determined by measuring an IC50. The frequency of colony formation was calculated as a ratio of the total number of colonies (consisting of more than 50 cells) to the total number of wells plated with a single cell. Medium and smal molecule inhibitors were replenished every three days. One parental single-cell derived clone treated with vehicle that was sensitive to gefitinib and two gefitinib-tolerant single-cell derived clones were randomly selected and applied for the following whole transcriptome analysis by microarray. Four single-cell clones from PC9 and HCC827 were established from the above were used for drug-tolerance assay.

Compounds. Osimertinib (S7297) and gefitinib (S1025) were purchased from Selleck Chemicals. DMOG (Jaakkola et al., Science 292(5516):468-472, 2001) (Cat #400091) was from Calbiochem. R59949 (Temes et al., J. Biol. Chem. 280(25):24238-24244, 2005) (Cat #D5794) and dimethyl malonate (Mills et al., Cell 167(2):457-470, 2016; Dervartanian et al., Biochim. Biophys. Acta. 92:233-247, 1964) (DMM, Cat #136441) were purchased from Sigma-Aldrich.

Compound Treatment. Cell viability experiments were performed in 96-well format using opaque white plates (Corning). For 2D monolayer cell cultures and organoids, cells were plated into 96-well plates with 100-2000 cells per well in three-four replicates on day 0. Twenty-four hours after seeding, cells or organoids were exposed to compounds at indicated concentrations for 72 hours. Cellular ATP levels (as a surrogate for viability) were measured using CeliTiter-Glo (Cat #G7570, Promega) or CeliTiter-Glo 3D (Cat #G9681, Promega). For co-treatment experiments, spent medium was removed 24 hours after cell seeding and replaced with medium containing a single concentration of the modulator of interest (for example, osimertinib).

To establish gefitinib and osimertinib tolerant cells, PC9 single-cells were treated with 20 nM osimertinib and 40 nM gefitinib for 12-14 days, HCC827 cell monolayers and organoids were treated with 20-160 nM osimertinib for 12-21 days, and H1975 cell monolayers and organoids were treated with 25 nM-1 μM osimertinib for 12-21 days. To study effects of organoid culture stages on outcome of drug-tolerance, both single-cells (grown for 1 day) and established organoids from HCC827 cells (grown for 24 days) were made first followed by 100 nM osimertinib treatment for additional 21 days. Medium was replenished every three days.

RNA Extraction and Real-Time PCR. Total RNA was extracted from solid tissues and cultured cells using mirVana™ miRNA Isolation Kit (Ambion #AM1561) according to the manufacturer's instructions. A total of 10 ng RNA each sample was input for consecutive reactions including Poly(A) Tail reaction, Ligation reaction, Reverse Transcription reaction, and miR-Amp reaction using the Taqman Advanced miRNA cDNA synthesis kit (Applied Biosystems #A28007). Then miRNA expression was assessed by Taqman Advanced microRNA Assay and the Taqman Fast Advanced miRNA master mix (Applied Biosystems #4444557). The PCR reaction plate was run in a real-time PCR instrument (Roche Lightcycler 480 system) according to the manufacturer's instructions. Three biological replicates were applied for each sample. MiRNA expression was assessed by Taqman Fast Advanced MicroRNA Assay, and the gene expression of mRNAs was evaluated by Taqman Probes (Applied Biosystems). Taqman miRNA probes were as follow: hsa-miR-147b (478717_mir) and hsa-miR-423-5p (478090_mir). hsa-miR-423-5p was used as endogenous control. Taqman gene-expression probes were as follow: ID2 (Hs04187239_m1), SFTPC (Hs00951326_g1), HOPX (Hs05028646_s1), NKX2.1 (Hs00968940_m1), CEACAM5 (Hs00944025_m1), LIN28B (Hs01013729_m1), EPAS1 (Hs01026149_m1), VHL (Hs03046964_s1), KRT17 (Hs00356958_m1), CA9 (Hs00154208_m1), WNT5A (Hs00998537_m1), WNT4 (Hs01573505_m1), EGLN3 (Hs00222966_m1), SLC2A1 (Hs00892681_m1), SLC2A3 (Hs00359840_m1), LOX (Hs00942483_m1), CS (Hs02574374_s1), TCEB1 (Hs00855349_g1), CAD (Hs00983188_m1), CDKN1A (Hs00355782_m1), IDH3A (Hs00194253_m1), SPRY4 (Hs01935412_s1), FZD7 (Hs00275833_s1), FZD2 (Hs00361432_s1), UBC (Hs05002522_g1), RAC1 (Hs01902432_s1), P4HA1 (Hs00914594_m1), P4HA2 (Hs00990001_m1), ADM (Hs00969450_g1), BNIP3L (Hs00188949_m1), ANKRD37 (Hs00699180_m1), NDRG1 (Hs00608387_m1), DCBLD1 (Hs00543575_m1), KCTD11 (Hs00922550_s1), BNIP3 (Hs00969291_m1), VEGFA (Hs00900055_m1), ALDOA (Hs00605108_g1), PFAS (Hs00389822_m1), GLS (Hs01014020_m1), GLUD1 (Hs03989560_s1), ASNSD1 (Hs00219383_m1), GMPS (Hs00269500_m1), NIT2 (Hs00252405_m1), ACLY (Hs00982738_m1), ACO2 (Hs00426616_g1), PDHA1 (Hs01049345_g1), OGDH (Hs01081865_m1), FH (Hs00264683_m1), SDHA (Hs00417200_m1), SDHB (Hs01042478_g1), SDHC (Hs01698067_s1), SDHD (Hs00829723_g1), SDHAF2 (Hs00215235_m1), DLAT (Hs00898876_m1), DLST (Hs04276516_g1), ISCU (Hs00384510_m1), TCEA3 (Hs00957468_m1), SLC1A4 (Hs00983079_m1), CDC14B (Hs00372920_m1), CDCA4 (Hs00937497_s1), GSTO2 (Hs01598184_m1), NDUFA4 (Hs00800172_s1), NDUFA11 (Hs00418300_m1), ACTB (Hs01060665_g1) and GAPDH (Hs02786624_g1). ACTB was used as endogenous control.

Pyrosequencing for quantitative analysis of sequence variations. The parental cells, gefitinib-tolerant cells, and gefitinib-resistant cells in PC9 were extracted for DNA (QIAamp DNA blood mini kit, Cat #51104, Qiagen) and analyzed for pyrosequencing. The methods were described as previously (Koontz et al., BMC Med. Genet. 10:80, 2009).

High-Throughput Sequencing. The total RNA samples (1 μg) were processed by LC Sciences for microRNA sequencing (miRNA-seq). All RNA samples were analyzed for quality on an Agilent 2100 Bioanalyzer.

For miRNA-seq, paired osimertinib-tolerant cells and parental cells (treated with 20 nM osimertinib or vehicle for 14 days) from HCC827 and PC9 cells were applied. The RNA samples were processed utilizing Illumina's TruSeq small RNA sample preparation protocol for small RNA library generation (Part #15004197 Rev. F, Cat #RS-200-9002DOC). The subsequent sequencing was performed on the HiSeq 2500 platform for 1×50-nt single-end sequencing and the sequencing adaptor was trimmed from the raw reads. The reads were then mapped to the miRBase v21 (http://www.mirbase.org/) and the human genome (GRCh37) using Bowtie (Langmead et al., Genome Biol. 10(3):R25, 2009). The mapping results were summarized using an in-house script to estimate the number of reads mapped to each miRNA. Normalization was done using the median of the ratio of the read count to the geometric mean of read counts across samples as implemented in DESeq (Anders et al., Genome Biol. 11(10):R106, 2010).

Whole Transcriptome Analysis by microarray. The Illumina Whole Human Genome Microarray Kit (HumanHT-12 v4 Expression BeadChip, Cat #BD-103-0204) was used to identify differentially expressed genes in single-cell clones from PC9. Amplification of RNA, hybridization, image processing, and raw data extraction: The Illumina TotalPrep RNA Amplification kit (Ambion, UK) was used for all samples using 200 ng of total RNA as starting material. Briefly, the procedure consisted of a reverse transcription step using an oligo (Dt) primer bearing a T7 promoter and the high yield ArrayScript™ reverse transcriptase. The cDNA then underwent second strand synthesis and clean-up to become a template for in vitro transcription with T7 RNA Polymerase and biotin-NTP mix. Labelled cRNA was then cleaned up and 1.5 μg were hybridized to humanHT12_V4 beadarrays (Illumina, CA, USA) for 16 hours at 55° C. Following hybridization, beadarrays were washed and stained with streptavidin-Cy3 (GE Healthcare, UK). Fluorescent images were obtained with a Beadarray reader and processed with the BeadScan software (Illumina, CA, USA). The whole transcriptome raw data were obtained from the GenomeStudio software with the subtraction of the background. All mRNA raw data were normalized based on the Cross-Correlation method (Chua et al., Nucleic Acids Res. 34(5):e38, 2006). Significantly changed mRNAs were identified based on average fold change cutoff of 1.5 and the cutoff of the p value cross all replicates at 0.05.

Two-Color Western Blot and Chemical Reagents. Cells were harvested and lysed with RIPA buffer (Radio Immuno Precipitation Assay buffer) supplemented with protease and phosphatase inhibitor cocktail (Roche). Protein concentrations of the extracts were measured using BCA assay (Pierce) and equalized with the extraction reagent. Equal amount of the extracts was loaded and subjected to SDS-PAGE, transferred onto Immobilon-FL PVDF membranes. The PVDF membranes were air-dried for 1 hour at room temperature followed by rehydration. The membranes were blocked with Odyssey Blocking Buffer for 1 hour and then incubated with primary antibodies in cold room overnight. Then IRDye 680RD goat anti-rabbit (1:20,000, LI-COR926-68171) and IRDye 800CW goat-anti-mouse (1:20,000, LI-COR827-08364) were used as secondary antibodies. Then the images were scanned with Odyssey Family Imaging System (LI-COR Biosciences). Western blot quantification was performed by Image Studio Lite (LI-COR Biosciences).

Transfection by LNAs in vitro. Tumor cells were plated at 2,000 cells in complete growth medium in a 96 well plate to reach 50-60% confluence. 0˜120 nM of fluorescein-conjugated LNA anti-miR-147b (Sequence: AGCAGAAGCATTTCCGCACA; SEQ ID NO: 890) (Cat #4100977-011) or negative control (Sequence: TAACACGTCTATACGCCCA; SEQ ID NO: 891) (Cat #199006-011, Exiqon) with PureFection (System Biosciences) were applied for transfection. The transfected cells were harvested after culturing for 48 and 72 hours.

HIF1A and EPAS1 shRNAs and cDNA transfection. H1975 cells were seeded in a 6-well plate at 100,000 cells per well one day prior to transfection. A mixture of 2.5 μg pGFP-C-shLenti vector targeting HIF1A (OrGene, Cat #320380), EPAS1 (OriGene, Cat #TL315484), scrambled negative control (Cat #TR30021), lentiviral vector targeting HIF1A mutantA588T (OrGene, Cat #RC402571), control vector and 7.5 μL of PureFection (System Biosciences, Cat #LV750A-1) were used for transfection. The transfected cells were selected and maintained in 0.5 μg/ml puromycin (for shRNAs) or 600 μg/ml neomycin (for HIF1A A5887) in DMEM containing with 10% FBS for 9 days. Then the stable cells were passaged into 96-well plate at 3,000 cells per well followed by treatment with 100 nM osimertinib for 3 days. hsa-HIF1A targeting sequences: shRNA 1: AGCTTGCTCATCAGTTGCCACTTCCACAT (SEQ ID NO: 892), shRNA 2: AGGCCACATTCACGTATATGATACCAACA (SEQ ID NO: 893), shRNA 3: TACGTTGTGAGTGGTATTATTCAGCACGA (SEQ ID NO: 894), shRNA 4: ACAAGAACCTACTGCTAATGCCACCACTA (SEQ ID NO: 895). hsa-EPAS1 targeting sequences: shRNA 1: GTATGAAGAGCAAGCCTTCCAGGACCTGA (SEQ ID NO: 896), shRNA 2: AGCACTGCTTCAGTGCCATGACAAACATC (SEQ ID NO: 897), shRNA 3: CCTGGTGGCAGCACCTCACATTTGATGTG (SEQ ID NO: 898), shRNA 4: GGCTGTGTCTGAGAAGAGTAACTTCCTAT (SEQ ID NO: 899).

Transient Transfection and Dual-Luciferase Assay. PureFection (System Biosciences) was used for transient transfection. 100 ng of wild-type or mutant 3′UTR reporter constructs of VHL or SDHD constructs (GeneCopoeia) were transfected into H1975 cells with 120 nM of LNA anti-miR-147b or negative control. Firefly and Renilla luciferase activities were measured 48 hours post-transfection using Dual-Luciferase Reporter System (Promega). The firefly luminescence was normalized to Renilla luminescence as an internal control for transfection efficiency. MiR-147b binding site CGCAC (SEQ ID NO: 900) was substituted with GCGTG (SEQ ID NO: 901) in mutated VHL and binding site CGCACA (SEQ ID NO: 28) was substituted with GCGTGT in mutated SDHD.

Lentiviral-mediated miRNA and VHL Overexpression or Knockdown Infection. For lentiviral overexpression or knockdown of miR-147b, cells (AALE, HCC827, H1975, and PC9ER) were infected with the lentiviral particles (Applied Biological Material Inc., ABM) for 48 hours in the presence of 1:100 Viralplus transduction enhancer (ABM) and 8 μg ml⁻¹ polybrene (Sigma). Two days after infection, puromycin was added to the media at 0.5 μg ml⁻¹, and cell populations were selected for 1-2 weeks. For lentiviral overexpression of VHL, cells (HCC827) at 70% confluence were transduced with VHL lentiviral particles (1.6×10⁸ TU ml⁻¹, ABM) or blank control lentiviral particles (2×10⁶ TU ml⁻¹, ABM) together with polybrene. Then the infected cells were passaged and selected by puromycin (Invitrogen) at 0.5 μg ml⁻¹ for 1-2 weeks.

crRNA:tracrRNA transfection. H1975-Cas9 cells were generated with plenti-EF1a-Cas9 lentiviral particles (ABM, Cat #K003) and maintained in 0.5 μg/ml puromycin in DMEM containing with 10% FBS. H1975-Cas9-intergrated cells were seeded in a 98-well plate at 3,000 cells per well one day prior to transfection. Edit-R-synthetic crRNA (CRISPR RNA) targeting MIR147B (GE Healthcare Dharmacon, Cat #crRNA-413428, 413429, 413430 and 413431), non-targeting control (Cat #U-007501-01-20) and tracrRNA (trans-activating CRISPR RNA) (Cat #U-002005-20) were individually resuspended in 10 mM Tris-HCl pH7.5 to a concentration of 100 uM. crRNA and tracrRNA were obtained at equimolar ratio and diluted to 2.5 μM using 10 mM Tris-HCl pH7.5. A final concentration of 50 nM crRNA-tracrRNA complex was used for transfection. Cells were transfected using 0.4 μL/well of DharmaFECT Duo transfection reagent (GE Healthcare Dharmacon, Cat #T-2010-02). hsa-miR-147b targeting sequences:

crRNA 1:
(SEQ ID NO: 902)
5′ AGAGTACTCTATAAATCTAG 3′,
crRNA 2:
(SEQ ID NO: 903)
5′ TTTCTGCACAAACTAGATTC 3′,
crRNA 3:
(SEQ ID NO: 904)
5′ AGATTCTGGACACCAGTGTG 3′,
and
crRNA 4:
(SEQ ID NO: 905)
5′ GCAGAAGCATTTCCGCACAC 3′.

H&E Staining and Immunofluorescence. Samples were formalin-fixed, paraffin-embedded, sectioned, and stained with hematoxylin-eosin (H&E) according to standard histopathological techniques. For immunofluorescence, organoids were fixed and then incubated with mouse anti-ZO-1 (Thermo Fisher Scientific), washed, then incubated with anti-mouse IgG-Alexa Fluor 488 (Invitrogen). The organoids were counterstained with Hoechst 33342. Z-stack images were acquired with 2 μm slice interval and 3-D projection was created with a confocal microscope (Zeiss LSM 880).

Metabolite extraction. For collecting adherent cells from 10-cm dishes, the metabolomics samples were prepared according to a previous method (Yuan et al., Nat. Protoc. 7(5):872-881, 2012). Briefly, the growing cells at 80% confluence were incubated with 80% methanol at −80° C. for 15 minutes. The cell lysate/methanol mixture were transferred to 15 mL conical tubes and centrifuged at 4500 g at 4° C. for 15 minutes in cold room to pellet cell debris and proteins. The centrifugation was repeated twice, and all three extractions were pooled together. The supernatants were completely dried by speedVac and were further processed for LC-MS analysis. Five biological replicates were used in each group and the analysis was normalized with the same number of cells of each group.

For collecting organoids, the above method was modified. Briefly, single cells mixed with geltrex were plated into six-well low attachment plates (Nunc) and incubated with complete media for 21 days. Next, the organoids/geltrex mixtures were incubated with TrypLE Express (Gibco) at 37° C. for 5 minutes to separate geltrex from organoids. The supernatants were aspirated after centrifuge at 188 g for 5 minutes. Then the organoid pellets were incubated with 80% methanol at −80° C. for 30 minutes. The cell lysate/methanol mixture were transferred to 15 mL conical tubes and centrifuged at 4500 g at 4° C. for 15 minutes to pellet cell debris and proteins. The centrifugation was repeated twice, and all three extractions were pooled. The supernatants were completely dried by speedVac and were further processed for LC-MS analysis. Five biological replicates were used in each group and the analysis was normalized with the same number of cells of each group.

Targeted Mass Spectrometry. Samples were re-suspended using 20 mL HPLC grade water for mass spectrometry. 5-7 μL were injected and analyzed using a hybrid 5500 QTRAP triple quadrupole mass spectrometer (AB/SCIEX) coupled to a Prominence UFLC HPLC system (Shimadzu) via selected reaction monitoring (SRM) of a total of 274 unique endogenous water-soluble metabolites for steady-state analyses of samples. Some metabolites were targeted in both positive and negative ion mode for a total of 306 SRM transitions using positive/negative ion polarity switching. ESI voltage was +4900V in positive ion mode and −4500V in negative ion mode. The dwell time was 3 ms per SRM transition and the total cycle time was 1.65 seconds. Approximately 9-13 data points were acquired per detected metabolite. Samples were delivered to the mass spectrometer via hydrophilic interaction chromatography (HILIC) using a 4.6 mm i.d.×10 cm Amide XBridge column (Waters) at 400 μL/minute. Gradients were run starting from 85% buffer B (HPLC grade acetonitrile) to 42% B from 0-5 minutes; 42% B to 0% B from 5-16 minutes; 0% B was held from 16-24 minutes; 0% B to 85% B from 24-25 minutes; 85% B was held for 7 minutes to re-equilibrate the column. Buffer A was comprised of 20 mM ammonium hydroxide/20 mM ammonium acetate (pH=9.0) in 95:5 water acetonitrile. Peak areas from the total ion current for each metabolite SRM transition were integrated using MultiQuant v2.1 software (AB/SCIEX). Further informatics analysis was performed with online MetaboAnalyst 3.0 software (Xia et al., Curr. Protoc. Bioinformatics 55:14.10.1-14.10.91, 2016).

Statistical Analysis. No statistical methods were used to predetermine sample size. For mouse experiments, the mice were not randomized. The investigators performing tumor volume measurements were blinded. All experiments were performed in two to five biological replicates, and independently reproduced as indicated in figure legends. Data are presented as the means t SEM. Unless otherwise stated, statistical significance was determined by a Student's two-tailed t-test by GraphPad Prism 6. P<0.05 was considered statistically significant. For two samples that are not normally distributed, Mann-Whitney test was applied for a comparison. Fisher's exact test was applied for an association analysis between miR-147b expression levels and EGFR/KRAS mutations in lung adenocarcinoma tissues from the TCGA dataset. Spearman correlation test was used for a correlation analysis between VHL and its upstream candidate VHL-regulating miRNAs emerging from the TargetScan analysis. The TIC frequencies were estimated using ELDA software (Hu et al., J. Immunol. Methods 347(1-2):70-78, 2009). The survival curves and hazard ratios were compared by log-rank test. The enrichment of Gene Ontology (version: releases/2016-09-30) functional annotations using DAVID Bioinformatics tool (v6.8, October 2016) was performed by modified Fisher's exact test on the microarray data from PC9 single-cell clones. The enrichments were based on all evidence codes.

### OTHER EMBODIMENTS

Various modifications and variations of the described invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention that are obvious to those skilled in the art are intended to be within the scope of the invention. Some embodiments are within the scope of the following numbered paragraphs.

1. A method of treating, reducing, preventing, or delaying tolerance or resistance to anti-receptor tyrosine kinase (RTK) therapy in a subject, the method comprising administering a miR-147b inhibitor to the subject.

2. The method of paragraph 1, wherein the RTK is selected from the group consisting of epidermal growth factor receptor (EGFR), human EGFR2 (HER2), HER3, anaplastic lymphoma kinase (ALK), ROS1, ERBB2/3/4, KIT, MET/hepatocyte growth factor receptor (HGFR), RON, platelet derived growth factor receptor (PDGFR), vascular endothelial cell growth factor receptor (VEGFR), VEGFR1, VEGFR2, fibroblast growth factor receptor (FGFR), insulin-like growth factor 1 receptor (IGF1R), and RET.

3. The method of paragraph 1, wherein the miR-147b inhibitor reduces a Von Hippel-Lindau (VHL)-pseudohypoxia response or counteracts metabolic changes in the tricarboxylic acid (TCA) cycle associated with drug tolerance in the subject.

4. The method of any one of paragraphs 1 to 3, wherein the subject has cancer.

5. A method of treating or preventing cancer in a subject, the method comprising administering a miR-147b inhibitor to the subject.

6. The method of paragraph 4 or 5, wherein the subject has a cancer selected from the group consisting of lung cancer, non-small cell lung cancer, colorectal cancer, anal cancer, glioblastoma, squamous cell carcinoma, squamous cell carcinoma of the head and neck, pancreatic cancer, breast cancer, renal cell carcinoma, thyroid cancer, gastroesophageal adenocarcinoma, and gastric cancer.

7. The method of any one of paragraphs 1 to 6, further comprising administering an anti-RTK therapy to the subject.

8. The method of paragraph 7, wherein the anti-RTK therapy is an anti-EGFR therapy.

9. The method of paragraph 8, wherein the anti-EGFR therapy comprises a tyrosine kinase inhibitor (TKI).

10. The method of paragraph 9, wherein the TKI is selected from the group consisting of gefitinib, erlotinib, afatinib, lapatinib, neratinib, osimertinib, vandetanib, crizotinib, dacomitinib, regorafenib, ponatinib, vismodegib, pazopanib, cabozantinib, bosutinib, axitinib, vemurafenib, ruxolitinib, nilotinib, dasatinib, imatinib, sunitinib, sorafenib, trametinib, cobimetanib, and dabrafenib.

11. The method of paragraph 8, wherein the anti-EGFR therapy comprises an anti-EGFR antibody or fragment thereof, or an anti-EGFR CAR T cell.

12. The method of paragraph 11, wherein the anti-EGFR therapy comprises an anti-EGFR antibody selected from the group consisting of cetuximab, necitumumab, panitumumab, nimotuzumab, futuximab, zatuximab, cetugex, and margetuximab.

13. The method of any one of paragraphs 7 to 12, wherein the miR-147b inhibitor is administered before, at the same time as, or after the anti-RTK therapy.

14. The method of any one of paragraphs 1 to 13, wherein the subject has or is at risk of developing tolerance or resistance to anti-RTK therapy.

15. The method of paragraph 14, wherein the anti-RTK therapy to which the subject has or is at risk of developing tolerance or resistance is an anti-EGFR therapy, an anti-AKL therapy, an anti-ROS1 therapy, an anti-ERBB2/3/4 therapy, an anti-KIT therapy, an anti-MET/hepatocyte growth factor receptor (HGFR) therapy, an anti-platelet derived growth factor receptor (PDGFR) therapy, an anti-vascular endothelial cell growth factor receptor (VEGFR) therapy, an anti-fibroblast growth factor receptor (FGFR) therapy, and an anti-RET therapy.

16. The method of paragraph 15, wherein the anti-RTK therapy to which the subject has or is at risk of developing tolerance or resistance comprises a TKI.

17. The method of paragraph 16, wherein the subject has or is at risk of developing tolerance or resistance to an anti-EGFR therapy selected from the group consisting of gefitinib, erlotinib, afatinib, lapatinib, neratinib, osimertinib, vandetanib, crizotinib, dacomitinib, regorafenib, ponatinib, vismodegib, pazopanib, cabozantinib, bosutinib, axitinib, vemurafenib, ruxolitinib, nilotinib, dasatinib, imatinib, sunitinib, sorafenib, trametinib, cobimetanib, and dabrafenib.

18. The method of paragraph 15, wherein the subject has or is at risk of developing tolerance or resistance to an anti-EGFR therapy comprising an anti-EGFR antibody or fragment thereof, or an anti-EGFR CAR T cell.

19. The method of paragraph 18, wherein the anti-EGFR therapy to which the subject has or is at risk of developing tolerance or resistance comprises an anti-EGFR antibody selected from the group consisting of cetuximab, necitumumab, panitumumab, nimotuzumab, futuximab, zatuximab, cetugex, and margetuximab.

20. The method of any one of paragraphs 1 to 19, wherein the miR-147b inhibitor comprises an inhibitory molecule selected from the group consisting of an antisense oligonucleotide, an antagomir, an anti-miRNA sponge, a competitive inhibitor, a triplex-forming oligonucleotide, a double-stranded oligonucleotide, a short interfering RNA, an siRNA, an shRNA, a guide sequence for RNAse P, a small molecule, a catalytic RNA, and a ribozyme; or the inhibition is carried out by the use of a gene editing approach, such as CRISPR-cas9.

21. The method of any one of paragraphs 1 to 20, wherein the miR-147b inhibitor is an inhibitor of the production or activity of pri-miR-147b, pre-miR147b, or mature miR-147b.

22. A single-stranded oligonucleotide comprising a total of 12 to 50 interlinked nucleotides and having a nucleobase sequence comprising at least 6 contiguous nucleobases complementary to an equal-length portion of a miR-147b target nucleic acid.

23. The oligonucleotide of paragraph 22, wherein the oligonucleotide comprises at least one modified nucleobase.

24. The oligonucleotide of paragraph 23, wherein the at least one modified nucleobase is selected from the group consisting of 5-methylcytosine, 7-deazaguanine, and 6-thioguanine.

25. The oligonucleotide of any one of paragraphs 22 to 24, wherein the oligonucleotide comprises at least one modified internucleoside linkage.

26. The oligonucleotide of paragraph 25, wherein the modified internucleoside linkage is a phosphorothioate linkage.

27. The oligonucleotide of paragraph 26, wherein the phosphorothioate linkage is a stereochemically enriched phosphorothioate linkage.

28. The oligonucleotide of any one of paragraphs 25 to 27, wherein at least 50% of the internucleoside linkages in the oligonucleotide are each independently a modified internucleoside linkage.

29. The oligonucleotide of paragraph 28, wherein at least 70% of the internucleoside linkages in the oligonucleotide are each independently a modified internucleoside linkage.

30. The oligonucleotide of any one of paragraphs 22 to 29, wherein the oligonucleotide comprises at least one modified sugar nucleoside.

31. The oligonucleotide of paragraph 30, wherein the at least one modified sugar nucleoside is a bridged nucleic acid.

32. The oligonucleotide of paragraph 31, wherein the bridged nucleic acid is a locked nucleic acid (LNA), an ethylene-bridged nucleic acid (ENA), or a cEt nucleic acid.

33. The oligonucleotide of paragraph 31 or 32, wherein the at least one modified sugar nucleoside is a 2′-modified sugar nucleoside.

34. The oligonucleotide of paragraph 33, wherein the at least one 2′-modified sugar nucleoside comprises a 2′-modification selected from the group consisting of 2′-fluoro, 2′-methoxy, and 2′-methoxyethoxy.

35. The oligonucleotide of any one of paragraphs 22 to 34, wherein the oligonucleotide comprises deoxyribonucleotides.

36. The oligonucleotide of any one of paragraphs 22 to 35, wherein the oligonucleotide comprises ribonucleotides.

37. The oligonucleotide of any one of paragraphs 22 to 24, wherein the oligonucleotide is a morpholino oligonucleotide.

38. The oligonucleotide of any one of paragraphs 22 to 24, wherein the oligonucleotide is a peptide nucleic acid.

39. The oligonucleotide of any one of paragraphs 22 to 38, wherein the oligonucleotide comprises a hydrophobic moiety covalently attached at its 5′-terminus, its 3′-terminus, or an internucleoside linkage of the oligonucleotide.

40. The oligonucleotide of any one of paragraphs 22 to 39, wherein the oligonucleotide comprises a sequence selected from the group consisting of SEQ ID NOs: 3 to 736 or a variant thereof.

41. The oligonucleotide of any one of paragraphs 22 to 40, wherein the oligonucleotide comprises at least 8 contiguous nucleobases complementary to an equal-length portion of a miR-147b target nucleic acid.

42. The oligonucleotide of any one of paragraphs 22 to 41, wherein the oligonucleotide comprises at least 12 contiguous nucleobases complementary to an equal-length portion of a miR-147b target nucleic acid.

43. The oligonucleotide of any one of paragraphs 22 to 42, wherein the oligonucleotide comprises 20 or fewer contiguous nucleobases complementary to an equal-length portion of a miR-147b target nucleic acid.

44. The oligonucleotide of any one of paragraphs 22 to 43, wherein the oligonucleotide comprises a total of at least 12 interlinked nucleotides.

45. The oligonucleotide of any one of paragraphs 22 to 44, wherein the oligonucleotide comprises a total of 24 or fewer interlinked nucleotides.

46. The oligonucleotide of any one of paragraphs 22 to 45, wherein the oligonucleotide is a gapmer, headmer, tailmer, altmer, blockmer, skipmer, or unimer.

47. A double-stranded oligonucleotide comprising the oligonucleotide of any one of paragraphs 22 to 48 hybridized to a complementary oligonucleotide.

48. A double-stranded oligonucleotide comprising a passenger strand hybridized to a guide strand comprising a nucleobase sequence comprising at least 6 contiguous nucleobases complementary to an equal-length portion of a miR-147b target nucleic acid, wherein each of the passenger strand and the guide strand comprises a total of 12 to 50 interlinked nucleotides.

49. The oligonucleotide of paragraph 48, wherein the passenger strand comprises at least one modified nucleobase.

50. The oligonucleotide of paragraph 49, wherein the at least one modified nucleobase is selected from the group consisting of 5-methylcytosine, 7-deazaguanine, and 6-thioguanine.

51. The oligonucleotide of any one of paragraphs 48 to 50, wherein the passenger strand comprises at least one modified internucleoside linkage.

52. The oligonucleotide of paragraph 51, wherein the modified internucleoside linkage is a phosphorothioate linkage.

53. The oligonucleotide of paragraph 52, wherein the phosphorothioate linkage is a stereochemically enriched phosphorothioate linkage.

54. The oligonucleotide of any one of paragraphs 51 to 53, wherein at least 50% of the internucleoside linkages in the passenger strand are each independently the modified internucleoside linkage.

55. The oligonucleotide of paragraph 54, wherein at least 70% of the internucleoside linkages in the passenger strand are each independently the modified internucleoside linkage.

56. The oligonucleotide of any one of paragraphs 48 to 55, wherein the passenger strand comprises at least one modified sugar nucleoside.

57. The oligonucleotide of paragraph 56, wherein the at least one modified sugar nucleoside is a bridged nucleic acid.

58. The oligonucleotide of paragraph 57, wherein the bridged nucleic acid is a locked nucleic acid (LNA), an ethylene-bridged nucleic acid (ENA), or a cEt nucleic acid.

59. The oligonucleotide of any one of paragraphs 56 to 58, wherein the at least one modified sugar nucleoside is a 2′-modified sugar nucleoside.

60. The oligonucleotide of paragraph 59, wherein the at least one 2′-modified sugar nucleoside comprises a 2′-modification selected from the group consisting of 2′-fluoro, 2′-methoxy, and 2′-methoxyethoxy.

61. The oligonucleotide of any one of paragraphs 48 to 60, wherein the passenger strand comprises deoxyribonucleotides.

62. The oligonucleotide of any one of paragraphs 48 to 61, wherein the passenger strand comprises ribonucleotides.

63. The oligonucleotide of any one of paragraphs 48 to 62, wherein the passenger strand comprises a hydrophobic moiety covalently attached at a 5′-terminus, a 3′-terminus, or an internucleoside linkage of the passenger strand.

64. The oligonucleotide of any one of paragraphs 48 to 63, wherein the guide strand comprises at least one modified nucleobase.

65. The oligonucleotide of paragraph 64, wherein the at least one modified nucleobase is selected from the group consisting of 5-methylcytosine, 7-deazaguanine, and 6-thioguanine.

66. The oligonucleotide of any one of paragraphs 48 to 65, wherein the guide strand comprises at least one modified internucleoside linkage.

67. The oligonucleotide of paragraph 66, wherein the modified internucleoside linkage is a phosphorothioate linkage.

68. The oligonucleotide of paragraph 67, wherein the phosphorothioate linkage is a stereochemically enriched phosphorothioate linkage.

69. The oligonucleotide of any one of paragraphs 66 to 68, wherein at least 50% of the internucleoside linkages in the guide strand are each independently the modified internucleoside linkage.

70. The oligonucleotide of paragraph 69, wherein at least 70% of the internucleoside linkages in the guide strand are each independently the modified internucleoside linkage.

71. The oligonucleotide of any one of paragraphs 48 to 70, wherein the guide strand comprises at least one modified sugar nucleoside.

72. The oligonucleotide of paragraph 71, wherein the at least one modified sugar nucleoside is a bridged nucleic acid.

73. The oligonucleotide of paragraph 72, wherein the bridged nucleic acid is a locked nucleic acid (LNA), an ethylene-bridged nucleic acid (ENA), or a cEt nucleic acid.

74. The oligonucleotide of any one of paragraphs 71 to 73, wherein the at least one modified sugar nucleoside is a 2′-modified sugar nucleoside.

75. The oligonucleotide of paragraph 74, wherein the at least one 2′-modified sugar nucleoside comprises a 2′-modification selected from the group consisting of 2′-fluoro, 2′-methoxy, and 2′-methoxyethoxy.

76. The oligonucleotide of any one of paragraphs 48 to 75, wherein the guide strand comprises deoxyribonucleotides.

77. The oligonucleotide of any one of paragraphs 48 to 76, wherein the guide strand comprises ribonucleotides.

78. The oligonucleotide of any one of paragraphs 48 to 77, wherein the guide strand comprises a hydrophobic moiety covalently attached at a 5′-terminus, a 3′-terminus, or an internucleoside linkage of the guide strand.

79. The oligonucleotide of any one of paragraphs 48 to 78, wherein the guide strand comprises a sequence selected from the group consisting of SEQ ID NOs: 3 to 736 or a variant thereof.

80. The oligonucleotide of any one of paragraphs 47 to 79, wherein the hybridized oligonucleotide comprises at least one 3′-overhang.

81. The oligonucleotide of any one of paragraphs 47 to 80, wherein the hybridized oligonucleotide comprises a blunt end.

82. The oligonucleotide of any one of paragraphs 47 to 80, wherein the hybridized oligonucleotide comprises two 3′-overhangs.

83. The oligonucleotide of any one of paragraphs 22 to 82, wherein the miR-147 target nucleic acid comprises pri-miR-147b, pre-miR-147b, or mature miR-147b.

84. An oligonucleotide that competes with miR-147b for binding to a target mRNA or pre-mRNA sequence, thereby inhibiting or reducing the effects of miR-147b on the mRNA or pre-mRNA.

85. The oligonucleotide of paragraph 84, comprising a sequence selected from SEQ ID NOs: 1, 2, or 737 to 889.

86. A vector comprising a sequence encoding an oligonucleotide of paragraph 22, wherein the vector optionally further comprises a promoter to direct transcription of the sequence.

87. The vector of paragraph 86, wherein the vector comprises a sequence encoding multiple oligonucleotides of paragraph 22.

88. The vector of paragraph 87, wherein the vector comprises a sequence encoding 2, 3, 4, 5, 6, 7, 8, 9, or 10 oligonucleotides of paragraph 22.

89. The vector of any one of paragraphs 86 to 88, wherein the vector is a virus, such as a lentivirus, an adenovirus, or an adeno-associated virus; or is a plasmid, a cosmid, or a phagemid.

90. A pharmaceutical composition comprising (i) an oligonucleotide of any one of paragraphs 22 to 85, a vector of any one of paragraphs 86-89, or a small molecule inhibitor of miR-147b, and (ii) a pharmaceutically acceptable excipient or carrier.

91. A method of treating a subject in need thereof, the method comprising administering to the subject a therapeutically effective amount of an oligonucleotide of any one of paragraphs 22 to 85, a vector of any one of paragraphs 86 to 89, or a pharmaceutical composition of paragraph 90.

92. The method of any one of paragraphs 1 to 21, wherein the miR-147b inhibitor comprises an oligonucleotide of any one of paragraphs 22 to 85.

93. The method of any one of paragraphs 1 to 21, 91, or 92, further comprising administration of an additional anti-cancer agent.

94. The method of paragraph 93, wherein the additional anti-cancer agent is an anti-RTK agent.

95. A method of determining whether tolerance or resistance of a cancer to anti-RTK therapy may be effectively treated, reduced, prevented, or delayed by anti-miR-147b therapy, the method comprising determining the level of miR-147b in the cancer, wherein detection of an increased level of miR-147b, relative to a control, indicates that tolerance or resistance of the cancer to anti-RTK therapy may be effectively treated, reduced, prevented, or delayed with anti-miR-147b therapy, optionally in combination with anti-RTK therapy.

96. A method of determining whether a cancer may be effectively treated or prevented with an anti-miR-147b therapy, the method comprising determining the level of miR-147b in the cancer, wherein detection of an increased level of miR-147b in the cancer, relative to a control, indicates that the cancer may effectively be treated or prevented with anti-miR-147b therapy, optionally in combination with anti-RTK therapy.

97. The method of paragraph 95 or 96, wherein the anti-miR-147 therapy is selected from an oligonucleotide of any one of paragraphs 22 to 85, a vector of any one of paragraphs 86-89, and a small molecule inhibitor of miR-147b and/or the anti-RTK therapy is selected from a TKI, an anti-RTK antibody, and a CAR T cell directed against an RTK.

98. The method of any one of paragraphs 95 to 97, wherein determination of the level of miR-147b in the cancer is carried out by detection of the level of miR-147b in a sample from the subject having the cancer.

99. The method of paragraph 98, wherein the sample comprises tumor tissue, tissue swab, sputum, serum, or plasma.

100. The method of any one of paragraphs 95 to 99, further comprising administering an anti-miR147b therapy to a subject having the cancer, if it is determined that (i) tolerance or resistance of the cancer to anti-RTK therapy may be effectively treated, reduced, prevented, or delayed by anti-miR-147b therapy, or (ii) the cancer may be effectively treated with anti-miR147b therapy.

101. A method of detecting a cancer cell in a sample, the method comprising determining the level of miR-147b in the sample, wherein detection of an increased level of miR-147b in the sample, relative to a control, indicates the presence of a cancer cell in the sample.

102. A method of determining whether a cancer cell in a sample may be tolerant or resistant to anti-RTK therapy, the method comprising determining the level of miR-147b in the sample, wherein detection of an increased level of miR-147b, relative to a control, indicates that the cancer cell may be tolerant or resistant to anti-RTK therapy.

103. The method of paragraph 102, wherein the anti-RTK therapy is anti-EGFR therapy.

104. The method of paragraph 102 or 103, wherein the sample comprises tumor tissue, tissue swab, sputum, serum, or plasma.

105. A method of making an organoid comprising lung cells, the method comprising the steps of:

a. culturing lung cells in a medium comprising epidermal growth factor (EGF), fibroblast growth factor 2 (FGF2), and fibroblast growth factor 10 (FGF10);

b. maintaining the cells in culture in a medium comprising Noggin and transforming growth factor-β (TGF-β); and

c. differentiating the cells in a medium comprising fibroblast growth factor 7 (FGF7) and platelet-derived growth factor (PDGF).

106. The method of paragraph 105, wherein the lung cells are lung epithelial cells obtained from a sample of lung tissue of a subject.

107. The method of paragraph 105 or 106, wherein the kung cells are immortalized lung epithelial cells.

108. The method of any one of paragraphs 105 to 107, wherein the lung cells are cancerous.

109. The method of any one of paragraphs 105 to 107, wherein the lung cells are non-cancerous.

110. The method of any one of paragraphs 105 to 109, wherein the lung cells are tolerant or resistant to an anti-RTK agent.

111. The method of any one of paragraphs 105 to 110, wherein the maintaining step is carried out on days 0-3 of the method, maintenance is carried out on days 4-6, and differentiation is carried out on days 7-24.

112. The method of any one of paragraphs 105 to 111, wherein the organoids show ring-like structures upon treatment with an anti-RTK agent.

113. A three-dimensional organoid comprising lung cells, wherein the organoid is optionally made by, or has features of organoids made using, the method of any one of paragraphs 105 to 112.

114. The organoid of paragraph 113, wherein the lung cells comprise lung cancer cells.

115. The organoid of paragraph 113 or 114, wherein the lung cells or lung cancer cells are primary cells, obtained or cultured from the cells of a subject.

116. A method for identifying an agent that may be used (i) to treat, reduce, prevent, or delay tolerance or resistance to anti-RTK therapy, or (ii) in the treatment or prevention of cancer, the method comprising contacting a cell with the agent and determining whether the agent decreases the level of miR-147b in the cell.

117. The method of paragraph 116, wherein the cell is comprised within an organoid.

118. The method of paragraph 117, wherein the organoid comprises lung cancer cells.

119. The method of paragraph 117 or 118, wherein the organoid is an organoid of any one of paragraphs 113 to 115, or is made by a method of any one of paragraphs 105 to 112.

120. The method of any one of paragraphs 116 to 119, wherein the lung cancer cells are resistant to an anti-RTK therapy.

121. The method of any one of paragraphs 116 to 120, wherein the cells are primary cells, obtained or cultured from the cells of a subject.

122. The method of any one of paragraphs 116 to 121, wherein the agent is a candidate compound, not previously known to be effective at treating, reducing, preventing, or delaying tolerance or resistance to anti-RTK therapy, or at treating or preventing cancer.

123. The method of any one of paragraphs 116 to 121, wherein the method is carried out to determine an optimal approach to treat, reduce, prevent, or delay tolerance or resistance of a cancer to anti-RTK therapy in a subject, or to treat or prevent a cancer in a subject.

124. A kit comprising an agent for detecting the level of miR-147b in a sample.

125. The kit of paragraph 124, wherein the agent comprises an oligonucleotide, which is optionally an oligonucleotide of any one of paragraphs 22 to 85.

126. A kit comprising a miR-147b inhibitor, which optionally is an oligonucleotide of any one of paragraphs 22 to 85, and a second agent for treating cancer.

127. The oligonucleotide of paragraph 22, wherein the oligonucleotide targets a sequence comprising or consisting of nucleotides 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 48-51, 47-52, 48-53, 49-54, 50-55, 51-56, 52-57, 53-58, 54-59, 55-80, 58-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, or 75-80 of SEQ ID NO: 1.

128. The oligonucleotide of paragraph 127, wherein the oligonucleotide targets said sequence and additionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 additional nucleotides of SEQ ID NO: 1, whether all on one side of the indicated fragment or wherein the fragment is between the one or more additional nucleotides.

129. The oligonucleotide of paragraph 48, wherein the oligonucleotide targets a sequence comprising or consisting of nucleotides 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 46-51, 47-52, 48-53, 49-54, 50-55, 51-56, 52-57, 53-58, 54-59, 55-40, 58-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, or 75-80 of SEQ ID NO: 1.

130. The oligonucleotide of paragraph 129, wherein the oligonucleotide targets said sequence and additionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 additional nucleotides of SEQ ID NO: 1, whether all on one side of the indicated fragment or wherein the fragment is between the one or more additional nucleotides.

Other embodiments are within the scope of the following claims.

1. A method of treating, reducing, preventing, or delaying tolerance or resistance to anti-receptor tyrosine kinase (RTK) therapy in a subject, the method comprising administering a miR-147b inhibitor to the subject.

2. The method of claim 1, wherein the RTK is selected from the group consisting of epidermal growth factor receptor (EGFR), human EGFR2 (HER2), HER3, anaplastic lymphoma kinase (ALK), ROS1, ERBB2/3/4, KIT, MET/hepatocyte growth factor receptor (HGFR), RON, platelet derived growth factor receptor (PDGFR), vascular endothelial cell growth factor receptor (VEGFR), VEGFR1, VEGFR2, fibroblast growth factor receptor (FGFR), insulin-like growth factor 1 receptor (IGF1R), and RET.

3. The method of claim 1, wherein the miR-147b inhibitor reduces a Von Hippel-Lindau (VHL)-pseudohypoxia response or counteracts metabolic changes in the tricarboxylic acid (TCA) cycle associated with drug tolerance in the subject.

4. The method of claim 1, wherein the subject has cancer.

5. A method of treating or preventing cancer in a subject, the method comprising administering a miR-147b inhibitor to the subject.

6. The method of claim 4, wherein the subject has a cancer selected from the group consisting of lung cancer, non-smal cell lung cancer, colorectal cancer, anal cancer, glioblastoma, squamous cell carcinoma, squamous cell carcinoma of the head and neck, pancreatic cancer, breast cancer, renal cell carcinoma, thyroid cancer, gastroesophageal adenocarcinoma, and gastric cancer.

7. The method claim 1, further comprising administering an anti-RTK therapy to the subject.

8. The method of claim 7, wherein the anti-RTK therapy is an anti-EGFR therapy.

9. The method of claim 8, wherein the anti-EGFR therapy comprises a tyrosine kinase inhibitor (TKI).

10. The method of claim 9, wherein the TKI is selected from the group consisting of gefitinib, erlotinib, afatinib, lapatinib, neratinib, osimertinib, vandetanib, crizotinib, dacomitinib, regorafenib, ponatinib, vismodegib, pazopanib, cabozantinib, bosutinib, axitinib, vemurafenib, ruxolitinib, nilotinib, dasatinib, imatinib, sunitinib, sorafenib, trametinib, cobimetanib, and dabrafenib.

11. The method of claim 8, wherein the anti-EGFR therapy comprises an anti-EGFR antibody or fragment thereof, or an anti-EGFR CAR T cell.

12. The method of claim 11, wherein the anti-EGFR therapy comprises an anti-EGFR antibody selected from the group consisting of cetuximab, necitumumab, panitumumab, nimotuzumab, futuximab, zatuximab, cetugex, and margetuximab.

13. The method of claim 7, wherein the miR-147b inhibitor is administered before, at the same time as, or after the anti-RTK therapy.

14. The method of claim 1, wherein the subject has or is at risk of developing tolerance or resistance to anti-RTK therapy.

15. The method of claim 14, wherein the anti-RTK therapy to which the subject has or is at risk of developing tolerance or resistance is an anti-EGFR therapy, an anti-AKL therapy, an anti-ROS1 therapy, an anti-ERBB2/3/4 therapy, an anti-KIT therapy, an anti-MET/hepatocyte growth factor receptor (HGFR) therapy, an anti-platelet derived growth factor receptor (PDGFR) therapy, an anti-vascular endothelial cell growth factor receptor (VEGFR) therapy, an anti-fibroblast growth factor receptor (FGFR) therapy, and an anti-RET therapy.

16. The method of claim 15, wherein the anti-RTK therapy to which the subject has or is at risk of developing tolerance or resistance comprises a TKI.

17. The method of claim 16, wherein the subject has or is at risk of developing tolerance or resistance to an anti-EGFR therapy selected from the group consisting of gefitinib, erlotinib, afatinib, lapatinib, neratinib, osimertinib, vandetanib, crizotinib, dacomitinib, regorafenib, ponatinib, vismodegib, pazopanib, cabozantinib, bosutinib, axitinib, vemurafenib, ruxolitinib, nilotinib, dasatinib, imatinib, sunitinib, sorafenib, trametinib, cobimetanib, and dabrafenib.

18. The method of claim 15, wherein the subject has or is at risk of developing tolerance or resistance to an anti-EGFR therapy comprising an anti-EGFR antibody or fragment thereof, or an anti-EGFR CAR T cell.

19. The method of claim 18, wherein the anti-EGFR therapy to which the subject has or is at risk of developing tolerance or resistance comprises an anti-EGFR antibody selected from the group consisting of cetuximab, necitumumab, panitumumab, nimotuzumab, futuximab, zatuximab, cetugex, and margetuximab.

20. The method of claim 1, wherein the miR-147b inhibitor comprises an inhibitory molecule selected from the group consisting of an antisense oligonucleotide, an antagomir, an anti-miRNA sponge, a competitive inhibitor, a triplex-forming oligonucleotide, a double-stranded oligonucleotide, a short interfering RNA, an siRNA, an shRNA, a guide sequence for RNAse P, a small molecule, a catalytic RNA, and a ribozyme; or the inhibition is carried out by the use of a gene editing approach, such as CRISPR-cas9.

21. The method of claim 1, wherein the miR-147b inhibitor is an inhibitor of the production or activity of pri-miR-147b, pre-miR147b, or mature miR-147b.

22. A single-stranded oligonucleotide comprising a total of 12 to 50 interlinked nucleotides and having a nucleobase sequence comprising at least 6 contiguous nucleobases complementary to an equal-length portion of a miR-147b target nucleic acid.

23. The oligonucleotide of claim 22, wherein the oligonucleotide comprises at least one modified nucleobase.

24. The oligonucleotide of claim 23, wherein the at least one modified nucleobase is selected from the group consisting of 5-methylcytosine, 7-deazaguanine, and 6-thioguanine.

25. The oligonucleotide of claim 22, wherein the oligonucleotide comprises at least one modified internucleoside linkage.

26. The oligonucleotide of claim 25, wherein the modified internucleoside linkage is a phosphorothioate linkage.

27. The oligonucleotide of claim 26, wherein the phosphorothioate linkage is a stereochemically enriched phosphorothioate linkage.

28. The oligonucleotide of claim 25, wherein at least 50% of the internucleoside linkages in the oligonucleotide are each independently a modified internucleoside linkage.

29. The oligonucleotide of claim 28, wherein at least 70% of the internucleoside linkages in the oligonucleotide are each independently a modified internucleoside linkage.

30. The oligonucleotide of claim 22, wherein the oligonucleotide comprises at least one modified sugar nucleoside.

31. The oligonucleotide of claim 30, wherein the at least one modified sugar nucleoside is a bridged nucleic acid.

32. The oligonucleotide of claim 31, wherein the bridged nucleic acid is a locked nucleic acid (LNA), an ethylene-bridged nucleic acid (ENA), or a cEt nucleic acid.

33. The oligonucleotide of claim 31, wherein the at least one modified sugar nucleoside is a 2′-modified sugar nucleoside.

34. The oligonucleotide of claim 33, wherein the at least one 2′-modified sugar nucleoside comprises a 2′-modification selected from the group consisting of 2′-fluoro, 2′-methoxy, and 2′-methoxyethoxy.

35. The oligonucleotide of claim 22, wherein the oligonucleotide comprises deoxyribonucleotides.

36. The oligonucleotide of claim 22, wherein the oligonucleotide comprises ribonucleotides.

37. The oligonucleotide of claim 22, wherein the oligonucleotide is a morpholino oligonucleotide.

38. The oligonucleotide of claim 22, wherein the oligonucleotide is a peptide nucleic acid.

39. The oligonucleotide of claim 22, wherein the oligonucleotide comprises a hydrophobic moiety covalently attached at its 5′-terminus, its 3′-terminus, or an internucleoside linkage of the oligonucleotide.

40. The oligonucleotide of claim 22, wherein the oligonucleotide comprises a sequence selected from the group consisting of SEQ ID NOs: 3 to 736 or a variant thereof.

41. The oligonucleotide of claim 22, wherein the oligonucleotide comprises at least 8 contiguous nucleobases complementary to an equal-length portion of a miR-147b target nucleic acid.

42. The oligonucleotide of claim 22, wherein the oligonucleotide comprises at least 12 contiguous nucleobases complementary to an equal-length portion of a miR-147b target nucleic acid.

43. The oligonucleotide of claim 22, wherein the oligonucleotide comprises 20 or fewer contiguous nucleobases complementary to an equal-length portion of a miR-147b target nucleic acid.

44. The oligonucleotide of claim 22, wherein the oligonucleotide comprises a total of at least 12 interlinked nucleotides.

45. The oligonucleotide of claim 22, wherein the oligonucleotide comprises a total of 24 or fewer interlinked nucleotides.

46. The oligonucleotide of claim 22, wherein the oligonucleotide is a gapmer, headmer, tailmer, altmer, blockmer, skipmer, or unimer.

47. A double-stranded oligonucleotide comprising the oligonucleotide of claim 22 hybridized to a complementary oligonucleotide.

48. A double-stranded oligonucleotide comprising a passenger strand hybridized to a guide strand comprising a nucleobase sequence comprising at least 6 contiguous nucleobases complementary to an equal-length portion of a miR-147b target nucleic acid, wherein each of the passenger strand and the guide strand comprises a total of 12 to 50 interlinked nucleotides.

49. The oligonucleotide of claim 48, wherein the passenger strand comprises at least one modified nucleobase.

50. The oligonucleotide of claim 49, wherein the at least one modified nucleobase is selected from the group consisting of 5-methylcytosine, 7-deazaguanine, and 6-thioguanine.

51. The oligonucleotide of claim 48, wherein the passenger strand comprises at least one modified internucleoside linkage.

52. The oligonucleotide of claim 51, wherein the modified internucleoside linkage is a phosphorothioate linkage.

53. The oligonucleotide of claim 52, wherein the phosphorothioate linkage is a stereochemically enriched phosphorothioate linkage.

54. The oligonucleotide of claim 51, wherein at least 50% of the internucleoside linkages in the passenger strand are each independently the modified internucleoside linkage.

55. The oligonucleotide of claim 54, wherein at least 70% of the internucleoside linkages in the passenger strand are each independently the modified internucleoside linkage.

56. The oligonucleotide of claim 48, wherein the passenger strand comprises at least one modified sugar nucleoside.

57. The oligonucleotide of claim 56, wherein the at least one modified sugar nucleoside is a bridged nucleic acid.

58. The oligonucleotide of claim 57, wherein the bridged nucleic acid is a locked nucleic acid (LNA), an ethylene-bridged nucleic acid (ENA), or a cEt nucleic acid.

59. The oligonucleotide of claim 56, wherein the at least one modified sugar nucleoside is a 2′-modified sugar nucleoside.

60. The oligonucleotide of claim 59, wherein the at least one 2′-modified sugar nucleoside comprises a 2′-modification selected from the group consisting of 2′-fluoro, 2′-methoxy, and 2′-methoxyethoxy.

61. The oligonucleotide of claim 48, wherein the passenger strand comprises deoxyribonucleotides.

62. The oligonucleotide of claim 48, wherein the passenger strand comprises ribonucleotides.

63. The oligonucleotide of claim 48, wherein the passenger strand comprises a hydrophobic moiety covalently attached at a 5′-terminus, a 3′-terminus, or an internucleoside linkage of the passenger strand.

64. The oligonucleotide of claim 48, wherein the guide strand comprises at least one modified nucleobase.

65. The oligonucleotide of claim 64, wherein the at least one modified nucleobase is selected from the group consisting of 5-methylcytosine, 7-deazaguanine, and 6-thioguanine.

66. The oligonucleotide of claim 48, wherein the guide strand comprises at least one modified internucleoside linkage.

67. The oligonucleotide of claim 66, wherein the modified internucleoside linkage is a phosphorothioate linkage.

68. The oligonucleotide of claim 67, wherein the phosphorothioate linkage is a stereochemically enriched phosphorothioate linkage.

69. The oligonucleotide of claim 66, wherein at least 50% of the internucleoside linkages in the guide strand are each independently the modified internucleoside linkage.

70. The oligonucleotide of claim 69, wherein at least 70% of the internucleoside linkages in the guide strand are each independently the modified internucleoside linkage.

71. The oligonucleotide of claim 48, wherein the guide strand comprises at least one modified sugar nucleoside.

72. The oligonucleotide of claim 71, wherein the at least one modified sugar nucleoside is a bridged nucleic acid.

73. The oligonucleotide of claim 72, wherein the bridged nucleic acid is a locked nucleic acid (LNA), an ethylene-bridged nucleic acid (ENA), or a cEt nucleic acid.

74. The oligonucleotide of claim 71, wherein the at least one modified sugar nucleoside is a 2′-modified sugar nucleoside.

75. The oligonucleotide of claim 74, wherein the at least one 2′-modified sugar nucleoside comprises a 2′-modification selected from the group consisting of 2′-fluoro, 2′-methoxy, and 2′-methoxyethoxy.

76. The oligonucleotide of claim 48, wherein the guide strand comprises deoxyribonucleotides.

77. The oligonucleotide of claim 48, wherein the guide strand comprises ribonucleotides.

78. The oligonucleotide of claim 48, wherein the guide strand comprises a hydrophobic moiety covalently attached at a 5′-terminus, a 3′-terminus, or an internucleoside linkage of the guide strand.

79. The oligonucleotide of claim 48, wherein the guide strand comprises a sequence selected from the group consisting of SEQ ID NOs: 3 to 736 or a variant thereof.

80. The oligonucleotide of claim 47, wherein the hybridized oligonucleotide comprises at least one 3′-overhang.

81. The oligonucleotide of claim 47, wherein the hybridized oligonucleotide comprises a blunt end.

82. The oligonucleotide of claim 47, wherein the hybridized oligonucleotide comprises two 3′-overhangs.

83. The oligonucleotide of claim 22, wherein the miR-147 target nucleic acid comprises pri-miR-147b, pre-miR-147b, or mature miR-147b.

84. An oligonucleotide that competes with miR-147b for binding to a target mRNA or pre-mRNA sequence, thereby inhibiting or reducing the effects of miR-147b on the mRNA or pre-mRNA.

85. The oligonucleotide of claim 84, comprising a sequence selected from SEQ ID NOs: 1, 2, or 737 to 889.

86. A vector comprising a sequence encoding an oligonucleotide of claim 22, wherein the vector optionally further comprises a promoter to direct transcription of the sequence.

87. The vector of claim 86, wherein the vector comprises a sequence encoding multiple oligonucleotides as described herein.

88. The vector of claim 87, wherein the vector comprises a sequence encoding 2, 3, 4, 5, 6, 7, 8, 9, or 10 oligonucleotides as described herein.

89. The vector of claim 86, wherein the vector is a virus, such as a lentivirus, an adenovirus, or an adeno-associated virus; or is a plasmid, a cosmid, or a phagemid.

90. A pharmaceutical composition comprising (i) an oligonucleotide of claim 22 or a vector comprising said oligonucleotide, or a small molecule inhibitor of miR-147b, and (ii) a pharmaceutically acceptable excipient or carrier.

91. A method of treating a subject in need thereof, the method comprising administering to the subject a therapeutically effective amount of an oligonucleotide of claim 22 or a vector comprising said oligonucleotide, or a pharmaceutical composition as described herein.

92. The method of claim 1, wherein the miR-147b inhibitor comprises an oligonucleotide as described herein.

93. The method of claim 1, further comprising administration of an additional anti-cancer agent.

94. The method of claim 93, wherein the additional anti-cancer agent is an anti-RTK agent.

95. A method of determining whether tolerance or resistance of a cancer to anti-RTK therapy may be effectively treated, reduced, prevented, or delayed by anti-miR-147b therapy, the method comprising determining the level of miR-147b in the cancer, wherein detection of an increased level of miR-147b, relative to a control, indicates that tolerance or resistance of the cancer to anti-RTK therapy may be effectively treated, reduced, prevented, or delayed with anti-miR-147b therapy, optionally in combination with anti-RTK therapy.

96. A method of determining whether a cancer may be effectively treated or prevented with an anti-miR-147b therapy, the method comprising determining the level of miR-147b in the cancer, wherein detection of an increased level of miR-147b in the cancer, relative to a control, indicates that the cancer may effectively be treated or prevented with anti-miR-147b therapy, optionally in combination with anti-RTK therapy.

97. The method of claim 95, wherein the anti-miR-147 therapy is selected from an oligonucleotide as described herein, a vector comprising the oligonucleotide, and a small molecule inhibitor of miR-147b and/or the anti-RTK therapy is selected from a TKI, an anti-RTK antibody, and a CAR T cell directed against an RTK.

98. The method of claim 95, wherein determination of the level of miR-147b in the cancer is carried out by detection of the level of miR-147b in a sample from the subject having the cancer.

99. The method of claim 98, wherein the sample comprises tumor tissue, tissue swab, sputum, serum, or plasma.

100. The method of claim 95, further comprising administering an anti-miR147b therapy to a subject having the cancer, if it is determined that (i) tolerance or resistance of the cancer to anti-RTK therapy may be effectively treated, reduced, prevented, or delayed by anti-miR-147b therapy, or (ii) the cancer may be effectively treated with anti-miR147b therapy.

101. A method of detecting a cancer cell in a sample, the method comprising determining the level of miR-147b in the sample, wherein detection of an increased level of miR-147b in the sample, relative to a control, indicates the presence of a cancer cell in the sample.

102. A method of determining whether a cancer cell in a sample may be tolerant or resistant to anti-RTK therapy, the method comprising determining the level of miR-147b in the sample, wherein detection of an increased level of miR-147b, relative to a control, indicates that the cancer cell may be tolerant or resistant to anti-RTK therapy.

103. The method of claim 102, wherein the anti-RTK therapy is anti-EGFR therapy.

104. The method of claim 102, wherein the sample comprises tumor tissue, tissue swab, sputum, serum, or plasma.

105. A method of making an organoid comprising lung cells, the method comprising the steps of:
a. culturing lung cells in a medium comprising epidermal growth factor (EGF), fibroblast growth factor 2 (FGF2), and fibroblast growth factor 10 (FGF10);
b. maintaining the cells in culture in a medium comprising Noggin and transforming growth factor-β (TGF-β); and
c. differentiating the cells in a medium comprising fibroblast growth factor 7 (FGF7) and platelet-derived growth factor (PDGF).

106. The method of claim 105, wherein the lung cells are lung epithelial cells obtained from a sample of lung tissue of a subject.

107. The method of claim 105, wherein the lung cells are immortalized lung epithelial cells.

108. The method of claim 105, wherein the lung cells are cancerous.

109. The method of claim 105, wherein the lung cells are non-cancerous.

110. The method of claim 105, wherein the lung cells are tolerant or resistant to an anti-RTK agent.

111. The method of claim 105, wherein the maintaining step is carried out on days 0-3 of the method, maintenance is carried out on days 4-6, and differentiation is carried out on days 7-24.

112. The method of claim 105, wherein the organoids show ring-like structures upon treatment with an anti-RTK agent.

113. A three-dimensional organoid comprising lung cells, wherein the organoid is optionally made by, or has features of organoids made using, the method of claim 105.

114. The organoid of claim 113, wherein the lung cells comprise lung cancer cells.

115. The organoid of claim 113, wherein the lung cells or lung cancer cells are primary cells, obtained or cultured from the cells of a subject.

116. A method for identifying an agent that may be used (i) to treat, reduce, prevent, or delay tolerance or resistance to anti-RTK therapy, or (ii) in the treatment or prevention of cancer, the method comprising contacting a cell with the agent and determining whether the agent decreases the level of miR-147b in the cell.

117. The method of claim 116, wherein the cell is comprised within an organoid.

118. The method of claim 117, wherein the organoid comprises lung cancer cells.

119. The method of claim 117, wherein the organoid is an organoid as described herein, or is made by a method as described herein.

120. The method of claim 116, wherein the lung cancer cells are resistant to an anti-RTK therapy.

121. The method of claim 116, wherein the cells are primary cells, obtained or cultured from the cells of a subject.

122. The method of claim 116, wherein the agent is a candidate compound, not previously known to be effective at treating, reducing, preventing, or delaying tolerance or resistance to anti-RTK therapy, or at treating or preventing cancer.

123. The method of claim 116, wherein the method is carried out to determine an optimal approach to treat, reduce, prevent, or delay tolerance or resistance of a cancer to anti-RTK therapy in a subject, or to treat or prevent a cancer in a subject.

124. A kit comprising an agent for detecting the level of miR-147b in a sample.

125. The kit of claim 124, wherein the agent comprises an oligonucleotide, which is optionally an oligonucleotide as described herein.

126. A kit comprising a miR-147b inhibitor, which optionally is an oligonucleotide as described herein, and a second agent for treating cancer.

127. The oligonucleotide of claim 22, wherein the oligonucleotide targets a sequence comprising or consisting of nucleotides 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 48-51, 47-52, 48-53, 49-54, 50-55, 51-56, 52-57, 53-58, 54-59, 55-80, 56-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, or 75-80 of SEQ ID NO: 1.

128. The oligonucleotide of claim 127, wherein the oligonucleotide targets said sequence and additionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 additional nucleotides of SEQ ID NO: 1, whether all on one side of the indicated fragment or wherein the fragment is between the one or more additional nucleotides.

129. The oligonucleotide of claim 48, wherein the oligonucleotide targets a sequence comprising or consisting of nucleotides 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, 28-33, 29-34, 30-35, 31-36, 32-37, 33-38, 34-39, 35-40, 36-41, 37-42, 38-43, 39-44, 40-45, 41-46, 42-47, 43-48, 44-49, 45-50, 48-51, 47-52, 48-53, 49-54, 50-55, 51-56, 52-57, 53-58, 54-59, 55-80, 56-61, 57-62, 58-63, 59-64, 60-65, 61-66, 62-67, 63-68, 64-69, 65-70, 66-71, 67-72, 68-73, 69-74, 70-75, 71-76, 72-77, 73-78, 74-79, or 75-80 of SEQ ID NO: 1.

130. The oligonucleotide of claim 129, wherein the oligonucleotide targets said sequence and additionally 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, or 74 additional nucleotides of SEQ ID NO: 1, whether all on one side of the indicated fragment or wherein the fragment is between the one or more additional nucleotides.
